(19) United States
(12) Patent Application Publication
(10) Pub. No.: US 2024/0366678 A1
(43) Pub. Date: Nov. 7, 2024

(54) EFFECTOR PROTEIN COMPOSITIONS AND METHODS OF USE THEREOF FOR MANUFACTURING ENGINEERED HSCS

(71) Applicant: Mammoth Biosciences, Inc., Brisbane, CA (US)

(72) Inventors: Mark Alan DEWITT, Millbrae, CA (US); Siming XU, Redwood City, CA (US); William Douglass WRIGHT, Fairfield, CA (US); Stepan TYMOSHENKO, Sacramento, CA (US); Benjamin Julius RAUCH, San Francisco, CA (US); Wiputra Jaya HARTONO, San Francisco, CA (US); Aaron DELOUGHERY, San Francisco, CA (US); Lucas Benjamin HARRINGTON, San Francisco, CA (US)

(21) Appl. No.: 18/653,793

(22) Filed: May 2, 2024

Related U.S. Application Data
(60) Provisional application No. 63/500,566, filed on May 5, 2023.
(60) Provisional application No. 63/514,342, filed on Jul. 18, 2023.
(60) Provisional application No. 63/580,980, filed on Sep. 6, 2023.

(51) Int. Cl. A61K 35/28; A61K 31/7105; A61K 38/46; A61K 48/00; C07K 14/00; C12N 5/0789; C12N 9/22; C12N 15/11; C12N 15/86; C12N 15/90

(52) U.S. Cl. CPC A61K 35/28; A61K 31/7105; A61K 38/465; A61K 48/0033; A61K 48/0066; C07K 14/00; C12N 5/0647; C12N 9/22; C12N 15/11; C12N 15/86; C12N 15/907; C12N 2310/20; C12N 2510/00; C12N 2750/14143

(57) ABSTRACT

Provided herein are compositions, systems, and methods for engineering hematopoietic stem cells that engineered by using polypeptides, such as effector proteins, and guide nucleic acids. Also, provided herein are the engineered hematopoietic stem cells, methods of manufacturing such hematopoietic stem cells, and methods of treating a disease or disorder using such hematopoietic stem cells.

### CROSS-REFERENCED APPLICATIONS

This application claims the benefit of priority to U.S. Provisional Application No. 63/500,566, filed on May 5, 2023, U.S. Provisional Application No. 63/514,342, filed on Jul. 18, 2023, and U.S. Provisional Application No. 63/580,980, filed on Sep. 6, 2023, the entire contents of each of which are incorporated herein by reference.

### INCORPORATION BY REFERENCE OF SEQUENCE LISTING

The instant application contains a Sequence Listing, which has been submitted via Patent Center. The Sequence Listing titled 203477-786201_US_SL.xml, which was created on May 1, 2024, and is 1,411,352 bytes in size, is hereby incorporated by reference in its entirety.

### FIELD

The present disclosure relates generally to polypeptides, such as effector proteins, compositions of such polypeptides and guide nucleic acids, systems, and methods of using such polypeptides and compositions, for manufacturing engineered hematopoietic stem cells (HSCs).

### BACKGROUND

Clustered Regularly Interspaced Short Palindromic Repeats (CRISPR) and associated proteins (Cas proteins), sometimes referred to as a CRISPR/Cas system, were first identified in certain bacterial species and are now understood to form part of a prokaryotic acquired immune system. CRISPR/Cas systems provide immunity in bacteria and archaea against viruses and plasmids by targeting the nucleic acids of the viruses and plasmids in a sequence-specific manner. Native systems contain a CRISPR array, which includes direct repeats flanking short spacer sequences that, in part, guide Cas proteins to their targets. The discovery of CRISPR/Cas systems has revolutionized the field of genomic manipulation and engineering, and therapeutic applications of these systems are being explored.

### SUMMARY

The present disclosure provides for polypeptides, such as effector proteins, compositions, systems, and methods comprising the same, and uses thereof. In general, compositions, systems, and methods comprise a guide nucleic acid or use thereof. Compositions, systems, and methods disclosed herein may leverage nucleic acid modification activities, such as nucleic acid editing. Editing may comprise: insertion, deletion, substitution, or a combination thereof of one or more nucleotides. In some embodiments, modification activities comprise nucleic acid cleavage activity, e.g., cleavage of a phosphodiester bond between two nucleotides. In some embodiments, compositions, systems and methods are useful for modifying the sequence of a target nucleic acid. In some embodiments, compositions, systems and methods are useful for engineering a hematopoietic stem cell (HSC). In some embodiments, the HSC is used for treating a disease or disorder associated with the target nucleic acid. In some embodiments, the target nucleic acid comprises a human gene. In some embodiments, the target nucleic acid comprises a human gene associated with a genetic blood disease or disorder (e.g., sickle cell anemia, sickle cell disease (SCD), and/or β-thalassemia). In some embodiments, the target nucleic acid comprises a human gene (e.g., HBB gene, BCL11A gene, HBG1 gene, HBG2 gene), or a portion thereof.

### Certain Embodiments

Provided herein are systems for modifying a target nucleic acid of a hematopoietic stem cell (HSC), the system comprising: (i) a polypeptide, or a nucleic acid encoding the polypeptide, wherein the polypeptide comprises an amino acid sequence that is at least 85% identical to any one of the amino acid sequences set forth in TABLE 1; and (ii) an engineered guide nucleic acid, or a nucleic acid encoding the engineered guide nucleic acid, wherein: (a) the engineered guide nucleic acid comprises a first region and a second region, (b) the polypeptide at least partially binds to the first region to form an RNP complex, (c) the second region comprises a nucleotide sequence that is at least 80% complementary to or at least 80% reverse complementary to a target sequence of the target nucleic acid, (d) the RNP complex, upon hybridization of the second region to the target nucleic, modifies the target nucleic acid of the HSC, (e) the first region and the second region are heterologous to each other, and (f) the HSC following modification by the RNP complex retains at least one of cell viability, cell proliferation, and multi-lineage development potential relative to an unmodified HSC. In some embodiments, the target nucleic acid is within a eukaryotic gene. In some embodiments, the target nucleic acid comprises a nucleotide sequence comprising any one of genes set forth in TABLE 10, a variant thereof or a portion thereof. In some embodiments, the target nucleic acid comprises a nucleotide sequence that is at least 85% identical to any one of the nucleotide sequences set forth in TABLE 11. In some embodiments, the target nucleic acid comprises any one of the following genes: BCL11A, B2M, PNPLA2, CHD4, CIITA, GATA1, HBA, HBA1, HBA2, HBB, HBD, HBE, HBE1, HBG1, HBG2, HBM, HBQ1, HBZ, HOXA9, KLF1, MBD3, MYB, TRAC1, ZBTB7A, a fragment thereof, a promoter thereof, an enhancer thereof, or a combination thereof. In some embodiments, the target nucleic acid comprises any one of the following genes: BCL11A, HBB, HBG1, HBG2, a fragment thereof, a promoter thereof, an enhancer thereof, or a combination thereof. In some embodiments, the HBG gene comprises γ-globin 1 gene (HBG1 gene), γ-globin 2 gene (HBG2 gene), G γ-globin gene, A γ-globin gene, a fragment thereof, a promoter thereof, an enhancer thereof, or a combination thereof. In some embodiments, the target nucleic acid comprises a HBB gene, a fragment thereof, an enhancer thereof or a promoter thereof. In some embodiments, the HBB gene comprises a EV6 mutation. In some embodiments, the target nucleic acid is associated with any one of diseases or disorders set forth in TABLE 12. In some embodiments, the target nucleic acid is associated with a genetic blood disease or disorder. In some embodiments, the target nucleic acid is associated with sickle cell anemia, sickle cell disease (SCD), and/or β-thalassemia. In some embodiments, the target nucleic acid comprises a single nuclear polymorphism (SNP). In some embodiments, the engineered guide nucleic acid comprises a crRNA. In some embodiments, the first region comprises a repeat sequence that at least partially binds to the polypeptide. In some embodiments, the repeat sequence comprises a nucleotide sequence that is at least 85% identical, at least 90% identical, at least 95% identical, or at least 100% identical to any one of the nucleotide sequences set forth in TABLE 5. In some embodiments, the first region comprises a handle sequence that at least partially binds to the polypeptide. In some embodiments, the handle sequence comprises a repeat sequence, an intermediary sequence, a linker, or combinations thereof. In some embodiments, the second region comprises at least 10 contiguous nucleotides that are complementary to a eukaryotic sequence. In some embodiments, the second region comprises at least 10 contiguous nucleotides that are reverse complement of the target sequence. In some embodiments, the second region comprises a spacer sequence that hybridizes to a target sequence of a target nucleic acid, and wherein optionally the spacer sequence comprises any one of the spacer sequences set forth in TABLE 7. In some embodiments, the engineered guide nucleic acid comprises one or more phosphorothioate (PS) backbone modifications, 2′-fluoro (2′-F) sugar modifications, or 2′-O-Methyl (2′OMe) sugar modifications. In some embodiments, the guide nucleic acid comprises a nucleotide sequence that is at least 85% identical to any one of the nucleotide sequences set forth in TABLE 9.

Also provided herein are systems for modifying a target nucleic acid of a hematopoietic stem cell (HSC) comprising a polypeptide, wherein the polypeptide comprises an amino acid sequence that is at least 90% identical, at least 95% identical, or at least 100% identical to any one of the amino acid sequences set forth in TABLE 1. In some embodiments, the polypeptide comprises a Type V Cas effector protein. In some embodiments, the polypeptide comprises a Cas.265466, a CasPhi.12, or a variant thereof. In some embodiments, the polypeptide comprises binding activity, nuclease activity, nickase activity, base editing activity, cleavage activity, or a combination thereof. In some embodiments, the polypeptide cleaves within or near the target sequence. In some embodiments, the polypeptide cleaves at least one strand of the target nucleic acid. In some embodiments, the polypeptide comprises at least one mutation or amino acid alteration that results in retaining, enhancing or reducing activity relative to corresponding reference amino acid sequence set forth in TABLE 1. In some embodiments, the at least one mutation comprises an amino acid substitution at a position as set forth in TABLE 2 relative to corresponding reference amino acid sequence of TABLE 1. In some embodiments, the polypeptide has nickase activity or nuclease activity. In some embodiments, the polypeptide comprises an effector protein, an effector partner, a fusion protein or a combination thereof. In some embodiments, the effector partner is selected from a polymerase, a deaminase, a reverse transcriptase, a transcriptional repressor, an integrase, a recombinase and a transcriptional activator. In some embodiments, the effector protein is fused to an effector partner, wherein the effector protein and the effector partner are heterologous to each other. In some embodiments, the effector protein is directly fused to N terminus or C terminus of the effector partner by an amide bond. In some embodiments, the polypeptide comprises at least one nuclear localization signal sequence. In some embodiments, the at least one nuclear localization signal sequence independently comprises an amino acid sequence that is identical to any one of nucleotide sequences set forth in TABLE 3. In some embodiments, the polypeptide recognizes a protospacer adjacent motif (PAM). In some embodiments, the polypeptide recognizes a PAM as set forth in TABLE 4. In some embodiments, the polypeptide comprises an amino acid sequence that is at least 90% identical to any one of SEQ ID NO: 1, 569-631. In some embodiments, the polypeptide comprises an amino acid sequence that is at least 90% identical to SEQ ID NO: 1. In some embodiments, the polypeptide comprises an amino acid substitution at position D220R, wherein the polypeptide comprises enhanced nuclease activity. In some embodiments, the engineered guide nucleic acid comprises a sgRNA or a crRNA. In some embodiments, the polypeptide comprises an amino acid sequence that is at least 90% identical to any one of SEQ ID NO: 2, 632-700. In some embodiments, the polypeptide comprises an amino acid sequence that is at least 90% identical to SEQ ID NO: 2. In some embodiments, the polypeptide comprises an amino acid substitution at position L26R, wherein the polypeptide comprises enhanced nuclease activity.

Also provided herein are systems for modifying a target nucleic acid of a hematopoietic stem cell (HSC) comprising an engineered guide nucleic acid, wherein the system comprises at least two engineered guide nucleic acids selected from: (i) a first engineered guide nucleic acid comprising a spacer sequence that is at least 90% identical or complementary to an equal length portion of a target sequence of a gene encoding the HBB gene, a promoter thereof, an enhancer thereof, or a fragment thereof; (ii) a second engineered guide nucleic acid comprising a spacer sequence that is at least 90% identical or complementary to an equal length portion of a target sequence of a gene encoding the BCL11A gene, a promoter thereof, an enhancer thereof, or a fragment thereof; (iii) a third engineered guide nucleic acid comprising a spacer sequence that is at least 90% identical or complementary to an equal length portion of a target sequence of a gene encoding the HBG1 gene, a promoter thereof, an enhancer thereof, or a fragment thereof; and (iv) a fourth engineered guide nucleic acid comprising a spacer sequence that is at least 90% identical or complementary to an equal length portion of a target sequence of a gene encoding the HBG1 gene, a promoter thereof, an enhancer thereof, or a fragment thereof. In some embodiments, the nucleic acid encoding the polypeptide, the nucleic acid encoding the engineered guide nucleic acid, or both are mRNA. In some embodiments, the systems described herein further comprising a donor nucleic acid, or a nucleic acid encoding a donor nucleic acid. In some embodiments, the donor nucleic acid comprises a nucleotide, a nucleotide sequence, a coding sequence, a gene, an exon, an intron, a gene regulatory region, a fragment thereof, or combinations thereof. In some embodiments, the donor nucleic acid comprises a nucleotide sequence of any one of the following genes: BCL11A, B2M, PNPLA2, CHD4, CIITA, GATA1, HBA, HBA1, HBA2, HBB, HBD, HBE1, HBG, HBG1, HBG2, HBM, HBQ1, HBZ, HOXA9, KLF1, MBD3, MYB, TRAC1, ZBTB7A, a fragment thereof, a promoter thereof, an enhancer thereof, or a combination thereof. In some embodiments, the donor nucleic acid encodes a HBB gene, a BCL11A gene, a HBG1 gene and/or a HBG2 gene, and comprises one or more nucleotide sequences for directing integration into the γ-globin gene. In some embodiments, the systems described herein further comprising an antibody, wherein the antibody is conjugated to one or more components of the system of any one of claims 1-50, and wherein the antibody recognizes and binds HSC. In some embodiments, the antibody recognizes CD34+ and CD117.

Also provided herein are vectors encoding one or more components of any one of the systems described herein. In some embodiments, the vector further comprises a nucleotide sequence encoding at least one HSC specific promoter. In some embodiments, the vector is a viral vector. In some embodiments, the viral vector is an adeno associated viral (AAV) vector. In some embodiments, the vector is formulated with a lipid or a lipid nanoparticle.

Also provided herein are libraries of nucleic acid vectors comprising at least one of the vectors described herein, wherein at least one of the vectors described herein is any one of the vectors described herein.

Also provided herein are pharmaceutical compositions comprising any one of the systems described herein, any one of the vectors described herein, and a pharmaceutically acceptable carrier. In some embodiments, a pharmaceutical composition comprising any one of the engineered HSCs described herein, and a pharmaceutically acceptable carrier.

Also provided herein are methods of producing an engineered HSC, the method comprising: (i) contacting a target nucleic acid of a HSC with the system of any one of claims 1-54, the vector of any one of claims 55-59, or the pharmaceutical composition of claim 61 for a first sufficient period of time to allow for transfection of the HSC; and (ii) culturing the HSC for a second sufficient period of time for indels to occur in the target nucleic acid for modifying target nucleic acid and, thereby, producing the engineered HSC. In some embodiments, the transfection comprises s electroporation, viral infection, transfection, lipofection, electroporation, calcium phosphate precipitation, direct microinjection, or combinations thereof. In some embodiments, the method is performed in vitro or in vivo. In some embodiments, the target nucleic acid comprises any one of the target nucleic acid sequences set forth in TABLE 10, a fragment thereof, a promoter thereof, an enhancer thereof, a variant thereof or a combination thereof. In some embodiments, the modifying comprises cleaving the target nucleic acid, deleting a nucleic acid of the target nucleic acid, inserting a donor nucleic acid into the target nucleic acid, substituting a nucleic acid of the target nucleic acid with a donor nucleic acid, more than one of the foregoing, or combinations thereof. In some embodiments, the modifying results in upregulation of gene expression, downregulation of gene expression, expression of one or more proteins, or a combination thereof. In some embodiments, the first sufficient time comprises at least about 1 hour, at least about 2 hours, at least about 3 hours, at least about 4 hours, at least about 5 hours or at least about 6 hours. In some embodiments, the second sufficient time at least 1 day, at least 2 days, at least 3 days, at least 4 days, at least 5 days, or at least 6 days. In some embodiments, the method further comprises freezing the HSC. In some embodiments, the method comprises no other agent that alters ability of the engineered HSC to self-renew and/or differentiate into different types of cells.

Also provided herein are engineered HSCs, wherein any one of the HSCs described herein are modified by any one of the systems or methods of modifying described herein. In some embodiments, the engineered HSCs self-renew and/or differentiate into different types of cells. In some embodiments, the different types of cells comprise a population of hematopoietic stem and progenitor cells (HSPCs), a blood cell, a myeloid cell, a lymphoid cell, a myeloid common progenitor cell, a megakaryocytes-erythrocyte progenitor cell, a granulocytes-macrophages progenitor cell, a monocytic-dendritic progenitor cell, or a lymphoid common progenitor cell.

Also provided herein are methods of treating a disease or disorder comprising administering any one of the engineered HSCs described herein or any one of the pharmaceutical compositions described herein. In some embodiments, the disease or disorder is any one of the diseases or disorders set forth in TABLE 12.

Also provided herein are methods of treating a disease or disorder comprising administering a cell modified by a system in a subject in need thereof, wherein the system comprises (a) an engineered guide nucleic acid, or a nucleic acid encoding the engineered guide nucleic acid, wherein (i) the engineered guide nucleic acid comprises a first region and a second region, (ii) the second region comprises a nucleotide sequence that is at least 80% identical to any one of repeat sequences recited in TABLE 5 or intermediary sequences recited in TABLE 8, (iii) the second region comprises a nucleotide sequence that is at least 80% identical to any one of spacer sequences recited in TABLE 7, and (iv) the first region and the second region are heterologous to each other, and (b) a polypeptide, or a nucleic acid encoding the polypeptide, wherein (i) the polypeptide at least partially binds to the first region to form an RNP complex, and (ii) the RNP complex, upon hybridization of the second region to the target nucleic, modifies the target nucleic acid of the cell, wherein the cell comprises a hematopoietic stem cell (HSC), a population of hematopoietic stem and progenitor cells (HSPCs), a blood cell, a myeloid cell, a lymphoid cell, a myeloid common progenitor cell, a megakaryocytes-erythrocyte progenitor cell, a granulocytes-macrophages progenitor cell, a monocytic-dendritic progenitor cell, or a lymphoid common progenitor cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows that the L26R CasPhi. 12 variant (delivered as mRNA via electroporation) can edit a target nucleic acid in hematopoietic stem cells (HSCs).

FIG. 2 shows indel formation in the promoters of HBG1 and HBG2 in HSCs with wildtype CasPhi. 12 and the L26R CasPhi. 12 variant in a dose dependent study.

FIG. 3 shows indel formation in the promoters of HBG1 and HBG2 in HSCs with wildtype CasPhi. 12 and the L26R CasPhi. 12 variant.

FIG. 4A shows hematopoietic stem and progenitor cells (HSPCs) proliferate after electroporation of effector protein (L26R CasPhi. 12 variant) and guide nucleic acid encoding plasmids. FIG. 4B demonstrates HSPC viability by a flow cytometry assay using a viability dye after electroporating plasmids encoding effector proteins and guide nucleic acids. FIG. 4C shows indel activity by the L26R CasPhi.12 variant in a dose dependent manner 4 days after electroporation.

FIG. 5A shows body weights of NBSGW (NOD.Cg-Kit^{W-41J} Tyr⁺ Prkdc^{scid} I12rg^{im1Wjl}/ThomJ) mice after injection of unedited HSCs in low (220,000 cells) and high (700,000 cells) dose concentrations. FIG. 5B shows baseline spleen weights of NBSGW mice 10 weeks after injection of unedited HSCs in low (220,000 cells) and high (700,000 cells) dose concentrations.

FIG. 6A shows an increase in the percentage of cells that are human CD45+ in peripheral blood of NBSGW mice 6 weeks after receiving low (220,000 cells) and high (700,000 cells) doses of unedited HSCs relative to baseline. FIG. 6B shows higher populations of human CD45+ cells in NBSGW mouse bone marrow 6 weeks after receiving both low (220,000 cells) and high (700,000 cells) doses of unedited HSCs relative to baseline.

FIG. 7A shows an increase in the percentage of cells that are human CD45+ in peripheral blood of NBSGW mice 10 weeks after receiving low (220,000 cells) and high (700,000 cells) doses of unedited HSCs relative to baseline. FIG. 7B shows higher populations of human CD45+ cells the blood of NBSGW mice 10 weeks after receiving both low (220,000 cells) and high (700,000 cells) doses of unedited HSCs relative to baseline. FIG. 7C shows higher populations of human CD45+ cells in NBSGW mouse bone marrow 10 weeks after receiving both low (220,000 cells) and high (700,000 cells) doses of unedited HSCs relative to baseline. FIG. 7D shows higher populations of human CD45+ cells in NBSGW mouse spleen 10 weeks after receiving both low (220,000 cells) and high (700,000 cells) doses of unedited HSCs.

FIG. 8A illustrates an exemplary vector construction for donor nucleic acid integration used in Example 13 for neon system flow cytometry analysis. FIG. 8B shows the relative viability of unedited HSPCs and HSPCs electroporated with the L26R CasPhi. 12 variant. FIG. 8C shows that exposure to increasing doses of AAV6 leads to progressive loss of proliferative potential of HSPCs. FIG. 8D shows 12% GFP integration was obtained with the L26R CasPhi.12 variant using ssAAV6 as a donor template. FIG. 8E shows mean fluorescence intensity as a result of GFP integration with 5 μg L26R CasPhi.12 variant as a function of ssAAV6 concentration.

FIG. 9A and FIG. 9B show indel activity of CasM.265466 effector protein and guide nucleic acids on the γ-globin gene promoter in HSPCs. FIG. 9A shows % indel generated for guide nucleic acids targeting a target sequence of the HBB gene, the BCL11A gene and the HBG1 promoter. FIG. 9B shows % indel generation of CasM.265466 with guide nucleic acid R11602 (BCL11A Enh-2 targeting).

FIG. 10 shows % indel of a smaller subset of guides targeting the HBB gene, the BCL11A gene, and the HBG1 promoter with the CasM.265466 effector protein.

FIG. 11 shows proliferation of HSPCs after electroporation with mRNA encoding CasM.265466 effector protein and R11602 (BCL11A Enh-2 targeting) guide nucleic acid.

FIG. 12A shows viability of HSPCs electroporated with mRNA encoding CasM.265466 effector protein and guide nucleic acid R11602 (BCL11A Enh-2 targeting). FIG. 12B shows viability of HSPCs electroporated with mRNA encoding a D220R variant of CasM.265466 effector protein and guide nucleic acid R11602 (BCL11A Enh-2 targeting).

FIG. 13 shows % indel generated with wildtype CasM.265466 effector protein and the D220R CasM.265466 variant in HSPCs from 0 to 5 days post-electroporation.

FIG. 14A and FIG. 14B shows indel patterns generated by wildtype CasM.265466 effector protein and the D220R CasM.265466 variant, respectively.

FIG. 15 shows viability of unedited and CasM.265466 effector protein edited HSPCs, wherein HSPCs were subject to a freeze-thaw cycle.

FIG. 16 shows a proliferation ratio of unedited and CasM.265466 effector protein edited HSPCs, wherein HSPCs were subject to a freeze-thaw cycle.

FIG. 17 shows total colony forming unit in unedited and CasM.265466 effector protein edited HSPCs, wherein HSPCs were subject to a freeze-thaw cycle.

FIG. 18 shows % indel observed 5 days post electroporation in unedited and CasM.265466 effector protein edited HSPCs, wherein HSPCs were subject to a freeze-thaw cycle.

FIG. 19A shows body weight of NBSGW (NOD.Cg-Kit^{W-41J} Tyr Prkdc^{scid} I12rg^{im1Wjl}/ThomJ) mice after injection of CasM.265466 effector protein edited HSPCs in low (220,000 cells) and high (700,000 cells) dose concentrations. FIG. 19B shows the spleen weight of NBSGW mice 10 weeks after injection of edited HSPCs in low (220,000 cells) and high (700,000 cells) dose concentrations. For a negative control, mice were injected with vehicle only.

FIG. 20A and FIG. 20B show the results of flow cytometry of cells collected from peripheral blood of NBSGW mice 6 weeks post-injection of low (220,000 cells) and high (700,000 cells) doses of CasM.265466 effector protein edited HSPCs. FIG. 20C shows results of negative control for cells collected from peripheral blood, wherein the mice were injected with vehicle only. FIG. 20D summarizes the results of FIGS. 20A-20C showing % hCD45+ cells in harvested white blood cells.

FIG. 21A and FIG. 21B show the results of flow cytometry of cells collected from peripheral blood of NBSGW mice 10 weeks after an injection of a low (220,000 cells) or high (700,000 cells) dose of CasM.265466 effector protein edited HSPCs. FIG. 21C shows results of negative control for cells collected from peripheral blood, wherein the mice were injected with vehicle only. FIG. 21D and FIG. 21E show the results of flow cytometry of cells collected from bone marrow of NBSGW mice 10 weeks after injection of a low (220,000 cells) or high (700,000 cells) dose of CasM.265466 effector protein edited HSPCs. FIG. 21F shows results of negative control for cells collected from bone marrow, wherein the mice were injected with vehicle only. FIG. 21G and FIG. 21H show the results of flow cytometry of cells collected from spleen of NBSGW mice 10 weeks after injection of a low (220,000 cells) or high (700,000 cells) dose of CasM.265466 effector protein edited HSPCs. FIG. 21I shows results of negative control for cells collected from spleen, wherein the mice were injected with vehicle only. FIG. 21J summarizes the results of FIGS. 21A-21I showing % hCD45+ cells in the collected cells.

FIG. 22A illustrates an exemplary vector construction for donor nucleic acid integration, used in Example 20 for neon system flow cytometry analysis. FIG. 22B shows a proliferation profile for unedited HSPCs and HSPCs 5 days after electroporation with mRNA encoding CasM.265466 effector proteins and guide nucleic acids, and transduction with AAV6 vector construct at varying dose from 0 to 5×10⁵ MOI, wherein the AAV6 vector construct was engineered according to FIG. 22A. FIG. 22C shows % viability for unedited and HSPCs 1, 5 and 8 days after electroporation with mRNA encoding CasM.265466 effector proteins and guide nucleic acids, and transduction with AAV6 vector construct at varying dose from 0 to 5E5 MOI, wherein the AAV6 vector construct was engineered according to FIG. 22A. FIG. 22D shows % GFP integration observed in unedited and HSPCs 8 days after electroporation with mRNA encoding CasM.265466 effector proteins and guide nucleic acids and AAV6 transduction, wherein AAV6 transduction was performed at a dose varying from 0 to 2E5 MOI. FIG. 22E shows relative fluorescence observed in unedited and HSPCs 8 days after electroporation with mRNA encoding CasM.265466 effector protein and guide nucleic acid, and AAV6 transduction, wherein AAV6 transduction was performed at a dose varying from 0 to 2E5 MOI.

FIG. 23A shows proliferative potential of frozen HSPCs after electroporation with mRNA encoding a D220R variant of CasM.265466 effector protein and a guide nucleic acid targeting BCL11A, or a L26R variant of CasPhi.12 effector protein and a guide nucleic acid targeting the promoter of HBG1 and HBG2.

FIG. 23B shows viability of frozen HSPCs after electroporation with mRNA encoding a D220R variant of CasM.265466 effector protein and a guide nucleic acid targeting BCL11A, or a L26R variant of CasPhi. 12 effector protein and a guide nucleic acid targeting the promoter of HBG1 and HBG2.

FIG. 24 shows indel activity of HSPCs edited using a D220R variant of CasM.265466 effector protein and guide nucleic acid targeting BCL11A, or a L26R variant of CasPhi.12 effector protein and guide nucleic acid targeting the promoter of HBG1 and HBG2, as compared to unedited HSPCs.

FIG. 25A and FIG. 25B show differentiation potential or multi-lineage development potential of HSPCs edited using a D220R variant of CasM.265466 effector protein and corresponding guide nucleic acid, as compared to unedited HSPCs. Five different densities (e.g., CFU-E or BFU-E, CFU-G, CFU-M, CFU-GM, CFU-GEMM) of HSPCs were plated onto methylcellulose gels for differentiation assay (e.g., CFU assay).

FIG. 26A shows body weight of NBSGW (NOD.Cg-Kit^{W-41J} Tyr Prkdc^{scid} I12rg^{im1Wjl}/ThomJ) mice after injection of unedited HSCs, HSPCs edited using a D220R variant of CasM.265466 effector protein, or HSPCs edited using a L26R variant of CasPhi.12 effector protein, as compared to uninjected mice.

FIG. 26B shows the spleen weight of NBSGW mice 12 weeks after injection of unedited HSCs, HSPCs edited using a D220R variant of CasM.265466 effector protein, or HSPCs edited using a L26R variant of CasPhi.12 effector protein, as compared to uninjected mice.

FIG. 27A shows % of cells that are human CD45+ in peripheral blood of NBSGW mice 6 weeks after injection of unedited HSCs, HSPCs edited using a D220R variant of CasM.265466 effector protein, or HSPCs edited using a L26R variant of CasPhi.12 effector protein, as compared to uninjected mice. Successful HSC engraftment was demonstrated by an increased % of human CD45+ cells in peripheral blood.

FIG. 27B shows % of cells that are human CD45+ in bone marrow, spleen and peripheral blood of NBSGW mice 12 weeks after injection of unedited HSCs, HSPCs edited using a D220R variant of CasM.265466 effector protein, or HSPCs edited using a L26R variant of CasPhi.12 effector protein, as compared to uninjected mice.

FIG. 28A shows indel activity of engrafted HSC recruited from NBSGW mice 12 weeks after injection of HSPCs edited using a L26R variant of CasPhi.12 effector protein.

FIG. 28B shows indel activity of engrafted HSC recruited from NBSGW mice 12 weeks after injection of HSPCs edited using a D220R variant of CasM.265466 effector protein.

FIG. 29 shows the results of a complete blood count (CBC) test performed using peripheral blood of NBSGW mice 12 weeks after injection of unedited HSCs, HSPCs edited using a D220R variant of CasM.265466 effector protein, or HSPCs edited using a L26R variant of CasPhi.12 effector protein, as compared to uninjected mice.

FIGS. 30A-30C show exemplary results of indel activity, proliferation rate and viability observed with CasM.265466 effector protein (NanoCas) versus Cas9 from Streptococcus pyogenes (SpCas9) in HSCs post-electroporation.

### DETAILED DESCRIPTION OF THE INVENTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### I. Definitions

Unless otherwise indicated, all technical terms used herein have the same meaning as commonly understood by one of ordinary skill in the art to which this invention belongs. Unless otherwise indicated or obvious from context, the following terms have the following meanings:

The terms, “a,” “an,” and “the,” as used herein, include plural references unless the context clearly dictates otherwise.

The terms, “or” and “and/or,” as used herein, include any and all combinations of one or more of the associated listed items.

The terms, “including,” “includes,” “included,” and other forms, are not limiting.

The terms, “comprise” and its grammatical equivalents, as used herein, specify the presence of stated features, integers, steps, operations, elements, and/or components, but do not preclude the presence or addition of one or more other features, integers, steps, operations, elements, components, and/or groups thereof.

The term, “about,” as used herein in reference to a number or range of numbers, is understood to mean the stated number and numbers +/−10% thereof, or 10% below the lower listed limit and 10% above the higher listed limit for the values listed for a range.

The terms, “% identical,” “% identity,” “percent identity,” and grammatical equivalents thereof, as used herein, in the context of an amino acid sequence or nucleotide sequence, refer to the percent of residues that are identical between respective positions of two sequences when the two sequences are aligned for maximum sequence identity. The % identity is calculated by dividing the total number of the aligned residues by the number of the residues that are identical between the respective positions of the at least two sequences and multiplying by 100. For the purposes of calculating % identity, thymine (T) may be considered the same as uracil (U). Generally, computer programs can be employed for such calculations. Illustrative programs that compare and align pairs of sequences, include ALIGN (Myers and Miller, Comput Appl Biosci. 1988 March; 4 (1): 11-7), FASTA (Pearson and Lipman, Proc Natl Acad Sci USA. 1988 April; 85 (8): 2444-8; Pearson, Methods Enzymol. 1990; 183:63-98) and gapped BLAST (Altschul et al., Nucleic Acids Res. 1997 Sep. 1; 25 (17): 3389-40), BLASTP, BLASTN, or GCG (Devereux et al., Nucleic Acids Res. 1984 Jan. 11; 12 (1 Pt 1): 387-95).

The terms, “% complementary”, “% complementarity”, “percent complementary”, “percent complementarity” and grammatical equivalents thereof, as used interchangeably herein, in the context of two or more nucleic acid molecules, refer to the percent of nucleotides in two nucleotide sequences in said nucleic acid molecules of equal length that can undergo cumulative base pairing at two or more individual corresponding positions in an antiparallel orientation. Accordingly, the terms include nucleic acid sequences that are not completely complementary over their entire length, which indicates that the two or more nucleic acid molecules include one or more mismatches. A “mismatch” is present at any position in the two opposed nucleotides that are not complementary. The % complementary is calculated by dividing the total number of the complementary residues by the total number of the nucleotides in one of the equal length sequences, and multiplying by 100. Complete or total complementarity describes nucleotide sequences in 100% of the residues of a nucleotide sequence are complementary to residues in a reference nucleotide sequence. “Partially complementarity” describes nucleotide sequences in which at least 20%, but less than 100%, of the residues of a nucleotide sequence are complementary to residues in a reference nucleotide sequence. In some instances, at least 50%, but less than 100%, of the residues of a nucleotide sequence are complementary to residues in a reference nucleotide sequence. In some instances, at least 70%, 80%, 90% or 95%, but less than 100%, of the residues of a nucleotide sequence are complementary to residues in a reference nucleotide sequence. “Noncomplementary” describes nucleotide sequences in which less than 20% of the residues of a nucleotide sequence are complementary to residues in a reference nucleotide sequence.

The term, “percent similarity,” or “% similarity,” as used herein, in the context of an amino acid sequence, refers to a value that is calculated by dividing a similarity score by the length of the alignment. The similarity of two amino acid sequences can be calculated by using a BLOSUM62 similarity matrix (Henikoff and Henikoff, Proc. Natl. Acad. Sci. USA., 89:10915-10919 (1992)) that is transformed so that any value ≥1 is replaced with +1 and any value ≤0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, if a substitution could be treated as conservative with any of the amino acids in that position of the multilevel consensus sequence, +1 point is assigned. For example, given the multilevel consensus sequence: RLG and YCK, the test sequence QIQ would receive three points. This is because in the transformed BLOSUM62 matrix, each combination is scored as: Q-R: +1; Q-Y: +0; I-L: +1; I-C: +0; Q-G: +0; Q-K: +1. For each position, the highest score is used when calculating similarity. The % similarity can also be calculated using commercially available programs, such as the Geneious Prime software given the parameters matrix=BLOSUM62 and threshold ≥1.

The terms, “bind,” “binding,” “interact” and “interacting,” as used herein, refer to a non-covalent interaction between macromolecules (e.g., between two polypeptides, between a polypeptide and a nucleic acid; between a polypeptide/guide nucleic acid complex and a target nucleic acid; and the like). While in a state of noncovalent interaction, the macromolecules are said to be “associated” or “interacting” or “binding” (e.g., when a molecule X is said to interact with a molecule Y, it is meant the molecule X binds to molecule Y in a non-covalent manner). Non-limiting examples of non-covalent interactions are ionic bonds, hydrogen bonds, van der Waals and hydrophobic interactions. Not all components of a binding interaction need be sequence-specific (e.g., contacts with phosphate residues in a DNA backbone), but some portions of a binding interaction may be sequence-specific.

The term, “base editor,” as used herein, refers to a polypeptide or fusion protein comprising a base editing activity. The polypeptide with base editing activity may be referred to as an effector partner. The base editor can differ from a naturally occurring base editing enzyme. It is understood that any reference to a base editor herein also refers to a base editing enzyme variant. The base editor is functional when the effector protein is coupled to a guide nucleic acid. The guide nucleic acid imparts sequence specific activity to the base editor. By way of non-limiting example, the effector protein may comprise a catalytically inactive effector protein (e.g., a catalytically inactive variant of an effector protein described herein). Also, by way of non-limiting example, the base editing enzyme comprises deaminase activity.

The term, “catalytically inactive effector protein,” as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some instances, the catalytically inactive effector protein is referred to as a catalytically inactive variant of an effector protein.

The term, “cis cleavage,” as used herein, refers to cleavage (hydrolysis of a phosphodiester bond) of a target nucleic acid by a complex of an effector protein and a guide nucleic acid (e.g., an RNP complex), wherein at least a portion of the guide nucleic acid is hybridized to at least a portion of the target nucleic acid. In some instances, cleavage occurs within or directly adjacent to the portion of the target nucleic acid that is hybridized to the portion of the guide nucleic acid.

The term, “codon optimized,” as used herein, refers to a mutation of a nucleotide sequence encoding a polypeptide, such as a nucleotide sequence encoding an effector protein, to mimic the codon preferences of the intended host organism or cell while encoding the same polypeptide. Thus, the codons can be changed, but the encoded polypeptide remains unchanged. For example, if the intended target cell was a human cell, a human codon-optimized nucleotide sequence encoding an effector protein could be used. As another non-limiting example, if the intended host cell were a mouse cell, then a mouse codon-optimized nucleotide sequence encoding an effector protein could be generated. As another non-limiting example, if the intended host cell were a eukaryotic cell, then a eukaryote codon-optimized nucleotide sequence encoding an effector protein could be generated. As another non-limiting example, if the intended host cell were a prokaryotic cell, then a prokaryote codon-optimized nucleotide sequence encoding an effector protein could be generated. Codon usage tables are readily available, for example, at the “Codon Usage Database” available at www.kazusa.or.jp/codon.

The terms, “complementary” and “complementarity,” as used herein, in the context of a nucleic acid molecule or nucleotide sequence, refer to the characteristic of a polynucleotide having nucleotides that can undergo cumulative base pairing with their Watson-Crick counterparts (C with G; or A with T) in a reference nucleic acid in antiparallel orientation. For example, when every nucleotide in a polynucleotide or a specified portion thereof forms a base pair with every nucleotide in an equal length sequence of a reference nucleic acid, that polynucleotide is said to be 100% complementary to the sequence of the reference nucleic acid. In a double stranded DNA or RNA sequence, the upper (sense) strand sequence is, in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the “reverse complement” sequence or the “reverse complementary” sequence is understood as the sequence of the lower strand in the direction of its 5′- to its 3′-end. Each nucleotide in a double stranded DNA or RNA molecule that is paired with its Watson-Crick counterpart can be referred to as its complementary nucleotide. The complementarity of modified or artificial base pairs can be based on other types of hydrogen bonding and/or hydrophobicity of bases and/or shape complementarity between bases.

The term, “cleavage assay,” as used herein, refers to an assay designed to visualize, quantitate or identify cleavage of a nucleic acid. In some instances, the cleavage activity comprises cis cleavage activity.

The terms, “cleave,” “cleaving” and “cleavage,” as used herein, in the context of a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The term, “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from another organism.

The term, “conservative substitution,” as used herein, refers to the replacement of one amino acid for another such that the replacement takes place within a family of amino acids that are related in their side chains. Conversely, the term “non-conservative substitution” as used herein refers to the replacement of one amino acid residue for another that does not have a related side chain. Genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser(S), Thr (T). Amino acids may be related by aliphatic side chains: Gly (G), Ala (A), Val (V), Leu (L), Ile (I), Ser(S), Thr (T), with Ser(S) and Thr (T) optionally being grouped separately as aliphatic-hydroxyl; Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

The terms, “CRISPR RNA” and “crRNA,” as used herein, refer to a type of guide nucleic acid that is RNA comprising a first sequence that is capable of hybridizing to a target sequence of a target nucleic acid and a second sequence that is capable of interacting with an effector protein either directly (by being bound by an effector protein) or indirectly (e.g., by hybridization with a second nucleic acid molecule that can be bound by an effector). The first sequence and the second sequence are directly connected to each other or by a linker.

The term, “donor nucleic acid,” as used herein, refers to a nucleic acid that is (designed or intended to be) incorporated into a target nucleic acid or target sequence.

The term, “edited target nucleic acid,” as used herein, refers to a target nucleic acid, wherein the target nucleic acid has undergone an editing, for example, after contact with an effector protein. In some instances, the editing is an alteration in the sequence of the target nucleic acid. In some instances, the edited target nucleic acid comprises an insertion, deletion, or substitution of one or more nucleotides compared to the unedited target nucleic acid.

The term, “effector protein,” as used herein, refers to a protein, polypeptide, or peptide that is capable of interacting with a nucleic acid, such as a guide nucleic acid, to form a complex (e.g., a RNP complex), wherein the complex interacts with a target nucleic acid. The term, “effector partner,” as used herein, refers to a protein, polypeptide or peptide that can, in combination with an effector protein and guide nucleic acid, impart some function or activity that can be used to effectuate modification(s) of a target nucleic acid described herein and/or change expression of the target nucleic acid or other nucleic acids associated with the target nucleic acid, when used in connection with compositions, systems, and methods described herein.

The term, “engineered modification,” as used herein, refers to a structural change of one or more nucleic acid residues of a nucleotide sequence or one or more amino acid residue of an amino acid sequence. The engineered modifications of a nucleotide sequence can include chemical modification of one or more nucleobases, or a chemical change to the phosphate backbone, a nucleotide, a nucleobase or a nucleoside. The engineered modifications can be made to an effector protein amino acid sequence or guide nucleic acid nucleotide sequence, or any sequence disclosed herein (e.g., a nucleic acid encoding an effector protein or a nucleic acid that encodes a guide nucleic acid). Methods of modifying a nucleic acid or amino acid sequence are known. One of ordinary skill in the art will appreciate that the engineered modification(s) may be located at any position(s) of a nucleic acid such that the function of the nucleic acid, protein, composition or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid.

The term, “functional domain,” as used herein, refers to a region of one or more amino acids in a protein that is required for an activity of the protein, or the full extent of that activity, as measured in an in vitro assay. Activities include, but are not limited to nucleic acid binding, nucleic acid editing, nucleic acid modifying, nucleic acid cleaving, protein binding. The absence of the functional domain, including mutations of the functional domain, would abolish or reduce activity.

The term, “functional fragment,” as used herein, refers to a fragment of a protein that retains some function relative to the entire protein. Non-limiting examples of functions are nucleic acid binding, nucleic acid editing, protein binding, nuclease activity, nickase activity, deaminase activity, demethylase activity, integrase activity, recombinase activity or acetylation activity. In some instances, a functional fragment comprises a recognized functional domain, e.g., a catalytic domain.

The term, “functional protein,” as used herein, refers to protein that retains at least some if not all activity relative to the wildtype protein. A functional protein can also include a protein having enhanced activity relative to the wildtype protein. Assays are known and available for detecting and quantifying protein activity, e.g., colorimetric and fluorescent assays. In some instances, a functional protein is a wildtype protein. In some instances, a functional protein is a functional portion of a wildtype protein.

The term, “fused,” as used herein, refers to at least two sequences that are connected together, such as by a covalent bond (e.g., an amide bond or a phosphodiester bond) or by a linker. The covalent bond can be formed by a conjugation (e.g., chemical conjugation or enzymatic conjugation) reaction.

The term, “fusion protein,” as used herein, refers to a protein comprising at least two heterologous polypeptides. In some instances, the fusion protein comprises one or more effector proteins and effector partners. In some instances, an effector protein and effector partner are not found connected to one another as a native protein or complex that occurs together in nature.

The term, “effector partner,” as used herein, refers to a protein or a fragment thereof that impart some function or activity to the fusion protein that is not provided by the effector protein. In some instances, the effector partner is fused, or linked by a linker, to one or more effector protein. In some instances, the effector partner is not fused or linked to one or more effector protein.

The term, “genetic disease,” as used herein, refers to a disease, disorder, condition, or syndrome associated with or caused by one or more mutations in the DNA of an organism having the genetic disease.

The term, “guide nucleic acid,” as used herein, refers to a nucleic acid that, when in a complex with one or more polypeptides described herein (e.g., an RNP complex) can impart sequence selectivity to the complex when the complex interacts with a target nucleic acid. In some instances, a guide nucleic acid is referred to interchangeably as a guide RNA, however it is understood that guide nucleic acids may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The term, “handle sequence,” as used herein, refers to a sequence of nucleotides in a single guide RNA (sgRNA), that is: 1) capable of being non-covalently bound by an effector protein and 2) connects the portion of the sgRNA capable of being non-covalently bound by an effector protein to a nucleotide sequence that is hybridizable to a target nucleic acid. In general, the handle sequence comprises an intermediary sequence, that is capable of being non-covalently bound by an effector protein. In some instances, the handle sequence further comprises a repeat sequence. In such instances, the intermediary sequence or a combination of the intermediary sequence and the repeat sequence is capable of being non-covalently bound by an effector protein.

The term, “heterologous,” as used herein, refers to at least two different polypeptide sequences that are not found similarly connected to one another in a native nucleic acid or protein. A protein that is heterologous to the effector protein is a protein that is not covalently linked by an amide bond to the effector protein in nature. In some instances, a protein is heterologous when the protein is not encoded by a species that encodes the effector protein. In some instances, a guide nucleic acid comprises “heterologous” sequences, which means that it includes a first nucleotide sequence and a second nucleotide sequence, wherein the first nucleotide sequence and the second nucleotide sequence are not found covalently linked by a phosphodiester bond in nature. Thus, the first nucleotide sequence is considered to be heterologous with the second nucleotide sequence, and, in some instances, the guide nucleic acid is referred to as a heterologous guide nucleic acid. A heterologous system comprises at least one component that is not naturally occurring together with remaining components of the heterologous system.

The terms, “hybridize,” “hybridizable” and grammatical equivalents thereof, refer to a nucleotide sequence that is able to noncovalently interact, i.e. form Watson-Crick base pairs and/or G/U base pairs, or anneal, to another nucleotide sequence in a sequence-specific, antiparallel, manner (i.e., a nucleotide sequence specifically interacts to a complementary nucleotide sequence) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) for both DNA and RNA. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, a guanine (G) can be considered complementary to both an uracil (U) and to an adenine (A). Accordingly, when a G/U base-pair can be made at a given nucleotide position, the position is not considered to be non-complementary, but is instead considered to be complementary. While hybridization typically occurs between two nucleotide sequences that are complementary, mismatches between bases are possible. It is understood that two nucleotide sequences need not be 100% complementary to be specifically hybridizable, hybridizable, partially hybridizable, or for hybridization to occur. Moreover, in some instances, a nucleotide sequence hybridizes over one or more segments such that intervening or adjacent segments are not involved in the hybridization event (e.g., a bulge, a loop structure or hairpin structure, etc.). The conditions appropriate for hybridization between two nucleotide sequences depend on the length of the sequence and the degree of complementarity, variables which are well known in the art. For hybridizations between nucleic acids with short stretches of complementarity (e.g., complementarity over 35 or less, 30 or less, 25 or less, 22 or less, 20 or less, or 18 or less nucleotides) the position of mismatches may become important. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Any suitable in vitro assay may be utilized to assess whether two sequences “hybridize”. One such assay is a melting point analysis where the greater the degree of complementarity between two nucleotide sequences, the greater the value of the melting temperature (Tm) for hybrids of nucleic acids having those sequences. The conditions of temperature and ionic strength determine the “stringency” of the hybridization. Temperature, wash solution salt concentration, and other conditions may be adjusted as necessary according to factors such as length of the region of complementation and the degree of complementation. Hybridization and washing conditions are well known and exemplified in Sambrook, J. and Russell, W., Molecular Cloning: A Laboratory Manual, Third Edition, Cold Spring Harbor Laboratory Press, Cold Spring Harbor (2001); and in Green, M. and Sambrook, J., Molecular Cloning: A Laboratory Manual, Fourth Edition, Cold Spring Harbor Laboratory Press, Cold Spring Harbor (2012).

The term, “indel,” as used herein, refers to an insertion-deletion or indel mutation, which is a type of genetic mutation that results from the insertion and/or deletion of one or more nucleotide in a target nucleic acid. An indel can vary in length (e.g., 1 to 1,000 nucleotides in length) and be detected by any suitable method, including sequencing.

The term, “indel percentage,” as used herein, refers to a percentage of sequencing reads that show at least one nucleotide has been edited from the insertion and/or deletion of nucleotides regardless of the size of insertion or deletion, or number of nucleotides edited. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are edited by a given effector protein.

The terms, “intermediary RNA” and “intermediary sequence,” as used herein, in a context of a single nucleic acid system, refers to a nucleotide sequence in a handle sequence, wherein the nucleotide sequence is capable of, at least partially, being non-covalently bound to an effector protein to form a complex (e.g., an RNP complex). An intermediary sequence is not a transactivating nucleic acid in systems, methods, and compositions described herein.

The term, “in vitro,” as used herein, refers to describing something outside an organism. In some instances, an in vitro system, composition or method takes place in a container for holding laboratory reagents such that it is separated from the biological source from which a material in the container is obtained. In vitro assays can encompass cell-based assays in which living or dead cells are employed. In vitro assays can also encompass a cell-free assay in which no intact cells are employed. The term “in vivo” is used to describe an event that takes place within an organism. The term “ex vivo” is used to describe an event that takes place in a cell that has been obtained from an organism. An ex vivo assay is not performed on a subject. Rather, it is performed upon a sample separate from a subject.

The terms, “length” and “linked” as used herein, refer to a nucleic acid (polynucleotide) or polypeptide, expressed as “kilobases” (kb) or “base pairs (bp)”. Thus, a length of 1 kb refers to a length of 1000 linked nucleotides, and a length of 500 bp refers to a length of 500 linked nucleotides. Similarly, a protein having a length of 500 linked amino acids is simply described as having a length of 500 amino acids.

The term, “linker,” as used herein, refers to a molecule that links a first polypeptide to a second polypeptide (e.g., by an amide bond) or a first nucleic acid to a second nucleic acid (e.g., by a phosphodiester bond).

The term, “mutation,” as used herein, refers to an alteration that changes an amino acid residue or a nucleotide as described herein. Such an alteration can include, for example, deletions, insertions, and/or substitutions. The mutation can refer to a change in structure of an amino acid residue or nucleotide relative to the starting or reference residue or nucleotide. A mutation of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. A mutation of a nucleotide includes, for example, changing one naturally occurring base for a different naturally occurring base, such as changing an adenine to a thymine or a guanine to a cytosine or an adenine to a cytosine or a guanine to a thymine. In some instances, a mutation of a nucleotide base results in a structural and/or functional alteration of the encoding peptide, polypeptide or protein by changing the encoded amino acid residue of the peptide, polypeptide or protein. In some instances, a mutation of a nucleotide base does not result in an alteration of the amino acid sequence or function of encoded peptide, polypeptide or protein, also known as a silent mutation. Methods of mutating an amino acid residue or a nucleotide are well known.

The terms, “mutation associated with a disease” and “mutation associated with a genetic disorder,” as used herein, refer to the co-occurrence of a mutation and the phenotype of a disease. In some instances, the mutation occurs in a gene, wherein transcription or translation products from the gene occur at a significantly abnormal level or in an abnormal form in a cell or subject harboring the mutation as compared to a non-disease control subject not having the mutation.

The term, “nickase,” as used herein, refers to an enzyme that possess catalytic activity for single stranded nucleic acid cleavage of a double stranded nucleic acid.

The term, “nickase activity,” as used herein, refers to catalytic activity that results in single stranded nucleic acid cleavage of a double stranded nucleic acid.

The terms, “non-naturally occurring” and “engineered,” as used herein, refer to indicate involvement of the hand of man. The terms, when referring to a nucleic acid, nucleotide, protein, polypeptide, peptide or amino acid, refer to a molecule, such as but not limited to, a nucleic acid, nucleotide, protein, polypeptide, peptide or amino acid refers to a modification of that molecule (e.g., chemical modification, nucleotide sequence, or amino acid sequence) that is not present in the naturally molecule. The terms, when referring to a composition or system described herein, refer to a composition or system having at least one component that is not naturally associated with the other components of the composition or system. By way of a non-limiting example, a composition includes an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The terms, “nuclease” and “endonuclease” as used herein, refer to an enzyme which possesses catalytic activity for nucleic acid cleavage.

The term, “nuclease activity,” as used herein, refers to catalytic activity that results in nucleic acid cleavage (e.g., ribonuclease activity (ribonucleic acid cleavage), or deoxyribonuclease activity (deoxyribonucleic acid cleavage), etc.).

The term, “nucleic acid,” as used herein, refers to a polymer of nucleotides. In some instances, a nucleic acid comprises ribonucleotides, deoxyribonucleotides, combinations thereof, and modified versions of the same. In some instances, a nucleic acid is single-stranded or double-stranded, unless specified. Non-limiting examples of nucleic acids are double stranded DNA (dsDNA), single stranded (ssDNA), messenger RNA, genomic DNA, cDNA, DNA-RNA hybrids, and a polymer comprising purine and pyrimidine bases or other natural, chemically or biochemically modified, non-natural, or derivatized nucleotide bases. Accordingly, in some instances, nucleic acids as described herein comprise one or more mutations, one or more engineered modifications, or both.

The term, “nucleic acid expression vector,” as used herein, refers to a plasmid that can be used to express a nucleic acid of interest.

The term, “nuclear localization signal (NLS),” as used herein, refers to an entity (e.g., peptide) that facilitates localization of a nucleic acid, protein, or small molecule to the nucleus, when present in a cell that contains a nuclear compartment.

The terms, “nucleotide(s)” and “nucleoside(s)”, as used herein, in the context of a nucleic acid molecule having multiple residues, refer to describing the sugar and base of the residue contained in the nucleic acid molecule. Similarly, a skilled artisan could understand that linked nucleotides and/or linked nucleosides, as used in the context of a nucleic acid having multiple linked residues, are interchangeable and describe linked sugars and bases of residues contained in a nucleic acid molecule. When referring to a “nucleobase(s)”, or linked nucleobase, as used in the context of a nucleic acid molecule, it can be understood as describing the base of the residue contained in the nucleic acid molecule, for example, the base of a nucleotide, nucleosides, or linked nucleotides or linked nucleosides. A person of ordinary skill in the art when referring to nucleotides, nucleosides, and/or nucleobases would also understand the differences between RNA and DNA (generally the exchange of uridine for thymidine or vice versa) and the presence of nucleoside analogs, such as modified uridines, do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, NI-methyl pseudouridine, or 5-methoxyuridine, is considered 100% identical to AUG in that both are perfectly complementary to the same sequence (5′-CAU).

The term, “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by suitable methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

The terms, “polypeptide” and “protein,” as used herein, refer to a polymeric form of amino acids. In some instances, a polypeptide includes coded and non-coded amino acids, chemically or biochemically modified or derivatized amino acids, and polypeptides having modified peptide backbones. Accordingly, in some instances, polypeptides as described herein comprise one or more mutations, one or more engineered modifications, or both. It is understood that when describing coding sequences of polypeptides described herein, said coding sequences do not necessarily require a codon encoding an N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some instances, when a heterologous peptide, such as an effector partner protein, protein tag or NLS, is located at the N terminus of the effector protein, a start codon for the heterologous peptide serves as a start codon for the effector protein as well. Thus, in some instances, the natural start codon encoding an amino acid residue sufficient for initiating translation (e.g., Methionine (M) or a Valine (V)) of the effector protein is removed or absent.

The term, “prime editing enzyme”, as used herein, refers to a protein, polypeptide, or fragment thereof that is capable of catalyzing the editing (insertion, deletion, or base-to-base conversion) of a target nucleotide or nucleotide sequence in a nucleic acid.

The terms, “promoter” and “promoter sequence,” as used herein, refer to a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters often, but not always, contain “TATA” boxes and “CAT” boxes. In some instances, various promoters, including inducible promoters, are used to drive expression by the various vectors of the present disclosure.

The terms, “protospacer adjacent motif” and “PAM,” as used herein, refer to a nucleotide sequence found in a target nucleic acid that directs an effector protein to edit the target nucleic acid at a specific location. In some instances, a PAM is required for a complex of an effector protein and a guide nucleic acid (e.g., an RNP complex) to hybridize to and edit the target nucleic acid. In some instances, the complex does not require a PAM to edit the target nucleic acid.

The term, “REC domain,” as used herein, refers to domain in an α-helical recognition region or lobe. In some instances, an effector protein contains at least one REC domain (e.g., REC1, REC2) which generally helps to accommodate and stabilize the guide nucleic acid and target nucleic acid hybrid.

The term, “recombinant,” as used herein, in the context of proteins, polypeptides, peptides and nucleic acids, refers to proteins, polypeptides, peptides and nucleic acids that are products of various combinations of cloning, restriction, and/or ligation steps resulting in a construct having a structural coding or non-coding sequence distinguishable from endogenous nucleic acids found in natural systems.

The term, “regulatory element,” used herein, refers to transcriptional and translational control sequences, such as promoters, enhancers, polyadenylation signals, terminators, protein degradation signals, and the like, that provide for and/or regulate transcription of a non-coding sequence (e.g., a guide nucleic acid) or a coding sequence (e.g., effector proteins, fusion proteins, and the like) and/or regulate translation of an encoded polypeptide.

The term, “repeat sequence,” as used herein, refers to a sequence of nucleotides in a guide nucleic acid that is capable of, at least partially, interacting with an effector protein.

The terms, “ribonucleotide protein complex” and “RNP” as used herein, refer to a complex of one or more nucleic acids and one or more polypeptides described herein. While the term utilizes “ribonucleotides,” it is understood that the one or more nucleic acids comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The terms, “RuvC” and “RuvC domain,” as used herein, refer to a region of an effector protein that is capable of cleaving a target nucleic acid, and in certain instances, of processing a pre-crRNA. In some instances, the RuvC domain is located near the C-terminus of the effector protein. In some instances, a single RuvC domain comprises RuvC subdomains, for example a RuvCI subdomain, a RuvCII subdomain and a RuvCIII subdomain. The term “RuvC” domain can also refer to a “RuvC-like” domain. Various RuvC-like domains are known in the art and are easily identified using online tools such as InterPro (https://www.ebi.ac.uk/interpro/). For example, in some instances, a RuvC-like domain is a domain which shares homology with a region of TnpB proteins of the IS605 and other related families of transposons.

The terms, “single guide nucleic acid”, “single guide RNA” and “sgRNA,” as used herein, in the context of a single nucleic acid system, refers to a guide nucleic acid, wherein the guide nucleic acid is a single polynucleotide chain having all the required sequence for a functional complex with an effector protein (e.g., being bound by an effector protein, including in some instances activating the effector protein, and hybridizing to a target nucleic acid, without the need for a second nucleic acid molecule). For example, a sgRNA can have two or more linked guide nucleic acid components (e.g., an intermediary sequence, a repeat sequence, a spacer sequence and optionally a linker, or a handle sequence and a spacer sequence).

The term, “spacer sequence,” as used herein, refers to a nucleotide sequence in a guide nucleic acid that is capable of, at least partially, hybridizing to an equal length portion of a sequence (e.g., a target sequence) of a target nucleic acid.

The term, “subject,” as used herein, refers to an animal. In some instances, the subject is a mammal. In some instances, the subject is a human. In some instances, the subject is diagnosed or at risk for a disease.

The term, “sufficiently complementary,” as used herein, refers to a first nucleotide sequence that is partially complementarity to a second nucleotide sequence while still allowing the first nucleotide sequence to hybridize to the second nucleotide sequence with enough affinity to permit a biological activity to occur. Depending on the context, in some instances, a biological activity comprises the formation of a complex between two or more components described herein, such as an effector protein and a guide nucleic acid. In some instances, a biological activity comprises bringing one or more components described herein into proximity of another component, such as bringing an effector protein-guide nucleic acid complex into proximity of a target nucleic acid. In some instances, a biological activity also comprises permitting a component described herein to act on another component described herein, such as permitting an effector protein to cleave a target nucleic acid. In some instances, sequences are said to be sufficiently complementary when at least 85% of the residues of a nucleotide sequence are complementary to residues in a reference nucleotide sequence.

The term, “syndrome,” as used herein, refers to a group of symptoms which, taken together, characterize a condition.

The term, “target nucleic acid,” as used herein, refers to a nucleic acid that is selected as the nucleic acid for editing, binding, hybridization or any other activity of or interaction with a nucleic acid, protein, polypeptide, or peptide described herein. In some instances, target nucleic acid comprises RNA, DNA, or a combination thereof. In some instances, a target nucleic acid is single-stranded (e.g., single-stranded RNA or single-stranded DNA) or double-stranded (e.g., double-stranded DNA).

The term, “target sequence,” as used herein, in the context of a target nucleic acid, refers to a nucleotide sequence found within a target nucleic acid. Such a nucleotide sequence can, for example, hybridize to a respective length portion of a guide nucleic acid.

The terms, “target strand” and “TS,” as used herein, in the context of a target nucleic acid being either a single stranded target nucleic acid or a double stranded target nucleic acid, refer to the nucleotide strand that comprises a target sequence to which at least a portion of a guide nucleic acid (e.g., a spacer sequence) is capable of, at least partially, hybridizing to an equal length portion of the target sequence. The terms, “non-target strand” and “NTS,” as used herein, in the context of a target nucleic acid being a double stranded target nucleic acid, refer to the nucleotide strand to which a guide nucleic acid is not capable of hybridizing to. The terms target strand and non-target strand differentiate between the strands of a double stranded target nucleic acid to which a guide nucleic acid is capable of or not capable of hybridizing. Reference may be made to a target sequence present in the target strand or the non-target strand of a double stranded target nucleic acid.

The term, “transcriptional activator,” as used herein, refers to a polypeptide or a fragment thereof that can activate or increase transcription of a target nucleic acid molecule.

The term, “transcriptional repressor,” as used herein, refers to a polypeptide or a fragment thereof that is capable of arresting, preventing, or reducing transcription of a target nucleic acid.

The term, “transgene,” as used herein, refers to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. The cell in which transgene expression occurs can be a target cell, such as a host cell.

The terms, “treatment” and “treating,” as used herein, refer to a pharmaceutical or other intervention regimen for obtaining beneficial or desired results in the recipient. Beneficial or desired results include but are not limited to a therapeutic benefit and/or a prophylactic benefit. In some instances, a therapeutic benefit refers to eradication or amelioration of symptoms or of an underlying disorder being treated. Also, in some instances, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, in some instances, a subject at risk of developing a particular disease, or to a subject reporting one or more of the physiological symptoms of a disease undergoes treatment, even though a diagnosis of this disease has not been made.

The term, “variant,” as used herein, refers to a form or version of a protein that differs from the wild-type protein. In some instances, a variant comprises a different function or activity relative to the wild-type protein.

The term, “viral vector,” as used herein, refers to a nucleic acid to be delivered into a host cell by a recombinantly produced virus or viral particle.

### II. Introduction

Disclosed herein are compositions, systems, and methods comprising at least one of: (a) a polypeptide or a nucleic acid encoding the polypeptide; and a guide nucleic acid or a nucleic acid encoding the guide nucleic acid. Also disclosed herein are methods of using such compositions and systems for modifying target nucleic acids in a hematopoietic stem cell (HSC). In some embodiments, a reference to the HSC also comprises a population of hematopoietic stem cells and progenitor cells (HSPCs). In some embodiments, the guide nucleic acid is capable of hybridizing to a target sequence of target nucleic acid present in the HSC.

Polypeptides described herein may bind and, optionally, cleave nucleic acids in a sequence-specific manner. Polypeptides described herein may also cleave the target nucleic acid within a target sequence or at a position adjacent to the target sequence. In some embodiments, a polypeptide is activated when it binds a certain sequence of a nucleic acid described herein, allowing the polypeptide to cleave a region of a target nucleic acid that is near, but not adjacent to the target sequence. A polypeptide may be an effector protein, such as a CRISPR-associated (Cas) protein, which may bind a guide nucleic acid that imparts activity or sequence selectivity to the polypeptide. An effector protein may also be referred to as a programmable nuclease because the nuclease activity of the protein may be directed to different target nucleic acids by way of revising the guide nucleic acid that the protein binds.

In some embodiments, compositions, systems, and methods comprising guide nucleic acids comprise a first region or nucleotide sequence, at least a portion of which interacts with a polypeptide. In some embodiments, the first nucleotide sequence comprises a sequence that is similar or identical to an intermediary nucleic acid sequence, a handle, a repeat sequence, or a combination thereof. In some embodiments, the guide nucleic acid does not comprise an intermediary nucleic acid. In some embodiments, compositions, systems, and methods comprising guide nucleic acids comprise a second nucleotide sequence that is at least partially complementary to a target nucleic acid, and which, in some embodiments, is referred to as a spacer sequence.

In some embodiments, effector proteins disclosed herein binds and/or cleaves nucleic acids, including double stranded RNA (dsRNA), single-stranded RNA (ssRNA), double stranded DNA (dsDNA), and single-stranded DNA (ssDNA). In some embodiments, polypeptides disclosed herein provides binding activity, cis cleavage activity, nickase activity, nuclease activity, integrase activity, recombinase activity or a combination thereof.

The compositions, systems, and methods described herein are non-naturally occurring. In some embodiments, compositions, systems, and methods comprise an engineered guide nucleic acid (also referred to herein as a guide nucleic acid) or a use thereof. In some embodiments, compositions, systems, and methods comprise an engineered protein or a use thereof. In some embodiments, compositions, systems, and methods comprise an isolated polypeptide or a use thereof. In general, compositions, methods, and systems described herein are not found in nature. In some embodiments, compositions, methods, and systems described herein comprise at least one non-naturally occurring component. For example, in some embodiments, disclosed compositions, methods, and systems comprise a guide nucleic acid, wherein the nucleotide sequence of the guide nucleic acid is different or modified from that of a naturally-occurring guide nucleic acid.

In some embodiments, compositions, systems, and methods comprise at least two components that do not naturally occur together. For example, in some embodiments, disclosed compositions, systems, and methods comprise a guide nucleic acid comprising a first region, at least a portion of which, interacts with a polypeptide, and a second region that is at least partially complementary to a target sequence in a target nucleic acid, wherein the first region and second region do not naturally occur together and/or are heterologous to each other. Also, by way of non-limiting example, in some embodiments, disclosed compositions, systems, and methods comprise a guide nucleic acid and an effector protein that do not naturally occur together. Likewise, by way of non-limiting example, disclosed compositions, systems, and methods comprise a ribonucleotide-protein (RNP) complex comprising an effector protein and a guide nucleic acid that do not occur together in nature. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, the guide nucleic acid comprises a non-natural nucleotide sequence. In some embodiments, the non-natural nucleotide sequence is a nucleotide sequence that is not found in nature. In some embodiments, the non-natural nucleotide sequence comprises a portion of a naturally-occurring nucleotide sequence, wherein the portion of the naturally-occurring sequence is not present in nature absent the remainder of the naturally-occurring nucleotide sequence. In some embodiments, the guide nucleic acid comprises two naturally-occurring nucleotide sequences arranged in an order or proximity that is not observed in nature. In some embodiments, compositions and systems comprise a ribonucleotide complex comprising an effector protein and a guide nucleic acid that do not occur together in nature. In some embodiments, compositions and systems comprise at least two components that do not occur together in nature, wherein the at least two components comprise at least one of an effector protein, an effector partner and a guide nucleic acid. In some embodiments, guide nucleic acids comprise a first nucleotide sequence and a second nucleotide sequence that do not occur naturally together. For example, in some embodiments, a guide nucleic acid comprises a naturally-occurring repeat sequence and a spacer sequence that is complementary to a naturally-occurring eukaryotic nucleotide sequence. In some embodiments, the guide nucleic acid comprises a repeat sequence that occurs naturally in an organism and a spacer sequence that does not occur naturally in that organism. In some embodiments, a guide nucleic acid comprises a first nucleotide sequence that occurs in a first organism and a second nucleotide sequence that occurs in a second organism, wherein the first organism and the second organism are different. In some embodiments, the guide nucleic acid comprises a third nucleotide sequence disposed at a 3′ or 5′ end of the guide nucleic acid, or between the first and second nucleotide sequences of the guide nucleic acid. In some embodiments, the guide nucleic acid comprises two heterologous nucleotide sequences arranged in an order or proximity that is not observed in nature. Therefore, compositions and systems described herein are not naturally occurring.

In some embodiments, compositions, systems, and methods described herein comprise a polypeptide (e.g., an effector protein, an effector partner, a fusion protein, or a combination thereof) that is similar to a naturally occurring polypeptide. In some embodiments, the polypeptide lacks a portion of the naturally occurring polypeptide. In some embodiments, the polypeptide comprises a mutation relative to the naturally-occurring polypeptide, wherein the mutation is not found in nature. In some embodiments, the polypeptide also comprises at least one additional amino acid relative to the naturally-occurring polypeptide. In some embodiments, the polypeptide comprises a heterologous polypeptide. For example, in some embodiments, the polypeptide comprises an addition of a nuclear localization signal relative to the natural occurring polypeptide. In some embodiments, a nucleotide sequence encoding the polypeptide is codon optimized (e.g., for expression in a eukaryotic cell) relative to the naturally occurring sequence.

### III. Polypeptide Systems

Provided herein are compositions, systems and methods comprising a polypeptide or polypeptide system, wherein the polypeptide or polypeptide system described herein comprises one or more effector proteins or variants thereof, one or more effector partners or variants thereof, one or more linkers for peptides, or combinations thereof.

Also provided herein are compositions, systems and methods comprising a polypeptide, wherein the polypeptide comprises an effector protein, an effector partner, a fusion protein or a combination thereof.

### Effector Proteins

Provided herein are compositions, systems, and methods comprising an effector protein or a use thereof. In some embodiments, an effector protein provided herein interacts with a guide nucleic acid to form a complex. In some embodiments, the complex interacts with a target nucleic acid. In some embodiments, an interaction between the complex and a target nucleic acid comprises one or more of: recognition of a protospacer adjacent motif (PAM) sequence within the target nucleic acid by the effector protein, hybridization of the guide nucleic acid to the target nucleic acid, modification of the target nucleic acid by the effector protein, or combinations thereof. In some embodiments, recognition of a PAM sequence within a target nucleic acid directs the modification activity of an effector protein. In some embodiments, recognition of a PAM sequence adjacent to a target sequence of a target nucleic acid directs the modification activity of an effector protein.

Modification activity of an effector protein or an engineered protein described herein comprises cleavage activity, binding activity, insertion activity, substitution activity, and the like. In some embodiments, modification activity of an effector protein results in: cleavage of at least one strand of a target nucleic acid, deletion of one or more nucleotides of a target nucleic acid, insertion of one or more nucleotides into a target nucleic acid, substitution of one or more nucleotides of a target nucleic acid with an alternative nucleotide, more than one of the foregoing, or any combination thereof. In some embodiments, modification of a target nucleic acid comprises introducing or removing epigenetic modification(s). In some embodiments, an ability of an effector protein to edit a target nucleic acid depends upon the effector protein being complexed with a guide nucleic acid, the guide nucleic acid being hybridized to a target sequence of the target nucleic acid, the distance between the target sequence and a PAM sequence, or combinations thereof. A target nucleic acid comprises a target strand and a non-target strand. Accordingly, in some embodiments, the effector protein edits a target strand and/or a non-target strand of a target nucleic acid.

The modification of a target nucleic acid generated by an effector protein, as a non-limiting example, results in modulation of the expression of the target nucleic acid (e.g., increasing or decreasing expression of the nucleic acid) or modulation of the activity of a translation product of the target nucleic acid (e.g., inactivation of a protein binding to an RNA molecule or hybridization). Accordingly, in some embodiments, provided herein are methods of editing a target nucleic acid using an effector protein of the present disclosure, or compositions or systems thereof. Also provided herein are methods of modulating expression of a target nucleic acid using an effector protein of the present disclosure, or compositions or systems thereof. Further provided herein are methods of modulating the activity of a translation product of a target nucleic acid using an effector protein of the present disclosure, or compositions or systems thereof.

In some embodiments, effector proteins disclosed herein provide nucleic acid cleavage activity. In some embodiments, effector proteins provide nuclease activity. In some embodiments, effector proteins provide nickase activity. In general, effector proteins described herein edit a target nucleic acid by cis cleavage activity on the target nucleic acid. In some embodiments, effector proteins disclosed herein comprise a RuvC domain capable of cleavage activity. Effector proteins disclosed herein cleave nucleic acids, including single stranded RNA (ssRNA), double stranded DNA (dsDNA), and single-stranded DNA (ssDNA).

In some embodiments, effector proteins disclosed herein provide catalytic activity (e.g., nuclease activity) similar to that of a naturally-occurring effector protein, such as, for example, a naturally-occurring effector protein with reduced cleavage activity including cis cleavage activity. In some embodiments, effector proteins disclosed herein are fused to effector partners or fusion proteins wherein the effector partners or fusion proteins are capable of some function or activity not provided by an effector protein.

In some embodiments, an effector protein comprises a CRISPR-associated (“Cas”) protein. In some embodiments, an effector protein functions as a single protein, including a single protein that is capable of binding to a guide nucleic acid and editing a target nucleic acid. Alternatively, in some embodiments, an effector protein functions as part of a multiprotein complex, including, for example, a complex having two or more effector proteins, including two or more of the same effector proteins (e.g., dimer or multimer). In some embodiments, an effector protein, when functioning in a multiprotein complex, comprises only one functional activity (e.g., binding to a guide nucleic acid), while other effector proteins present in the multiprotein complex are capable of the other functional activity (e.g., editing a target nucleic acid). In some embodiments, an effector protein, when functioning in a multiprotein complex, comprises differing and/or complementary functional activity to other effector proteins in the multiprotein complex. Multimeric complexes, and functions thereof, are described in further detail below. In some embodiments, an effector protein comprises a modified effector protein having increased modification activity and/or increased substrate binding activity (e.g., substrate selectivity, specificity, and/or affinity). Alternatively, or in addition, an effector protein comprises a catalytically inactive effector protein having reduced modification activity or no modification activity.

TABLE 1 provides illustrative amino acid sequences of effector proteins that are useful in the compositions, systems and methods described herein. In some embodiments, an effector protein, or a recombinant nucleic acid encoding an effector protein, comprises an amino acid sequence that is at least 85% identical to any one of the amino acid sequences set forth in TABLE 1. In some embodiments, the recombinant nucleic acid encoding the effector protein is operably linked to a promoter, wherein the promoter is functional in a eukaryotic cell or a prokaryotic cell. In some embodiments, the promoter is any one or more of: a constitutive promoter, an inducible promoter, a cell type-specific promoter, and a tissue-specific promoter. In some embodiments, the recombinant nucleic acid described herein wherein the promoter is functional in any one of: a plant cell, a fungal cell, an animal cell, cell of an invertebrate, a fly cell, a cell of a vertebrate, a mammalian cell, a primate cell, a non-human primate cell, and a human cell. In some embodiments, the recombinant nucleic acid is a nucleic acid expression vector as described herein.

In some embodiments, compositions, systems and methods described herein comprise an effector protein, or a nucleic acid encoding the effector protein, wherein the amino acid sequence of the effector protein comprises at least about 200 contiguous amino acids or more of any one of the amino acid sequences recited in TABLE 1. In some embodiments, the amino acid sequence of an effector protein provided herein comprises at least about 200, at least about 220, at least about 240, at least about 260, at least about 280, at least about 300, at least about 320, at least about 340, at least about 360, at least about 380, at least about 400 contiguous amino acids, at least about 420 contiguous amino acids, at least about 440 contiguous amino acids, at least about 460 contiguous amino acids, at least about 480 contiguous amino acids, at least about 500 contiguous amino acids, at least about 520 contiguous amino acids, at least about 540 contiguous amino acids, at least about 560 contiguous amino acids, at least about 580 contiguous amino acids, at least about 600 contiguous amino acids, at least about 620 contiguous amino acids, at least about 640 contiguous amino acids, at least about 660 contiguous amino acids, at least about 680 contiguous amino acids, at least about 700 contiguous amino acids of any one of the amino acid sequences of TABLE 1.

In some embodiments, compositions, systems and methods described herein comprise an effector protein or a nucleic acid encoding the effector protein, wherein the effector protein comprises a portion of any one of the amino acid sequences recited in TABLE 1. In some embodiments, the effector protein comprises a portion of any one of the amino acid sequences recited in TABLE 1, wherein the portion does not comprise at least the first 10 amino acids, at least the first 20 amino acids, at least the first 40 amino acids, at least the first 60 amino acids, at least the first 80 amino acids, at least the first 100 amino acids, at least the first 120 amino acids, at least the first 140 amino acids, at least the first 160 amino acids, at least the first 180 amino acids, or at least the first 200 amino acids of any one of the amino acid sequences recited in TABLE 1. In some embodiments, the effector protein comprises a portion of any one of the amino acid sequences recited in TABLE 1, wherein the portion does not comprise the last 10 amino acids, the last 20 amino acids, the last 40 amino acids, the last 60 amino acids, the last 80 amino acids, the last 100 amino acids, the last 120 amino acids, the last 140 amino acids, the last 160 amino acids, the last 180 amino acids, or the last 200 amino acids of any one of the amino acid sequences recited in TABLE 1.

In some embodiments, compositions, systems, and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 97%, at least 98%, at least 99%, or 100% identical to any one of the amino acid sequences as set forth in TABLE 1. In some embodiments, an effector protein provided herein comprises an amino acid sequence that is at least 65% identical to any one of the amino acid sequences as set forth in TABLE 1. In some embodiments, an effector protein provided herein comprises an amino acid sequence that is at least 70% identical to any one of the amino acid sequences as set forth in TABLE 1. In some embodiments, an effector protein provided herein comprises an amino acid sequence that is at least 75% identical to any one of the amino acid sequences as set forth in TABLE 1. In some embodiments, an effector protein provided herein comprises an amino acid sequence that is at least 80% identical to any one of the amino acid sequences as set forth in TABLE 1. In some embodiments, an effector protein provided herein comprises an amino acid sequence that is at least 85% identical to any one of the amino acid sequences as set forth in TABLE 1. In some embodiments, an effector protein provided herein comprises an amino acid sequence that is at least 90% identical to any one of the amino acid sequences as set forth in TABLE 1. In some embodiments, an effector protein provided herein comprises an amino acid sequence that is at least 95% identical to any one of the amino acid sequences as set forth in TABLE 1. In some embodiments, an effector protein provided herein comprises an amino acid sequence that is at least 97% identical to any one of the amino acid sequences as set forth in TABLE 1. In some embodiments, an effector protein provided herein comprises an amino acid sequence that is at least 98% identical to any one of the amino acid sequences as set forth in TABLE 1. In some embodiments, an effector protein provided herein comprises an amino acid sequence that is at least 99% identical to any one of the amino acid sequences as set forth in TABLE 1. In some embodiments, an effector protein provided herein comprises an amino acid sequence that is identical to any one of the amino acid sequences as set forth in TABLE 1.

In some embodiments, compositions, systems, and methods described herein comprise an effector protein, or a nucleic acid encoding the effector protein, wherein the effector protein comprises an amino acid sequence that is at least 80%, at least 85%, at least 90%, at least 95%, at least 97%, at least 98%, at least 99%, or 100% similar to any one of the amino acid sequences as set forth in TABLE 1. In some embodiments, an effector protein provided herein comprises an amino acid sequence that is at least 80% similar to any one of the amino acid sequences as set forth in TABLE 1. In some embodiments, an effector protein provided herein comprises an amino acid sequence that is at least 85% similar to any one of the amino acid sequences as set forth in TABLE 1. In some embodiments, an effector protein provided herein comprises an amino acid sequence that is at least 90% similar to any one of the amino acid sequences as set forth in TABLE 1. In some embodiments, an effector protein provided herein comprises an amino acid sequence that is at least 95% similar to any one of the amino acid sequences as set forth in TABLE 1. In some embodiments, an effector protein provided herein comprises an amino acid sequence that is at least 97% similar to any one of the amino acid sequences as set forth in TABLE 1. In some embodiments, an effector protein provided herein comprises an amino acid sequence that is at least 98% similar to any one of the amino acid sequences as set forth in TABLE 1. In some embodiments, an effector protein provided herein comprises an amino acid sequence that is at least 99% similar to any one of the amino acid sequences as set forth in TABLE 1. In some embodiments, an effector protein provided herein comprises an amino acid sequence that is 100% similar to any one of the amino acid sequences as set forth in TABLE 1.

In some embodiments, compositions, systems, and methods described herein comprise an effector protein, or a nucleic acid encoding the effector protein, wherein the effector protein comprises one or more amino acid alterations relative to any one of the amino acid sequences recited in TABLE 1. In some embodiments, the one or more alterations comprises at least one, at least two, at least three, at least four, at least five, at least six, at least seven, at least eight, at least nine, at least ten, at least twelve, at least sixteen, at least twenty, or more amino acid alterations relative to any one of the amino acid sequences recited in TABLE 1. In some embodiments, the one or more alterations comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty amino acid alterations relative to any one of the amino acid sequences recited in TABLE 1. In some embodiments, the one or more alterations comprises one, two, three, four, five, six, seven, eight, nine, ten, or more amino acid alterations relative to any one of the amino acid sequences recited in TABLE 1. In some embodiments, the effector protein comprising one or more amino acid alterations is a variant of an effector protein described herein. It is understood that any reference to an effector protein herein also refers to an effector protein variant as described herein. In some embodiments, the one or more amino acid alterations comprises conservative substitutions, non-conservative substitutions, deletions, or combinations thereof. In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to any one of the amino acid sequences recited in TABLE 1.

In some embodiments, compositions, systems, and methods described herein comprise an effector protein, or a nucleic acid encoding the effector protein, wherein the effector protein comprises one or more substitutions relative to any one of the amino acid sequences recited in TABLE 1. In some embodiments, the one or more substitutions comprises at least one, at least two, at least three, at least four, at least five, at least six, at least seven, at least eight, at least nine, at least ten, at least twelve, at least sixteen, at least twenty, or more substitutions relative to any one of the amino acid sequences recited in TABLE 1. In some embodiments, the one or more substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty substitutions relative to any one of the amino acid sequences recited in TABLE 1. In some embodiments, the one or more substitutions comprise one, two, three, four, five, six, seven, eight, nine, ten or more substitutions relative to any one of the amino acid sequences recited in TABLE 1. In some embodiments, the one or more substitutions comprise one or more conservative substitutions, one or more non-conservative substitutions, or combinations thereof. As a non-limiting example, a conservative substitution of a basic amino acid of any one of the amino acid sequences recited in TABLE 1 is a substitution by another basic (positively charged) amino acid (e.g., Lys (K), Arg (R), or His (H)). As a non-limiting example, a non-conservative substitution of acidic (negatively charged) amino acid of any one of the amino acid sequences recited in TABLE 1 is a substitution by a basic (positively charged) amino acid (e.g., Lys (K), Arg (R), or His (H)).

In some embodiments, compositions, systems, and methods described herein comprise an effector protein, or a nucleic acid encoding the effector protein, wherein the effector protein comprises one or more conservative substitutions relative to any one of the amino acid sequences recited in TABLE 1. In some embodiments, the one or more conservative substitutions comprises at least one, at least two, at least three, at least four, at least five, at least six, at least seven, at least eight, at least nine, at least ten, at least twelve, at least sixteen, at least twenty, or more conservative substitutions relative to any one of the amino acid sequences recited in TABLE 1. In some embodiments, the one or more conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty conservative substitutions relative to any one of the amino acid sequences recited in TABLE 1. In some embodiments, the one or more conservative substitutions comprise one, two, three, four, five, six, seven, eight, nine, ten or more conservative substitutions relative to any one of the amino acid sequences recited in TABLE 1.

In some embodiments, compositions, systems, and methods described herein comprise an effector protein, or a nucleic acid encoding the effector protein, wherein the effector protein comprises one or more non-conservative substitutions relative to any one of the amino acid sequences recited in TABLE 1. In some embodiments, the one or more non-conservative substitutions comprises at least one, at least two, at least three, at least four, at least five, at least six, at least seven, at least eight, at least nine, at least ten, at least twelve, at least sixteen, at least twenty, or more non-conservative substitutions relative to any one of the amino acid sequences recited in TABLE 1. In some embodiments, the one or more non-conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty non-conservative substitutions relative to any one of the amino acid sequences recited in TABLE 1. In some embodiments, the one or more non-conservative substitutions comprise one, two, three, four, five, six, seven, eight, nine, ten or more non-conservative substitutions relative to any one of the amino acid sequences recited in TABLE 1.

In some embodiments, an effector protein disclosed herein comprises an amino acid sequence that is at least about 80%, at least about 85%, at least about 90%, at least about 95%, at least about 98%, or at least about 99% identical to any one of the amino acid sequences recited in TABLE 1, wherein all but 1, 2, 3, 4, 5, 6, 7, 8, 9 or 10 amino acids alterations relative to the sequence in TABLE 1 are conservative amino acid substitutions. In some embodiments, an effector protein disclosed herein comprises an amino acid sequence that is at least about 80%, at least about 85%, at least about 90%, at least about 95%, at least about 98%, or at least about 99% identical to any one of the amino acid sequences recited in TABLE 1, wherein all but 1, 2, 3, 4, 5, 6, 7, 8, 9 or 10 amino acids alterations relative to the sequence in TABLE 1 are non-conservative amino acid substitutions. In some embodiments, an effector protein disclosed herein comprises an amino acid sequence that is identical to any one of the amino acid sequences recited in TABLE 1 with the exception of 1, 2, 3, 4, 5, 6, 7, 8, 9 or 10 conservative amino acid alterations. In some embodiments, an effector protein disclosed herein comprises an amino acid sequence that is identical to any one of the amino acid sequences recited in TABLE 1 with the exception of 1, 2, 3, 4, 5, 6, 7, 8, 9 or 10 non-conservative amino acid alterations.

In some embodiments, an effector protein disclosed herein comprises an amino acid sequence that is at least about 80%, at least about 85%, at least about 90%, at least about 95%, at least about 98%, at least about 99% or 100% similar to any one of the amino acid sequences recited in TABLE 1, wherein all but 1, 2, 3, 4, 5, 6, 7, 8, 9 or 10 amino acids alterations relative to the sequence in TABLE 1 are conservative amino acid substitutions. In some embodiments, an effector protein disclosed herein comprises an amino acid sequence that is at least about 80%, at least about 85%, at least about 90%, at least about 95%, at least about 98%, at least about 99% or 100% similar to any one of the amino acid sequences recited in TABLE 1, wherein all but 1, 2, 3, 4, 5, 6, 7, 8, 9 or 10 amino acids alterations relative to the sequence in TABLE 1 are non-conservative amino acid substitutions. In some embodiments, an effector protein disclosed herein comprises an amino acid sequence that is similar to any one of the amino acid sequences recited in TABLE 1 with the exception of 1, 2, 3, 4, 5, 6, 7, 8, 9 or 10 conservative amino acid alterations. In some embodiments, an effector protein disclosed herein comprises an amino acid sequence that is similar to any one of the amino acid sequences recited in TABLE 1 with the exception of 1, 2, 3, 4, 5, 6, 7, 8, 9 or 10 non-conservative amino acid alterations.

In some embodiments, the one or more amino acid alterations result in a change in activity of the effector protein relative to a naturally-occurring counterpart (a WT effector protein (e.g., SEQ ID NO: 1 or 2)). For example, and as described in further detail below, the one or more amino acid alteration increases or decreases binding activity of the effector protein relative to a naturally-occurring counterpart. In some embodiments, the one or more amino acid alteration increases or decreases catalytic activity of the effector protein relative to a naturally-occurring counterpart (a WT effector protein (e.g., SEQ ID NO: 1 or 2)). In some embodiments, the one or more amino acid alterations results in a catalytically inactive effector protein variant. In some embodiments, the effector proteins comprising the one or more amino acid alterations can carry out a similar enzymatic reaction as the naturally-occurring counterpart (a WT effector protein (e.g., SEQ ID NO: 1 or 2)).

In some embodiments, the variants of the effector protein as described herein can include alterations that provide a beneficial characteristic to effector proteins described herein, including but not limited to, increased activity (e.g., indel activity, catalytic activity, specificity or selectivity and/or affinity for a substrate, such as a target nucleic acid and/or a guide nucleic acid). In some embodiments, variants of effector proteins described herein can exhibit an activity that is at least the same or higher than the WT effector protein (e.g., SEQ ID NO: 1 or 2), that is, it has one or more activities that are the same or higher than the effector protein (e.g., SEQ ID NO: 1 or 2) without the variant at the same amino acid position(s). For example, variants can have one or more activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher over a WT effector protein (e.g., SEQ ID NO: 1 or 2). In some embodiments, activity of effector proteins described herein or variants thereof can be measured relative to a WT effector protein (e.g., SEQ ID NO: 1 or 2) in a cleavage assay.

In some embodiments, the effector proteins described herein comprises a substitution of at least one, at least two, at least three, at least four, at least five, at least six, at least seven, at least eight, at least nine, or at least ten amino acids with positively charged amino acids. In some embodiments, the effector proteins described herein comprises a substitution of one, two, three, four, five, six, seven, eight, nine, or ten amino acids with positively charged amino acids. In some embodiments, the effector protein comprises an amino acid sequence that is at least 65%, at least 70%, at least 75%, at least 80%, at least 85%, at least 90%, at least 95%, at least 98%, at least 99%, or 100% identical to SEQ ID NO: 1, wherein the effector protein comprises one or more amino acid alterations independently at the positions selected from K58, I80, T84, K105, N193, C202, S209, G210, A218, D220, E225, C246, N286, M295, M298, A306, Y315, Q360, or a combination thereof. In some embodiments, the effector protein comprises an amino acid sequence that is at least 60%, at least 65%, at least 70%, at least 75%, at least 80%, at least 85%, at least 90%, at least 92%, at least 95%, at least 97%, at least 98%, at least 99%, or 100% similar to SEQ ID NO: 1, and wherein the effector protein also comprises one or more alterations independently at the positions selected from K58, I80, T84, K105, N193, C202, S209, G210, A218, D220, E225, C246, N286, M295, M298, A306, Y315, Q360, or a combination thereof. In some embodiments, the one or more amino acid alterations comprise one or more substitutions independently selected from K58R, K58K, K58H, I80R, I80K, I80H, T84R, T84K, T84H, K105R, K105K, K105H, N193R, N193K, N193H, C202R, C202K, C202H, S209R, S209K, S209H, G210R, G210K, G210H, A218R, A218K, A218H, D220R, D220K, D220H, E225R, E225K, E225H, C246R, C246K, C246H, N286R, N286K, N286H, M295R, M295K, M295H, M298R, M298K, M298H, A306R, A306K, A306H, Y315R, Y315K, Y315H, Q360R, Q360K, Q360H, N58K, or a combination thereof relative to the amino acid sequence of SEQ ID NO: 1. In some embodiments, the one or more amino acid alterations comprise one or more substitutions independently selected from I80R, T84R, K105R, C202R, G210R, A218R, D220R, E225R, C246R, Q360R, or a combination thereof relative to the amino acid sequence of SEQ ID NO: 1. In some embodiments, the one or more amino acid alterations comprise one or more substitutions independently selected from I80K, T84K, C202K, G210K, A218K, D220K, E225K, C246K, Q360K, or a combination thereof relative to the amino acid sequence of SEQ ID NO: 1. In some embodiments, the one or more amino acid alteration comprises one or more substitutions independently selected from I80H, T84H, K105H, G210H, C202H, A218H, D220H, E225H, C246H, Q360H, or a combination thereof relative to the amino acid sequence of SEQ ID NO: 1. In some embodiments, the one or more amino acid alterations comprise one or more substitutions independently selected from K58W, I80R, T84R, K105R, N193K, C202R, S209F, G210R, A218K, A218R, D220R, E225K, E225R, C246R, N286K, M295W, M298L, A306K, Y315M, Q360R, or a combination thereof relative to the amino acid sequence of SEQ ID NO: 1. In some embodiments, the one or more alterations comprise one or more substitutions independently selected from K58W, I80K, N193K, S209F, A218R, E225K, N286K, M295W, M298L, A306K, Y315M, or a combination thereof relative to the amino acid sequence of SEQ ID NO: 1. In some embodiments, the one or more alterations comprise one or more substitutions independently selected from D237A, D418A, D418N, E335A, and E335Q, or a combination thereof relative to the amino acid sequence of SEQ ID NO: 1. In some embodiments, the one or more amino acid alterations comprise two simultaneous amino acid substitutions selected from D220R/A306K, D220R/K250N, D418A/K58W, and D418N/K58W relative to SEQ ID NO: 1. In some embodiments, the one or more amino acid alterations comprise one or more substitutions relative to SEQ ID NO: 1, wherein the effector protein comprising one or more substitutions has enhanced nuclease activity

In some embodiments, the effector proteins described herein comprises a substitution of at least one, at least two, at least three, at least four, at least five, at least six, at least seven, at least eight, at least nine, or at least ten amino acids with positively charged amino acids. In some embodiments, the effector proteins described herein comprises a substitution of one, two, three, four, five, six, seven, eight, nine, or ten amino acids with positively charged amino acids. In some embodiments, the effector protein comprises an amino acid sequence that is at least 65%, at least 70%, at least 75%, at least 80%, at least 85%, at least 90%, at least 95%, at least 98%, at least 99%, or 100% identical to SEQ ID NO: 2, wherein the effector protein comprises one or more amino acid alterations independently at the positions selected from I2, T5, K15, R18, H20, S21, L26, N30, E33, E34, A35, K37, K38, R41, N43, Q54, Q79R, K92E, K99R, S108, E109, H110, G111, D113, T114, P116, K118, E119, A121, N132, K135, Q138, V139, L149, E157, E164, E166, E170, Y180, L182, Q183, K184, S186, K189, S196, S198, K200, I203, S205, K206, Y207, H208, N209, Y220, S223, E258, K281, K348, N355, N406, K435, 1471, 1489, Y490, F491, D495, K496, K498, K500, D501, V502, K504, S505, D506, V521, N568, S579, Q612, S638, F701, P707, or any combinations thereof. In some embodiments, the effector protein comprises an amino acid sequence that is at least 65%, at least 70%, at least 75%, at least 80%, at least 85%, at least 90%, at least 95%, at least 98%, at least 99%, or 100% similar to SEQ ID NO: 2, wherein the effector protein comprises one or more amino acid alterations independently at the positions selected from I2, T5, K15, R18, H20, S21, L26, N30, E33, E34, A35, K37, K38, R41, N43, Q54, Q79R, K92E, K99R, S108, E109, H110, G111, D113, T114, P116, K118, E119, A121, N132, K135, Q138, V139, L149, E157, E164, E166, E170, Y180, L182, Q183, K184, S186, K189, S196, S198, K200, 1203, S205, K206, Y207, H208, N209, Y220, S223, E258, K281, K348, N355, N406, K435, 1471, 1489, Y490, F491, D495, K496, K498, K500, D501, V502, K504, S505, D506, V521, N568, S579, Q612, S638, F701, P707, or any combinations thereof. In some embodiments, the one or more amino acid alterations comprise one or more substitutions independently selected from I2R, I2K, I2H, 5K, T5H, T11R, G13R, G13H, G13K, F14R, F14H, F14K, K15R, K15H, L16R, L16H, L16K, L16F, I17R, I17H, I17K, R18H, R18K, R18R, N19R, N19H, N19K, N19V, H20R, H20K, S21R, S21H, S21K, R22H, R22K, R22R, T23R, T23H, T23K, A24R, A24H, A24K, G25R, G25H, G25K, L26H, L28R, L28H, L28K, K29R, K29H, N30R, N30H, N30K, E31R, E31H, E31K, G32R, G32H, G32K, E33R, E33H, E33K, E34R, E34H, E34K, A35R, A35H, A35K, C36R, C36H, C36K, K37R, K37H, K38R, K38H, F39R, F39H, F39K, V40R, V40H, V40K, R41H, R41K, R41R, E42R, E42H, E42K, N43R, N43H, N43K, N43A, E44R, E44H, E44K, P46S, D48Y, C50K, C50P, P51R, P51H, P51K, N52R, N52H, N52K, F53R, F53H, F53K, F509A, Q54H, Q54K, G55R, G55H, G55K, G56R, G56H, G56K, P57R, P57H, P57K, A58W, A58F, 159K, I59R, I62K, K65W, K65F, E68P, T70E, E71K, E73W, E73F, E73P, 174W, 174F, Y75W, Y75F, S77V, S78K, S78D, L79R, A80K, E83K, V84Y, V84K, T87G, T87D, P89T, P89K, K90E, P94E, P94Q, P94K, E95K, P96K, 197R, K99R, K99H, K99S, E100K, E101K, W102T, W106N, W106K, L107F, S108R, S108H, S108K, E109R, E109H, E109K, E109A, H110R, H110K, H110T, G111R, G111H, G111K, L112R, L112H, L112K, L112E, D113R, D113H, D113K, T114R, T114H, T114K, V115R, V115H, V115K, P116R, P116H, P116K, P116G, Y117R, Y117H, Y117K, K118R, K118H, E119R, E119H, E119K, A120R, A120H, A120K, A121R, A121H, A121K, G122R, G122H, G122K, L123R, L123H, L123K, N124R, N124H, N124K, L125R, L125H, L125K, 1126R, 1126H, 1126K, 1127R, 1127H, 1127K, K128R, K128H, N129R, N129H, N129K, A130R, A130H, A130K, V131R, V131H, V131K, N132R, N132H, N132K, T133R, T133H, T133K, Y134R, Y134H, Y134K, K135R, K135H, G136R, G136H, G136K, V137R, V137H, V137K, Q138R, Q138H, Q138K, V139H, V139K, K146N, N147K, N148E, L149R, L149H, L149K, A150K, K151E, N153R, E157R, I158K, A159K, A159N, K160D, G163E, S168W, F169F, E171K, F175K, G179R, G179H, G179K, Y180R, Y180H, Y180K, L181R, L181H, L181K, L182R, L182H, L182K, Q183R, Q183H, Q183K, K184R, K184H, K184P, P185R, P185H, P185K, S186H, S186K, S186G, S186E, S186R, P187R, P187H, P187K, P187I, N188R, N188H, N188K, K189R, K189H, S190R, S190H, S190K, S190V, I191R, I191H, I191K, Y192R, Y192H, Y192K, C193R, C193H, C193K, Y194R, Y194H, Y194K, Q195R, Q195H, Q195K, S196R, S196H, S196K, V197R, V197H, V197K, S198H, S198K, S198E, P199R, P199H, P199K, K200R, K200H, P201R, P201H, P201K, F202R, F202H, F202K, I203R, I203H, I203K, I203G, T204R, T204H, T204K, S205R, S205H, S205K, K206R, K206H, Y207R, Y207H, Y207K, H208R, H208K, N209R, N209H, N209K, N209D, N209W, N209F, V210R, V210H, V210K, G218S, G218K, Y220K, S223K, S223V, E225K, I227R, V228R, S229R, P230R, Y231K, Y231G, Y231R, Q232R, F233R, D234R, L236R, I238R, P239R, I240K, G241R, E242R, P243R, G244R, Y245R, V246K, V246R, P247R, K248E, K248R, Q250N, Q250R, T252D, T252R, F253R, L254R, S255R, K256E, K256R, K257R, E258R, N259R, K260R, R262D, L264R, S265R, K266R, I268R, S272Q, P273A, G276V, C279R, I280K, K281R, K281H, C285V, V286K, T295N, T295F, T295W, H297A, H297R, W298L, Y301L, H302F, H302W, P304E, P304K, D306K, D306F, L311T, F312L, F315M, F315K, T316R, T316H, T316K, V328N, R329T, M334E, 1338S, N340S, N340F, K348R, K348H, N355R, N355H, N355K, 1356W, 1356F, C357L, C357E, C357K, N360K, G361R, C363V, A366V, V368K, V370L, K384T, L390H, T391W, K392A, K392E, T393K, T393E, 1395Q, R397K, P399F, P399Q, T400L, C405L, C405R, K407E, F445S, S472R, K480L, V483G, G497K, G497R, D501K, M503K, W509A, Q511R, Q511H, Q511K, D512R, D512H, D512K, Y513R, Y513H, Y513K, K514R, K514H, P515R, P515H, P515K, K516R, K516H, L517R, L517H, L517K, D523K, S526N, E529K, E529L, E536A, R531E, S536A, N540R, N540H, N540K, K541R, K541H, L542R, L542H, L542K, S543R, S543H, S543K, K544R, K544H, S545R, S545H, S545K, R546H, R546K, R546R, D549L, G577H, G585R, Y590R, Y590H, Y590K, K591R, K591H, P592R, P592H, P592K, K593R, K593H, K594R, K594H, E595R, E595H, E595K, N596R, N596H, N596K, W599F, A602R, A602H, A602K, I603R, 1603H, 1603K, H604R, H604K, K605R, K605H, A606R, A606H, A606K, L607R, L607H, L607K, L607F, T608R, T608H, T608K, Q612R, R617Y, L620E, M624A, K634G, K639E, I653A, A673G, A673R, Q674R, Q674K, K678R, P679R, E682R, S684R, G685R, A696R, P699R, D703R, P707H, P707K, Y709R, E715R, A716R, K15K, H20H, K37K, K38K, Q79K, Q79H, K92R, K92K, K92H, K99K, H110H, K118K, K135K, N148R, N148K, N148H, E157K, E157H, E164R, E164K, E164H, E166R, E166K, E166H, E170R, E170K, E170H, K184K, K189K, K200K, K206K, H208H, Y220R, Y220H, S223R, S223H, E258H, K281K, K348K, S362R, S362K, S362H, N406R, N406H, K435R, K435K, K435H, I471R, I471K, I471H, 1489R, 1489K, 1489H, Y490R, Y490K, Y490H, F491R, F491K, F491H, D495R, D495K, D495H, K496R, K496K, K496H, K498R, K498K, K498H, K500R, K500K, K500H, D501R, D501H, V502R, V502K, V502H, K504R, K504K, K504H, S505R, S505K, S505H, D506R, D506K, D506H, V521R, V521K, V521H, N568R, N568K, N568H, S579K, S579H, Q612K, Q612H, S638R, S638H, F701K, F701H, or any combinations thereof relative to the amino acid sequence of SEQ ID NO: 2. In some embodiments, the one or more amino acid alterations comprise one or more substitutions independently selected from L26R, E157A, E164A, E164L, E166A, E166I, E170A, 1489A, 1489S, Y490S, Y490A, F491A, F491S, F491G, D495G, D495R, D495K, K496A, K496S, K498A, K498S, K500A, K500S, D501R, D501G, D501K, V502A, V502S, K504A, K504S, S505R, D506A, or any combinations thereof relative to the amino acid sequence of SEQ ID NO: 2. In some embodiments, the one or more amino acid alteration comprises T5R, L26K, A121Q, S198R, S223P, E258K, I471T, S579R, F701R, L26R, V139R, P707R, K189P, S638K, Q54R, Q79R, Y220S, N406K, E119S, K92E, K435Q, N568D, V521T, or any combinations thereof relative to the amino acid sequence of SEQ ID NO: 2. In some embodiments, the one or more amino acid alteration comprises D369A, D369N, D658A, D658N, E567A, E567Q, or any combinations thereof relative to the amino acid sequence of SEQ ID NO: 2. In some embodiments, the one or more amino acid alteration comprises E567A or E567Q relative to the amino acid sequence of SEQ ID NO: 2. In some embodiments, the one or more amino acid alterations comprise one or more substitutions independently selected from T5R, L26R, L26K, A121Q, V139R, S198R, S223P, E258K, I471T, S579R, F701R, P707R, K189P, S638K, Q54R, Q79R, Y220S, N406K, E119S, K92E, K435Q, N568D, and V521T, or any combinations thereof relative to the amino acid sequence of SEQ ID NO: 2. In some embodiments, the one or more amino acid alterations comprises two simultaneous amino acid substitutions selected from L26R/E109R, L26R/H208R, L26R/K184R, L26R/K38R, L26R/L182R, L26R/Q183R, L26R/S108R, L26R/S198R, L26R/T114R, E109R/H208R, E109R/K184R, E109R/K38R, E109R/L182R, E109R/Q183R, E109R/S108R, E109R/S198R, E109R/T114R, H208R/K184R, H208R/K38R, H208R/L182R, H208R/Q183R, H208R/S108R, H208R/S198R, H208R/T114R, K184R/K38R, K184R/E109R, K184R/H208R, K184R/L182R, K184R/Q183R, K184R/S108R, K184R/S198R, K184R/T114R, K38R/L182R, K38R/Q183R, K38R/S108R, K38R/S198R, K38R/T114R, L182R/Q183R, L182R/S108R, L182R/S198R, L182R/T114R, Q183R/S108R, Q183R/S198R, Q183R/T114R, S108R/S198R, S108R/T114R, S198R/T114R, S198R/K92E, L26R/1268R, L26R/G244R, F701R/K189P, Q612R/E119S, F701R/N406K, L26R/E109K, F701R/K435Q, L26R/Y220S, L26R/G57R, L26R/A716R, S579R/E119S, K348R/V521T, Q612R/S638K, K118R/K189P, F701R/V521T, K118R/K92E, Q612R/A121Q, F701R/E119S, L26R/P239R, L26R/Y231R, K348R/S223P, L26R/C279R, K348R/E119S, L26R/E100K, K348R/N406K, Q612R/E258K, L26R/S255R, F701R/K92E, Q612R/N406K, K348R/K189P, S186R/E258K, Q612R/Y220S, L26R/N568D, L26R/G241R, F701R/N568D, Q612R/K435Q, T5R/N568D, Q612R/V521T, S186R/V521T, K348R/A121Q, L26R/D234R, K348R/K92E, S579R/N568D, L26R/A59R, Q612R/K92E, L26R/H297R, L26R/E258R, K348R/K435Q, L26R/P94K, K348R/1471T, L26R/E119S, S579R/K435Q, L26R/Y709R, S198R/N568D, S186R/N568D, L26R/G497R, Q612R/N568D, K118R/N568D, L26R/L254R, K348R/E258K, L26R/P96K, L26R/Y245R, L26R/1238R, L26R/P243R, L26R/L16F, L26R/P230R, L26R/P199R, L26R/G585R, K348R/N568D, L26R/A673R, L26R/V246R, L26R/S229R, L26R/S265R, L26R/C50P, L26R/F253R, L26R/L264R, L26R/1227R, L26R/P247R, L26R/Q250R, L26R/F253R, L26R/L264R, L26R/1227R, L26R/P247R, L26K/A121Q, L26R/A121Q, K99R/L149R, K99R/N148R, L149R/H208R, S362R/L26R L26R/N148R, L26R/H208R, N30R/N148R, L26R/K99R, L26R/P707R, L26R/L149R, L26R/N30R, L26R/N355R, L26R/K281R, L26R/S108R, L26R/K348R, T5R/V139R, I2R/V139R, K99R/S186R, L26R/A673G, L26R/Q674R, S579R/L26K, F701R/E258K, T5R/L26K, L26R/K435Q, L26R/G685R, L26R/Q674K, L26R/P699R, L26R/T70E, L26R/Q232R, L26R/T252R, L26R/P679R, L26R/E83K, L26R/E73P, L26R/K248E, L26R, T5R/S223P, S579R/S223P, L26R/S223P, T5R/A121Q, L26R/A696R, S198R/1471T, L26R/N153R, L26R/E682R, L26R/D703R, Q612R/L26K, L26R/1471T, K348R/L26K, S579R/1471T, L26R/V228R, T5R/S638K, S579R/K189P, S579R/E258K, L26R/K260R, L26R/S638K, S579R/Y220S, T5R/1471T, L26R/F233R, L26R/V521T, F701R/A121Q, L26R/G361R, S198R/E258K, L26R/S472R, T5R/Y220S, L26R/A150K, L26R/S684R, L26R/E157R, L26R/K248R, F701R/L26K, S198R/N406K, S198R/Y220S, S198R/S638K, S198R/V521T, S579R/A121Q, K348R/Y220S, S198R/K189P, L26R/E242R, L26R/K678R, T5R/N406K, L26R/I158K, T5R/V521T, L26R/N259R, L26R/K257R, L26R/K256R, T5R/K189P, L26R/C405R, S579R/V521T, S579R/N406K, T5R/K92E, T5R/E258K, L26R/197R, S579R/S638K, T5R/K435Q, F701R/S638K, L26R/L236R, F701R/1471T, Q612R/S223P, F701R/S223P, S198R/E119S, S579R/K92E, L26R/E715R, Q612R/1471T, F701R/Y220S, S198R/S223P, and L26R/K266R relative to SEQ ID NO: 2. In some embodiments, the one or more amino acid alterations comprises three simultaneous amino acid substitutions selected from L26R/E109R/H208R, L26R/E109R/K184R, L26R/E109R/K38R, L26R/E109R/L182R, L26R/E109R/Q183R, L26R/E109R/S108R, L26R/E109R/S198R, L26R/E109R/T114R, L26R/H208R/K184R, L26R/H208R/K38R, L26R/H208R/L182R, L26R/H208R/Q183R, L26R/H208R/S108R, L26R/H208R/S198R, L26R/H208R/T114R, L26R/K184R/H208R, L26R/K184R/K38R, L26R/K184R/L182R, L26R/K184R/Q183R, L26R/K184R/S108R, L26R/K184R/S198R, L26R/K184R/T114R, L26R/K38R/L182R, L26R/K38R/Q183R, L26R/K38R/S108R, L26R/K38R/S198R, L26R/K38R/T114R, L26R/L182R/Q183R, L26R/L182R/S108R, L26R/L182R/S198R, L26R/L182R/T114R, L26R/Q183R/K184R, L26R/Q183R/H208R, L26R/Q183R/S108R, L26R/Q183R/S198R, L26R/Q183R/T114R, L26R/S108R/S198R, L26R/S108R/T114R, L26R/S198R/T114R, E109R/H208R/K184R, E109R/H208R/K38R, E109R/H208R/L182R, E109R/H208R/Q183R, E109R/H208R/S108R, E109R/H208R/S198R, E109R/H208R/T114R, E109R/K184R/K38R, E109R/K184R/L182R, E109R/K184R/Q183R, E109R/K184R/S108R, E109R/K184R/S198R, E109R/K184R/T114R, E109R/K38R/L182R, E109R/K38R/Q183R, E109R/K38R/S108R, E109R/K38R/S198R, E109R/K38R/T114R, E109R/L182R/Q183R, E109R/L182R/S108R, E109R/L182R/S198R, E109R/L182R/T114R, E109R/Q183R/S108R, E109R/Q183R/S198R, E109R/Q183R/T114R, E109R/S108R/S198R, E109R/S108R/T114R, E109R/S198R/T114R, H208R/K184R/K38R, H208R/K184R/L182R, H208R/K184R/Q183R, H208R/K184R/S108R, H208R/K184R/S198R, H208R/K184R/T114R, H208R/K38R/L182R, H208R/K38R/Q183R, H208R/K38R/S108R, H208R/K38R/S198R, H208R/K38R/T114R, H208R/L182R/Q183R, H208R/L182R/S108R, H208R/L182R/S198R, H208R/L182R/T114R, H208R/Q183R/S108R, H208R/Q183R/S198R, H208R/Q183R/T 114R, H208R/S108R/S198R, H208R/S108R/T 114R, H208R/S198R/T114R, K184R/K38R/L182R, K184R/K38R/Q183R, K184R/K38R/S108R, K184R/K38R/S198R, K184R/K38R/T114R, K184R/L182R/Q183R, K184R/L182R/S108R, K184R/L 182R/S198R, K184R/L182R/T114R, K184R/Q183R/S108R, K184R/Q183R/S198R, K184R/Q183R/T114R, K184R/S108R/S198R, K184R/S108R/T114R, K184R/S198R/T 114R, K38R/L182R/Q183R, K38R/L182R/S108R, K38R/L182R/S198R, K38R/L182R/T 114R, K38R/Q183R/S108R, K38R/Q183R/S198R, K38R/Q183R/T 114R, K38R/S108R/S198R, K38R/S108R/T114R, K38R/S198R/T114R, L182R/Q183R/S108R, L182R/Q183R/S198R, L182R/Q183R/T114R, L182R/S108R/S198R, L182R/S108R/T114R, L182R/S198R/T114R, Q183R/S108R/S198R, Q183R/S108R/T114R, Q183R/S198R/T114R, and S108R/S198R/T 114R relative to SEQ ID NO: 2. In some embodiments, the one or more amino acid alterations comprises four simultaneous amino acid substitutions selected from L26R/E109R/H208R/K184R, L26R/E109R/H208R/K38R, L26R/E109R/H208R/L182R, L26R/E109R/H208R/Q183R, L26R/E109R/H208R/S108R, L26R/E109R/H208R/S198R, L26R/E109R/H208R/T114R, L26R/E109R/K184R/K38R, L26R/E109R/K184R/L182R, L26R/E109R/K184R/Q183R, L26R/E109R/K184R/S108R, L26R/E109R/K184R/S198R, L26R/E109R/K184R/T114R, L26R/E109R/K38R/L182R, L26R/E109R/K38R/Q183R, L26R/E109R/K38R/S108R, L26R/E109R/K38R/S198R, L26R/E109R/K38R/T114R, L26R/E109R/L182R/Q183R, L26R/E109R/L182R/S108R, L26R/E109R/L182R/S198R, L26R/E109R/L182R/T114R, L26R/E109R/Q183R/S108R, L26R/E109R/Q183R/S198R, L26R/E109R/Q183R/T114R, L26R/E109R/S108R/S198R, L26R/E109R/S108R/T114R, L26R/E109R/S198R/T114R, L26R/H208R/K184R/K38R, L26R/H208R/K184R/L182R, L26R/H208R/K184R/Q183R, L26R/H208R/K184R/S108R, L26R/H208R/K184R/S198R, L26R/H208R/K184R/T114R, L26R/H208R/K38R/L182R, L26R/H208R/K38R/Q183R, L26R/H208R/K38R/S108R, L26R/H208R/K38R/S198R, L26R/H208R/K38R/T114R, L26R/H208R/L182R/Q183R, L26R/H208R/L182R/S108R, L26R/H208R/L182R/S198R, L26R/H208R/L182R/T114R, L26R/H208R/Q183R/S108R, L26R/H208R/Q183R/S198R, L26R/H208R/S108R/S198R, L26R/H208R/Q183R/T114R, L26R/H208R/S108R/T114R, L26R/K184R/K38R/L182R, L26R/K184R/K38R/Q183R, L26R/H208R/S198R/T114R, L26R/K184R/K38R/S198R, L26R/K184R/K38R/T114R, L26R/K184R/K38R/S108R, L26R/K184R/L182R/S108R, L26R/K184R/L182R/S198R, L26R/K184R/L182R/Q183R, L26R/K184R/Q183R/S108R, L26R/K184R/Q183R/S198R, L26R/K184R/L182R/T114R, L26R/K184R/S108R/S198R, L26R/K184R/S108R/T114R, L26R/K184R/Q183R/T114R, L26R/K38R/L182R/Q183R, L26R/K38R/L182R/S108R, L26R/K184R/S198R/T114R, L26R/K38R/Q183R/S108R, L26R/K38R/L182R/S198R, L26R/K38R/L182R/T114R, L26R/K38R/S108R/S198R, L26R/K38R/Q183R/T114R, L26R/K38R/Q183R/S198R, L26R/L182R/Q183R/S108R, L26R/K38R/S198R/T114R, L26R/K38R/S108R/T114R, L26R/L182R/S108R/S198R, L26R/L182R/Q183R/T114R, L26R/L182R/Q183R/S198R, L26R/Q183R/S108R/S198R, L26R/L182R/S198R/T114R, L26R/L182R/S108R/T114R, L26R/S108R/S198R/T114R, L26R/Q183R/S198R/T114R, L26R/Q183R/S108R/T114R, E109R/H208R/K184R/K38R, E109R/H208R/K184R/L182R, E109R/H208R/K184R/Q183R, E109R/H208R/K184R/S108R, E109R/H208R/K184R/S198R, E109R/H208R/K184R/T114R, E109R/H208R/K38R/L182R, E109R/H208R/K38R/Q183R, E109R/H208R/K38R/S108R, E109R/H208R/K38R/S198R, E109R/H208R/K38R/T114R, E109R/H208R/L182R/Q183R, E109R/H208R/L182R/S108R, E109R/H208R/L182R/S198R, E109R/H208R/L182R/T114R, E109R/H208R/Q183R/S108R, E109R/H208R/Q183R/S198R, E109R/H208R/Q183R/T114R, E109R/H208R/S108R/S198R, E109R/H208R/S108R/T114R, E109R/H208R/S198R/T114R, E109R/K184R/K38R/L182R, E109R/K184R/K38R/Q183R, E109R/K184R/K38R/S108R, E109R/K184R/K38R/S198R, E109R/K184R/K38R/T114R, E109R/K184R/L182R/Q183R, E109R/K184R/L182R/S108R, E109R/K184R/L182R/S198R, E109R/K184R/L182R/T114R, E109R/K184R/Q183R/S108R, E109R/K184R/Q183R/S198R, E109R/K184R/Q183R/T114R, E109R/K184R/S108R/S198R, E109R/K184R/S108R/T114R, E109R/K184R/S198R/T114R, E109R/K38R/L182R/Q183R, E109R/K38R/L182R/S108R, E109R/K38R/L182R/S198R, E109R/K38R/L182R/T114R, E109R/K38R/Q183R/S108R, E109R/K38R/Q183R/S198R, E109R/K38R/Q183R/T114R, E109R/K38R/S108R/S198R, E109R/K38R/S108R/T114R, E109R/K38R/S198R/T114R, E109R/L182R/Q183R/S108R, E109R/L182R/Q183R/S198R, E109R/L182R/Q183R/T114R, E109R/L182R/S108R/S198R, E109R/L182R/S108R/T114R, E109R/L182R/S198R/T114R, E109R/Q183R/S108R/S198R, E109R/Q183R/S108R/T114R, E109R/Q183R/S198R/T114R, E109R/S108R/S198R/T114R, H208R/K184R/K38R/L182R, H208R/K184R/K38R/Q183R, H208R/K184R/K38R/S108R, H208R/K184R/K38R/S198R, H208R/K184R/K38R/T114R, H208R/K184R/L182R/Q183R, H208R/K184R/L182R/S108R, H208R/K184R/L182R/S198R, H208R/K184R/L182R/T114R, H208R/K184R/Q183R/S198R, H208R/K184R/Q183R/S108R, H208R/K184R/Q183R/T114R, H208R/K184R/S108R/S198R, H208R/K184R/S108R/T114R, H208R/K184R/S198R/T114R, H208R/K38R/L182R/Q183R, H208R/K38R/L182R/S108R, H208R/K38R/L182R/S198R, H208R/K38R/L182R/T114R, H208R/K38R/Q183R/S108R, H208R/K38R/Q183R/S198R, H208R/K38R/Q183R/T114R, H208R/K38R/S108R/S198R, H208R/K38R/S108R/T114R, H208R/K38R/S198R/T114R, H208R/L182R/Q183R/S108R, H208R/L182R/Q183R/S198R, H208R/L182R/Q183R/T114R, H208R/L182R/S108R/S198R, H208R/L182R/S108R/T114R, H208R/L182R/S198R/T114R, H208R/Q183R/S108R/S198R, H208R/Q183R/S108R/T114R, H208R/Q183R/S198R/T114R, H208R/S108R/S198R/T114R, K184R/K38R/L182R/Q183R, K184R/K38R/L182R/S108R, K184R/K38R/L182R/S198R, K184R/K38R/L182R/T114R, K184R/K38R/Q183R/S108R, K184R/K38R/Q183R/S198R, K184R/K38R/Q183R/T114R, K184R/K38R/S108R/S198R, K184R/K38R/S108R/T114R, K184R/K38R/S198R/T114R, K184R/L182R/Q183R/S108R, K184R/L182R/Q183R/S198R, K184R/L182R/Q183R/T114R, K184R/L182R/S108R/S198R, K184R/L182R/S108R/T114R, K184R/L182R/S198R/T114R, K184R/Q183R/S108R/S198R, K184R/Q183R/S108R/T114R, K184R/Q183R/S198R/T114R, K184R/S108R/S198R/T114R, K38R/L182R/Q183R/S108R, K38R/L182R/Q183R/S198R, K38R/L182R/Q183R/T114R, K38R/L182R/S108R/S198R, K38R/L182R/S108R/T114R, K38R/L182R/S198R/T114R, K38R/Q183R/S108R/S198R, K38R/Q183R/S108R/T114R, K38R/Q183R/S198R/T114R, K38R/S108R/S198R/T114R, L182R/Q183R/S108R/S198R, L182R/Q183R/S108R/T114R, L182R/Q183R/S198R/T114R, L182R/S108R/S198R/T114R, Q183R/S108R/S198R/T114R, L26R/Q183R/K184R/T114R relative to SEQ ID NO: 2.

In some embodiments, the one or more amino acid alterations relative to SEQ ID NO: 2, wherein the effector protein comprising one or more alterations has nickase activity. In some embodiments, the one or more amino acid alterations comprise one or more amino acid substitutions independently selected from E157A, E164A, E164L, E166A, E166I, E170A, I489A, 1489S, Y490S, Y490A, F491A, F491S, F491G, D495G, D495R, D495K, K496A, K496S, K498A, K498S, K500A, K500S, D501R, D501G, D501K, V502A, V502S, K504A, K504S, S505R, D506A, or a combination thereof relative to SEQ ID NO: 2. In some embodiments, the one or more amino acid alterations comprise a deletion of S478-S505 and insertion of: SDLYIERGGDPRDVHQQVETKPKGKRKSEIRILKIR (SEQ ID NO: 897) or SDYIVDHGGDPEKVFFETKSKKDKTKRYKRR (SEQ ID NO: 898) relative to SEQ ID NO: 2.

In some embodiments, the one or more amino acid alterations comprise one or more substitutions relative to SEQ ID NO: 2, wherein the effector protein comprising one or more substitutions has reduced nuclease activity. In some embodiments, the one or more amino acid alterations comprise one or more amino acid substitutions independently selected from D369A, D369N, D658A, D658N, E567A, E567Q, or a combination thereof relative to SEQ ID NO: 2.

In some embodiments, the one or more amino acid alterations comprise one or more substitutions wherein a substituted amino acid is substituted with R, K, or H.

In some embodiments, effector proteins provided herein are a variant of a WT effector protein (e.g., TABLE 2), wherein the WT effector protein has an amino acid sequence of any one of the amino acid sequences set forth in TABLE 1, and the effector protein comprises one or more amino acid alterations in one or more regions that interact with a substrate, such as a target nucleic acid, an engineered guide nucleic acid, or a guide nucleic acid-target nucleic acid heteroduplex. In some embodiments, effector proteins provided herein are variants of a WT effector protein (e.g., TABLE 2), wherein the WT effector protein has an amino acid sequence of any one of the amino acid sequences set forth in TABLE 1, and the effector protein comprises one or more amino acid alterations in a region of the effector protein that comprises a substrate binding activity, a catalytic activity, and/or a binding affinity for a substrate, such as a target nucleic acid, an engineered guide nucleic acid, or a guide nucleic acid-target nucleic acid heteroduplex. In some embodiments, effector proteins provided herein are a variant of a reference effector protein (e.g., TABLE 2), wherein the WT effector protein comprises an amino acid sequence of any one of the amino acid sequences set forth in TABLE 1, and the effector protein comprises one or more amino acid alterations in a RuvC domain, a REC domain, TPID, NTPID), or a combination thereof.

In some embodiments, the variant comprises an amino acid sequence that is at least about 65%, at least about 70%, at least about 75%, at least about 80%, at least about 85%, at least about 90%, at least about 95%, at least about 97%, or at least 98%, or at least about 99% identical to the amino acid sequence of SEQ ID NO: 1 and 569-631, wherein the variant further comprises one or more mutations described in TABLE 2 relative to the amino acid sequence of SEQ ID NO: 1 and 569-631. In some embodiments, the variant comprises an amino acid sequence that is at least about 65%, at least about 70%, at least about 75%, at least about 80%, at least about 85%, at least about 90%, at least about 95%, at least about 97%, or at least 98%, at least about 99%, or 100% similar to the amino acid sequence of SEQ ID NO: 1 and 569-631, wherein the variant further comprises one or more mutations described in TABLE 2 relative to the amino acid sequence of SEQ ID NO: 1 and 569-631. In some embodiments, the variant comprises an amino acid sequence that is at least about 65%, at least about 70%, at least about 75%, at least about 80%, at least about 85%, at least about 90%, at least about 95%, at least about 97%, or at least 98%, or at least about 99% identical to the amino acid sequence of SEQ ID NO: 2 and 632-700, wherein the variant further comprises one or more mutations described in TABLE 2 relative to the amino acid sequence of SEQ ID NO: 2 and 632-700. In some embodiments, the variant comprises an amino acid sequence that is at least about 65%, at least about 70%, at least about 75%, at least about 80%, at least about 85%, at least about 90%, at least about 95%, at least about 97%, or at least 98%, at least about 99%, or 100% similar to the amino acid sequence of SEQ ID NO: 2 and 632-700, wherein the variant further comprises one or more mutations described in TABLE 2 relative to the amino acid sequence of SEQ ID NO: 2 and 632-700.

In certain embodiments, compositions, systems and methods described herein comprise a nucleic acid encoding the effector protein, wherein the nucleic acid encoding the effector protein is a messenger RNA.

In some embodiments, compositions, systems, and methods described herein comprise a polypeptide, a nucleic acid encoding the polypeptide, or polypeptide system, wherein the polypeptide the polypeptide described herein comprises an amino acid sequence that is at least 85% identical to any one of the amino acid sequences set forth in TABLE 1. In some embodiments, compositions, systems, and methods described herein comprise a polypeptide, a nucleic acid encoding the polypeptide, or polypeptide system, wherein the polypeptide the polypeptide described herein comprises an amino acid sequence that is at least 90% identical, at least 95% identical, or at least 100% identical to any one of the amino acid sequences set forth in TABLE 1. In some embodiments, the polypeptide comprises a Type V Cas effector protein. In some embodiments, the polypeptide comprises a Cas.265466, a CasPhi.12, or a variant thereof. In some embodiments, the polypeptide comprises binding activity, nuclease activity, nickase activity, base editing activity, cleavage activity, or a combination thereof. In some embodiments, the polypeptide cleaves within or near the target sequence. In some embodiments, the polypeptide cleaves at least one strand of the target nucleic acid. In some embodiments, the polypeptide comprises at least one mutation or amino acid alteration that results in retaining, enhancing or reducing activity relative to corresponding reference amino acid sequence recited in TABLE 1. In some embodiments, the at least one mutation comprises an amino acid substitution at a position as set forth in TABLE 2 relative to corresponding reference amino acid sequence of TABLE 1. In some embodiments, the polypeptide has nickase activity. In some embodiments, the polypeptide has nuclease activity.

### Effector Partners

Provided herein are compositions, systems, and methods comprising one or more effector partners or uses thereof. In some embodiments, the effector partner is a heterologous protein an effector protein described herein. In some embodiments, the effector partner is not an effector protein as described herein. In some embodiments, the effector partner is capable of imparting a function or activity that is not provided by an effector protein as described herein. In some embodiments, the effector partner comprises a second effector protein or a multimeric form thereof. In some embodiments, the effector protein is fused to an effector partner, wherein the effector protein and the effector partner are heterologous to each other. In some embodiments, the effector protein is directly fused to N terminus or C terminus of the effector partner by an amide bond.

In some embodiments, an effector partner imparts a function or activity to a fusion protein comprising an effector protein that is not provided by the effector protein, including but not limited to nuclease activity, methyltransferase activity, demethylase activity, DNA repair activity, DNA damage activity, deamination activity, dismutase activity, alkylation activity, depurination activity, oxidation activity, dimer forming activity (e.g., pyrimidine dimer forming activity), integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity, modification of a polypeptide associated with target nucleic acid (e.g., a histone), and/or signaling activity. In some embodiments, the effector partner is selected from a nuclease, a nickase, a polymerase, a deaminase, a reverse transcriptase, a transcriptional repressor, an integrase, a recombinase and a transcriptional activator.

In some embodiments, the effector partner is fused or linked to an effector protein described herein. In some embodiments, the amino terminus of the effector partner is linked to the carboxy terminus of the effector protein directly or by a linker. In some embodiments, the carboxy terminus of the effector partner is linked to the amino terminus of the effector protein directly or by a linker. In some embodiments, the effector partner is functional when the effector protein is coupled to a guide nucleic acid. In some embodiments, the effector partner is functional when the effector protein is coupled to a target nucleic acid. In some embodiments, the guide nucleic acid imparts sequence specific activity to the effector partner. By way of non-limiting example, the effector protein comprises a catalytically inactive effector protein (e.g., a catalytically inactive variant of an effector protein described herein) when fused or linked to an effector partner.

In some embodiments, the effector partner directly or indirectly edits a target nucleic acid. Edits can be of a nucleobase, nucleotide, or nucleotide sequence of a target nucleic acid. In some embodiments, the effector partner interacts with additional proteins, or functional fragments thereof, to make modifications to a target nucleic acid. In other embodiments, the effector partner modifies proteins associated with a target nucleic acid. In some embodiments, an effector partner modulates transcription (e.g., inhibits transcription, increases transcription) of a target nucleic acid. In yet another example, an effector partner directly or indirectly inhibits, reduces, activates or increases expression of a target nucleic acid.

### Multimeric Complex Formation Modification Activity

In some embodiments, an effector partner inhibits the formation of a multimeric complex of an effector protein. Alternatively, the effector partner promotes the formation of a multimeric complex of the effector protein.

### Reverse Transcriptase (RT) Editing System

In some embodiments, systems and methods comprise components or uses of an RT editing system to modify a target nucleic acid. In some embodiments, RT editing is also referred to as prime editing or precise nucleobase editing. In some embodiments, an RT editing system comprises an effector protein and an effector partner comprising an RT editing enzyme. In some embodiments, the effector protein that is linked to the RT editing enzyme. In some embodiments, an RT editing enzyme comprises a polymerase. In some embodiments, an RT editing enzyme comprises a reverse transcriptase. A non-limiting example of a reverse transcriptase is an M-MLV RT enzyme and variants thereof having polymerase activity. In some embodiments, the M-MLV RT enzyme comprises at least one mutation selected from D200N, L603W, T330P, T306K, and W313F relative to wildtype M-MLV RT enzyme. In some embodiments, systems and methods comprise an RT editing enzyme, wherein the RT editing enzyme is not fused or linked to the effector protein. In some embodiments, the RT editing enzyme comprises a recruiting moiety that recruits the RT editing enzyme to the target nucleic acid. By way of non-limiting example, the RT editing enzyme comprises a peptide that binds an aptamer, wherein the aptamer is located on a guide RNA, template RNA, or combination thereof. Also, by way of non-limiting example, the RT editing enzyme is linked to a protein that binds to (or is bound by) the effector protein or a protein linked/fused to the effector protein. In some embodiments, an RT editing enzyme requires an RT editing guide RNA (pegRNA) to catalyze editing. In some embodiments, the pegRNA is capable of identifying a target nucleotide or target sequence in a target nucleic acid to be edited and encoding a new genetic information that replaces the target nucleotide or target sequence in the target nucleic acid. In some embodiments, an RT editing enzyme requires a pegRNA and a guide RNA, such as a single guide RNA, to catalyze the editing. In some embodiments, the RT editing system comprises a template RNA comprising a primer binding sequence that hybridizes to a primer sequence of the dsDNA molecule that is formed when target nucleic acid is cleaved, and a template sequence that is complementary to at least a portion of the target sequence of the dsDNA molecule except for at least one nucleotide. In some embodiments, the template RNA is covalently linked to a guide RNA. In some embodiments, the template RNA is not covalently linked to a guide RNA. In some embodiments, at least a portion of the template RNA hybridizes to the target nucleic acid. In some embodiments, the target nucleic acid is a dsDNA molecule. In some embodiments, at least a portion of the template RNA hybridizes to a first strand of the target nucleic acid and at least a portion of the guide RNA hybridizes to a second strand of the target nucleic acid. In some embodiments, the pegRNA comprises: a guide RNA comprising a second region that is bound by the effector protein, and a first region comprising a spacer sequence that is complementary to a target sequence of the dsDNA molecule; and a template RNA comprising a primer binding sequence that hybridizes to a primer sequence of the dsDNA molecule that is formed when target nucleic acid is cleaved, and a template sequence that is complementary to at least a portion of the target sequence of the dsDNA molecule with the exception of at least one nucleotide. In some embodiments, the at least one nucleotide is incorporated into the target nucleic acid by activity of the RT editing enzyme, thereby modifying the target nucleic acid. In some embodiments, the spacer sequence is complementary to the target sequence on a target strand of the dsDNA molecule. In some embodiments, the spacer sequence is complementary to the target sequence on a non-target strand of the dsDNA molecule. In some embodiments, the primer binding sequence hybridizes to a primer sequence on the non-target strand of the dsDNA molecule. In some embodiments, the primer binding sequence hybridizes to a primer sequence on the target strand of the dsDNA molecule. In some embodiments, the target strand is cleaved. In some embodiments, the non-target strand is cleaved.

### Nucleic Acid Modification Activity

In some embodiments, effector partners have enzymatic activity that modifies a nucleic acid, such as a target nucleic acid. In some embodiments, the target nucleic acid comprises or consists of a ssRNA, dsRNA, ssDNA, or a dsDNA. Examples of enzymatic activity that modifies the target nucleic acid include, but are not limited to: nuclease activity, which comprises the enzymatic activity of an enzyme which allows the enzyme to cleave the phosphodiester bonds between the nucleotide subunits of nucleic acids, such as that provided by a restriction enzyme, or a nuclease (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y, human immunodeficiency virus type 1 integrase (IN), Tn3 resolvase); transposase activity; recombinase activity such as that provided by a recombinase (e.g., catalytic domain of Gin recombinase); polymerase activity; ligase activity; helicase activity; photolyase activity; and glycosylase activity.

In some embodiments, effector partners target a ssRNA, dsRNA, ssDNA, or a dsDNA. In some embodiments, effector partners target ssRNA. Non-limiting examples of effector partners for targeting ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins.

It is understood that an effector partner comprises an entire protein, or a fragment of the protein (e.g., a functional domain). In some embodiments, the functional domain binds or interacts with a nucleic acid, such as ssRNA, including intramolecular and/or intermolecular secondary structures thereof (e.g., hairpins, stem-loops, etc.). In some embodiments, the functional domain interacts transiently or irreversibly, directly, or indirectly. In some embodiments, a functional domain comprises a region of one or more amino acids in a protein that is required for an activity of the protein, or the full extent of that activity, as measured in an in vitro assay. Activities include but are not limited to nucleic acid binding, nucleic acid editing, nucleic acid mutating, nucleic acid modifying, nucleic acid cleaving, protein binding or combinations thereof. The absence of the functional domain, including mutations of the functional domain, would abolish or reduce activity.

Accordingly, in some embodiments, effector partners comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP S1, Y14, DEK, REF2, and SRm160); protein domains responsible for stabilizing RNA (e.g., PABP); proteins and protein domains responsible for polyadenylation of RNA (e.g., PAP1, GLD-2, and Star-PAP); proteins and protein domains responsible for polyuridinylation of RNA (e.g., CI D1 and terminal uridylate transferase); and other suitable domains that affect nucleic acid modifications.

In some embodiments, effector partner comprises a chromatin-modifying enzyme. In some embodiments, the effector partner chemically modifies a target nucleic acid, for example by methylating, demethylating, or acetylating the target nucleic acid in a sequence specific or non-specific manner.

### Base Editing Enzymes

In some embodiments, effector partners edit a nucleobase of a target nucleic acid. In some embodiments, the effector partner is referred to as a base editing enzyme. In some embodiments, a base editing enzyme variant that differs from a naturally occurring base editing enzyme, but it is understood that any reference to a base editing enzyme herein also refers to a base editing enzyme variant.

In some embodiments, a base editor is a system comprising an effector protein and a base editing enzyme. In some embodiments, the base editor comprises a base editing enzyme and an effector protein as independent components. In some embodiments, the base editor comprises a fusion protein comprising a base editing enzyme fused or linked to an effector protein. In some embodiments, the amino terminus of the effector partner is linked to the carboxy terminus of the effector protein by the linker. In some embodiments, the carboxy terminus of the effector partner is linked to the amino terminus of the effector protein by the linker. In some embodiments, the base editor is functional when the effector protein is coupled to a guide nucleic acid. In some embodiments, the base editor is functional when the effector protein is coupled to a target nucleic acid. In some embodiments, the target nucleic acid comprises a gene associated a genetic blood disease or disorder. In some embodiments, the genetic blood disease or disorder comprises sickle cell anemia, sickle cell disease (SCD), β-thalassemia, or a combination thereof. The guide nucleic acid imparts sequence specific activity to the base editor. By way of non-limiting example, the effector protein comprises a catalytically inactive effector protein (e.g., a catalytically inactive variant of an effector protein described herein). Also, by way of non-limiting example, the base editing enzyme comprises deaminase activity. Additional base editors are described herein.

In some embodiments, base editing enzymes are capable of catalyzing editing (e.g., a chemical modification) of a nucleobase of a nucleic acid molecule, such as DNA or RNA (single stranded or double stranded). In some embodiments, a base editing enzyme, and therefore a base editor, is capable of converting an existing nucleobase to a different nucleobase, such as: an adenine (A) to guanine (G); cytosine (C) to thymine (T); cytosine (C) to guanine (G); uracil (U) to cytosine (C); guanine (G) to adenine (A); hydrolytic deamination of an adenine or adenosine, or methylation of cytosine (e.g., CpG, CpA, CpT or CpC). In some embodiments, base editing enzymes edit a nucleobase on a ssDNA. In some embodiments, base editing enzymes edit a nucleobase on both strands of dsDNA. In some embodiments, base editing enzymes edit a nucleobase of an RNA.

In some embodiments, a base editing enzyme itself binds or does not bind to the nucleic acid molecule containing the nucleobase. In some embodiments, upon binding to its target locus in the target nucleic acid (e.g., a DNA molecule), base pairing between the guide nucleic acid and target strand leads to displacement of a small segment of ssDNA in an “R-loop”. In some embodiments, DNA bases within the R-loop are edited by the base editing enzyme having the deaminase enzyme activity. In some embodiments, base editing systems for improved efficiency in eukaryotic cells comprise a base editing enzyme, and a catalytically inactive effector protein that generate a nick in the non-edited strand and induce repair of the non-edited strand using the edited strand as a template.

In some embodiments, a base editing enzyme comprises a deaminase enzyme. Exemplary deaminases s are described in US20210198330, WO2021041945, WO2021050571A1, and WO2020123887, all of which are incorporated herein by reference in their entirety. Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3: eaao4774 (2017), and Rees et al., Nat Rev Genet. 2018 December; 19 (12): 770-788. doi: 10.1038/s41576-018-0059-1, which are hereby incorporated by reference in their entirety. In some embodiments, the deaminase functions as a monomer. In some embodiments, the deaminase functions as heterodimer with an additional protein. In some embodiments, base editing enzymes comprise a DNA glycosylase inhibitor (e.g., an uracil glycosylase inhibitor (UGI) or uracil N-glycosylase (UNG)). In some embodiments, the effector partner is a deaminase, e.g., ADAR1/2, ADAR-2, AID, or any functional variant thereof.

In some embodiments, the base editor is a cytosine base editor (CBE), wherein the base editing enzyme is a cytosine base editing enzyme. In some embodiments, the cytosine base editing enzyme, and therefore CBE, converts a cytosine to a thymine. In some embodiments, a cytosine base editing enzyme accepts ssDNA as a substrate but is not capable of cleaving dsDNA, wherein the CBE comprises a catalytically inactive effector protein. In some embodiments, when bound to its cognate DNA, the catalytically inactive effector protein of the CBE performs local denaturation of the DNA duplex to generate an R-loop in which the DNA strand not paired with a guide nucleic acid exists as a disordered single-stranded bubble. In some embodiments, the catalytically inactive effector protein generated ssDNA R-loop enables the CBE to perform efficient and localized cytosine deamination in vitro. In some embodiments, deamination activity is exhibited in a window of about 4 to about 10 base pairs. In some embodiments, the catalytically inactive effector protein presents a target site to the cytosine base editing enzyme in high effective molarity, which enables the CBE to deaminate cytosines located in a variety of different sequence motifs, with differing efficacies. In some embodiments, the CBE is capable of mediating RNA-programmed deamination of target cytosines in vitro or in vivo. In some embodiments, the cytosine base editing enzyme is a cytidine deaminase. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C⋅G-to-G⋅C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, the effector partner comprises a uracil glycosylase inhibitor (UGI). In some embodiments, the CBE described herein comprises UGI. Base excision repair (BER) of U⋅G in DNA is initiated by a uracil N-glycosylase (UNG), which recognizes a U⋅G mismatch generated by a CBE and cleaves the glycosidic bond between a uracil and a deoxyribose backbone of DNA. BER results in the reversion of the U⋅G intermediate created by the cytosine base editing enzyme back to a C⋅G base pair. Accordingly, in some embodiments, the UNG is inhibited by fusion of a UGI to the effector protein. In some embodiments, the UGI is a small protein from bacteriophage PBS. In some embodiments, the UGI is a DNA mimic that potently inhibits both human and bacterial UNG. In some embodiments, the UGI inhibitor is any protein or polypeptide that inhibits UNG.

In some embodiments, the CBE described herein mediates efficient base editing in bacterial cells and moderately efficient editing in mammalian cells, enabling conversion of a C⋅G base pair to a T⋅A base pair through a U⋅G intermediate. In some embodiments, the CBE is modified to increase base editing efficiency while editing more than one strand of DNA.

In some embodiments, the CBE described herein nicks a non-edited DNA strand. In some embodiments, the non-edited DNA strand nicked by the CBE biases cellular repair of a U⋅G mismatch to favor a U⋅A outcome, elevating base editing efficiency.

In some embodiments, a base editor described herein comprising one or more base editing enzymes (e.g., APOBEC1, nickase, and UGI) that efficiently edits in mammalian cells, while minimizing frequency of non-target indels. In some embodiments, base editors do not comprise a functional fragment of the base editing enzyme. In some embodiments, base editors do not comprise a function fragment of a UGI, where such a fragment is capable of excising a uracil residue from DNA by cleaving an N-glycosidic bond.

In some embodiments, the effector partner comprises a non-protein uracil-DNA glycosylase inhibitor (npUGI). In some embodiments, the npUGI is selected from a group of small molecule inhibitors of uracil-DNA glycosylase (UDG), or a nucleic acid inhibitor of UDG. In some embodiments, the npUGI is a small molecule derived from uracil. Examples of small molecule non-protein uracil-DNA glycosylase inhibitors, fusion proteins, and Cas-CRISPR systems comprising base editing activity are described in WO2021087246, which is incorporated by reference in its entirety.

In some embodiments, the base editor is a cytosine base editor, wherein the based editing enzyme is a cytosine base editing enzyme. In some embodiments, the cytosine base editing enzyme is a cytidine deaminase. In some embodiments, the base editor comprising the cytidine deaminase is generated by ancestral sequence reconstruction as described in WO2019226953, which is hereby incorporated by reference in its entirety. Non-limiting exemplary cytidine deaminases suitable for use with effector proteins described herein include: APOBEC1, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9 (A840H)-UGI), BE3-Gam, saBE3, saBE4-Gam, BE4, BE4-Gam, saBE4, and saBE4-Gam as described in WO2021163587, WO2021087246, WO2021062227, and WO2020123887, which are incorporated herein by reference in their entirety.

In some embodiments, a base editor is a cytosine to guanine base editor (CGBE), wherein the base editing enzyme is a cytosine to guanine base editing enzyme. In some embodiments, the CGBE, converts a cytosine into a guanine.

In some embodiments, a base editor is an adenine base editor (ABE), wherein the base editing enzyme is an adenine base editing enzyme. In some embodiments, the adenine base editing enzyme, and therefore the ABE, converts an adenine to a guanine. In some embodiments, the adenine base editing enzyme converts an A⋅T base pair to a G⋅C base pair. In some embodiments, the adenine base editing enzyme converts a target A⋅T base pair to G⋅C in vivo or in vitro. In some embodiments, the adenine base editing enzymes provided herein reverse spontaneous cytosine deamination, which has been linked to pathogenic point mutations. In some embodiments, the adenine base editing enzymes provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A, U, or C in mRNA during translation. Non-limiting exemplary adenine base editing enzymes suitable for use with effector proteins described herein include: ABE8e, ABE8.20m, APOBEC3A, Anc APOBEC (a.k.a. AncBE4Max), and BtAPOBEC2. Non-limiting exemplary ABEs suitable for use herein include: ABE7, ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, and ABE8.24d. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871.

In some embodiments, the ABE described herein is capable of targeting polyA signals, splice site acceptors, and start codons. In some embodiments, the ABE cannot create stop codons for knock-down.

In some embodiments, an adenine base editing enzyme is an adenosine deaminase. Non-limiting exemplary adenosine base editors suitable for use herein include ABE9. In some embodiments, the ABE comprises an engineered adenosine deaminase enzyme capable of acting on ssDNA. In some embodiments, the engineered adenosine deaminase enzyme comprises an adenosine deaminase variant that differs from a naturally occurring deaminase. Relative to the naturally occurring deaminase, the adenosine deaminase variant comprises one or more amino acid alterations, including a V82S alteration, a T166R alteration, a Y147T alteration, a Y147R alteration, a Q154S alteration, a Y123H alteration, a Q154R alteration, or a combination thereof.

In some embodiments, the base editor comprises an adenine deaminase (e.g., TadA). In some embodiments, the adenosine deaminase is a TadA monomer (e.g., Tad*7.10, TadA*8 or TadA*9). In some embodiments, the adenosine deaminase is a TadA*8 variant (e.g., any one of TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety). In some embodiments, the base editor comprises TadA.

In some embodiments, a base editing enzyme is a deaminase dimer. In some embodiments, the ABE comprises the effector protein, the adenine base editing enzyme and the deaminase dimer. In some embodiments, the deaminase dimer comprises an adenosine deaminase. In some embodiments, the deaminase dimer comprises TadA and a suitable adenine base editing enzyme including an: ABE8e, ABE8.20m, APOBEC3A, Anc APOBEC (a.k.a. AncBE4Max), BtAPOBEC2, and variants thereof. In some embodiments, the adenine base editing enzyme is fused to amino-terminus or the carboxy-terminus of TadA.

In some embodiments, a base editor is an RNA base editor, wherein the base editing enzyme is an RNA base editing enzyme. In some embodiments, the RNA base editing enzyme comprises an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editing enzymes, and therefore base editors, are used for treating a subject having or a subject suspected of having a disease related to a gene of interest. In some embodiments, base editing enzymes, and therefore base editors, are useful for treating a disease or a disorder caused by a point mutation in a gene of interest. In some embodiments, compositions, systems, and methods described herein comprise a base editor and a guide nucleic acid, wherein the base editor comprises an effector protein and a base editing enzyme, and wherein the guide nucleic acid directs the base editor to a sequence in a target gene.

### Protein Modification Activity

In some embodiments, an effector partner provides enzymatic activity that modifies a protein associated with a target nucleic acid. In some embodiments, the protein comprises a histone, an RNA binding protein, or a DNA binding protein. Examples of such protein modification activities include: methyltransferase activity, such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity; phosphatase activity; ubiquitin ligase activity; deubiquitinating activity; adenylation activity; deadenylation activity; SUMOylating activity; deSUMOylating activity; ribosylation activity; deribosylation activity; myristoylation activity; and demyristoylation activity.

### CRISPRa Fusions and CRISPRi Fusions

In some embodiments, effector partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some embodiments, effector partners that increase or decrease transcription include a transcription activator domain or a transcription repressor domain, respectively.

In some embodiments, effector partners activate or increase expression of a target nucleic acid. In some embodiments, effector partners increase expression of the target nucleic acid relative to its expression in the absence of the effector partners. Relative expression, including transcription and RNA levels, in some embodiments, is assessed, quantified, and compared, e.g., by RT-qPCR. In some embodiments, effector partners comprise a transcriptional activator. In some embodiments, the transcriptional activators promote transcription by: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof.

Non-limiting examples of effector partners that promote or increase transcription include: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof. Other non-limiting examples of suitable effector partners include: proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for stimulation of RNA splicing (e.g., Serine/Arginine-rich (SR) domains); and proteins and protein domains responsible for stimulating transcription (e.g., CDK7 and HIV Tat).

In some embodiments, effector partners inhibit or reduce expression of a target nucleic acid. In some embodiments, effector partners reduce expression of the target nucleic acid relative to its expression in the absence of the effector partners. Relative expression, including transcription and RNA levels, In some embodiments, is assessed, quantified, and compared, e.g., by RT-qPCR. In some embodiments, effector partners comprise a transcriptional repressor. In some embodiments, the transcriptional repressors inhibit transcription by: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

Non-limiting examples of effector partners that decrease or inhibit transcription include: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof. Other non-limiting examples of suitable effector partners include: proteins and protein domains responsible for repressing translation (e.g., Ago2 and Ago4); proteins and protein domains responsible for repression of RNA splicing (e.g., PTB, Sam68, and hnRNP A1); proteins and protein domains responsible for reducing the efficiency of transcription (e.g., FUS (TLS)).

In some embodiments, fusion proteins comprising the described effector partners and an effector protein are referred to as CRISPRa fusions, wherein the effector partners activate or increase expression of a target nucleic acid. In some embodiments, fusion proteins comprising the described effector partners and an effector protein are referred to as CRISPRi fusions, wherein the effector partners inhibit or reduce expression of a target nucleic acid. In some embodiments, fusion proteins are targeted by a guide nucleic acid (e.g., guide RNA) to a specific location in a target nucleic acid and exert locus-specific regulation such as blocking RNA polymerase binding to a promoter (which selectively inhibits transcription activator function), and/or changes a local chromatin status (e.g., when a fusion sequence is used that edits the target nucleic acid or modifies a protein associated with the target nucleic acid). In some embodiments, the modifications are transient (e.g., transcription repression or activation). In some embodiments, the modifications are inheritable. For example, epigenetic modifications made to a target nucleic acid, or to proteins associated with the target nucleic acid, e.g., nucleosomal histones, in a cell, can be observed in a successive generation.

In some embodiments, effector partner comprises an RNA splicing factor. In some embodiments, the RNA splicing factor is used (in whole or as fragments thereof) for modular organization, with separate sequence-specific RNA binding modules and splicing effector domains. In some embodiments, the RNA splicing factors comprise members of the Serine/Arginine-rich (SR) protein family containing N-terminal RNA recognition motifs (RRMs) that bind to exonic splicing enhancers (ESEs) in pre-mRNAs and C-terminal RS domains that promote exon inclusion. In some embodiments, a hnRNP protein hnRNP A1 binds to exonic splicing silencers (ESSs) through its RRM domains and inhibits exon inclusion through a C-terminal Glycine-rich domain. In some embodiments, the RNA splicing factors regulate alternative use of splice site (ss) by binding to regulatory sequences between two alternative sites. For example, in some embodiments, ASF/SF2 recognizes ESEs and promote the use of intron proximal sites. In some embodiments, hnRNP A1 binds to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. Long splicing isoform Bcl-xL is a potent apoptosis inhibitor expressed in long-lived postmitotic cells and is up-regulated in many cancer cells, protecting cells against apoptotic signals. Short isoform Bcl-xS is a pro-apoptotic isoform and expressed at high levels in cells with a high turnover rate (e.g., developing lymphocytes). A ratio of the two Bcl-x splicing isoforms is regulated by multiple c\acute{ω}-elements that are located in either core exon region or exon extension region (i.e., between the two alternative 5′ splice sites). For more examples, see WO2010075303, which is hereby incorporated by reference in its entirety.

### Recombinases

In some embodiments, effector partners comprise a recombinase. In some embodiments, provided herein is a recombinase system comprising effector proteins described herein and the recombinase. In some embodiments, the effector proteins have reduced nuclease activity or no nuclease activity. In some embodiments, the recombinase is a site-specific recombinase.

In some embodiments, the recombinase system comprises a catalytically inactive effector protein, wherein the recombinase can be a site-specific recombinase. Such systems can be used for site-directed transgene insertion. Non-limiting examples of site-specific recombinases include a tyrosine recombinase (e.g., Cre, Flp or lambda integrase), a serine recombinase (e.g., gamma-delta resolvase, Tn3 resolvase, Sin resolvase, Gin invertase, Hin invertase, Tn5044 resolvase, IS607 transposase and integrase), or mutants or variants thereof. In some embodiments, the recombinase is a serine recombinase. Non-limiting examples of serine recombinases include gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include: Bxb1, wBeta, BL3, phiR4, A118, TG1, MR11, phi370, SPBc, TP901-1, phiRV, FC1, K38, phiBT1, and phiC31. Further discussion and examples of suitable recombinase effector partners are described in U.S. Pat. No. 10,975,392, which is incorporated herein by reference in its entirety. In some embodiments, the fusion protein comprises a linker that links the recombinase to the Cas-CRISPR domain of the effector protein. In some embodiments, the linker is The-Ser.

### Linkers for Peptides

In some embodiments, a linker comprises a bond or molecule that links a first polypeptide to a second polypeptide. Accordingly, in some embodiments, effector proteins, effector partners, or combinations thereof are connected by linkers. In some embodiments, the linker comprises or consists of a covalent bond. In some embodiments, the linker comprises or consists of a chemical group. In some embodiments, the linker comprises an amino acid. In some embodiments, a peptide linker comprises at least two amino acids linked by an amide bond. In general, the linker connects a terminus of the effector protein to a terminus of the effector partner. In some embodiments, carboxy terminus of the effector protein is linked to the amino terminus of the fusion effector. In some embodiments, carboxy terminus of the effector partner is linked to the amino terminus of the effector protein. In some embodiments, the effector protein and the effector partner are directly linked by a covalent bond.

In some embodiments, linkers comprise one or more amino acids. In some embodiments, linker is a protein. In some embodiments, a terminus of the effector protein is linked to a terminus of the effector partner through an amide bond. In some embodiments, a terminus of the effector protein is linked to a terminus of the effector partner through a peptide bond. In some embodiments, linkers comprise an amino acid. In some embodiments, linkers comprise a peptide. In some embodiments, an effector protein is coupled to an effector partner by a linker protein. In some embodiments, the linker comprises any of a variety of amino acid sequences. In some embodiments, the linker comprises a region of rigidity (e.g., beta sheet, alpha helix), a region of flexibility, or any combination thereof. In some embodiments, the linker comprises small amino acids, such as glycine and alanine, that impart high degrees of flexibility. The ordinarily skilled artisan will recognize that design of a peptide conjugated to any desired element comprises linkers that are all or partially flexible, such that the linker comprises a flexible linker as well as one or more portions that confer less flexible structure. Suitable linkers include proteins of 4 linked amino acids to 40 linked amino acids in length, or between 4 linked amino acids and 25 linked amino acids in length. In some embodiments, linked amino acids described herein comprise at least two amino acids linked by an amide bond.

In some embodiments, linkers are produced by using synthetic, linker-encoding oligonucleotides to couple proteins, or are encoded by a nucleic acid sequence encoding a fusion protein (e.g., an effector protein coupled to an effector partner). In some embodiments, the linker is from 1 to 300, from 1 to 250, from 1 to 200, from 1 to 150, from 1 to 100, from 1 to 50, from 1 to 25, from 1 to 10, from 10 to 300, from 10 to 250, from 10 to 200, from 10 to 150, from 10 to 100, from 10 to 50, from 10 to 25, from 25 to 300, from 25 to 250, from 25 to 200, from 25 to 150, from 25 to 100, from 25 to 50, from 50 to 300, from 50 to 250, from 50 to 200, from 50 to 150, from 50 to 100, from 100 to 300, from 100 to 250, from 100 to 200, from 100 to 150, from 150 to 300, from 150 to 250, from 150 to 200, from 200 to 300, from 200 to 250, or from 250 to 300 amino acids in length. In some embodiments, the linker is from 1 to 100 amino acids in length. In some embodiments, the linker is more 100 amino acids in length. In some embodiments, the linker is from 10 to 27 amino acids in length. In some embodiments, linker proteins include glycine polymers (G)ₙ, glycine-serine polymers (including, for example, (GS)ₙ, GSGGSₙ (SEQ ID NO: 899), GGSGGSₙ (SEQ ID NO: 900), and GGGSₙ (SEQ ID NO: 901), where n is an integer of at least one), glycine-alanine polymers, and alanine-serine polymers. In some embodiments, linkers comprise amino acid sequences including, but not limited to, GGSG (SEQ ID NO: 902), GGSGG (SEQ ID NO: 903), GSGSG (SEQ ID NO: 904), GSGGG (SEQ ID NO: 905), GGGSG (SEQ ID NO: 907), and GSSSG (SEQ ID NO: 907). In some embodiments, the linker comprises one or more repeats a tri-peptide GGS. In some embodiments, the linker is an XTEN linker. In some embodiments, the XTEN linker is an XTEN80 linker. In some embodiments, the XTEN linker is an XTEN20 linker. In some embodiments, the XTEN20 linker has an amino acid sequence of GSGGSPAGSPTSTEEGTSESATPGSG (SEQ ID NO: 896).

In some embodiments, linkers do not comprise an amino acid. In some embodiments, linkers do not comprise a peptide. In some embodiments, linkers comprise a nucleotide, a polynucleotide, a polymer, or a lipid. In some embodiments, a linker comprises a polyethylene glycol (PEG), polypropylene glycol (PPG), co-poly (ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

In some embodiments, a linker is recognized and cleaved by a protein. In some embodiments, a linker comprises a recognition sequence. In some embodiments, the recognition sequence is recognized and cleaved by the protein. In some embodiments, a guide nucleic acid comprises an aptamer. In some embodiments, the aptamer serves a similar function as a linker, bringing an effector protein and an effector partner protein into proximity. In some embodiments, the aptamer functionally connects two proteins (e.g., effector protein, effector partner) by interacting non-covalently with both, thereby bringing both proteins into proximity of the guide nucleic acid. In some embodiments, the first protein and/or the second protein comprise or is covalently linked to an aptamer binding moiety. In some embodiments, the aptamer is a short single stranded DNA (ssDNA) or RNA (ssRNA) molecule capable of being bound be the aptamer binding moiety. In some embodiments, the aptamer is a molecule that is capable of mimicking antibody binding activity. In some embodiments, the aptamer is classified as a chemical antibody. In some instances, the aptamer described herein refers to artificial oligonucleotides that bind one or more specific molecules. In some embodiments, aptamers exhibit a range of affinities (K_{D} in the pM to μM range) with little or no off-target binding.

### Engineered Proteins

In some embodiments, proteins (e.g., effector protein or effector partner) described herein have been modified (also referred to as an engineered protein). In some embodiments, a modification of the proteins comprises addition of one or more amino acids, deletion of one or more amino acids, substitution of one or more amino acids, or combinations thereof. In some embodiments, the proteins disclosed herein are engineered proteins. Unless otherwise indicated, reference to the proteins throughout the present disclosure include engineered proteins thereof.

In some embodiments, proteins (e.g., effector protein or effector partner) described herein can be modified with the addition of one or more heterologous peptides. In some embodiments, the protein modified with the addition of one or more heterologous peptides is referred to herein as a fusion protein. Such fusion proteins are described herein and throughout.

In some embodiments, a heterologous peptide comprises a subcellular localization signal. In some embodiments, a subcellular localization signal can be a nuclear localization signal (NLS). In some embodiments, the NLS facilitates localization of a nucleic acid, protein, or small molecule to the nucleus, when present in a cell that contains a nuclear compartment. TABLE 3 lists exemplary NLS sequences. In some embodiments, the subcellular localization signal is a nuclear export signal (NES), a sequence to keep the protein retained in the cytoplasm, a mitochondrial localization signal for targeting to the mitochondria, a chloroplast localization signal for targeting to a chloroplast, an ER retention signal, and the like. In some embodiments, the protein described herein is not modified with a subcellular localization signal so that the protein is not targeted to the nucleus, which can be advantageous depending on the circumstance (e.g., when the target nucleic acid is an RNA that is present in the cytosol).

In some embodiments, a heterologous peptide comprises a chloroplast transit peptide (CTP), also referred to as a chloroplast localization signal or a plastid transit peptide, which targets the protein to a chloroplast. Chromosomal transgenes from bacterial sources require a sequence encoding a CTP sequence fused to a sequence encoding an expressed protein (e.g., effector protein, effector partner) if the expressed protein is to be compartmentalized in the plant plastid (e.g., chloroplast). In some embodiments, the CTP is removed in a processing step during translocation into the plastid. Accordingly, localization of the protein to a chloroplast is often accomplished by means of operably linking a polynucleotide sequence encoding a CTP sequence to the 5′ region of a polynucleotide encoding the exogenous protein.

In some embodiments, the heterologous peptide is an endosomal escape peptide (EEP). An EEP is an agent that quickly disrupts the endosome in order to minimize the amount of time that a delivered molecule, such protein, spends in the endosome-like environment, and to avoid getting trapped in the endosomal vesicles and degraded in the lysosomal compartment. An exemplary EEP is set forth in TABLE 3.

In some embodiments, the heterologous peptide is a cell penetrating peptide (CPP), also known as a Protein Transduction Domain (PTD). A CPP or PTD is a polypeptide, polynucleotide, carbohydrate, or organic or inorganic compound that facilitates traversing a lipid bilayer, micelle, cell membrane, organelle membrane, or vesicle membrane.

Further suitable heterologous peptides include, but are not limited to, proteins (or fragments/domains thereof) that are boundary elements (e.g., CTCF), proteins and fragments thereof that provide periphery recruitment (e.g., Lamin A, Lamin B, etc.), and protein docking elements (e.g., FKBP/FRB, Pil1/Aby1, etc.).

In some embodiments, a heterologous peptide or heterologous peptide comprises a protein tag. In some embodiments, the protein tag is referred to as purification tag or a fluorescent protein. In some embodiments, the protein tag is detectable for use in detection of the protein and/or purification of the protein. Accordingly, in some embodiments, compositions, systems and methods comprise a protein tag or use thereof. Any suitable protein tag may be used depending on the purpose of its use. Non-limiting examples of protein tags include a fluorescent protein, a histidine tag, e.g., a 6×His tag (SEQ ID NO: 908); a hemagglutinin (HA) tag; a FLAG tag; a Myc tag; and maltose binding protein (MBP). In some embodiments, the protein tag is a portion of MBP that can be detected and/or purified. Non-limiting examples of fluorescent proteins include green fluorescent protein (GFP), yellow fluorescent protein (YFP), red fluorescent protein (RFP), cyan fluorescent protein (CFP), mCherry, and tdTomato.

In some embodiments, a heterologous peptide is located at or near the amino terminus (N-terminus) of the protein (e.g., effector protein, effector partner, fusion protein, or combinations thereof) disclosed herein. In some embodiments, a heterologous peptide is located at or near the carboxy terminus (C-terminus) of the proteins disclosed herein. In some embodiments, a heterologous peptide is located internally in the protein described herein (i.e., is not at the N- or C-terminus of the protein described herein) at a suitable insertion site.

In some embodiments, protein (e.g., effector protein or effector partner) described herein comprises 1, 2, 3, 4, 5, 6, 7, 8, 9, 10, or more heterologous peptides at or near the N-terminus, 1, 2, 3, 4, 5, 6, 7, 8, 9, 10, or more heterologous peptides at or near the C-terminus, or a combination of these (e.g., one or more heterologous peptides at the amino-terminus and one or more heterologous peptides at the carboxy terminus). When more than one heterologous peptide is present, each is selected independently of the others, such that a single heterologous peptide is present in more than one copy and/or in combination with one or more other heterologous peptides present in one or more copies. In some embodiments, a heterologous peptide is considered near the N- or C-terminus when the nearest amino acid of the heterologous peptide is within about 1, 2, 3, 4, 5, 10, 15, 20, 25, 30, 40, 50, or more amino acids along the polypeptide chain from the N- or C-terminus.

In some embodiments, a heterologous peptide described herein comprises a heterologous peptide sequence recited in TABLE 3. In some embodiments, effector proteins described herein comprise an amino acid sequence that is at least about 65%, at least about 70%, at least about 75%, at least about 80%, at least about 85%, at least about 90%, at least about 95%, at least about 97%, at least about 98%, at least about 99%, or about 100% identical to any one of the amino acid sequences recited in TABLE 1 and TABLE 2, and further comprises one or more of the amino acid sequences set forth in TABLE 3. In some embodiments, a heterologous peptide described herein comprises an effector partner as described en supra.

In some embodiments, proteins (e.g., effector protein, effector partner, fusion protein, or combinations thereof) described herein are encoded by a codon optimized nucleic acid. In some embodiments, a nucleic acid sequence encoding the protein described herein, is codon optimized. In some embodiments, the proteins described herein is codon optimized for expression in a specific cell, for example, a bacterial cell, a plant cell, a eukaryotic cell, an animal cell, a mammalian cell, or a human cell. In some embodiments, the effector protein is codon optimized for a human cell.

In some embodiments, proteins (e.g., effector protein, effector partner, fusion protein, or combinations thereof) comprise one or more modifications that provide altered activity as compared to an activity of naturally-occurring counterpart (e.g., a naturally-occurring nuclease or nickase which is a naturally-occurring protein). In some embodiments, activity (e.g., nickase, nuclease, binding, deaminase, activity) of proteins described herein is measured relative to a naturally-occurring protein or compositions containing the same in a cleavage assay.

For example, proteins (e.g., effector protein, effector partner, fusion protein, or combinations thereof) comprise one or more modifications that provide increased activity (e.g., catalytic or binding activity) as compared to a naturally-occurring counterpart. As another example, proteins provide increased catalytic activity (e.g., nickase activity, nuclease activity, binding activity, deaminase activity, integrase activity or recombination activity) as compared to a naturally-occurring counterpart. In some embodiments, proteins provide enhanced nucleic acid binding activity (e.g., enhanced binding of a guide nucleic acid, and/or target nucleic acid) as compared to a naturally-occurring counterpart. In some embodiments, proteins have a 1%, 5%, 10%, 20%, 30%, 40%, 50%, 60%, 70%, 80%, 90%, 100%, 120%, 140%, 160%, 180%, 200%, or more, increase of the activity of a naturally-occurring counterpart.

Alternatively, proteins (e.g., effector protein, effector partner, fusion protein, or combinations thereof) comprise one or more modifications that provide reduced catalytic activity (e.g., nickase activity, nuclease activity, binding activity, deaminase activity, integrase activity or recombination activity) as compared to a naturally-occurring counterpart. In some embodiments, proteins have a 100%, 90%, 80%, 70%, 60%, 50%, 40%, 30%, 20%, 10%, 5%, 1%, or less, decrease of the activity of a naturally occurring counterpart.

dCAS Proteins

In some embodiments, an effector protein that has decreased catalytic activity is referred to as catalytically or enzymatically inactive, catalytically or enzymatically dead, as a dead protein or a dCas protein. In some embodiments, such a protein comprises an enzymatically inactive domain (e.g., inactive nuclease domain). For example, in some embodiments, a nuclease domain (e.g., RuvC domain, HNH domain) of an effector protein is deleted or mutated relative to a wildtype counterpart so that it is no longer functional or comprises reduced nuclease activity. In some embodiments, a catalytically inactive effector protein comprises one or more amino acid substitutions as set forth in TABLE 2 relative to the corresponding amino acid sequence of the effector protein as set for the in TABLE 1. In some embodiments, a catalytically inactive effector protein binds to a guide nucleic acid and/or a target nucleic acid but does not cleave the target nucleic acid. In some embodiments, a catalytically inactive effector protein associates with a guide nucleic acid to activate or repress transcription of a target nucleic acid. In some embodiments, a catalytically inactive effector protein is fused to an effector partner that confers an alternative activity to an effector protein activity. Such fusion proteins are described herein and throughout.

### Fusion Proteins

In some embodiments, compositions, systems, and methods comprise a fusion protein or uses thereof. A fusion protein generally comprises at least one effector protein, at least one effector partner, or a combination thereof. In some embodiments, the effector partner is fused or linked to the effector protein. In some embodiments, the effector partner is fused to the N-terminus of the effector protein. In some embodiments, the effector partner is fused to the C-terminus of the effector protein.

In some embodiments, the fusion proteins are multimeric proteins. In some embodiments, the multimeric protein is a homomeric protein. In some embodiments, the multimeric protein is a heteromeric protein. In some embodiments, the fusion protein comprising the effector partner is an effector protein. Accordingly, in such embodiments, the fusion protein can comprise at least two effector proteins that are same. In some embodiments, the fusion protein comprises at least two effector proteins that are different. Unless otherwise indicated, reference to effector proteins throughout the present disclosure include fusion proteins described herein.

In some embodiments, the effector partner is a heterologous protein capable of imparting some function or activity that is not provided by an effector protein. In some embodiments, the effector partner is capable of cleaving or modifying the target nucleic acid. In some embodiments, the fusion proteins disclosed herein provide cleavage activity, such as cis cleavage activity, nickase activity, nuclease activity, integrase activity, recombinase activity, other activity, or a combination thereof. In some embodiments, fusion proteins disclosed herein comprise a RuvC domain capable of cleavage activity. In some embodiments, fusion proteins disclosed herein cleave nucleic acids, including single stranded RNA (ssRNA), double stranded DNA (dsDNA), and single-stranded DNA (ssDNA). In some embodiments, fusion proteins cleave the target nucleic acid at the target sequence or adjacent to the target sequence.

In some embodiments, the fusion protein complexes with a guide nucleic acid and the complex interacts with the target nucleic acid. In some embodiments, the interaction comprises one or more of: recognition of a protospacer adjacent motif (PAM) sequence within the target nucleic acid by the effector protein, hybridization of the guide nucleic acid to the target nucleic acid, modification of the target nucleic acid by the fusion protein, or combinations thereof. In some embodiments, recognition of a PAM sequence within a target nucleic acid directs the modification activity of a fusion protein.

In some embodiments, modification activity of a fusion protein described herein comprises cleavage activity, binding activity, insertion activity, substitution activity, and the like. In some embodiments, modification activity of an effector protein results in: cleavage of at least one strand of a target nucleic acid, deletion of one or more nucleotides of a target nucleic acid, insertion of one or more nucleotides into a target nucleic acid, substitution of one or more nucleotides of a target nucleic acid with an alternative nucleotide, more than one of the foregoing, or any combination thereof. In some embodiments, an ability of a fusion protein to edit a target nucleic acid depends upon the effector protein being complexed with a guide nucleic acid, the guide nucleic acid being hybridized to a target sequence of the target nucleic acid, the distance between the target sequence and a PAM sequence, or combinations thereof. A target nucleic acid comprises a target strand and a non-target strand. Accordingly, in some embodiments, the fusion protein edits a target strand and/or a non-target strand of a target nucleic acid.

In some embodiments, the fusion protein described herein comprises a heterologous amino acid sequence that affects formation of a multimeric complex of the fusion protein. By way of non-limiting example, the fusion protein comprises an effector protein described herein and an effector partner comprising a Calcineurin A tag, wherein the fusion protein dimerizes in the presence of Tacrolimus (FK506). Also, by way of non-limiting example, the fusion protein comprises an effector protein described herein and a SpyTag configured to dimerize or associate with another effector protein in a multimeric complex. Multimeric complex formation is further described herein.

### IV. Multimeric Complexes

Compositions, systems, and methods of the present disclosure comprise a multimeric complex or uses thereof, wherein the multimeric complex comprises one or more polypeptides (e.g., effector proteins, effector partners, fusion proteins, or combinations thereof) that non-covalently interact with one another. In some embodiments, a multimeric complex comprises enhanced activity relative to the activity of any one of its polypeptides (e.g., effector proteins, effector partners, fusion proteins, or combinations thereof) alone. For example, in some embodiments, a multimeric complex comprises two effector proteins (e.g., in dimeric form), wherein the multimeric complex comprises greater nucleic acid binding affinity and/or nuclease activity than that of either of the effector proteins provided in monomeric form. In some embodiments, a multimeric complex comprises one or more heterologous proteins fused to one or more effector proteins, wherein the fusion proteins are capable of different activity than that of the one or more effector proteins. In another example, a multimeric complex comprises an effector protein and a partner protein, wherein the multimeric complex comprises an effector partner, and wherein the multimeric complex comprises greater nucleic acid binding affinity and/or nuclease activity than that of either of the effector protein or effector partner provided in monomeric form. In some embodiments, a multimeric complex comprises an affinity for a target sequence of a target nucleic acid and is capable of catalytic activity (e.g., cleaving, nicking, inserting or otherwise editing the nucleic acid) at or near the target sequence. In some embodiments, a multimeric complex comprises an affinity for a donor nucleic acid and is capable of catalytic activity (e.g., cleaving, nicking, editing or otherwise modifying the nucleic acid by creating cuts) at or near one or more ends of the donor nucleic acid. In some embodiments, multimeric complexes are active when complexed with a guide nucleic acid. In some embodiments, multimeric complexes are active when complexed with a target nucleic acid. In some embodiments, multimeric complexes are active when complexed with a guide nucleic acid, a target nucleic acid, and/or a donor nucleic acid. In some embodiments, the multimeric complex cleaves the target nucleic acid. In some embodiments, the multimeric complex nicks the target nucleic acid.

Various aspects of the present disclosure include compositions and methods comprising multiple polypeptides (e.g., effector proteins, effector partners, fusion proteins, or combinations thereof), and uses thereof, respectively. An effector protein comprising an amino acid sequence that is at least 65%, at least 70%, at least 75%, at least 80%, at least 85%, at least 90%, at least 95% or 100% identical to any one of the amino acid sequences of TABLE 1 are provided with a second effector protein. Two effector proteins target different nucleic acid sequences. Two effector proteins target different types of nucleic acids (e.g., a first effector protein targets double- and single-stranded nucleic acids, and a second effector protein only targets single-stranded nucleic acids). It is understood that when discussing the use of more than one effector protein in compositions, systems, and methods provided herein, the multimeric complex form is also described.

In some embodiments, multimeric complexes comprise at least one polypeptide (e.g., effector protein, effector partner, or fusion protein) as described herein. In some embodiments, the multimeric complex is a dimer comprising a first polypeptide and a second polypeptide. In some embodiments, the first polypeptide and the second polypeptide comprise identical amino acid sequences. In some embodiments, the first polypeptide and the second polypeptide comprise amino acid sequences that are at least 90%, at least 92%, at least 94%, at least 96%, at least 98%, at least 99%, or 100% identical to each other. In some embodiments, the first polypeptide and the second polypeptide comprise similar amino acid sequences. In some embodiments, the first polypeptide and the second polypeptide comprise amino acid sequences that are at least 90%, at least 92%, at least 94%, at least 96%, at least 98%, at least 99%, or 100% similar to each other.

In some embodiments, the multimeric complex is a heterodimeric complex comprising at least two polypeptides (e.g., effector proteins, effector partners, fusion proteins, or combinations thereof) of different amino acid sequences. In some embodiments, the multimeric complex comprises two, three, four, five, six, seven, eight, nine, or ten polypeptides. In some embodiments, the multimeric complex is a heterodimeric complex comprising a first effector protein and a second effector protein, wherein the amino acid sequence of the first effector protein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second effector protein.

In some embodiments, at least one effector protein of the multimeric complex comprises an amino acid sequence that is at least 65%, at least 70%, at least 75%, at least 80%, at least 85%, at least 90%, at least 95% or 100% identical to any one of the amino acid sequences of TABLE 1. In some embodiments, each effector protein of the multimeric complex independently comprises an amino acid sequence with at least 65%, at least 70%, at least 75%, at least 80%, at least 85%, at least 90%, at least 95% or 100% identity to any one of the amino acid sequences of TABLE 1.

In some embodiments, the multimeric complex described herein is capable of targeting polyA signals, splice site acceptors, and start codons. In some embodiments, the multimeric complex cannot create stop codons for knock-down. In some embodiments, the multimeric complex is a dimer comprising fusion protein described herein. In some embodiments, the fusion protein comprises the effector protein described herein, and the effector partner described herein. In some embodiments, the dimer is formed due to non-covalent interactions between the effector proteins of monomers. In some embodiments, N- and C-termini of “formerly active” monomer is closer to 5′ region of non-target strand, while the termini of the “other” monomer is closer to 3′ region, which results in a larger editing window of the multimeric complex having a larger editing window on the non-target strand. In some embodiments, the multimeric complex has a lower editing window for a target strand due to in accessibility for the effector partner.

### Protospacer Adjacent Motif (PAM) Sequences

In some embodiments, polypeptide (e.g., effector protein, effector partner, fusion protein, or a combination thereof) of the present disclosure cleaves or nicks a target nucleic acid within or near a protospacer adjacent motif (PAM) sequence of the target nucleic acid. In some embodiments, the target nucleic acid is a double stranded nucleic acid comprising a target strand and a non-target strand. In some embodiments, cleavage occurs within 1, 2, 3, 4, 5, 6, 7, 8, 9, 10, 15, 20, 25, 30, 35, 40, 45, or 50 nucleotides of a 5′ or 3′ terminus of a PAM sequence. In some embodiments, polypeptides described herein recognize a PAM sequence. In some embodiments, recognizing a PAM sequence comprises interacting with a sequence adjacent to the PAM. In some embodiments, a target nucleic acid comprises a target sequence that is adjacent to a PAM sequence. In some embodiments, the polypeptide does not require a PAM to bind and/or cleave a target nucleic acid.

In some embodiments, a target nucleic acid is a single stranded target nucleic acid comprising a target sequence. Accordingly, in some embodiments, the single stranded target nucleic acid comprises a PAM sequence described herein that is adjacent (e.g., within 1, 2, 3, 4, 5, 6, 7, 8, 9, 10, 15, 20, 25, 30, 35, 40, 45, or 50 nucleotides) or directly adjacent to the target sequence. In some embodiments, an RNP cleaves the single stranded target nucleic acid.

In some embodiments, a target nucleic acid is a double stranded nucleic acid comprising a target strand and a non-target strand, wherein the target strand comprises a target sequence. In some embodiments, the PAM sequence is located on the target strand. In some embodiments, the PAM sequence is located on the non-target strand. In some embodiments, the PAM sequence described herein is adjacent (e.g., within 1, 2, 3, 4, 5, 6, 7, 8, 9, 10, 15, 20, 25, 30, 35, 40, 45, or 50 nucleotides) to the target sequence on the target strand or the non-target strand. In some embodiments, the PAM sequence is located 5′ of a reverse complement of the target sequence on the non-target strand. In some embodiments, such a PAM described herein is directly adjacent to the target sequence on the target strand or the non-target strand. In some embodiments, an RNP cleaves the target strand or the non-target strand. In some embodiments, the RNP cleaves both, the target strand and the non-target strand. In some embodiments, an RNP recognizes the PAM sequence, and hybridizes to a target sequence of the target nucleic acid. In some embodiments, the RNP cleaves the target nucleic acid, wherein the RNP has recognized the PAM sequence and is hybridized to the target sequence.

In some embodiments, an effector protein described herein, or a multimeric complex thereof, recognizes a PAM on a target nucleic acid. In some embodiments, multiple effector proteins of the multimeric complex recognize a PAM on a target nucleic acid. In some embodiments, at least two of the multiple effector proteins recognize the same PAM sequence. In some embodiments, at least two of the multiple effector proteins recognize different PAM sequences. In some embodiments, only one effector protein of the multimeric complex recognizes a PAM on a target nucleic acid.

In some embodiments, an effector protein of the present disclosure, or a multimeric complex thereof, cleaves or nicks a target nucleic acid within or near a protospacer adjacent motif (PAM) sequence of the target nucleic acid. In some embodiments, cleavage occurs within 1, 2, 3, 4, 5, 6, 7, 8, 9 or 10 nucleotides of a 5′ or 3′ terminus of a PAM sequence.

In some embodiments, a PAM sequence provided herein comprises any one of the nucleotide sequences recited in TABLE 4. In some embodiments, polypeptide or effector protein recognizes a PAM as set forth in TABLE 4. In PAMs used in compositions, systems, and methods herein are further described throughout the application.

### V. Guide Nucleic Acids

The compositions, systems, and methods of the present disclosure comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid. Accordingly, compositions, systems, and methods of the present disclosure comprise a guide nucleic acid or a nucleotide sequence encoding the guide nucleic acid. Guide nucleic acids are also referred to herein as “guide RNA.” A guide nucleic acid, as well as any components thereof (e.g., spacer sequence, repeat sequence, linker nucleotide sequence, handle sequence, intermediary sequence etc.) comprise one or more deoxyribonucleotides, ribonucleotides, biochemically or chemically modified nucleotides (e.g., one or more engineered modifications as described herein), or any combinations thereof. Such nucleotide sequences described herein may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the nucleotide sequence that encodes a guide nucleic acid, such as a nucleotide sequence described herein for a vector. Similarly, disclosure of the nucleotide sequences described herein also discloses the complementary nucleotide sequence, the reverse nucleotide sequence, and the reverse complement nucleotide sequence, any one of which can be a nucleotide sequence for use in a guide nucleic acid as described herein. In some embodiments, a guide nucleic acid sequence(s) comprises one or more nucleotide alterations at one or more positions in any one of the nucleotide sequences described herein. Alternative nucleotides can be any one or more of A, C, G, T or U, or a deletion, or an insertion.

In some embodiments, a guide nucleic acid comprises a naturally occurring sequence. In some embodiments, a guide nucleic acid comprises a non-naturally occurring sequence, wherein the nucleotide sequence of the guide nucleic acid, or any portion thereof, is different from the nucleotide sequence of a naturally occurring guide nucleic acid. A guide nucleic acid of the present disclosure comprises one or more of the following: a) a single nucleic acid molecule; b) a DNA base; c) an RNA base; d) a modified base; e) a modified sugar; f) a modified backbone; and the like. Modifications are described herein and throughout the present disclosure (e.g., in the section entitled “Engineered Modifications”). In some embodiments, a guide nucleic acid is chemically synthesized or recombinantly produced by any suitable methods. In some embodiments, guide nucleic acids and portions thereof are found in or identified from a CRISPR array present in the genome of a host organism or cell.

In general, the guide nucleic acid comprises a nucleotide sequence that is at least 80%, at least 85%, at least 90%, at least 95%, at least 98%, at least 99%, or 100% complementary to the target sequence. In some embodiments, the guide nucleic acid comprises at least 10 contiguous nucleotides that are complementary to the target sequence in the target nucleic acid. In some embodiments, guide nucleic acid comprises a spacer sequence that is at least 80%, at least 85%, at least 90%, at least 95%, at least 98%, at least 99%, or 100% complementary to the target sequence.

In general, a guide nucleic acid comprises a first region that is not complementary to a target nucleic acid (FR) and a second region is complementary to the target nucleic acid (SR), wherein the FR and the SR are heterologous to each other. In some embodiments, FR is located 5′ to SR (FR-SR). In some embodiments, SR is located 5′ to FR (SR-FR). In some embodiments, the FR comprises one or more repeat sequence, handle sequence, intermediary sequence, or combinations thereof. In some embodiments, at least a portion of the FR interacts or binds to an effector protein. In some embodiments, the SR comprises a spacer sequence, wherein the spacer sequence can interact in a sequence-specific manner with (e.g., has complementarity with, or can hybridize to a target sequence in) a target nucleic acid.

In some embodiments, the first region, the second region, or both are about 8 linked nucleotides, about 10 linked nucleotides, about 12 linked nucleotides, about 14 linked nucleotides, about 16 linked nucleotides, about 18 linked nucleotides, about 20 linked nucleotides, about 22 linked nucleotides, about 24 linked nucleotides, about 26 linked nucleotides, about 28 linked nucleotides, about 30 linked nucleotides, about 32 linked nucleotides, about 34 linked nucleotides, about 36 linked nucleotides, about 38 linked nucleotides, about 40 linked nucleotides, about 42 linked nucleotides, about 44 linked nucleotides, about 46 linked nucleotides, about 48 linked nucleotides, or about 50 linked nucleotides.

In some embodiments, the first region, the second region, or both comprise from about 8 to about 12, from about 8 to about 16, from about 8 to about 20, from about 8 to about 24, from about 8 to about 28, from about 8 to about 30, from about 8 to about 32, from about 8 to about 34, from about 8 to about 36, from about 8 to about 38, from about 8 to about 40, from about 8 to about 42, from about 8 to about 44, from about 8 to about 48, or from about 8 to about 50 linked nucleotides.

In some embodiments, the first region, the second region, or both comprise a GC content of about 1%, about 5%, about 10%, about 15%, about 20%, about 25%, about 30%, about 40%, about 50%, about 60%, about 70%, about 80%, about 90%, about 95%, or about 99%. In some embodiments, the first region, the second region, or both comprise a GC content of from about 1% to about 95%, from about 5% to about 90%, from about 10% to about 80%, from about 15% to about 70%, from about 20% to about 60%, from about 25% to about 50%, or from about 30% to about 40%.

In some embodiments, the first region, the second region, or both have a melting temperature of about 38° C., about 40° C., about 42° C., about 44° C., about 46° C., about 48° C., about 50° C., about 52° C., about 54° C., about 56° C., about 58° C., about 60° C., about 62° C., about 64° C., about 66° C., about 68° C., about 70° C., about 72° C., about 74° C., about 76° C., about 78° C., about 80° C., about 82° C., about 84° C., about 86° C., about 88° C., about 90° C., or about 92° C. In some embodiments, the first region, the second region, or both have a melting temperature of from about 35° C. to about 40° C., from about 35° C. to about 45° C., from about 35° C. to about 50° C., from about 35° C. to about 55° C., from about 35° C. to about 60° C., from about 35° C. to about 65° C., from about 35° C. to about 70° C., from about 35° C. to about 75° C., from about 35° C. to about 80° C., or from about 35° C. to about 85° C.

In some embodiments, the guide nucleic acid is an engineered guide nucleic acid or a nucleic acid encoding the engineered guide nucleic acid, wherein the engineered guide nucleic acid comprises a first region and a second region, the first region non-covalently binds and activates the polypeptide, and the second region comprises a nucleic acid that is complementary to or the reverse complement of the target nucleic acid. In some embodiments, the first region and the second region are heterologous to each other. In some embodiments, the engineered guide nucleic acid comprises a nucleotide sequence that is at least 85% identical, at least 90% identical, at least 95% identical, or at least 100% identical to any one of the nucleotide sequences set forth in TABLE 9. In some embodiments, the first region comprises a nucleotide sequence that at least partially interacts with the polypeptide. In some embodiments, the first region comprises a nucleotide sequence that is at least 85% identical, at least 90% identical, at least 95% identical, or at least 100% identical to any one of the nucleotide sequences set forth in TABLE 5, TABLE 8, or a combination thereof. In some embodiments, the first region comprises a repeat sequence that at least partially interacts with the polypeptide. In some embodiments, the first region comprises an intermediary sequence that at least partially interacts with the polypeptide. In some embodiments, the first region comprises a handle sequence that at least partially interacts with the polypeptide. In some embodiments, the handle sequence comprises an intermediary sequence, a repeat sequence, or combinations thereof. In some embodiments, the handle sequence further comprises a linker sequence that is directly linked to the intermediary sequence, the repeat sequence, or both. In some embodiments, the second region comprises at least 10 contiguous nucleotides that are complementary to a eukaryotic sequence. In some embodiments, the second region comprises at least 10 contiguous nucleotides that are the reverse complement of the target sequence. In some embodiments, the second region comprises a spacer sequence that hybridizes to a target sequence of a target nucleic acid.

In some embodiments, the compositions, systems, and methods of the present disclosure further comprise an additional nucleic acid, wherein a portion of the additional nucleic acid at least partially hybridizes to the first region of the guide nucleic acid. In some embodiments, the additional nucleic acid is at least partially hybridized to the 5′ end of the second region of the guide nucleic acid. In some embodiments, an unhybridized portion of the additional nucleic acid, at least partially, interacts with an effector protein or polypeptide. In some embodiments, the compositions, systems, and methods of the present disclosure comprise a dual nucleic acid system comprising the guide nucleic acid and the additional nucleic acid as described herein.

In some embodiments, the guide nucleic acid also forms complexes as described through herein. For example, in some embodiments, a guide nucleic acid hybridizes to another nucleic acid, such as target nucleic acid, or a portion thereof. In another example, a guide nucleic acid complexes with an effector protein. In such embodiments, a guide nucleic acid-effector protein complex is described herein as an RNP. In some embodiments, when in a complex, at least a portion of the complex binds, recognizes, and/or hybridizes to a target nucleic acid. For example, when a guide nucleic acid and an effector protein are complexed to form an RNP, at least a portion of the guide nucleic acid hybridizes to a target sequence in a target nucleic acid. Those skilled in the art in reading the below specific examples of guide nucleic acids as used in RNPs described herein, will understand that in some embodiments, a RNP hybridizes to one or more target sequences in a target nucleic acid, thereby allowing the RNP to modify and/or recognize a target nucleic acid or sequence contained therein (e.g., PAM) or to modify and/or recognize non-target sequences depending on the guide nucleic acid, and in some embodiments, the effector protein, used.

In some embodiments, a guide nucleic acid comprises or forms intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the guide nucleic acid comprises a pseudoknot (e.g., a secondary structure comprising a stem, at least partially, hybridized to a second stem or half-stem secondary structure). In some embodiments, an effector protein recognizes a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, the compositions, systems, and methods of the present disclosure comprise two or more guide nucleic acids (e.g., 2, 3, 4, 5, 6, 7, 9, 10 or more guide nucleic acids), and/or uses thereof. In some embodiments, multiple guide nucleic acids target an effector protein to different locations in the target nucleic acid by hybridizing to different target sequences. In some embodiments, a first guide nucleic acid hybridizes within a location of the target nucleic acid that is different from where a second guide nucleic acid hybridizes the target nucleic acid. In some embodiments, the first loci and the second loci of the target nucleic acid are located at least 1, at least 10, at least 20, at least 30, at least 40, at least 50, at least 60, at least 70, at least 80, at least 90 or at least 100 nucleotides apart. In some embodiments, the first loci and the second loci of the target nucleic acid are located between 100 and 200, 200 and 300, 300 and 400, 400 and 500, 500 and 600, 600 and 700, 700 and 800, 800 and 900 or 900 and 1000 nucleotides apart. In some embodiments, the first loci and/or the second loci of the target nucleic acid are located in an intron of a gene. In some embodiments, the first loci and/or the second loci of the target nucleic acid are located in an exon of a gene. In some embodiments, the first loci and/or the second loci of the target nucleic acid span an exon-intron junction of a gene. In some embodiments, the first portion and/or the second portion of the target nucleic acid are located on either side of an exon and cutting at both sites results in deletion of the exon. In some embodiments, composition, systems, and methods comprise a donor nucleic acid that is inserted in replacement of a deleted or cleaved sequence of the target nucleic acid. In some embodiments, compositions, systems, and methods comprising multiple guide nucleic acids or uses thereof comprise multiple effector proteins, wherein the effector proteins are identical, non-identical, or combinations thereof.

In some embodiments, a guide nucleic acid comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, a guide nucleic acid comprises at least: 10, 15, 20, 25, 30, 35, 40, 45, 50, 55, 60 linked nucleotides. In some embodiments, the guide nucleic acid has about 10 to about 60, about 20 to about 50, or about 30 to about 40 linked nucleotides.

In some embodiments, a guide nucleic acid comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a nucleotide sequence that is present in a host eukaryotic cell. Such a nucleotide sequence is distinguished from nucleotide sequences present in other host cells, such as prokaryotic cells, or viruses. Said sequences present in a eukaryotic cell can be located in a gene, an exon, an intron, a non-coding (e.g., promoter or enhancer) region, a selectable marker, tag, signal, and the like. In some embodiments, a target sequence is a eukaryotic sequence.

In some embodiments, a length of a guide nucleic acid is about 30 to about 120 linked nucleotides. In some embodiments, the length of a guide nucleic acid is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a guide nucleic acid is about 40, about 45, about 50, about 55, about 60, about 65, about 70 or about 75 linked nucleotides. In some embodiments, the length of a guide nucleic acid is greater than about 15, about 20, about 25, about 30, about 35, about 40, about 45, about 50, about 55, about 60, about 65, about 70 or about 75 linked nucleotides. In some embodiments, the length of a guide nucleic acid is not greater than about 40, about 45, about 50, about 55, about 60, about 65, about 70, about 75, about 80, about 85, about 90, about 95, about 100, about 105, about 110, about 115, about 120, or about 125 linked nucleotides.

In some embodiments, guide nucleic acids comprise additional elements that contribute additional functionality (e.g., stability, heat resistance, etc.) to the guide nucleic acid. In some embodiments, the elements comprise one or more nucleotide alterations, nucleotide sequences, intermolecular secondary structures, or intramolecular secondary structures (e.g., one or more hair pin regions, one or more bulges, etc.).

In some embodiments, guide nucleic acids comprise one or more linkers connecting different nucleotide sequences as described herein. In some embodiments, a linker comprises 1, 2, 3, 4, 5, 6, 7, 8, 9, 10, or more nucleotides. In some embodiments, a linker comprises any suitable linker, examples of which are described herein.

In some embodiments, guide nucleic acids comprise one or more nucleotide sequences as described herein (e.g., TABLE 5, TABLE 6, TABLE 7, TABLE 8 and TABLE 9). In some embodiments, such nucleotide sequences described herein (e.g., TABLE 5, TABLE 6, TABLE 7, TABLE 8 and TABLE 9) are DNA or RNA, however, no matter the form of the nucleotide sequence described, it is readily understood that such nucleotide sequences may be revised to be RNA or DNA, as needed, for describing a sequence within a guide nucleic acid itself or the nucleotide sequence that encodes a guide nucleic acid, such as a nucleotide sequence described herein for a vector. Similarly, disclosure of the nucleotide sequences described herein (e.g., TABLE 5, TABLE 6, TABLE 7, TABLE 8 and TABLE 9) also discloses the complementary nucleotide sequence, the reverse nucleotide sequence, and the reverse complement nucleotide sequence, any one of which is a nucleotide sequence for use in a guide nucleic acid as described herein. In some embodiments, guide nucleic acids described herein comprise at least two nucleotide sequences as described herein (e.g., TABLE 9). In some embodiments, guide nucleic acid sequence(s) comprises one or more nucleotide alterations at one or more positions in any one of the nucleotide sequences described herein. Alternative nucleotides may be any one or more of A, C, G, T or U, or a deletion, or an insertion. In some embodiments, guide nucleic acids comprise an intermediary sequence, a linker, a spacer, a repeat, or combinations thereof. In some embodiments, guide nucleic acids comprise an intermediary sequence (SEQ ID No. 257) and a repeat sequence (any one of SEQ ID No. 251-254, 703-790, 792-811, 815-835). In some embodiments, guide nucleic acids comprise an intermediary sequence (SEQ ID No. 257) and a spacer sequence (any one of SEQ ID No. 860-893). In some embodiments, guide nucleic acids comprise an intermediary sequence (SEQ ID NO: 257), a linker sequence (SEQ ID NO: 255), a repeat sequence (SEQ ID NO: 251), and a spacer sequence (any one of SEQ ID NO: 870-893).

In some embodiments, the guide nucleic acid comprises a nucleotide sequence that is capable of hybridizing to a target sequence in a target nucleic acid, wherein the target nucleic acid is any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence, and combinations thereof.

In some embodiments, the guide nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell.

In some embodiments, the guide nucleic acid comprises a handle (sgRNA) or a repeat (crRNA). In some embodiments, the guide nucleic acid comprises a repeat (crRNA). In some embodiments, the guide nucleic acid is an engineered guide nucleic acid, wherein the engineered guide nucleic acid comprises a sgRNA or a crRNA. In some embodiments, the guide nucleic acid is an engineered guide nucleic acid, wherein the engineered guide nucleic acid comprises a crRNA. In some embodiments, the engineered guide nucleic acid comprises one or more phosphorothioate (PS) backbone modifications, 2′-fluoro (2′-F) sugar modifications, or 2′-O-Methyl (2′OMe) sugar modifications. In some embodiments, the nucleic acid encoding the polypeptide, the nucleic acid encoding the engineered guide nucleic acid, or both are mRNA.

### Spacer Sequence

In some embodiments, guide nucleic acids described herein comprise one or more spacer sequences. In some embodiments, a spacer sequence is capable of hybridizing to a target sequence of a target nucleic acid. In some embodiments, a spacer sequence comprises a nucleotide sequence that is, at least partially, hybridizable to an equal length of a sequence (e.g., a target sequence) of a target nucleic acid. Exemplary hybridization conditions are described herein. In some embodiments, the spacer sequence functions to direct an RNP complex comprising the guide nucleic acid to the target nucleic acid for detection and/or modification. In some embodiments, the spacer sequence functions to direct a RNP to the target nucleic acid for detection and/or modification. In some embodiments, a spacer sequence is complementary to a target sequence that is adjacent to a PAM that is recognizable by an effector protein described herein.

In some embodiments, a spacer sequence comprises at least 5 to about 50 contiguous nucleotides that are complementary to a target sequence in a target nucleic acid. In some embodiments, a spacer sequence comprises at least 5 to about 50 linked nucleotides. In some embodiments, a spacer sequence comprises at least 5 to about 50, at least 5 to about 25, at least about 10 to about 25, or at least 15 to about 25 linked nucleotides. In some embodiments, the spacer sequence comprises 15-28 linked nucleotides. In some embodiments, a spacer sequence comprises 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides. In some embodiments, the spacer sequence comprises 10, 11, 12, 13, 14, 15, 16, 17, 18, 19, 20, 21, 22, 23, 24, 25, 26, 27, 28, 29, 30, or more nucleotides.

In some embodiments, a spacer sequence comprises a nucleotide sequence that is at least 65%, at least 70%, at least 75%, at least 80%, at least 85%, at least 90%, at least 95% or 100% identical to any one of the spacer sequences listed in TABLE 7.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. In some embodiments, linkers comprise any suitable linker. In some embodiments, the spacer sequence(s) and the repeat sequence(s) of the guide nucleic acid are present in separate molecules, which are joined to one another by base pairing interactions.

In some embodiments, a spacer sequence is capable of hybridizing to an equal length portion of a target nucleic acid (e.g., a target sequence). In some embodiments, a target nucleic acid, such as DNA or RNA, comprises a gene associated with a genetic disorder, a cancer gene, or an amplicon thereof, as described herein. In some embodiments, a target nucleic acid comprises any one of the genes recited in TABLE 10, a variant thereof, a promoter thereof, an enhancer thereof, or a portion thereof. In some embodiments, a spacer sequence comprises a nucleotide sequence that is at least 65%, at least 70%, at least 75%, at least 80%, at least 85%, at least 90%, at least 95% or 100% complementary to or the reverse complement of a target sequence of a target nucleic acid selected from TABLE 11. In some embodiments, the spacer sequence comprises a nucleotide sequence that is at least 65%, at least 70%, at least 75%, at least 80%, at least 85%, at least 90%, at least 95% or 100% complementary to or the reverse complement of to an equal length portion of a target sequence of a gene encoding the HBB gene, a promoter thereof, an enhancer thereof, or a fragment thereof. In some embodiments, the spacer sequence comprises a nucleotide sequence that is at least 65%, at least 70%, at least 75%, at least 80%, at least 85%, at least 90%, at least 95% or 100% complementary to or the reverse complement of to an equal length portion of a target sequence of a gene encoding the BCL11A gene, a promoter thereof, an enhancer thereof, or a fragment thereof. In some embodiments, the spacer sequence comprises a nucleotide sequence that is at least 65%, at least 70%, at least 75%, at least 80%, at least 85%, at least 90%, at least 95% or 100% complementary to or the reverse complement of to an equal length portion of a target sequence of a gene encoding the HBG1 gene, a promoter thereof, an enhancer thereof, or a fragment thereof. In some embodiments, the spacer sequence comprises a nucleotide sequence that is at least 65%, at least 70%, at least 75%, at least 80%, at least 85%, at least 90%, at least 95% or 100% complementary to or the reverse complement of to an equal length portion of a target sequence of a gene encoding the HBG2 gene, a promoter thereof, an enhancer thereof, or a fragment thereof.

In some embodiments, a target nucleic acid is a nucleic acid associated with a disease or syndrome set forth in TABLE 12. In some embodiments, a spacer sequence comprises a nucleotide sequence that is at least 65%, at least 70%, at least 75%, at least 80%, at least 85%, at least 90%, at least 95% or 100% complementary to or the reverse complement of to a target sequence of a target nucleic acid associated with a disease or syndrome set forth in TABLE 12. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are capable of hybridizing to the target sequence. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are complementary to or the reverse complement of to the target sequence.

It is understood that the spacer sequence of a spacer sequence need not be 100% complementary to that of a target sequence of a target nucleic acid to hybridize or hybridize specifically to the target sequence. For example, the spacer sequence, in some embodiments, comprises at least one alteration, such as a substituted or modified nucleotide, that is not complementary to the corresponding nucleotide of the target sequence. Alternative nucleotides can be any one or more of A, C, G, T or U, or a deletion, or an insertion.

### Repeat Sequence

In some embodiments, guide nucleic acids described herein comprise one or more repeat sequences. In some embodiments, a repeat sequence comprises a nucleotide sequence that is not complementary to a target sequence of a target nucleic acid. In some embodiments, a repeat sequence comprises a nucleotide sequence that interacts with an effector protein. In some embodiments, a repeat sequence is connected to another sequence of a guide nucleic acid, such as an intermediary sequence, that is capable of non-covalently interacting with an effector protein. In some embodiments, a repeat sequence includes a nucleotide sequence that is capable of forming a guide nucleic acid-effector protein complex (e.g., a RNP complex).

In some embodiments, the repeat sequence is between 10 and 50, 12 and 48, 14 and 46, 16 and 44, and 18 and 42 nucleotides in length. In some embodiments, the repeat sequence is at least 13, at least 15, at least 18, at least 21, at least 24, at least 27, at least 30, at least 33, or at least 36 linked nucleotides in length.

In some embodiments, a repeat sequence is adjacent to a spacer sequence. In some embodiments, a repeat sequence is followed by a spacer sequence in the 5′ to 3′ direction. In some embodiments, a repeat sequence is preceded by a spacer sequence in the 5′ to 3′ direction. In some embodiments, a repeat sequence is adjacent to an intermediary sequence. In some embodiments, a repeat sequence is 3′ to an intermediary sequence. In some embodiments, an intermediary sequence is followed by a repeat sequence, which is followed by a spacer sequence in the 5′ to 3′ direction. In some embodiments, a repeat sequence is linked to a spacer sequence and/or an intermediary sequence. In some embodiments, a guide nucleic acid comprises a repeat sequence linked to a spacer sequence and/or to an intermediary sequence by a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence comprises a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences comprise 65% to 100% complementarity (e.g., 65%, 70%, 75%, 80%, 85%, 90%, 95%, or 100% complementarity). In some embodiments, a guide nucleic acid comprises a nucleotide sequence that, when involved in hybridization events, hybridizes over one or more segments of a target nucleic acid such that intervening or adjacent segments are not involved in the hybridization event (e.g., a bulge, a loop structure or hairpin structure, etc.).

In some embodiments, a repeat sequence comprises a nucleotide sequence that is at least 65%, at least 70%, at least 75%, at least 80%, at least 85%, at least 90%, at least 95%, at least 97%, or at least 98%, at least 99%, or 100% identical to an equal length portion of any one of the repeat sequences in TABLE 5. In some embodiments, the repeat sequence is at least 85% identical to any one of sequences set forth in TABLE 5. In some embodiments, a repeat sequence comprises at least 5, at least 6, at least 7, at least 8, at least 9, at least 10, at least 11, at least 12, at least 13, at least 14, at least 15, at least 16, at least 17, at least 18, at least 19, at least 20, or at least 21 contiguous nucleotides of any one of the nucleotide sequences recited in TABLE 5.

In some embodiments, a repeat sequence comprises one or more nucleotide alterations at one or more positions in the nucleotide sequence recited in TABLE 5. Alternative nucleotides can be any one or more of A, C, G, T or U, or a deletion, or an insertion.

### Linker for Nucleic Acids

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises more than one linker. In some embodiments, at least two of the more than one linker are the same. In some embodiments, at least two of the more than one linker are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence recited in TABLE 6.

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### Intermediary Sequence

In some embodiments, guide nucleic acids described herein comprise one or more intermediary sequences. In some embodiments, intermediary RNA is a nucleotide sequence in a handle sequence, wherein the nucleotide sequence is capable of, at least partially, being non-covalently bound to an effector protein to form a complex (e.g., an RNP complex). In general, an intermediary sequence used in the present disclosure is not transactivated or transactivating. In some embodiments, an intermediary sequence is also be referred to as an intermediary RNA, although it comprises deoxyribonucleotides instead of or in addition to ribonucleotides, and/or modified bases. In general, the intermediary sequence non-covalently binds to an effector protein. In some embodiments, the intermediary sequence forms a secondary structure, for example in a cell, and an effector protein binds the secondary structure.

In some embodiments, a length of the intermediary sequence is at least 30, 40, or 50 linked nucleotides. In some embodiments, a length of the intermediary sequence is not greater than 30, 40, 50 or 60 linked nucleotides. In some embodiments, the length of the intermediary sequence is about 30 to about 60, about 40 to about 60, or about 50 to about 60 linked nucleotides.

In some embodiments, an intermediary sequence also comprises or forms a secondary structure (e.g., one or more hairpin loops) that facilitates the binding of an effector protein to a guide nucleic acid and/or modification activity of an effector protein on a target nucleic acid (e.g., a hairpin region). In some embodiments, an intermediary sequence comprises from 5′ to 3′, a 5′ region, a hairpin region, and a 3′ region. In some embodiments, the 5′ region hybridizes to the 3′ region. In some embodiments, the 5′ region of the intermediary sequence does not hybridize to the 3′ region.

In some embodiments, the hairpin region comprises a first nucleotide sequence, a second nucleotide sequence that is the reverse complement of the first nucleotide sequence, and a stem-loop linking the first nucleotide sequence and the second nucleotide sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). In some embodiments, an effector protein interacts with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

In some embodiments, an intermediary sequence comprises a nucleotide sequence that is at least 65%, at least 70%, at least 75%, at least 80%, at least 85%, at least 90%, at least 95%, at least 97%, or at least 98%, at least 99%, or 100% identical to the intermediary sequence in TABLE 8. In some embodiments, an intermediary sequence comprises at least 5, at least 6, at least 7, at least 8, at least 9, at least 10, at least 20, at least 30, at least 40, or at least 50 contiguous nucleotides of the intermediary sequence recited in TABLE 8.

In some embodiments, an intermediary sequence comprises one or more nucleotide alterations at one or more positions in the nucleotide sequence recited in TABLE 8. Alternative nucleotides can be any one or more of A, C, G, T or U, or a deletion, or an insertion.

### Handle Sequence

In some embodiments, guide nucleic acids described herein comprise one or more handle sequences. In some embodiments, the handle sequence comprises an intermediary sequence. In such instances, at least a portion of an intermediary sequence non-covalently bonds with an effector protein. In some embodiments, the intermediary sequence is at the 3′-end of the handle sequence. In some embodiments, the intermediary sequence is at the 5′-end of the handle sequence. Additionally, or alternatively, in some embodiments, the handle sequence further comprises one or more of linkers and repeat sequences. In such instances, at least a portion of an intermediary sequence, or both of at least a portion of the intermediary sequence and at least a portion of repeat sequence, non-covalently interacts with an effector protein. In some embodiments, an intermediary sequence and repeat sequence are directly linked (e.g., covalently linked, such as through a phosphodiester bond). In some embodiments, the intermediary sequence and repeat sequence are linked by a suitable linker, examples of which are provided herein. In some embodiments, the linker comprises the nucleotide sequence recited in TABLE 6. In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker. In general, a single guide nucleic acid, also referred to as a single guide RNA (sgRNA), comprises a handle sequence comprising an intermediary sequence, and optionally one or more of a repeat sequence and a linker.

In some embodiments, a handle sequence comprises or forms a secondary structure (e.g., one or more hairpin loops) that facilitates the binding of an effector protein to a guide nucleic acid and/or modification activity of an effector protein on a target nucleic acid (e.g., a hairpin region). In some embodiments, handle sequences comprise a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the handle sequence comprises a pseudoknot (e.g., a secondary structure comprising a stem at least partially hybridized to a second stem or half-stem secondary structure). In some embodiments, an effector protein recognizes a handle sequence comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the handle sequence comprises at least 2, at least 3, at least 4, or at least 5 stem regions.

In some embodiments, a length of the handle sequence is at least 30, 40, 50, 60, or 70 linked nucleotides. In some embodiments, a length of the handle sequence is not greater than 30, 40, 50, 60, or 70 linked nucleotides. In some embodiments, the length of the handle sequence is about 30 to about 70, about 40 to about 70, about 50 to about 70, or about 60 to about 70 linked nucleotides.

In some embodiments, the handle sequence comprises an intermediary sequence that is at least 65%, at least 70%, at least 75%, at least 80%, at least 85%, at least 90%, at least 95%, at least 97%, at least 98%, at least 99%, or 100% identical to any one of the nucleotide sequences as set forth in TABLE 8. In some embodiments, the handle sequence further comprises a linker sequence as set forth in TABLE 6. In some embodiments, the handle sequence further comprises a repeat sequence that is at least 65%, at least 70%, at least 75%, at least 80%, at least 85%, at least 90%, at least 95%, at least 97%, at least 98%, at least 99%, or 100% identical to any one of the nucleotide sequences as set forth in TABLE 5. In some embodiments, the handle sequence comprises an intermediary sequence that is at least 65%, at least 70%, at least 75%, at least 80%, at least 85%, at least 90%, at least 95%, at least 97%, at least 98%, at least 99%, or 100% identical to SEQ ID No. 257. In some embodiments, the handle sequence comprises a linker sequence that is at least 65%, at least 70%, at least 75%, at least 80%, at least 85%, at least 90%, at least 95%, at least 97%, at least 98%, at least 99%, or 100% identical to SEQ ID No. 255. In some embodiments, the handle sequence comprises a repeat sequence that is at least 65%, at least 70%, at least 75%, at least 80%, at least 85%, at least 90%, at least 95%, at least 97%, at least 98%, at least 99%, or 100% identical to SEQ ID No. 251 or 784. In some embodiments, the handle sequence comprises an intermediary sequence (SEQ ID No. 257), a linker sequence (SEQ ID No. 255), and a repeat sequence (SEQ ID No. 251). In some embodiments, the handle sequence comprises an intermediary sequence (SEQ ID No. 257), a linker sequence (SEQ ID No. 255), and a repeat sequence (SEQ ID No. 784). In some embodiments, the handle sequence comprises one or more nucleotide alterations at one or more positions in the nucleotide sequence. Alternative nucleotides can be any one or more of A, C, G, T or U, or a deletion, or an insertion.

### VI. A Single Nucleic Acid System

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, a first region (FR) of the guide nucleic acid non-covalently interacts with the one or more polypeptides described herein. In some embodiments, a second region (SR) of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA or a sgRNA.

crRNA

In some embodiments, a guide nucleic acid comprises a crRNA. In some embodiments, the guide nucleic acid is the crRNA. In general, a crRNA comprises a first region (FR) and a second region (SR), wherein the FR of the crRNA comprises a repeat sequence, and the SR of the crRNA comprises a spacer sequence. In some embodiments, the repeat sequence and the spacer sequences are directly connected to each other (e.g., covalent bond (phosphodiester bond)). In some embodiments, the repeat sequence and the spacer sequence are connected by a linker.

In some embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In such embodiments, a single nucleic acid system comprises a guide nucleic acid comprising a crRNA wherein, a repeat sequence of a crRNA is capable of connecting a crRNA to an effector protein. In some embodiments, a single nucleic acid system comprises a guide nucleic acid comprising a crRNA linked to another nucleotide sequence that is capable of being non-covalently bond by an effector protein. In such embodiments, a repeat sequence of a crRNA can be linked to an intermediary sequence. In some embodiments, a single nucleic acid system comprises a guide nucleic acid comprising a crRNA and an intermediary sequence.

In some embodiments, a crRNA comprises deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

In some embodiments, a crRNA sequence comprises a repeat sequence that is at least 65%, at least 70%, at least 75%, at least 80%, at least 85%, at least 90%, at least 95%, at least 97%, or at least 98%, at least 99%, or 100% identical to any one of the nucleotide sequences set forth in TABLE 5 and a spacer sequence. In some embodiments, a crRNA sequence comprises at least 5, at least 6, at least 7, at least 8, at least 9, or at least 10 contiguous nucleotides of any one of the repeat sequences recited in TABLE 5 and a spacer sequence.

sgRNA

In some embodiments, a guide nucleic acid comprises a sgRNA. In some embodiments, a guide nucleic acid is a sgRNA. In some embodiments, a sgRNA comprises a first region (FR) and a second region (SR), wherein the FR comprises a handle sequence and the SR comprises a spacer sequence. In some embodiments, the handle sequence and the spacer sequences are directly connected to each other (e.g., covalent bond (phosphodiester bond)). In some embodiments, the handle sequence and the spacer sequence are connected by a linker.

In some embodiments, a sgRNA comprises one or more of a handle sequence, an intermediary sequence, a crRNA, a repeat sequence, a spacer sequence, a linker, or combinations thereof. For example, a sgRNA comprises a handle sequence and a spacer sequence; an intermediary sequence and an crRNA; an intermediary sequence, a repeat sequence and a spacer sequence; and the like.

In some embodiments, a sgRNA comprises an intermediary sequence and an crRNA. In some embodiments, an intermediary sequence is 5′ to a crRNA in a sgRNA. In some embodiments, a sgRNA comprises a linked intermediary sequence and crRNA. In some embodiments, an intermediary sequence and a crRNA are linked in a sgRNA directly (e.g., covalently linked, such as through a phosphodiester bond) In some embodiments, an intermediary sequence and a crRNA are linked in a sgRNA by any suitable linker, examples of which are provided herein.

In some embodiments, a sgRNA comprises a handle sequence and a spacer sequence. In some embodiments, a handle sequence is 5′ to a spacer sequence in a sgRNA. In some embodiments, a sgRNA comprises a linked handle sequence and spacer sequence. In some embodiments, a handle sequence and a spacer sequence are linked in a sgRNA directly (e.g., covalently linked, such as through a phosphodiester bond) In some embodiments, a handle sequence and a spacer sequence are linked in a sgRNA by any suitable linker, examples of which are provided herein.

In some embodiments, a sgRNA comprises an intermediary sequence, a repeat sequence, and a spacer sequence. In some embodiments, an intermediary sequence is 5′ to a repeat sequence in a sgRNA. In some embodiments, a sgRNA comprises a linked intermediary sequence and repeat sequence. In some embodiments, an intermediary sequence and a repeat sequence are linked in a sgRNA directly (e.g., covalently linked, such as through a phosphodiester bond) In some embodiments, an intermediary sequence and a repeat sequence are linked in a sgRNA by any suitable linker, examples of which are provided herein. In some embodiments, a repeat sequence is 5′ to a spacer sequence in a sgRNA. In some embodiments, a sgRNA comprises a linked repeat sequence and spacer sequence. In some embodiments, a repeat sequence and a spacer sequence are linked in a sgRNA directly (e.g., covalently linked, such as through a phosphodiester bond) In some embodiments, a repeat sequence and a spacer sequence are linked in a sgRNA by any suitable linker, examples of which are provided herein.

In some embodiments, a sgRNA sequence comprises a nucleotide sequence that is at least 65%, at least 70%, at least 75%, at least 80%, at least 85%, at least 90%, at least 95%, at least 97%, or at least 98%, at least 99%, or 100% identical to any one of the nucleotide sequences recited in TABLE 5, TABLE 6, TABLE 7, TABLE 8 and TABLE 9.

### VII. Engineered Modifications

Polypeptides (e.g., effector proteins) and nucleic acids (e.g., engineered guide nucleic acids) can be further modified as described herein. Examples are modifications that do not alter the primary sequence of the polypeptides or nucleic acids, such as chemical derivatization of polypeptides (e.g., acylation, acetylation, carboxylation, amidation, etc.), or modifications that do alter the primary sequence of the polypeptide or nucleic acid. Also included are polypeptides that have a modified glycosylation pattern (e.g., those made by: modifying the glycosylation patterns of a polypeptide during its synthesis and processing or in further processing steps; by exposing the polypeptide to enzymes which affect glycosylation, such as mammalian glycosylating or deglycosylating enzymes). Also embraced are polypeptides that have phosphorylated amino acid residues (e.g., phosphotyrosine, phosphoserine, or phosphothreonine).

Modifications disclosed herein can also include modification of described polypeptides and/or guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable for their intended purpose (e.g., in vivo administration, in vitro methods, or ex vivo applications). Analogs of such polypeptides include those containing residues other than naturally occurring L-amino acids, e.g., D-amino acids or non-naturally occurring synthetic amino acids. In some embodiments, D-amino acids is substituted for some or all of the amino acid residues. Modifications can also include modifications with non-naturally occurring unnatural amino acids. The particular sequence and the manner of preparation can be determined by convenience, economics, purity required, and the like.

Modifications can further include the introduction of various groups to polypeptides and/or guide nucleic acids described herein. For example, groups can be introduced during synthesis or during expression of a polypeptide (e.g., an effector protein), which allow for linking to other molecules or to a surface. Thus, in some embodiments, cysteines are used to make thioethers, histidines for linking to a metal ion complex, carboxyl groups for forming amides or esters, amino groups for forming amides, and the like.

Modifications can further include changing of nucleic acids described herein (e.g., engineered guide nucleic acids) to provide the nucleic acid with a new or enhanced feature, such as improved stability. Such modifications of a nucleic acid include a base editing, a base modification, a backbone modification, a sugar modification, or combinations thereof. In some embodiments, the modifications can be of one or more nucleotides, nucleosides, or nucleobases in a nucleic acid.

In some embodiments, nucleic acids (e.g., nucleic acids encoding effector proteins, engineered guide nucleic acids, or nucleic acids encoding engineered guide nucleic acids) described herein comprise one or more modifications comprising: 2′O-methyl modified nucleotides (e.g., 2′-O-Methyl (2′OMe) sugar modifications); 2′ fluoro modified nucleotides (e.g., 2′-fluoro (2′-F) sugar modifications); locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH₂—NH—O—CH₂—, —CH₂—N(CH₃)—O—CH₂—(known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH₂ component parts; and combinations thereof.

### VIII. Vectors and Multiplexed Expression Vectors

Compositions, systems, and methods described herein comprise a vector or a use thereof. A vector can comprise a nucleic acid of interest. In some embodiments, the nucleic acid of interest comprises one or more components of a composition or system described herein. In some embodiments, the nucleic acid of interest comprises a nucleotide sequence that encodes one or more components of the composition or system described herein. In some embodiments, one or more components comprises a polypeptide(s) (e.g., effector protein(s), effector partner(s), fusion protein(s), or combinations thereof), guide nucleic acid(s), target nucleic acid(s), and donor nucleic acid(s). In some embodiments, the component comprises a nucleic acid encoding a polypeptide (e.g., effector protein(s), effector partner(s), fusion protein(s), or combinations thereof), a donor nucleic acid, and a guide nucleic acid or a nucleic acid encoding the guide nucleic acid. In some embodiments, a vector is a part of a vector system. In some embodiments, the vector system comprises a library of vectors each encoding one or more component of a composition or system described herein. In some embodiments, components described herein (e.g., an effector protein, a guide nucleic acid, and/or a donor nucleic acid) are encoded by the same vector. In some embodiments, components described herein (e.g., an effector protein, a guide nucleic acid, and/or a donor nucleic acid) are each encoded by different vectors of the system. In some embodiments, a vector encoding a donor nucleic acid further encodes a target nucleic acid.

In some embodiments, a vector comprises a nucleotide sequence encoding one or more polypeptides (e.g., effector proteins, effector partners, fusion proteins, or combinations thereof as described herein. In some embodiments, the one or more polypeptides comprise at least two polypeptides. In some embodiments, the at least two polypeptides are the same. In some embodiments, the at least two polypeptides are different from each other. In some embodiments, the nucleotide sequence is operably linked to a promoter that is operable in a target cell, such as a eukaryotic cell. In some embodiments, the vector comprises the nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more polypeptides.

In some embodiments, a vector encodes one or more of any system components, including but not limited to polypeptides (e.g., effector proteins, effector partners, fusion proteins, or combinations thereof), guide nucleic acids, and target nucleic acids as described herein. In some embodiments, a system component encoding sequence is operably linked to a promoter that is operable in a target cell, such as a eukaryotic cell. In some embodiments, a vector encodes 1, 2, 3, 4 or more of any system components. For example, in some embodiments, a vector encodes two or more guide nucleic acids, wherein each guide nucleic acid comprises a different sequence. In some embodiments, a vector encodes the polypeptide and the guide nucleic acid. In some embodiments, a vector encodes the polypeptide, a guide nucleic acid, a donor nucleic acid, or combinations thereof.

In some embodiments, a vector comprises one or more guide nucleic acids, or a nucleotide sequence encoding the one or more guide nucleic acids as described herein. In some embodiments, the one or more guide nucleic acids comprise at least two guide nucleic acids. In some embodiments, the at least two guide nucleic acids are the same. In some embodiments, the at least two guide nucleic acids are different from each other. In some embodiments, the guide nucleic acid or the nucleotide sequence encoding the guide nucleic acid is operably linked to a promoter that is operable in a target cell, such as a eukaryotic cell. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids. In some embodiments, the vector comprises a nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids.

In some embodiments, a vector comprises one or more donor nucleic acids as described herein. In some embodiments, the one or more donor nucleic acids comprise at least two donor nucleic acids. In some embodiments, the at least two donor nucleic acids are the same. In some embodiments, the at least two donor nucleic acids are different from each other. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more donor nucleic acids.

In some embodiments, a vector comprises or encodes one or more regulatory elements. Regulatory elements, in some embodiments, are refer to as transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector comprises or encodes for one or more additional elements, such as, for example, replication origins, antibiotic resistance (or a nucleic acid encoding the same), a tag (or a nucleic acid encoding the same), selectable markers, and the like. In some embodiments, a vector comprises or encodes for one or more elements, such as, for example, ribosome binding sites, and RNA splice sites.

Vectors described herein can encode a promoter—a regulatory region on a nucleic acid, such as a DNA sequence, capable of initiating transcription of a downstream (3′ direction) coding or non-coding sequence. A promoter can be linked at its 3′ terminus to a nucleic acid, the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. The promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. In some embodiments, various promoters, including inducible promoters, are used to drive expression, i.e., transcriptional activation, of the nucleic acid of interest. Accordingly, in some embodiments, the nucleic acid of interest can be operably linked to a promoter.

In some embodiments, promotors comprise any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human H1 promoter (H1). By transcriptional activation, it is intended to increase transcription above basal levels in the target cell by 2 fold, 5 fold, 10 fold, 50 fold, by 100 fold, 500 fold, or by 1000 fold, or more. In addition, vectors used for providing a nucleic acid that, when transcribed, produces a guide nucleic acid and/or a nucleic acid that encodes a polypeptide (e.g., an effector protein, an effector partner, a fusion protein, or a combination thereof) to a cell comprising nucleic acid sequences that encode for selectable markers in the target cells, so as to identify cells that have taken up the guide nucleic acid and/or the polypeptide.

In general, vectors provided herein comprise at least one promotor or a combination of promoters driving expression or transcription of one or more genome editing tools described herein. In some embodiments, the vector comprises a nucleotide sequence of a promoter. In some embodiments, the vector comprises two promoters. In some embodiments, the vector comprises three promoters. In some embodiments, a length of the promoter is less than about 500, less than about 400, less than about 300, or less than about 200 linked nucleotides. In some embodiments, a length of the promoter is at least 100, at least 200, at least 300, at least 400, or at least 500 linked nucleotides. Non-limiting examples of promoters include CMV, 7SK, EF1a, RPBSA, hPGK, EFS, SV40, PGK1, Ubc, human beta actin, TRE, UAS, Ac5, Polyhedrin, CaMKIIa, GAL1-10, H1, TEF1, GDS, ADH1, CaMV35S, HSV TK, Ubi, U6, MNDU3, MSCV, MND and CAG.

In some embodiments, some promoters (e.g., U6, enhanced U6, H1 and 7SK) prefers the nucleic acid being transcribed having “g” nucleotide at the 5′ end of the coding sequence. Accordingly, when such coding sequence is expressed, it comprises an additional “g” nucleotide at 5′ end. In some embodiments, vectors provided herein comprise a promotor driving expression or transcription of any one of the guide nucleic acids described herein (e.g., TABLE 5, TABLE 6, TABLE 7, TABLE 8 and TABLE 9) further comprises “g” nucleotide at 5′ end of the guide nucleic acid, wherein the promotor is selected from U6, enhanced U6, H1 and 7SK.

In some embodiments, the promoter is a constitutive promoter. In some embodiments, the promoter is an inducible promoter. In some embodiments, the inducible promoter only drives expression of its corresponding coding sequence (e.g., polypeptide or guide nucleic acid) when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter. In some embodiments, the promoter for expressing a polypeptide (e.g., an effector protein, an effector partner, a fusion protein, or a combination thereof) is a ubiquitous promoter. In some embodiments, the ubiquitous promoter comprises MND or CAG promoter sequence.

In some embodiments, the promoters are prokaryotic promoters (e.g., drive expression of a gene in a prokaryotic cell). In some embodiments, the promoters are eukaryotic promoters, (e.g., drive expression of a gene in a eukaryotic cell). In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a nucleic acid expression vector. In some embodiments, the vector encodes one or more components of any one of the systems described herein. In some embodiments, the vector further comprises a nucleotide sequence encoding a cell specific promoter. In some embodiments, the cell specific promoter is a HSC specific promoter. Also, provided herein is a library of vectors. In some embodiments, the library of vectors comprises at least one vector as described herein.

In some embodiments, a vector described herein is a recombinant expression vector. In some embodiments, a vector described herein is a messenger RNA. In some embodiments, a vector comprising the recombinant nucleic acid as described herein, wherein the vector is a viral vector, an adeno associated viral (AAV) vector, a retroviral vector, or a lentiviral vector. In some embodiments, a vector described herein or a recombinant nucleic acid described herein is comprised in a cell. In some embodiments, a recombinant nucleic acid integrated into a genomic DNA sequence of the cell, wherein the cell is a eukaryotic cell or a prokaryotic cell.

In some embodiments, a vector described herein is a delivery vector. In some embodiments, the delivery vector is a eukaryotic vector, a prokaryotic vector (e.g., a bacterial vector) a viral vector, or any combination thereof. In some embodiments, the delivery vehicle is a non-viral vector. In some embodiments, the delivery vector is a plasmid. In some embodiments, the plasmid comprises DNA. In some embodiments, the plasmid comprises RNA. In some embodiments, the plasmid comprises circular double-stranded DNA. In some embodiments, the plasmid is linear. In some embodiments, the plasmid comprises one or more coding sequences of interest and one or more regulatory elements. In some embodiments, the plasmid comprises a bacterial backbone containing an origin of replication and an antibiotic resistance gene or other selectable marker for plasmid amplification in bacteria. In some embodiments, the plasmid is a minicircle plasmid. In some embodiments, the plasmid contains one or more genes that provide a selective marker to induce a target cell to retain the plasmid. In some examples, the plasmid is formulated for delivery through injection by a needle carrying syringe. In some examples, the plasmid is formulated for delivery via electroporation. In some examples, the plasmids are engineered through synthetic or other suitable means known in the art. For example, in some embodiments, the genetic elements are assembled by restriction digest of the desired genetic sequence from a donor plasmid or organism to produce ends of the DNA which is then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I.

### Administration of a Non-Viral Vector

In some embodiments, an administration of a non-viral vector comprises contacting a cell, such as a host cell, with the non-viral vector. In some embodiments, a physical method or a chemical method is employed for delivering the vector into the cell. Exemplary physical methods include electroporation, gene gun, sonoporation, magnetofection, or hydrodynamic delivery. Exemplary chemical methods include delivery of the recombinant polynucleotide by liposomes such as, cationic lipids or neutral lipids; lipofection; dendrimers; lipid nanoparticle (LNP); or cell-penetrating peptides.

In some embodiments, a vector is administered as part of a method of nucleic acid detection, editing, and/or treatment as described herein. In some embodiments, a vector is administered in a single vehicle, such as a single expression vector. In some embodiments, at least two of the three components, a nucleic acid encoding one or more polypeptides (e.g., effector proteins, effector partners, fusion proteins, or combinations thereof), one or more donor nucleic acids, and one or more guide nucleic acids or a nucleic acid encoding the one or more guide nucleic acid, are provided in the single expression vector. In some embodiments, components, such as a guide nucleic acid and a polypeptide (e.g., effector protein, effector partner, fusion protein, or combinations thereof), are encoded by the same vector. In some embodiments, a polypeptide (e.g., effector protein, effector partner, fusion protein, or combinations thereof) (or a nucleic acid encoding same) and/or an engineered guide nucleic acid (or a nucleic acid that, when transcribed, produces same) are not co-administered with donor nucleic acid in a single vehicle. In some embodiments, a polypeptides (e.g., effector protein, effector partner, fusion protein, or combinations thereof) (or a nucleic acid encoding same), an engineered guide nucleic acid (or a nucleic acid that, when transcribed, produces same), and/or donor nucleic acid are administered in one or more or two or more vehicles, such as one or more, or two or more expression vectors.

In some embodiments, a vector system is administered as part of a method of nucleic acid detection, editing, and/or treatment as described herein, wherein at least two vectors are co-administered. In some embodiments, the at least two vectors comprise different components. In some embodiments, the at least two vectors comprise the same component having different sequences. In some embodiments, at least one of the three components, a nucleic acid encoding one or more polypeptides (e.g., effector proteins, effector partners, fusion proteins, or combinations thereof), one or more donor nucleic acids, and one or more guide nucleic acids or a nucleic acid encoding the one or more guide nucleic acids, or a variant thereof is provided in a different vector. In some embodiments, the nucleic acid encoding the polypeptides (e.g., effector proteins, effector partners, fusion proteins, or combinations thereof), and a guide nucleic acid or a nucleic acid encoding the guide nucleic acid are provided in different vectors. In some embodiments, the donor nucleic acid is encoded by a different vector than the vector encoding the effector protein and the guide nucleic acid.

### Lipid Particles and Non-Viral Vectors

In some embodiments, compositions and systems provided herein comprise a lipid or a lipid particle. In some embodiments, a lipid particle is a lipid nanoparticle (LNP). In some embodiments, a lipid or a lipid nanoparticle can encapsulate a nucleic acid (e.g., DNA or RNA) encoding one or more of the components as described herein. In some embodiments, a lipid or a lipid nanoparticle can encapsulate an expression vector as described herein. LNPs are a non-viral delivery system for delivery of the composition and/or system components described herein. LNPs are particularly effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28 (3): 146-157). In some embodiments, compositions and methods comprise a lipid, polymer, nanoparticle, or a combination thereof, or use thereof, to introduce one or more effector proteins, one or more guide nucleic acids, one or more donor nucleic acids, or any combinations thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidic pH. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N1,N3,N5-tris(3-(didodecylamino) propyl) benzene-1,3,5-tricarboxamide (TT3), 1,2-Dioleoyl-sn-glycero-3-phosphocholine (DOPC), 2-dioleoyl-sn-glycero-3-phosphoethanolamine (DOPE), 1-palmitoyl-2-oleoylsn-glycero-3-phosphoethanolamine (POPE), 1,2-distearoyl-sn-glycero-3-phosphocholine (DSPC), cholesterol (Chol), 1,2-dimyristoyl-sn-glycerol, methoxypolyethylene glycol (DMG-PEChooo), 1,2-dimyristoyl-rac-glycero-3-methoxypolyethylene glycol-2000 (DMG-PEG 2000), derivatives, analogs, or variants thereof or any combination of the foregoing. In some embodiments, the LNP comprises one or more ionizable lipid. Such ionizable lipids include, but are not limited to: 4-(dimethylamino)-butanoic acid, (10Z,13Z)-1-(9Z,12Z)-9,12-octadecadien-1-yl-10,13-nonadecadien-1-yl ester (DLin-MC3-DMA, CAS No. 1224606-06-7); N,N-dimethyl-2,2-di-(9Z,12Z)-9,12-octadecadien-1-yl-1,3-dioxolane-4-ethanamine (DLin-KC2-DMA, CAS No. 1190197-97-7); 8-[(2-hydroxyethyl) [6-oxo-6-(undecyloxy) hexyl]amino]-octanoic acid, 1-octylnonyl ester (SM-102, CAS No. 2089251-47-6); 8-[(2-hydroxyethyl) [8-(nonyloxy)-8-oxooctyl]amino]-octanoic acid, 1-octylnonyl ester (Lipid 5, CAS No. 2089251-33-0); 1,1′-[[2-[4-[2-[[2-[bis(2-hydroxydodecyl)amino]ethyl](2-hydroxydodecyl)amino]ethyl]-1-piperazinyl]ethyl]imino]bis-2-dodecanol (C12-200, CAS No. 1220890-25-4); 2-hexyl-decanoic acid, 1,1′-[[(4-hydroxybutyl)imino]di-6,1-hexanediyl]ester (ALC-0315, CAS No. 2036272-55-4); 9,12-octadecadienoic acid, (9Z,12Z)-1,1′,1″,1″-[(3,6-dioxo-2,5-piperazinediyl)bis(4,1-butanediylnitrilodi-4,1-butanediyl)]ester (OF-C4-Deg-Lin, CAS No. 1853203-01-6); bis(2-(dodecyldisulfaneyl)ethyl) 3,3′-((3-methyl-9-oxo-10-oxa-13,14-dithia-3,6-diazahexacosyl) azanediyl) dipropionate (BAMEA-016B, CAS No. 2490668-30-7); 3,6-bis[4-[bis[(9Z,12Z)-2-hydroxy-9,12-octadecadien-1-yl]amino]butyl]-2,5-piperazinedione (OF-02, CAS No. 1883431-67-1); tetrakis(8-methylnonyl) 3,3′,3″,3′″-(((methylazanediyl)bis(propane-3,1-diyl))bis(azanetriyl))tetrapropionate (3060i10, CAS No. 2322290-93-5); tetrakis(2-(octyldisulfaneyl)ethyl) 3,3′,3″,3″-(((methylazanediyl)bis(propane-3,1-diyl))bis(azanetriyl))tetrapropionate (306-012B, CAS No. 2566523-06-4); bis(2-butyloctyl) 10-(N-(3-(dimethylamino) propyl) nonanamido) nonadecanedioate (Lipid A9, CAS No. 2036272-50-9); Arcturus Lipid 2,2 (8,8) 4C CH3 (ATX-0114, CAS No. 2230647-28-4)); di((Z)-non-2-en-1-yl) 8,8′-((2-((2-(dimethylamino)ethyl) thio) acetyl) azanediyl) dioctanoate (ATX-001, CAS No. 1777792-33-2); di((Z)-non-2-en-1-yl) 8,8′-((((2-(dimethylamino)ethyl) thio) carbonyl) azanediyl) dioctanoate (ATX-002, CAS No. 1777792-34-3); Genevant CLI (CAS No. 1450888-71-7); LP01; hexa (octan-3-yl) 9,9′,9″,9′″,9″″,9′″″-((((benzene-1,3,5-tricarbonyl)yris(azanediyl))tris(propane-3,1-diyl)) tris(azanetriyl))hexanonanoate (FTT5); 5A2-SC8 (CAS No. 1857341-90-2); COATSOME® SS-OP; derivatives; analogs; or variants thereof. In some embodiments, the LNP comprise a combination of two, three, four, five or more of the foregoing ionizable lipids.

In some embodiments, the LNP has a negative net overall charge prior to complexation with one or more of a guide nucleic acid, a nucleic acid encoding the one or more guide nucleic acid, a nucleic acid encoding a polypeptide (e.g., effector protein, effector partner, fusion protein, or combinations thereof), and/or a donor nucleic acid. In some embodiments, the inner core is a hydrophobic core. In some embodiments, the one or more of a guide nucleic acid, the nucleic acid encoding the one or more guide nucleic acid, the nucleic acid encoding the polypeptide, and/or the donor nucleic acid forms a complex with one or more of the cationic lipids and the ionizable lipids. In some embodiments, the nucleic acid encoding the polypeptide or the nucleic acid encoding the guide nucleic acid is self-replicating.

In some embodiments, a LNP comprises one or more of cationic lipids, ionizable lipids, and modified versions thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in Table 2 and Table 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells, Kupffer cells, hepatocytes).

In some embodiments, the LNP described herein comprises nucleic acids (e.g., DNA or RNA) encoding an effector protein described herein, an effector partner described herein, a fusion protein described herein, a guide nucleic acid described herein, or combinations thereof. In some embodiments, the LNP comprises an mRNA that produces an effector protein described herein, an effector partner described herein, or a fusion protein described herein when translated. In some embodiments, the LNP comprises chemically modified guide nucleic acids.

### Delivery of Viral Vectors

In some embodiments, a vector described herein comprises a viral vector. In some embodiments, the viral vector comprises a nucleic acid to be delivered into a host cell by a recombinantly produced virus or viral particle. In some embodiments, the nucleic acid comprises single-stranded or double stranded, linear or circular, segmented or non-segmented. In some embodiments, the nucleic acid comprises DNA, RNA, or a combination thereof. In some embodiments, the vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. In some embodiments, the vector is an adeno-associated viral (AAV) vector. In some embodiments, the viral vector is a recombinant viral vector. In some embodiments, the vector is a retroviral vector. In some embodiments, the retroviral vector is a lentiviral vector. In some embodiments, the retroviral vector comprises gamma-retroviral vector. In some embodiments, a viral vector provided herein is derived from or based on any such virus. For example, in some embodiments, the gamma-retroviral vector is derived from a Moloney Murine Leukemia Virus (MoMLV, MMLV, MuLV, or MLV) or a Murine Stem cell Virus (MSCV) genome. In some embodiments, the lentiviral vector is derived from the human immunodeficiency virus (HIV) genome. In some embodiments, the viral vector is a chimeric viral vector. In some embodiments, the chimeric viral vector comprises viral portions from two or more viruses. In some embodiments, the viral vector corresponds to a virus of a specific serotype.

In some embodiments, a viral vector is an adeno-associated viral vector (AAV vector). In some embodiments, a viral particle that delivers a viral vector described herein is an AAV. In some embodiments, the AAV comprises any AAV known in the art. In some embodiments, the viral vector corresponds to a virus of a specific AAV serotype. In some embodiments, the AAV serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, an AAV12 serotype, an AAV-rh10 serotype, and any combination, derivative, or variant thereof. In some embodiments, the AAV vector is a recombinant vector, a hybrid AAV vector, a chimeric AAV vector, a self-complementary AAV (scAAV) vector, a single-stranded AAV, or any combination thereof. scAAV genomes are generally known in the art and contain both DNA strands which can anneal together to form double-stranded DNA.

In some embodiments, an AAV vector described herein is a chimeric AAV vector. In some embodiments, the chimeric AAV vector comprises an exogenous amino acid or an amino acid substitution, or capsid proteins from two or more serotypes. In some embodiments, a chimeric AAV vector is genetically engineered to increase transduction efficiency, selectivity, or a combination thereof.

In some embodiments, AAV vector described herein comprises two inverted terminal repeats (ITRs). According, in some embodiments, the viral vector provided herein comprises two inverted terminal repeats of AAV. A nucleotide sequence between the ITRs of an AAV vector provided herein comprises a sequence encoding genome editing tools. In some embodiments, the genome editing tools comprise a nucleic acid encoding one or more polypeptides (e.g., effector proteins, effector partners, fusion proteins, or combinations thereof), a nucleic acid encoding the one or more polypeptides comprising a heterologous peptide (e.g., a nuclear localization signal (NLS), polyA tail), one or more guide nucleic acids, a nucleic acid encoding the one or more guide nucleic acids, respective promoter(s), one or more donor nucleic acid, or any combinations thereof. In some embodiments, viral vectors provided herein comprise at least one promotor or a combination of promoters driving expression or transcription of one or more genome editing tools described herein. In some embodiments, a coding region of the AAV vector forms an intramolecular double-stranded DNA template thereby generating the AAV vector that is a self-complementary AAV (scAAV) vector. In some embodiments, the scAAV vector comprises the nucleotide sequence encoding genome editing tools that has a length of about 2 kb to about 3 kb. In some embodiments, the AAV vector provided herein is a self-inactivating AAV vector. In some embodiments, the AAV vector provided herein comprises a modification, such as an insertion, deletion, chemical alteration, or synthetic modification, relative to a wild-type AAV vector.

### Producing AAV Delivery Vectors

In some embodiments, methods of producing AAV delivery vectors herein comprise packaging a nucleic acid encoding a polypeptide (e.g., effector protein, effector partner, fusion protein, or combinations thereof) and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging the polypeptide encoding nucleic acid into the AAV particle, thereby generating an AAV delivery vector. In some embodiments, promoters, stuffer sequences, and any combination thereof are packaged in the AAV vector. In some embodiments, the AAV vector is package 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes are not the same. In some examples, the Rep gene and ITR from a first AAV serotype (e.g., AAV2) is used in a capsid from a second AAV serotype (e.g., AAV9), wherein the first and second AAV serotypes are not the same. As a non-limiting example, a hybrid AAV serotype comprising the AAV2 ITRs and AAV9 capsid protein is indicated AAV2/9. In some examples, the hybrid AAV delivery vector comprises an AAV2/1, AAV2/2, AAV2/4, AAV2/5, AAV2/8, or AAV2/9 vector.

### Producing AAV Particles

In some embodiments, AAV particles described herein are recombinant AAV (rAAV). In some embodiments, rAAV particles are generated by transfecting AAV producing cells with an AAV-containing plasmid carrying the nucleotide sequence encoding the genome editing tools, a plasmid that carries viral encoding regions, i.e., Rep and Cap gene regions; and a plasmid that provides the helper genes such as E1A, E1B, E2A, E4ORF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a variant thereof, or a combination thereof. In some embodiments, rAAV virus particles can be produced in the mammalian cell culture system by providing the rAAV plasmid to the mammalian cell. In some embodiments, producing rAAV virus particles in a mammalian cell comprises transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31 (4): 317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, the insect cell for producing rAAV viral particles comprises a Sf9 cell. In some embodiments, production of rAAV virus particles in insect cells comprises infecting the insect cells with baculovirus. In some embodiments, production of rAAV virus particles in insect cells comprises infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3 (12): 2156-65; Urabe et al., (2002), Hum. Gene. Ther., 1; 13 (16): 1935-43; and Benskey et al., (2019), Methods Mol Biol., 1937:3-26, each of which is incorporated by reference in its entirety.

### IX. Target Nucleic Acids

Disclosed herein are compositions, systems and methods for detecting and/or editing a target nucleic acid. In some embodiments, the target nucleic acid is a double stranded nucleic acid. In some embodiments, the target nucleic acid is a single stranded nucleic acid. Alternatively, or in combination, the target nucleic acid is a double stranded nucleic acid and is prepared into single stranded nucleic acids before or upon contacting an RNP. In some embodiments, the single stranded nucleic acid comprises a RNA, wherein the RNA comprises a mRNA, a rRNA, a tRNA, a non-coding RNA, a long non-coding RNA, a microRNA (miRNA), and a single-stranded RNA (ssRNA). In some embodiments, the target nucleic acid is complementary DNA (cDNA) synthesized from a single-stranded RNA template in a reaction catalyzed by a reverse transcriptase. In some embodiments, the target nucleic acid comprises an RNA, a DNA, or combination thereof. In some embodiments, guide nucleic acids described herein hybridize to a portion of the target nucleic acid. In some embodiments, the target nucleic acid is from a virus, a parasite, or a bacterium described herein.

In some embodiments, a target nucleic acid comprising a target sequence comprises a PAM sequence. In some embodiments, the PAM sequence is adjacent to the target sequence. In some embodiments, the PAM sequence is 3′ to the target sequence. In some embodiments, the PAM sequence is directly 3′ to the target sequence. In some embodiments, the PAM sequence 5′ to the target sequence. In some embodiments, the PAM sequence is directly 5′ to the target sequence. In some embodiments, the target nucleic acid as described in the methods herein does not initially comprise a PAM sequence. However, any target nucleic acid of interest that is generated using the methods described herein to comprise a PAM sequence, and thus be a PAM target nucleic acid. A PAM target nucleic acid, as used herein, refers to a target nucleic acid that has been amplified to insert a PAM sequence that is recognized by a polypeptide system.

In some embodiments, a target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides. In some embodiments, the target nucleic acid comprises 10 to 90, 20 to 80, 30 to 70, or 40 to 60 linked nucleotides. In some embodiments, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some embodiments, the target nucleic acid comprises at least 10, at least 20, at least 30, at least 40, at least 50, at least 60, at least 70, at least 80, at least 90, or at least 100 linked nucleotides. In some embodiments, the target sequence in the target nucleic acid comprises at least 10 contiguous nucleotides that are complementary to the guide nucleic acid or engineered guide nucleic acid.

In some embodiments, compositions, systems, and methods described herein comprise a target nucleic acid that comprises a nucleotide, a nucleotide sequence, a coding sequence, a gene, an exon, an intron, a gene regulatory region, a fragment thereof, or combinations thereof. In some embodiments, the gene regulatory region comprises a promotor, an enhancer, or a combination thereof.

In some embodiments, compositions, systems, and methods described herein comprise a target nucleic acid that is responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides). In some embodiments, the target nucleic acid has undergone a modification (e.g., an editing) after contacting with an RNP. In some embodiments, the editing is a change in the nucleotide sequence of the target nucleic acid. In some embodiments, the change comprises an insertion, deletion, or substitution of one or more nucleotides compared to the target nucleic acid that has not undergone any modification.

In some embodiments, a target nucleic acid comprises a portion or a specific region of a nucleic acid from a genomic locus, any DNA amplicon of, a reverse transcribed mRNA, or a cDNA from a gene described herein. In some embodiments, the target nucleic acid is an amplicon of at least a portion of a gene. Non-limiting examples of genes are recited in TABLE 10. In some embodiments, the target nucleic acid comprises a nucleotide sequence comprising any one of the genes recited in TABLE 10, a variant thereof, a promoter thereof, an enhancer thereof, or a portion thereof. Nucleic acid sequences of target nucleic acids and/or corresponding genes are readily available in public databases as known and used in the art. In some embodiments, the target nucleic acid is selected from any one of the genes recited in TABLE 10, a variant thereof, a promoter thereof, an enhancer thereof, or a portion thereof. In some embodiments, the target nucleic acid comprises one or more target sequences. In some embodiments, the one or more target sequence is within the target nucleic acid of any one of the genes recited in TABLE 10.

Non-limiting examples of nucleotide sequences of genes are recited in TABLE 11. In some embodiments, the target nucleic acid comprises a nucleotide sequence that is at least 65%, at least 70%, at least 75%, at least 80%, at least 85%, at least 90%, at least 95%, at least 97%, at least 98%, at least 99%, or 100% identical to any one of the nucleotide sequences set forth in TABLE 11. In some embodiments, the target nucleic acid comprises one or more target sequences. In some embodiments, the one or more target sequence is within any one of the nucleotide sequences recited in TABLE 11. In some embodiments, the target nucleic acid comprises any one of the following genes: BCL11A, B2M, PNPLA2, CHD4, CIITA, GATA1, HBA, HBA1, HBA2, β-globin gene (HBB), HBD, HBE, HBE1, HBG1, HBG2, HBM, HBQ1, HBZ, HOXA9, KLF1, MBD3, MYB, TRAC1, ZBTB7A, a fragment thereof, a promoter thereof, an enhancer thereof, or a combination thereof. In some embodiments, the target nucleic acid comprises any one of the following genes: BCL11A, HBB, HBG1, HBG2, a fragment thereof, a promoter thereof, an enhancer thereof, or a combination thereof. In some embodiments, the HBG gene comprises γ-globin 1 gene (HBG1 gene), γ-globin 2 gene (HBG2 gene), G γ-globin gene, A γ-globin gene, a fragment thereof, a promoter thereof, an enhancer thereof, or a combination thereof. In some embodiments, the target nucleic acid comprises a HBB gene, a fragment thereof, an enhancer thereof or a promoter thereof. In some embodiments, the HBB gene comprises a EV6 mutation.

In some embodiments, the target nucleic acid is a naturally occurring eukaryotic sequence, an engineered eukaryotic sequence, a fragment of a naturally occurring sequence, a fragment of an engineered sequence, or combinations thereof.

Nucleic acids, such as DNA and pre-mRNA, described herein can contain at least one intron and at least one exon, wherein as read in the 5′ to the 3′ direction of a nucleic acid strand, the 3′ end of an intron can be adjacent to the 5′ end of an exon, and wherein said intron and exon correspond for transcription purposes. If a nucleic acid strand contains more than one intron and exon, the 5′ end of the second intron is adjacent to the 3′ end of the first exon, and 5′ end of the second exon is adjacent to the 3′ end of the second intron. The junction between an intron and an exon can be referred to herein as a splice junction, wherein a 5′ splice site (SS) can refer to the +1/+2 position at the 5′ end of intron and a 3′SS can refer to the last two positions at the 3′ end of an intron. Alternatively, a 5′ SS can refer to the 5′ end of an exon and a 3′SS can refer to the 3′ end of an exon. In some embodiments, nucleic acids can contain one or more elements that act as a signal during transcription, splicing, and/or translation. In some embodiments, signaling elements include a 5′SS, a 3′SS, a premature stop codon, U1 and/or U2 binding sequences, and cis acting elements such as branch site (BS), polypyridine tract (PYT), exonic and intronic splicing enhancers (ESEs and ISEs) or silencers (ESSs and ISSs). In some embodiments, nucleic acids also comprise an untranslated region (UTR), such as a 5′ UTR or a 3′ UTR. In some embodiments, the start of an exon or intron is referred to interchangeably herein as the 5′ end of an exon or intron, respectively. Likewise, in some embodiments, the end of an exon or intron is referred to interchangeably herein as the 3′ end of an exon or intron, respectively.

In some embodiments, at least a portion of at least one target sequence is within 1, about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 to about 300 nucleotides adjacent to: the 5′ end of an exon; the 3′ end of an exon; the 5′ end of an intron; the 3′ end of an intron; one or more signaling element comprising a 5′SS, a 3′SS, a premature stop codon, U1 binding sequence, U2 binding sequence, a BS, a PYT, ESE, an ISE, an ESS, an ISS; a 5′ UTR; a 3′ UTR; more than one of the foregoing, or any combination thereof. In some embodiments, the target nucleic acid comprises a target locus. In some embodiments, the target nucleic acid comprises more than one target loci. In some embodiments, the target nucleic acid comprises two target loci. Accordingly, in some embodiments, the target nucleic acid can comprise one or more target sequences.

In some embodiments, compositions, systems, and methods described herein comprise an edited target nucleic acid which can describe a target nucleic acid wherein the target nucleic acid has undergone a change, for example, after contact with a polypeptide (e.g., an effector protein, an effector partner, a fusion protein, or a combination thereof). In some embodiments, the editing is an alteration in the nucleotide sequence of the target nucleic acid. In some embodiments, the edited target nucleic acid comprises a nicked target strand or a nicked non-target strand. In some embodiments, the edited target nucleic acid comprises an insertion, deletion, or replacement of one or more nucleotides compared to the unedited target nucleic acid. In some embodiments, the editing is a mutation.

In some embodiments, the target nucleic acid comprises at least one mutation relative to corresponding wildtype nucleotide sequence. In some embodiments, the at least one mutation results in a single nuclear polymorphism (SNP). In some embodiments, the at least one mutation is associated with any one of diseases or disorders listed in TABLE 12. In some embodiments, the at least one mutation is associated with a genetic blood disease or disorder. In some embodiments, the at least one mutation is associated with sickle cell anemia, sickle cell disease (SCD), and/or β-thalassemia. In some embodiments, the target nucleic acid is within a eukaryotic gene (e.g., a human gene, a mammal gene).

In some embodiments, the target nucleic acid comprises any one of the genes recited in TABLE 11, a fragment thereof, a promoter thereof, an enhancer thereof, or a combination thereof. In some embodiments, the target nucleic acid comprises a nucleotide sequence that is at least 85% identical to any one of the nucleotide sequences set forth in TABLE 11. In some embodiments, the target nucleic acid comprises any one of BCL11A gene, B2M gene, PNPLA2 gene, (HD) 4 gene, CIITA gene, GATA1 gene, HBA gene, HBA1 gene, HBA2 gene, HBB gene, HBD gene, HBE1 gene, HBG gene, HBG1 gene, HBG2 gene, HBM gene, HBQ1 gene, HBZ gene, HOXA9 gene, KLF1 gene, MBD3 gene, MYB gene, TRAC gene, ZBTB7A gene, a variant thereof, a fragment thereof, a promoter thereof, an enhancer thereof, or a combination thereof. In some embodiments, the target nucleic acid comprises any one of the following genes: BCL11A, B2M, PNPLA2, CHD4, CIITA, GATA1, HBA, HBA1, HBA2, β-globin gene (HBB gene), HBD, HBE, HBE1, HBG, γ-globin 1 gene (HBG1 gene), γ-globin 2 gene (HBG2 gene), HBM, HBQ1, HBZ, HOXA9, KLF1, MBD3, MYB, TRAC1, ZBTB7A, a fragment thereof, a promoter thereof, an enhancer thereof, or a combination thereof. A sequence representing a human wildtype allele of any one of the target nucleic acids described above may be found in the NCBI database. In some embodiments, a target nucleic acid comprises a gene encoding hemoglobin protein, a hemoglobin subunit, or a portion thereof. In some embodiments, the target nucleic acids comprise a nucleotide sequence encoding C2H2 type zinc-finger protein, Chromodomain Helicase DNA Binding Protein 4 (CHD4) GATA1 protein, Glioma pathogenesis-related protein 1 (GLIPR1), globin protein, alpha-globin protein, beta globin protein, gamma globin chain, hemoglobin A (HbA) protein, fetal hemoglobin (HbF), HBE1 protein, HBZ protein, hemoglobin subunit epsilon, hemoglobin subunit zeta, HBM protein, HBQ1 protein, HOXA9 protein, KLF1 protein, MBD3 protein, MYB protein, TRAC protein, ZBTB protein, a functional fragment thereof, a promoter thereof, an enhancer thereof, or a combination thereof.

In some embodiments, a target nucleic acid described herein comprises HBB gene, a variant thereof, a fragment thereof, a promoter thereof, an enhancer thereof, or a combination thereof. Accordingly, in some embodiments, the target nucleic acid encodes beta-globin protein (e.g., a subunit of the larger hemoglobin protein), or a functional portion thereof. HBB gene encodes β-globin protein. The HBB gene contains 3 exons and is located on chromosome 11, at cytogenetic location 11p15.4. A sequence representing a human wildtype allele of HBB may be found in the NCBI database with gene accession ID: NC_000011.10. sequence representing human wildtype HBB mRNA (also a sense strand of human HBB cDNA) may be found in the Ensembl database with accession number: ENST00000335295.4. In some embodiments, a variant of the HBB gene comprises at least on mutation in the HBB gene. In some embodiments, the at least one mutation results in a Single Nuclear Polymorphism (SNP). In some embodiments, the at least one mutation in the HBB gene is E6V mutation. In some embodiments, the at least one mutation in the HBB gene is associated with a genetic blood disease or disorder. In some embodiments, the genetic disease or disorder comprises sickle cell anemia, sickle cell disease, β-thalassemia, or a combination thereof.

In some embodiments, a target nucleic acid described herein comprises HBG1 gene and/or HBG2 gene. The HBA1 gene and the HBA2 gene together forms the alpha-globin locus. In some embodiments, the target nucleic acid comprises HBA gene. The HBA gene encodes alpha-globin protein. β-globin protein and alpha-globin protein together forms hemoglobin A (HbA) protein. Accordingly, in some embodiments, the target nucleic acid comprises the HBB gene and the HBA gene. Accordingly, in some embodiments, the target nucleic acid comprises a nucleotide sequence encoding the alpha-globin locus, hemoglobin A (HbA) protein, a variant thereof, or a functional portion thereof.

In some embodiments, a target nucleic acid described herein comprises HBG1 gene or HBG2 gene. Each, the HBG1 gene and the HBG2 gene, encodes gamma globin chains involved in creating fetal hemoglobin (HbF). Two gamma globin chains and two alpha globin chains forms fetal hemoglobin (HbF). Accordingly, in some embodiments, the target nucleic acid comprises the HBG1 gene and the HBB gene. Alternatively, in some embodiments, the target nucleic acid comprises the HBG2 gene and the HBB gene. Accordingly, in some embodiments, the target nucleic acid comprises a nucleotide sequence encoding the gamma globin chain, fetal hemoglobin (HbF), a variant thereof, or a functional portion thereof.

In some embodiments, a target nucleic acid described herein comprises HBD gene. The HBD gene encodes hemoglobin subunit delta protein. Accordingly, in some embodiments, the target nucleic acid comprises a nucleotide sequence encoding the hemoglobin subunit delta protein, a variant thereof, or a functional portion thereof.

In some embodiments, a target nucleic acid described herein comprises BCL11A gene. The BCL11A gene encodes a C2H2 type zinc-finger protein, which is a transcriptional repressor that decreases fetal hemoglobin (HbF) expression in adult tissues. Accordingly, in some embodiments, the target nucleic acid comprises a nucleotide sequence encoding the C2H2 type zinc-finger protein, a variant thereof, or a functional portion thereof.

In some embodiments, a target nucleic acid described herein comprises a CHI) 4 (Chromodomain Helicase DNA Binding Protein 4) gene. The CHD4 gene encodes ATP-Dependent Helicase CHD4. Accordingly, in some embodiments, the target nucleic acid comprises a nucleotide sequence encoding the CHD4 protein, a variant thereof, or a functional portion thereof.

In some embodiments, a target nucleic acid described herein comprises a GATA1 gene. The GATA1 gene encodes a GATA1 protein, which is involved in the specialization (differentiation) of immature blood cells. Accordingly, in some embodiments, the target nucleic acid comprises a nucleotide sequence encoding the GATA1 protein, a variant thereof, or a functional portion thereof.

In some embodiments, a target nucleic acid described herein comprises a B2M (beta-2-microglobulin) gene. The B2M gene encodes a B2M protein. Accordingly, in some embodiments, the target nucleic acid comprises a nucleotide sequence encoding the B2M protein, a variant thereof, or a functional portion thereof.

In some embodiments, a target nucleic acid described herein comprises a HBD) (Hemoglobin Subunit Delta) gene. The HBD gene encodes hemoglobin subunit delta protein. Accordingly, in some embodiments, the target nucleic acid comprises a nucleotide sequence encoding the HBD protein, a variant thereof, or a functional portion thereof.

In some embodiments, a target nucleic acid described herein comprises a HBE1 gene, a HBZ gene, or a combination thereof. The HBE1 gene encodes HBE1 protein. The HBZ gene encodes a HBZ protein. The HBE1 protein and the HBZ protein together forms hemoglobin subunits that is found in the early embryo. Accordingly, in some embodiments, the target nucleic acid comprises a nucleotide sequence encoding the HBE1 protein, HBZ protein, a variant thereof, a functional portion thereof or a combination thereof.

In some embodiments, a target nucleic acid described herein comprises a HBM (Hemoglobin Subunit Mu) gene. The HBM gene encodes HBM protein. Accordingly, in some embodiments, the target nucleic acid comprises a nucleotide sequence encoding the HBM protein, a variant thereof, or a functional portion thereof.

In some embodiments, a target nucleic acid described herein comprises a HBQ1 (Hemoglobin Subunit Theta 1) gene. The HBQ1 gene encodes a HBQ1 protein. Accordingly, in some embodiments, the target nucleic acid comprises a nucleotide sequence encoding the HBQ1 protein, a variant thereof, or a functional portion thereof.

In some embodiments, a target nucleic acid described herein comprises a HOXA9 (Homeobox A9) gene. The HOXA9 gene encodes HOXA9 protein. The HOXA9 protein is a homeodomain-containing transcription factor which is involved in enhancing blood formation (e.g., stem cell expansion, differentiation, B and T-cell development). Accordingly, in some embodiments, the target nucleic acid comprises a nucleotide sequence encoding the HOXA9 protein, a variant thereof, or a functional portion thereof.

In some embodiments, a target nucleic acid described herein comprises a KLF1 (Krueppel-like factor 1) gene. The KLF1 gene encodes a KLF1 protein. The KLF1 is a hematopoietic-specific transcription factor that induces high-level expression of adult beta-globin and other erythroid (red blood) genes. Accordingly, in some embodiments, the target nucleic acid comprises a nucleotide sequence encoding the KLF1 protein, a variant thereof, or a functional portion thereof.

In some embodiments, a target nucleic acid described herein comprises a MBD3 (Methyl-CpG Binding Domain Protein 3) gene. The MBD3 gene encodes a MBD3 protein. The MBD3 protein is an epigenetic regulator, which facilitates lineage commitment of pluripotent cells and is important for stem cell homeostasis in blood and skin). Accordingly, in some embodiments, the target nucleic acid comprises a nucleotide sequence encoding the MBD3 protein, a variant thereof, or a functional portion thereof.

In some embodiments, a target nucleic acid described herein comprises an oncogene. In some embodiments, the oncogene comprises a MYB (MYB Proto-Oncogene, Transcription Factor) gene. The MYB gene encodes a MYB protein (member of the MYB (myeloblastosis) family of transcription factors). Accordingly, in some embodiments, the target nucleic acid comprises a nucleotide sequence encoding the MYB protein, a variant thereof, or a functional portion thereof.

In some embodiments, a target nucleic acid described herein comprises a TRAC (T Cell Receptor Alpha Constant) gene. The TRAC gene encodes a TRAC protein. The TRAC protein is a T cell receptor that recognizes foreign antigens. Accordingly, in some embodiments, the target nucleic acid comprises a nucleotide sequence encoding the TRAC protein, a variant thereof, or a functional portion thereof.

In some embodiments, a target nucleic acid described herein comprises a ZBTB7A (Zinc Finger And BTB Domain Containing 7A) gene. The ZBTB7A gene encodes ZBTB protein. The ZBTB protein is a transcription factor (member of the C2H2 zinc finger protein family) involved in cell differentiation and proliferation. Accordingly, in some embodiments, the target nucleic acid comprises a nucleotide sequence encoding a protein from C2H2 zinc finger protein family. In some embodiments, the C2H2 zinc finger protein family comprises ZBTB protein, a variant thereof, or a functional portion thereof.

### Donor Nucleic Acid

In some embodiments, a donor nucleic acid comprises a nucleic acid that is incorporated into a target nucleic acid or target sequence.

In some embodiments, a donor nucleic acid comprises a transgene. Accordingly, provided herein are transgene for use with the compositions, systems and methods of the disclosure. In some embodiments, the transgene comprises a nucleotide sequence that is inserted into a cell for expression of said nucleotide sequence in the cell. In some embodiments, the transgene comprises (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. A donor nucleic acid can comprise a transgene. The cell in which transgenes expression occur can be a target cell, such as a host cell.

In some embodiments, the donor nucleic acid comprises single-stranded DNA or linear double-stranded DNA. In some embodiments, the donor nucleic acid comprises a nucleotide sequence encoding a functional polypeptide and/or wherein the donor nucleic acid comprises a wildtype sequence. In some embodiments, the donor nucleic acid comprises a naturally occurring sequence. In some embodiments, the naturally occurring sequence does not contain a mutation.

In some embodiments, the donor nucleic acid comprises a nucleotide, a nucleotide sequence, a coding sequence, a gene, an exon, an intron, a gene regulatory region, a fragment thereof, or combinations thereof. In some embodiments, the fragment is at least 10, at least 20, at least 30, at least 40, at least 50, at least 60, at least 70, or at least 80 contiguous nucleotides.

In some embodiments, a donor nucleic acid of any suitable size is integrated into a target nucleic acid or a genome. In some embodiments, the donor nucleic acid integrated into the target nucleic acid or the genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some embodiments, the donor nucleic acid is more than 500 kilobases (kb) in length.

In some embodiments, a viral vector comprising a donor nucleic acid introduces the donor nucleic acid into a cell following transfection. In some embodiments, the donor nucleic acid is introduced into the cell by any mechanism of the transfecting viral vector, including, but not limited to, integration into the genome of the cell or introduction of an episomal plasmid or viral genome.

In some embodiments, the donor nucleic acid comprises any one of the following human genes: HBA gene, HBA1 gene, HBA2 gene, HBB gene, HBD gene, HBE1 gene, HBG gene, HBG1 gene, HBG2 gene, HBM gene, HBQ1 gene, HBZ gene, HOXA9 gene, KLF1 gene, MBD3 gene, a fragment thereof, a promoter thereof, an enhancer thereof, or a combination thereof. Accordingly, in some embodiments, a transgene described herein comprises any one of HBA gene, HBA1 gene, HBA2 gene, HBB gene, HBD) gene, HBE1 gene, HBG gene, HBG1 gene, HBG2 gene, HBM gene, HBQ1 gene, HBZ gene, HOXA9 gene, KLF1 gene, MBD3 gene, KIT gene, a variant thereof, a fragment thereof, a promoter thereof, an enhancer thereof, or a combination thereof. In some embodiments, the transgene comprise a nucleotide sequence encoding globin protein, alpha-globin protein, beta globin protein, gamma globin chain, hemoglobin A (HbA) protein, fetal hemoglobin (HbF), HBE1 protein, HBZ protein, hemoglobin subunit epsilon, hemoglobin subunit zeta, HBM protein, HBQ1 protein, HOXA9 protein, KLF1 protein, MBD3 protein, CD117 protein, MYB protein, a functional fragment thereof, a promoter thereof, an enhancer thereof, or a combination thereof.

In some embodiments, an effector protein as described herein facilitates insertion of a donor nucleic acid at a site of cleavage or between two cleavage sites by cleaving (hydrolysis of a phosphodiester bond) of a nucleic acid resulting in a nick or double strand break-nuclease activity.

In some embodiments, a donor nucleic acid serves as a template in the process of homologous recombination, which carries an alteration that is to be or has been introduced into a target nucleic acid. By using the donor nucleic acid as a template, the genetic information, including the alteration, is copied into the target nucleic acid by way of homologous recombination.

In some embodiments, a donor nucleic acid is inserted at a cleavage site within the target nucleic acid, wherein the cleavage site is generated by an effector protein or fusion protein described herein. In some embodiments, the donor nucleic acid encodes an amino acid sequence of a functional human protein. In some embodiments, the functional human protein has at least 50%, at least 60%, at least 70%, at least 80%, at least 90%, at least 100%, at least 110%, at least 120%, at least 130%, at least 140%, at least 150%, at least 180%, at least 200%, at least 300%, at least 400% enzymatic activity compared to wildtype.

In some embodiments, the functional human protein comprises wildtype protein. Specific examples of functional human proteins include wildtype and engineered versions of proteins that is deficient, under-expressed or aberrantly expressed in certain human subjects. In some embodiments, these subjects suffer from disorders that result in deficiency, or aberrant expression of these proteins. Compositions and methods provided herein are useful for treating conditions characterized by such deficiency, or aberrant expression.

In some embodiments, the wildtype protein comprises human wildtype protein sequence. In some embodiments, the human protein comprises an amino acid sequence that is associated with any one of diseases recited in TABLE 12.

### Mutations

In some embodiments, target nucleic acids described herein comprise a mutation. In some embodiments, a composition, system or method described herein can be used to edit a target nucleic acid comprising a mutation such that the mutation is edited to be the wild-type nucleotide or nucleotide sequence. In some embodiments, a composition, system or method described herein can be used to detect a target nucleic acid comprising a mutation. In some embodiments, a mutation results in the insertion of at least one amino acid in a protein encoded by the target nucleic acid. In some embodiments, a mutation results in the deletion of at least one amino acid in a protein encoded by the target nucleic acid. In some embodiments, a mutation results in the substitution of at least one amino acid in a protein encoded by the target nucleic acid. A mutation that results in the deletion, insertion, or substitution of one or more amino acids of a protein encoded by the target nucleic acid results in misfolding of a protein encoded by the target nucleic acid. In some embodiments, a mutation results in a premature stop codon, thereby resulting in a truncation of the encoded protein.

Non-limiting examples of mutations are insertion-deletion (indel), a point mutation, single nucleotide polymorphism (SNP), a chromosomal mutation, a copy number mutation or variation, and frameshift mutations. In some embodiments, an indel mutation is an insertion or deletion of one or more nucleotides. In some embodiments, a point mutation comprises a substitution, insertion, or deletion. In some embodiments, a frameshift mutation occurs when the number of nucleotides in the insertion/deletion is not divisible by three, and it occurs in a protein coding region. In some embodiments, a chromosomal mutation can comprise an inversion, a deletion, a duplication, or a translocation of one or more nucleotides. In some embodiments, a copy number variation can comprise a gene amplification or an expanding trinucleotide repeat. In some embodiments, an SNP is associated with a phenotype of the sample or a phenotype of the organism from which the sample was taken. In some embodiments, an SNP is associated with altered phenotype from wild type phenotype. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution. In some embodiments, the mutation is associated with one or more of protein expression, protein activity, and protein structural stability.

In some embodiments, a target nucleic acid described herein comprises a mutation of one or more nucleotides. In some embodiments, the one or more nucleotides comprises 1, 2, 3, 4, 5, 6, 7, 8, 9, 10, 11, 12, 13, 14, 15, 16, 17, 18, 19, 20 or more nucleotides. In some embodiments, the mutation comprises a deletion, insertion, and/or substitution of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. In some embodiments, the mutation comprises a deletion, insertion, and/or substitution of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. In some embodiments, the mutation is located in a non-coding region or a coding region of a gene, wherein the gene is a target nucleic acid. In some embodiments, a mutation is in an open reading frame of a target nucleic acid. In some embodiments, guide nucleic acids described herein hybridize to a portion of the target nucleic acid comprising or adjacent to the mutation.

In some embodiments, the target nucleic acid comprises one or more mutations. In some embodiments, the target nucleic acid comprises one or more, two or more, three or more, four or more, five or more, six or more, seven or more, eight or more, nine or more, or ten or more mutations as compared to the unmutated target nucleic acid. In some embodiments, the target nucleic acid comprises a sequence comprising one or more, two or more, three or more, four or more, five or more, six or more, seven or more, eight or more, nine or more, or ten or more mutations as compared to the wildtype sequence. In some embodiments, the target nucleic acid comprises a mutation associated with a disease or disorder.

In some embodiments, target nucleic acids comprise a mutation, wherein the mutation is a SNP. In some embodiments, the single nucleotide mutation or SNP is associated with a phenotype of the sample or a phenotype of the organism from which the sample was taken. In some embodiments, the SNP is associated with altered phenotype from wild type phenotype. In some embodiments, a single nucleotide mutation, SNP, or deletion described herein is associated with a disease, such as a genetic disease. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution. In some embodiments, the mutation is a deletion of one or more nucleotides. In some embodiments, the single nucleotide mutation, SNP, or deletion is associated with a disease such as a genetic disorder. In some embodiments, the mutation, such as a single nucleotide mutation, a SNP, or a deletion, is encoded in the nucleotide sequence of a target nucleic acid from the germline of an organism or is encoded in a target nucleic acid from a diseased cell.

In some embodiments, the mutation is associated with a disease, such as a genetic disorder. In some embodiments, the mutation is encoded in the nucleotide sequence of a target nucleic acid from the germline of an organism or is encoded in a target nucleic acid from a diseased cell. In some embodiments, a target nucleic acid described herein comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. In some embodiments, a mutation associated with a disease is also refer to any mutation which generates transcription or translation products at an abnormal level, or in an abnormal form, in cells affected by a disease relative to a control without the disease. In some examples, a mutation associated with a disease refers to a mutation whose presence in a subject indicates that the subject is susceptible to, or suffers from, a disease, disorder, or pathological state. In some embodiments, a mutation associated with a disease, comprises the co-occurrence of a mutation and the phenotype of a disease. In some embodiments, the mutation occurs in a gene, wherein transcription or translation products from the gene occur at a significantly abnormal level or in an abnormal form in a cell or subject harboring the mutation as compared to a non-disease control subject not having the mutation.

In some embodiments, a target nucleic acid described herein comprises a mutation associated with a disease, wherein the target nucleic acid comprises any one of the following genes: BCL11A, B2M, PNPLA2, CHD4, CIITA, GATA1, HBA, HBA1, HBA2, 8-globin gene (HBB gene), HBD, HBE, HBE1, HBG, γ-globin 1 gene (HBG1 gene), γ-globin 2 gene (HBG2 gene), HBM, HBQ1, HBZ, HOXA9, KLF1, MBD3, MYB, TRAC1, ZBTB7A, a fragment thereof, a promoter thereof, an enhancer thereof, or a combination thereof. In some embodiments, the target nucleic acid comprises any one of the following genes: BCL11A, HBB, HBG1, HBG2, a fragment thereof, a promoter thereof, an enhancer thereof, or a combination thereof. In some embodiments, the target nucleic acid comprises a ε-globin gene, γ-globin gene (HBG1 gene), G γ-globin gene, A γ-globin gene, δ-globin gene, β-globin gene (HBB gene), a fragment thereof, a promoter thereof, an enhancer thereof, or a combination thereof. In some embodiments, the target nucleic acid comprises a HBB gene, a fragment thereof, a promoter thereof, an enhancer thereof, or combinations thereof. In some embodiments, the target nucleic acid comprises a EV6 mutation in the HBB gene. In some embodiments, the target nucleic acid comprises HBG1 gene, HBG2 gene, HBB gene, BCL11A gene, human B2M gene, human CIITA gene, human TRAC1 gene or a combination thereof. In some embodiments, the target nucleic acid comprises one or more nucleotide alterations at one or more positions relative to corresponding reference nucleotide sequence of TABLE 11. In some embodiments, the target nucleic acid comprises one or more nucleotide substitution at one or more positions relative to corresponding reference nucleotide sequence of TABLE 11. In some embodiments, the BCL11A gene, the HBB gene, the HBG1 gene and the HBG2 gene comprises one or more nucleotide alterations at one or more positions relative to SEQ ID NO: 836, 844, 847 and 848, respectively. In some embodiments, the BCL11A gene, the HBB gene, the HBG1 gene and the HBG2 gene comprises one or more nucleotide substitutions at one or more positions relative to SEQ ID NO: 836, 844, 847 and 848, respectively. Alternative nucleotides can be any one or more of A, C, G, T or U, or a deletion, or an insertion.

Disclosed herein are compositions comprising one or more polypeptides (e.g., effector proteins, effector partners, fusion proteins, or combinations thereof) described herein or nucleic acids encoding the one or more polypeptides, one or more guide nucleic acids described herein or nucleic acids encoding the one or more guide nucleic acids described herein, or combinations thereof. In some embodiments, repeat sequences of the one or more guide nucleic acids are capable of interacting with the one or more of the effector proteins. In some embodiments, spacer sequences of the one or more guide nucleic acids hybridizes with a target sequence of a target nucleic acid. In some embodiments, the compositions comprise one or more donor nucleic acids described herein. In some embodiments, the compositions are capable of editing a target nucleic acid in a cell or a subject. In some embodiments, the compositions are capable of editing a target nucleic acid or the expression thereof in a cell, in a tissue, in an organ, in vitro, in vivo, or ex vivo. In some embodiments, the compositions are capable of editing a target nucleic acid in a sample comprising the target nucleic.

In some embodiments, compositions described herein comprise plasmids described herein, viral vectors described herein, non-viral vectors described herein, or combinations thereof. In some embodiments, compositions described herein comprise the viral vectors. In some embodiments, compositions described herein comprise an AAV. In some embodiments, compositions described herein comprise liposomes (e.g., cationic lipids or neutral lipids), dendrimers, lipid nanoparticle (LNP), or cell-penetrating peptides. In some embodiments, compositions described herein comprise an LNP.

### X. Pharmaceutical Compositions and Modes of Administration

Described herein are formulations of introducing compositions or components of a system described herein to a host.

In some embodiments, compositions described herein are pharmaceutical compositions. In some embodiments, the pharmaceutical compositions comprise compositions described herein, systems described herein, vectors described herein, engineered HSCs described herein, or combinations thereof. In some embodiments, the pharmaceutical compositions further comprise pharmaceutically acceptable carrier. In some embodiments, the pharmaceutical composition comprises a pharmaceutically acceptable salt, one or more of a vehicle, adjuvant, excipient, or carrier, such as a filler, disintegrant, a surfactant, a binder, a lubricant, or combinations thereof. Remington: The Science and Practice of Pharmacy, 21st edition, 2005, ed. D. B. Troy, Lippincott Williams & Wilkins, Philadelphia; Encyclopedia of Pharmaceutical Technology, eds. J. Swarbrick and J. C. Boylan, 1988-1999, Marcel Dekker, New York; and Encyclopedia of Pharmaceutical Technology, eds. J. Swarbrick, 2015, CRC Press, Boca Raton disclose various carriers used in formulating pharmaceutically acceptably compositions and known techniques for the preparation thereof. Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives. In some embodiments, the vector is formulated for delivery through injection by a needle carrying syringe. In some embodiments, the composition is formulated for delivery by electroporation. In some embodiments, the composition is formulated for delivery by chemical method. In some embodiments, the pharmaceutical compositions comprise a virus vector or a non-viral vector.

Pharmaceutical compositions described herein comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO₃. In some embodiments, the salt is Mg²⁺SO₄²⁻.

Pharmaceutical compositions described herein are in the form of a solution (e.g., a liquid). In some embodiments, the solution is formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some cases, the pH of the solution is less than 7. In some cases, the pH is greater than 7.

### XI. Systems

Disclosed herein, in some aspects, are systems for modifying, or editing a target nucleic acid, comprising (i) a polypeptide or a nucleic acid encoding the polypeptide, wherein the polypeptide comprises an effector protein, an effector partner, a fusion protein or a combination thereof, and (ii) a guide nucleic acid, or a nucleic acid encoding the guide nucleic acid described herein. In some embodiments systems for modifying, or editing a target nucleic acid, comprising the effector proteins or nucleic acids encoding the effector proteins described herein, or a multimeric complex thereof.

In some embodiments, the one or more components individually comprises one or more of the following: (i) an effector protein, or a nucleic acid encoding the effector protein; (ii) an effector partner, or a nucleic acid encoding the effector partner; (iii) a fusion protein, or a nucleic acid encoding the fusion protein; and (iv) a guide nucleic acid, or a nucleic acid encoding the guide nucleic acid. In some embodiments, systems are used for modifying or editing a target nucleic acid. In some embodiments, systems are used for inserting a donor nucleic acid into a target nucleic acid. In some embodiments, systems comprise an effector protein or a nucleic acid encoding the effector protein described herein, a guide nucleic acid or a nucleic acid encoding the guide nucleic acid a reagent described herein, a donor nucleic acid described herein, support medium, or a combination thereof. In some embodiments, the effector protein comprises an effector protein, or a fusion protein thereof, described herein. In some embodiments, effector proteins comprise an amino acid sequence that is at least 70%, at least 75%, at least 80%, at least 85%, at least 90%, at least 95%, or 100% identical to any one of the nucleotide sequences recited in TABLE 1. In some embodiments, effector proteins comprise an amino acid sequence that is at least 70%, at least 75%, at least 80%, at least 85%, at least 90%, at least 95%, or 100% similar to any one of the nucleotide sequences recited in TABLE 1. In some embodiments, the guide nucleic acid comprises at least one nucleotide sequence selected from the nucleotide sequences recited in any one of TABLE 5, TABLE 6, TABLE 7, TABLE 8 and TABLE 9. In some embodiments, the target nucleic acid comprises a nucleotide sequence that is at least 70%, at least 75%, at least 80%, at least 85%, at least 90%, at least 95%, or 100% identical to any one of the nucleotide sequences recited in TABLE 11. In some embodiments, the donor nucleic acid comprises a nucleotide sequence encoding an amino acid sequence that is associated with any one of the diseases recited in TABLE 12. In some embodiments, the system edits the target nucleic acid in vivo.

Also disclosed herein are systems for modifying a target nucleic acid in cells. In some embodiments, systems for modifying a target nucleic acid in a cell, the system comprising: (i) a polypeptide, or a nucleic acid encoding the polypeptide, wherein the polypeptide comprises an amino acid sequence that is at least 85% identical to any one of the amino acid sequences set forth in TABLE 1; and (ii) an engineered guide nucleic acid, or a nucleic acid encoding the engineered guide nucleic acid, wherein: (a) the engineered guide nucleic acid comprises a first region and a second region, (b) the first region at least partially binds to the polypeptide to form an RNP complex, (c) the second region comprises a nucleic acid that is complementary or the reverse complement of the target nucleic acid, (d) the second region hybridizes to the target nucleic acid, (e) the RNP complex upon hybridization of the second region to the target nucleic modifies the target nucleic acid of the cell, and (f) the first region and the second region are heterologous to each other. In some embodiments, the nucleic acid encoding the polypeptide, the nucleic acid encoding the engineered guide nucleic acid, or both are mRNA. In some embodiments, the system further comprises a donor nucleic acid.

Also disclosed herein are systems for modifying a target nucleic acid in a cell. In some embodiments, the systems comprise: (a) an engineered guide nucleic acid, or a nucleic acid encoding the engineered guide nucleic acid, wherein: (i) the engineered guide nucleic acid comprises a first region and a second region, (ii) the second region comprises a nucleotide sequence that is at least 80% identical to any one of repeat sequences recited in TABLE 5 or intermediary sequences recited in TABLE 8, (iii) the second region comprises a nucleotide sequence that is at least 80% identical to any one of spacer sequences recited in TABLE 7, and (iv) the first region and the second region are heterologous to each other; and (b) a polypeptide, or a nucleic acid encoding the polypeptide, wherein: (i) the polypeptide at least partially binds to the first region to form an RNP complex, (ii) the RNP complex, upon hybridization of the second region to the target nucleic, modifies the target nucleic acid of the cell. In some embodiments, the polypeptide comprises an amino acid sequence that is at least 85% identical to any one of the amino acid sequences set forth in TABLE 1. In some embodiments, the nucleic acid encoding the polypeptide, the nucleic acid encoding the engineered guide nucleic acid, or both are mRNA. In some embodiments, the system further comprises a donor nucleic acid.

Also disclosed herein are systems for modifying a target nucleic acid in a hematopoietic stem cell (HSC). In some embodiments, systems for modifying a target nucleic acid in a HSC, the system comprising: (i) a polypeptide, or a nucleic acid encoding the polypeptide, wherein the polypeptide comprises an amino acid sequence that is at least 85% identical to any one of the amino acid sequences set forth in TABLE 1; and (ii) an engineered guide nucleic acid, or a nucleic acid encoding the engineered guide nucleic acid, wherein: (a) the engineered guide nucleic acid comprises a first region and a second region, (b) the first region at least partially binds to the polypeptide to form an RNP complex, (c) the second region comprises a nucleic acid that is complementary or the reverse complement of the target nucleic acid, (d) the second region hybridizes to the target nucleic acid, (e) the RNP complex upon hybridization of the second region to the target nucleic modifies the target nucleic acid of the HSC, and (f) the first region and the second region are heterologous to each other. In some embodiments, the nucleic acid encoding the polypeptide, the nucleic acid encoding the engineered guide nucleic acid, or both are mRNA. In some embodiments, the system further comprises a donor nucleic acid.

In some embodiments, the systems for modifying a target nucleic acid in a HSC described herein comprise an RNP complex comprising a polypeptide, and an engineered guide nucleic acid, wherein the polypeptide at least partially binds to a first region of the engineered guide nucleic acid. In some embodiments, the RNP complex, upon hybridization of a second region of the engineered guide nucleic acid to the target nucleic acid, modifies the target nucleic acid in the HSC. In some embodiments, the HSC, following modification of the target nucleic acid by the RNP complex, retains cell viability relative to an unmodified HSC. In some embodiments, the HSC, following modification of the target nucleic acid by the RNP complex, retains cell proliferation relative to an unmodified HSC. In some embodiments, the HSC, following modification of the target nucleic acid by the RNP complex, retains multi-lineage development potential relative to an unmodified HSC.

In some embodiments, the systems for modifying a target nucleic acid in a HSC, the system comprising: (i) a polypeptide, or a nucleic acid encoding the polypeptide, wherein the polypeptide comprises an amino acid sequence that is at least 85% identical to any one of the amino acid sequences set forth in TABLE 1; and (ii) an engineered guide nucleic acid, or a nucleic acid encoding the engineered guide nucleic acid, wherein: (a) the engineered guide nucleic acid comprises a first region and a second region, (b) the polypeptide at least partially binds to the first region to form an RNP complex, (c) the second region comprises a nucleotide sequence that is complementary or the reverse complement of a target sequence of the target nucleic acid, (d) the RNP complex, upon hybridization of the second region to the target nucleic, modifies the target nucleic acid of the HSC, and (e) the first region and the second region are heterologous to each other, and (f) the HSC following modification by the RNP complex retains at least one of cell viability, cell proliferation, and multi-lineage development potential relative to an unmodified HSC.

In some embodiments, a system described herein comprises at least two engineered guide nucleic acids selected from: (a) a first engineered guide nucleic acid comprising a spacer sequence that is at least 90% identical or complementary to an equal length portion of a target sequence of a gene encoding the HBB gene, a promoter thereof, an enhancer thereof, or a fragment thereof; (b) a second engineered guide nucleic acid comprising a spacer sequence that is at least 90% identical or complementary to an equal length portion of a target sequence of a gene encoding the BCL11A gene, a promoter thereof, an enhancer thereof, or a fragment thereof; (c) a third engineered guide nucleic acid comprising a spacer sequence that is at least 90% identical or complementary to an equal length portion of a target sequence of a gene encoding the HBG1 gene, a promoter thereof, an enhancer thereof, or a fragment thereof; and (d) a fourth engineered guide nucleic acid comprising a spacer sequence that is at least 90% identical or complementary to an equal length portion of a target sequence of a gene encoding the HBG2 gene, a promoter thereof, an enhancer thereof, or a fragment thereof.

In some embodiments, the system further comprises an antibody conjugated to one or more components of any one of the systems described herein, wherein the antibody recognizes and binds HSC. In some embodiments, the antibody recognizes CD34+ and CD117.

### Additional System Components

In some embodiments, systems include a package, carrier, or container that is compartmentalized to receive one or more containers such as vials, tubes, and the like, each of the container(s) comprising one of the separate elements to be used in a method described herein. Suitable containers include, for example, test wells, bottles, vials, syringes, and test tubes. In some embodiments, the containers are formed from a variety of materials such as glass, plastic, or polymers. In some embodiments, the system or systems described herein contain packaging materials. Examples of packaging materials include, but are not limited to, pouches, blister packs, bottles, tubes, bags, containers, bottles, and any packaging material suitable for intended mode of use.

In some embodiments, systems described herein include labels listing contents and/or instructions for use, or package inserts with instructions for use. In some embodiments, the systems include a set of instructions and/or a label is on or associated with the container. In some embodiments, the label is on a container when letters, numbers or other characters forming the label are attached, molded, or etched into the container itself; a label is associated with a container when it is present within a receptacle or carrier that also holds the container (e.g., as a package insert). In some embodiments, the label is used to indicate that the contents are to be used for a specific therapeutic application. In some embodiments, the label indicates directions for use of the contents, such as in the methods described herein. In some embodiments, after packaging the formed product and wrapping or boxing to maintain a sterile barrier, the product is terminally sterilized by heat sterilization, gas sterilization, gamma irradiation, or by electron beam sterilization. Alternatively, in some embodiments, the product is prepared and packaged by aseptic processing.

In some embodiments, systems comprise a solid support. In some embodiments, an RNP or effector protein is attached to a solid support. In some embodiments, the solid support comprises an electrode or a bead. In some embodiments, the bead comprises a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

### XII. Methods and Formulations for Introducing System Components and Compositions into a Target Cell

Disclosed herein, in some aspects, are systems and methods for introducing systems and components of such systems into a target cell. Such systems may comprise, as described herein, one or more components having any one of the polypeptides (e.g., effector proteins, effector partners, fusion proteins, or combination thereof) or a nucleic acid comprising a nucleotide sequence encoding same. In some embodiments, such systems comprise, as described herein, one or more components having a guide nucleic acid or a nucleic acid comprising a nucleotide sequence encoding same. In some embodiments, systems comprise one or more components having a guide nucleic acid and an additional nucleic acid. Systems and components thereof may be used to introduce the polypeptides, guide nucleic acids, or combinations thereof into a target cell. Such methods may be used to modify or edit a target nucleic acid. In some embodiments, systems comprise the polypeptide, one or more guide nucleic acids, and a reagent for facilitating the introduction of the polypeptide and the one or more guide nucleic acids. In some embodiments, system components for the methods comprise a solution, a buffer, a reagent for facilitating the introduction of the polypeptide and the one or more guide nucleic acids, or combinations thereof. A guide nucleic acid (or a nucleic acid comprising a nucleotide sequence encoding same) and/or a polypeptide (e.g., effector protein, effector partner, fusion protein, or combination thereof) (or a nucleic acid comprising a nucleotide sequence encoding same) described herein may be introduced into a host cell by any of a variety of well-known methods. As a non-limiting example, the guide nucleic acid and/or polypeptide may be combined with a lipid. As another non-limiting example, the guide nucleic acid and/or polypeptide may be combined with a particle or formulated into a particle.

### Methods for Introducing Systems and Compositions to a Host

Described herein are methods of introducing various components described herein to a host. A host may be any suitable host. In some embodiments, a host comprises a host cell. When described herein, a host cell comprises an in vivo or in vitro eukaryotic cell, a prokaryotic cell (e.g., bacterial or archaeal cell), or a cell from a multicellular organism (e.g., a cell line) cultured as a unicellular entity. In some embodiments, eukaryotic or prokaryotic cells are, or have been, used as recipients for methods of introduction described herein. In some embodiments, eukaryotic or prokaryotic cells comprise the progeny of the original cell which has been transformed by the methods of introduction described herein. It is understood that the progeny of a single cell is not necessarily be completely identical in morphology or in genomic or total DNA complement as the original parent, due to natural, accidental, or deliberate mutation. In some embodiments, a host cell comprises a recombinant host cell or a genetically modified host cell, if a heterologous nucleic acid, e.g., an expression vector, has been introduced into the cell.

Methods of introducing a nucleic acid and/or protein into a host cell are known in the art, and any convenient method may be used to introduce a subject nucleic acid (e.g., an expression construct/vector) into a target cell (e.g., a human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X (12) 00283-9. doi: 10.1016/j.addr.2012.09.023), and the like. In some embodiments, the nucleic acid and/or protein(s) are introduced into a disease cell comprised in a pharmaceutical composition comprising the guide nucleic acid, the polypeptide, a pharmaceutically acceptable excipient, or combinations thereof.

In some embodiments, molecules of interest, such as nucleic acids of interest, are introduced to a host. In some embodiments, polypeptides are introduced to a host. In some embodiments, vectors, such as lipid particles and/or viral vectors are introduced to a host. In some embodiments, introduction is for contact with a host or for assimilation into the host, for example, introduction into a host cell.

In some embodiments, described herein are methods of introducing one or more nucleic acids, such as a nucleic acid encoding a polypeptide, a nucleic acid that, when transcribed, produces an engineered guide nucleic acid, or combinations thereof, into a host cell. Any suitable method may be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. Further methods are described throughout.

In some embodiments, introducing one or more nucleic acids into a host cell occurs in any culture media and under any culture conditions that promote the survival of the cells. In some embodiments, introducing one or more nucleic acids into a host cell is carried out in vivo or ex vivo. In some embodiments, introducing one or more nucleic acids into a host cell is carried out in vitro.

In some embodiments, polypeptides (e.g., effector proteins, effector partners, fusion proteins, or combination thereof) are provided as RNA. In some embodiments, the RNA is provided by direct chemical synthesis or is transcribed in vitro from a DNA (e.g., encoding the polypeptide). Once synthesized, the RNA is introduced into a cell by way of any suitable technique for introducing nucleic acids into cells (e.g., microinjection, electroporation, transfection, etc.). In some embodiments, introduction of one or more nucleic acid is through the use of a vector and/or a vector system, accordingly, in some embodiments, compositions and system described herein comprise a vector and/or a vector system.

In some embodiments, vectors are introduced directly to a host. In some embodiments, host cells are contacted with one or more vectors as described herein, and in some embodiments, said vectors are taken up by the cells. Methods for contacting cells with vectors include but are not limited to electroporation, calcium chloride transfection, microinjection, lipofection, micro-injection, contact with the cell or particle that comprises a molecule of interest, or a package of cells or particles that comprise molecules of interest.

In some embodiments, components described herein are introduced directly to a host. For example, in some embodiments, an engineered guide nucleic acid is introduced to a host, specifically introduced into a host cell. Methods of introducing nucleic acids, such as RNA into cells include, but are not limited to direct injection, transfection, or any other method used for the introduction of nucleic acids.

In some embodiments, polypeptides (e.g., effector proteins, effector partners, fusion proteins, or combination thereof) described herein are introduced directly to a host. In some embodiments, polypeptides described herein are modified to promote introduction to a host. For example, in some embodiments, polypeptides described herein are modified to increase the solubility of the polypeptide. In some embodiments, the polypeptide is optionally fused to a polypeptide domain that increases solubility. In some embodiments, the domain is linked to the polypeptide through a defined protease cleavage site, such as TEV sequence which is cleaved by TEV protease. In some embodiments, the linker comprises one or more flexible sequences, e.g., from 1 to 10 glycine residues. In some embodiments, the cleavage of the polypeptide is performed in a buffer that maintains solubility of the product, e.g., in the presence of from 0.5 to 2 M urea, in the presence of polypeptides and/or polynucleotides that increase solubility, and the like. Domains of interest include endosomolytic domains, e.g., influenza HA domain; and other polypeptides that aid in production, e.g., IF2 domain, GST domain, GRPE domain, and the like. In another example, the polypeptide is modified to improve stability. For example, the polypeptides is PEGylated, where the polyethyleneoxy group provides for enhanced lifetime in the blood stream. In some embodiments, polypeptides are modified to promote uptake by a host, such as a host cell. For example, a polypeptide described herein is fused to a polypeptide permeant domain to promote uptake by a host cell. Any suitable permeant domains may be used in the non-integrating polypeptides of the present disclosure, including peptides, peptidomimetics, and non-peptide carriers. Examples include penetratin, a permeant peptide that is derived from the third alpha helix of Drosophila melanogaster transcription factor Antennapaedia; the HIV-1 tat basic region amino acid sequence, e.g., amino acids 49-57 of a naturally-occurring tat protein; and poly-arginine motifs, for example, the region of amino acids 34-56 of HIV-1 rev protein, nonaarginine, octa-arginine, and the like. In some embodiments, the site at which the fusion is made is selected in order to optimize the biological activity, secretion or binding characteristics of the polypeptide. In some embodiments, the optimal site is determined by suitable methods.

### Formulations for Introducing Systems and Compositions to a Host

Described herein are formulations of introducing compositions or components of a system described herein to a host. In some embodiments, such formulations, systems and compositions described herein comprise polypeptides (e.g., effector proteins, effector partners, fusion proteins, or combination thereof) and a carrier (e.g., excipient, diluent, vehicle, or filling agent). In some aspects of the present disclosure, the polypeptides are provided in a pharmaceutical composition comprising the polypeptides and any pharmaceutically acceptable excipient, carrier, or diluent.

### XIII. Methods of Modifying a Nucleic Acid

Provided herein are compositions, methods, and systems for modifying (e.g., editing) target nucleic acids. In general, modifying refers to changing the physical composition of a target nucleic acid. However, compositions, methods, and systems disclosed herein are capable of modifying target nucleic acids, such as making epigenetic modifications of target nucleic acids, which does not change the nucleotide sequence of the target nucleic acids per se. In some embodiments, polypeptides (e.g., effector proteins, effector partners, fusion proteins, or combination thereof), compositions and systems described herein are used for modifying a target nucleic acid, which includes editing a target nucleic acid sequence. In some embodiments, modifying a target nucleic acid comprises one or more of: cleaving the target nucleic acid, deleting one or more nucleotides of the target nucleic acid, inserting one or more nucleotides into the target nucleic acid, mutating one or more nucleotides of the target nucleic acid, or otherwise changing one or more nucleotides of the target nucleic acid. In some embodiments, modifying a target nucleic acid comprises one or more of: methylating, demethylating, deaminating, or oxidizing one or more nucleotides of the target nucleic acid.

In some embodiments, compositions, methods, and systems described herein modify a coding portion of a gene, a non-coding portion of a gene, or a combination thereof. In some embodiments, modifying at least one gene using the compositions, methods or systems described herein reduce or increase expression of one or more genes. In some embodiments, the compositions, methods or systems reduce expression of one or more genes by at least 10%, at least 20%, at least 30%, at least 40%, at least 50%, at least 60%, at least 70%, at least 80%, at least 90%, or at least 95%. In some embodiments, the compositions, methods or systems remove all expression of a gene, also referred to as genetic knock out. In some embodiments, the compositions, methods or systems increase expression of one or more genes by at least 10%, at least 20%, at least 30%, at least 40%, at least 50%, at least 60%, at least 70%, at least 80%, at least 90%, or at least 100%.

In some embodiments, the compositions, methods or systems comprise a nucleic acid expression vector, or use thereof, to introduce polypeptides (e.g., effector proteins, effector partners, fusion proteins, or combination thereof), guide nucleic acid, donor template or any combination thereof to a cell. In some embodiments, the nucleic acid expression vector is a viral vector. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions and methods comprise a lipid, polymer, nanoparticle, or a combination thereof, or use thereof, to introduce the polypeptide, guide nucleic acid, donor template or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

In some embodiments, methods of modifying comprise contacting a target nucleic acid with one or more components, compositions or systems described herein. In some embodiments, a method of modifying comprises contacting a target nucleic acid with at least one of: a) one or more polypeptides (e.g., effector proteins, effector partners, fusion proteins, or combination thereof), or one or more nucleic acids encoding the one or more polypeptides; or b) one or more guide nucleic acids, or one or more nucleic acids encoding one or more guide nucleic acids. In some embodiments, a method of modifying comprises contacting a target nucleic acid with a system described herein wherein the system comprises components comprising at least one of: a) one or more polypeptides (e.g., effector proteins, effector partners, fusion proteins, or combination thereof), or one or more nucleic acids encoding the one or more polypeptides; or b) one or more guide nucleic acids, or one or more nucleic acids encoding one or more guide nucleic acids. In some embodiments, a method of modifying comprises contacting a target nucleic acid with a composition described herein comprising at least one of: a) one or more polypeptides (e.g., effector proteins, effector partners, fusion proteins, or combination thereof), or one or more nucleic acids encoding the one or more polypeptides; or b) one or more guide nucleic acids, or one or more nucleic acids encoding one or more guide nucleic acids; in a composition. In some embodiments, a method of modifying as described herein produces a modified target nucleic acid.

In some embodiments, editing a target nucleic acid sequence introduces a mutation (e.g., point mutations, deletions) in a target nucleic acid relative to a corresponding wildtype nucleotide sequence. In some embodiments, editing removes or corrects a disease-causing mutation in a nucleic acid sequence to produce a corresponding wildtype nucleotide sequence. In some embodiments, editing a target nucleic acid sequence removes/corrects point mutations, deletions, null mutations, or tissue-specific mutations in a target nucleic acid. In some embodiments, editing a target nucleic acid sequence is used for generating gene knock-out, gene knock-in, gene editing, gene tagging, or a combination thereof. In some embodiments, methods of the disclosure are targeted to any locus in a genome of a cell.

In some embodiments, modifying comprises single stranded cleavage, double stranded cleavage, donor nucleic acid insertion, epigenetic modification (e.g., methylation, demethylation, acetylation, or deacetylation), or a combination thereof. In some embodiments, cleavage (single-stranded or double-stranded) is site-specific, meaning cleavage occurs at a specific site in the target nucleic acid, often within the region of the target nucleic acid that hybridizes with the guide nucleic acid spacer sequence. In some embodiments, the polypeptides (e.g., effector proteins, effector partners, fusion proteins, or combination thereof) introduce a single-stranded break in a target nucleic acid to produce a cleaved nucleic acid. In some embodiments, the polypeptide is capable of introducing a break in a single stranded RNA (ssRNA). In some embodiments, the polypeptide is coupled to a guide nucleic acid that targets a particular region of interest in the ssRNA. In some embodiments, the target nucleic acid, and the resulting cleaved nucleic acid is contacted with a nucleic acid for homologous recombination (e.g., homology directed repair (HDR)) or non-homologous end joining (NHEJ). In some embodiments, a double-stranded break in the target nucleic acid is repaired (e.g., by NHEJ or HDR) without insertion of a donor template, such that the repair results in an indel in the target nucleic acid at or near the site of the double-stranded break. In some embodiments, an indel, sometimes referred to as an insertion-deletion or indel mutation, is a type of genetic mutation that results from the insertion and/or deletion of one or more nucleotide in a target nucleic acid. In some embodiments, an indel varies in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation. Indel percentage is the percentage of sequencing reads that show at least one nucleotide has been mutation that results from the insertion and/or deletion of nucleotides regardless of the size of insertion or deletion, or number of nucleotides mutated. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are edited by a given polypeptide.

In some embodiments, methods of modifying described herein cleave a target nucleic acid at one or more locations to generate a cleaved target nucleic acid. In some embodiments, the cleaved target nucleic acid undergoes recombination (e.g., NHEJ or HDR). In some embodiments, cleavage in the target nucleic acid is repaired (e.g., by NHEJ or HDR) without insertion of a donor nucleic acid, such that the repair results in an indel in the target nucleic acid at or near the site of the cleavage site. In some embodiments, cleavage in the target nucleic acid is repaired (e.g., by NHEJ or HDR) with insertion of a donor nucleic acid, such that the repair results in an indel in the target nucleic acid at or near the site of the cleavage site.

In some embodiments, wherein the compositions, systems, and methods of the present disclosure restore a wild-type reading frame. In some embodiments, a wild-type reading frame comprises a reading frame that produces at least a partially, or fully, functional protein. In some embodiments, a non-wild-type reading frame comprises a reading frame that produces a non-functional or partially non-functional protein.

Accordingly, in some embodiments, compositions, systems, and methods described herein edit 1 to 1,000 nucleotides or any integer in between, in a target nucleic acid. In some embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, are edited by the compositions, systems, and methods described herein. In some embodiments, 1, 2, 3, 4, 5, 6, 7, 8, 9, 10, or more nucleotides are edited by the compositions, systems, and methods described herein. In some embodiments, 10, 20, 30, 40, 50, 60, 70, 80 90, 100 or more nucleotides, or any integer in between, are edited by the compositions, systems, and methods described herein. In some embodiments, 100, 200, 300, 400, 500, 600, 700, 800, 900 or more nucleotides, or any integer in between, are edited by the compositions, systems, and methods described herein.

In some embodiments, methods comprise use of two or more polypeptides (e.g., effector proteins, effector partners, fusion proteins, or combination thereof). An illustrative method for introducing a break in a target nucleic acid comprises contacting the target nucleic acid with: (a) a first engineered guide nucleic acid comprising a region that binds to a first polypeptide described herein; and (b) a second engineered guide nucleic acid comprising a region that binds to a second polypeptide described herein, wherein the first engineered guide nucleic acid comprises an additional region that hybridizes to the target nucleic acid and wherein the second engineered guide nucleic acid comprises an additional region that hybridizes to the target nucleic acid. In some embodiments, the first and second polypeptide are identical. In some embodiments, the first and second polypeptide are not identical.

In some embodiments, editing a target nucleic acid comprises genome editing. In some embodiments, genome editing comprises editing a genome, chromosome, plasmid, or other genetic material of a cell or organism. In some embodiments, the genome, chromosome, plasmid, or other genetic material of the cell or organism is modified in vivo. In some embodiments, the genome, chromosome, plasmid, or other genetic material of the cell or organism is modified in a cell. In some embodiments, the genome, chromosome, plasmid, or other genetic material of the cell or organism is modified in vitro. For example, in some embodiments, a plasmid is edited in vitro using a composition described herein and introduced into a cell or organism.

In some embodiments, editing a target nucleic acid comprises deleting a sequence from a target nucleic acid. For example, in some embodiments, a mutated sequence or a sequence associated with a disease is removed from a target nucleic acid. In some embodiments, editing a target nucleic acid comprises replacing a sequence in a target nucleic acid with a second nucleotide sequence. For example, in some embodiments, a mutated sequence or a sequence associated with a disease is replaced with a second nucleotide sequence lacking the mutation or that is not associated with the disease. In some embodiments, editing a target nucleic acid comprises deleting or replacing a sequence comprising markers associated with a disease or disorder. In some embodiments, editing a target nucleic acid comprises introducing a sequence into a target nucleic acid. For example, in some embodiments, a beneficial sequence or a sequence that reduces or eliminates a disease is inserted into the target nucleic acid.

In some embodiments, methods comprise inserting a donor nucleic acid into a cleaved target nucleic acid. In some embodiments, the donor nucleic acid is inserted at a specified (e.g., effector protein targeted) point within the target nucleic acid. In some embodiments, the cleaved target nucleic acid is cleaved at a single location. In such embodiments, the methods comprise contacting a target nucleic acid with an effector protein described herein, thereby introducing a single-stranded break in the target nucleic acid; and contacting the target nucleic acid with a donor nucleic acid for homologous recombination, optionally by HDR or NHEJ, thereby introducing a new sequence into the target nucleic acid (e.g., at a cleavage site). In some embodiments, the cleaved target nucleic acid is cleaved at two locations. In such embodiments, the methods comprise contacting a target nucleic acid with an effector protein described herein, thereby introducing a single-stranded break in the target nucleic acid; contacting the target nucleic acid with a second effector protein described herein, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally by HDR or NHEJ, thereby introducing a new sequence into the target nucleic acid (e.g., in between two cleavage sites).

In some embodiments, methods comprise editing a target nucleic acid with two or more polypeptides (e.g., effector proteins, effector partners, fusion proteins, or combination thereof). In some embodiments, editing a target nucleic acid comprises introducing a two or more single-stranded breaks in a target nucleic acid. In some embodiments, a break is introduced by contacting a target nucleic acid with an effector protein and a guide nucleic acid. In some embodiments, the guide nucleic acid binds to the effector protein and hybridizes to a region of the target nucleic acid, thereby recruits the effector protein to the region of the target nucleic acid. In some embodiments, binding of the effector protein to the guide nucleic acid and the region of the target nucleic acid activate the effector protein, and activated effector protein introduces a break (e.g., a single stranded break) in the region of the target nucleic acid. In some embodiments, editing a target nucleic acid comprises introducing a first break in a first region of the target nucleic acid and a second break in a second region of the target nucleic acid. For example, in some embodiments, editing a target nucleic acid comprise contacting a target nucleic acid with a first guide nucleic acid that binds to a first effector protein and hybridizes to a first region of the target nucleic acid and a second guide nucleic acid that binds to a second effector protein or programmable nickase and hybridizes to a second region of the target nucleic acid. In some embodiments, the first effector protein introduces a first break in a first strand at the first region of the target nucleic acid, and the second effector protein introduces a second break in a second strand at the second region of the target nucleic acid. In some embodiments, a segment of the target nucleic acid between the first break and the second break is removed, thereby the target nucleic acid is edited. In some embodiments, a segment of the target nucleic acid between the first break and the second break is replaced (e.g., with donor nucleic acid), thereby the target nucleic acid is edited.

In some embodiments, methods, systems and compositions described herein edit a target nucleic acid wherein such editing results in one or more indels. In some embodiments, where compositions, systems, and/or methods described herein effect one or more indels, the impact on the transcription and/or translation of the target nucleic acid is predicted depending on: 1) the amount of indels generated; and 2) the location of the indel on the target nucleic acid. For example, as described herein, in some embodiments, if the amount of indels is not divisible by three, and the indels occur within or along a protein coding region, then the edit or mutation is a frameshift mutation. In some embodiments, if the amount of indels is divisible by three, then a frameshift mutation is not effected, but a splicing disruption mutation and/or sequence skip mutation is effected, such as an exon skip mutation. In some embodiments, if the amount of indels is not evenly divisible by three, then a frameshift mutation is effected.

In some embodiments, methods, systems and compositions described herein edit a target nucleic acid wherein such editing is measured by indel activity. Indel activity measures the amount of change in a target nucleic acid (e.g., nucleotide deletion(s) and/or insertion(s)) compared to a target nucleic acid that has not been contacted by a polypeptide described in compositions, systems, and methods described herein. For example, in some embodiments, indel activity is detected by next generation sequencing of one or more target loci of a target nucleic acid where indel percentage is calculated as the fraction of sequencing reads containing insertions or deletions relative to an unedited reference sequence. In some embodiments, methods, systems, and compositions comprising polypeptides (e.g., effector proteins, effector partners, fusion proteins, or combination thereof) and guide nucleic acid described herein exhibit about 0.0001% to about 65% or more indel activity upon contact to a target nucleic acid compared to a target nucleic acid non-contacted with compositions, systems, or by methods described herein. For example, in some embodiments, methods, systems, and compositions comprising polypeptides (e.g., effector proteins, effector partners, fusion proteins, or combination thereof) and guide nucleic acid described herein exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In some embodiments, editing of a target nucleic acid as described herein effects one or more mutations comprising splicing disruption mutations, frameshift mutations (e.g., 1+ or 2+ frameshift mutation), sequence deletion, sequence skipping, sequence reframing, sequence knock-in, or any combination thereof. In some embodiments, the splicing disruption can be an editing that disrupts a splicing of a target nucleic acid or a splicing of a sequence that is transcribed from a target nucleic acid relative to a target nucleic acid without the splicing disruption. In some embodiments, the frameshift mutation can be an editing that alters the reading frame of a target nucleic acid relative to a target nucleic acid without the frameshift mutation. In some embodiments, the frameshift mutation can be a +2 frameshift mutation, wherein a reading frame is edited by 2 bases. In some embodiments, the frameshift mutation can be a +1 frameshift mutation, wherein a reading frame is edited by 1 base. In some embodiments, the frameshift mutation is an editing that alters the number of bases in a target nucleic acid so that it is not divisible by three. In some embodiments, the frameshift mutation can be an editing that is not a splicing disruption. In some embodiments a sequence as described in reference to the nucleotide sequence deletion, sequence skipping, sequence reframing, and sequence knock-in can be a DNA sequence, a RNA sequence, an edited DNA or RNA sequence, a mutated sequence, a wild-type sequence, a coding sequence, a non-coding sequence, an exonic sequence (exon), an intronic sequence (intron), or any combination thereof. In some embodiments, the nucleotide sequence deletion is an editing where one or more sequences in a target nucleic acid are deleted relative to a target nucleic acid without the nucleotide sequence deletion. In some embodiments, the nucleotide sequence deletion can result in or effect a splicing disruption or a frameshift mutation. In some embodiments, the nucleotide sequence deletion result in or effect a splicing disruption. In some embodiments, the nucleotide sequence skipping is an editing where one or more sequences in a target nucleic acid are skipped upon transcription or translation of the target nucleic acid relative to a target nucleic acid without the nucleotide sequence skipping. In some embodiments, the nucleotide sequence skipping can result in or effect a splicing disruption or a frameshift mutation. In some embodiments, the nucleotide sequence skipping can result in or effect a splicing disruption. In some embodiments, the nucleotide sequence reframing is an editing where one or more bases in a target are edited so that the reading frame of the nucleotide sequence is reframed relative to a target nucleic acid without the nucleotide sequence reframing. In some embodiments, the nucleotide sequence reframing can result in or effect a splicing disruption or a frameshift mutation. In some embodiments, the nucleotide sequence reframing can result in or effect a frameshift mutation. In some embodiments, the nucleotide sequence knock-in is an editing where one or more sequences is inserted into a target nucleic acid relative to a target nucleic acid without the nucleotide sequence knock-in. In some embodiments, the nucleotide sequence knock-in can result in or effect a splicing disruption or a frameshift mutation. In some embodiments, the nucleotide sequence knock-in can result in or effect a splicing disruption.

In some embodiments, editing of a target nucleic acid can be locus specific, wherein compositions, systems, and methods described herein can edit a target nucleic acid at one or more specific loci to effect one or more specific mutations comprising splicing disruption mutations, frameshift mutations, sequence deletion, sequence skipping, sequence reframing, sequence knock-in, or any combination thereof. For example, editing of a specific locus can affect any one of a splicing disruption, frameshift (e.g., 1+ or 2+ frameshift), sequence deletion, sequence skipping, sequence reframing, sequence knock-in, or any combination thereof. In some embodiments, editing of a target nucleic acid can be locus specific, modification specific, or both. In some embodiments, editing of a target nucleic acid can be locus specific, modification specific, or both, wherein compositions, systems, and methods described herein comprise polypeptides (e.g., effector proteins, effector partners, fusion proteins, or combination thereof) described herein and a guide nucleic acid described herein.

In some embodiments, methods of editing a target nucleic acid or modulating the expression of a target nucleic acid is performed in vivo. In some embodiments, methods of editing a target nucleic acid or modulating the expression of a target nucleic acid is performed in vitro. For example, in some embodiments, a plasmid is edited in vitro using a composition described herein and introduced into a cell or organism. In some embodiments, methods of editing a target nucleic acid or modulating the expression of a target nucleic acid is performed ex vivo. For example, in some embodiments, methods comprise obtaining a cell from a subject, editing a target nucleic acid in the cell with methods described herein, and returning the cell to the subject.

In some embodiments, methods of modifying described herein comprise contacting a target nucleic acid with one or more components, compositions or systems described herein. In some embodiments, the one or more components, compositions or systems described herein comprise at least one of: a) one or more polypeptides (e.g., effector proteins, effector partners, fusion proteins, or combination thereof), or one or more nucleic acids encoding the one or more polypeptides; and b) one or more guide nucleic acids, or one or more nucleic acids encoding the one or more guide nucleic acids. In some embodiments, the one or more effector proteins introduce a single-stranded break or a double-stranded break in the target nucleic acid. In some embodiments, methods of modifying described herein produce a modified target nucleic acid comprising an engineered nucleic acid sequence that expresses polypeptide having new activity as compared to an unmodified target nucleic acid, or alters expression of an endogenous polypeptide as compared to an unmodified target nucleic acid.

In some embodiments, methods of modifying described herein comprise using one or more guide nucleic acids or uses thereof, wherein the methods modify a target nucleic acid at a single location. In some embodiments, the methods comprise contacting an RNP comprising polypeptides (e.g., effector proteins, effector partners, fusion proteins, or combination thereof) and a guide nucleic acid to the target nucleic acid. In some embodiments, the methods introduce a mutation (e.g., point mutations, deletions) in the target nucleic acid relative to a corresponding wildtype nucleotide sequence. In some embodiments, the methods remove or correct a disease-causing mutation in a nucleic acid sequence to produce a corresponding wildtype nucleotide sequence. In some embodiments, the methods remove/correct point mutations, deletions, null mutations, or tissue-specific mutations in a target nucleic acid. In some embodiments, the methods introduce a single stranded cleavage, a nick, a deletion of one or two nucleotides, an insertion of one or two nucleotides, a substitution of one or two nucleotides, an epigenetic modification (e.g., methylation, demethylation, acetylation, or deacetylation), or a combination thereof to the target nucleic acid. In some embodiments, the methods comprise using an effector protein and two guide nucleic acids, wherein two RNPs cleave the target nucleic acid at the same location, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

In some embodiments, methods of modifying described herein comprise using one or more guide nucleic acids or uses thereof, wherein the methods modify a target nucleic acid at two different locations. In some embodiments, the methods introduce two cleavage sites in the target nucleic acid, wherein a first cleavage site and a second cleavage site comprise one or more nucleotides therebetween. In some embodiments, the methods cause deletion of the one or more nucleotides. In some embodiments, the deletion restores a wild-type reading frame. In some embodiments, the wild-type reading frame produces at least a partially functional protein. In some embodiments, the deletion causes a non-wild-type reading frame. In some embodiments, a non-wild-type reading frame produces a partially functional protein or non-functional protein. In some embodiments, the at least partially functional protein has at least 50%, at least 60%, at least 70%, at least 80%, at least 90%, at least 100%, at least 110%, at least 120%, at least 130%, at least 140%, at least 150%, at least 180%, at least 200%, at least 300%, at least 400% activity compared to a corresponding wildtype protein. In some embodiments, the methods comprise using an effector protein and two guide nucleic acids, wherein two RNPs cleave the target nucleic acid at different locations, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

In some embodiments, methods of editing described herein comprise inserting a donor nucleic acid into a cleaved target nucleic acid. In some embodiments, the cleaved target nucleic acid formed by introducing a single-stranded break into a target nucleic acid. In some embodiments, the donor nucleic acid is inserted at a specified (e.g., effector protein targeted) point within the target nucleic acid. In some embodiments, the cleaved target nucleic acid is cleaved at a single location. In such embodiments, the methods comprise contacting a target nucleic acid with an effector protein described herein, thereby introducing a single-stranded break in the target nucleic acid; and contacting the target nucleic acid with a donor nucleic acid for homologous recombination, optionally by HDR or NHEJ, thereby introducing a new sequence into the target nucleic acid (e.g., at a cleavage site). In some embodiments, the cleaved target nucleic acid is cleaved at two locations. In such embodiments, the methods comprise contacting a target nucleic acid with an effector protein described herein, thereby introducing a single-stranded break in the target nucleic acid; contacting the target nucleic acid with a second effector protein described herein, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally by HDR or NHEJ, thereby introducing a new sequence into the target nucleic acid (e.g., in between two cleavage sites).

In some embodiments, methods of modifying described herein comprise methods of increasing, reducing, or eliminating the expression of a target nucleic acid (e.g., an aberrant gene) as set forth in TABLE 10 by at least 10%, at least 20%, at least 30%, at least 40%, at least 50%, at least 60%, at least 70%, at least 80%, at least 90%, at least 95% or more relative to expression of a WT gene. For example, in some embodiments, compositions, systems and methods reduce expression of an aberrant HBB gene by at least 10%, at least 20%, at least 30%, at least 40%, at least 50%, at least 60%, at least 70%, at least 80%, at least 90%, at least 95% or more relative to expression of a WT HBB gene. In some embodiments, methods of modifying described herein comprise methods of modulating expression of an aberrant HBB gene by at least 10%, at least 20%, at least 30%, at least 40%, at least 50%, at least 60%, at least 70%, at least 80%, at least 90%, at least 95% or more relative to expression of a WT HBB gene.

### Method of Manufacturing an Engineered HSC

Provided herein are compositions, methods, and systems for modifying (e.g., editing) target nucleic acids comprised in HSCs. HSCs comprising modified target nucleic acids may comprise epigenetic and/or sequence modifications as compared to a WT HSC. In some embodiments, compositions, methods, and systems for modifying (e.g., editing) target nucleic acids comprised in HSCs yield to the manufacturing of engineered HSCs. In some embodiments, a method of manufacturing an engineered HSC comprising: (i) contacting a HSC with any one of the systems described herein, any one of the vectors described herein, or any one of the pharmaceutical compositions described herein for a first sufficient period of time to allow for transfection of the HSC; and (ii) culturing the HSC for a second sufficient period of time for indels to occur in the target nucleic acid for modifying target nucleic acid and, thereby, producing the engineered HSC. In some embodiments, the transfection comprises electroporation, viral infection, transfection, lipofection, electroporation, calcium phosphate precipitation, direct microinjection, or combinations thereof. In some embodiments, the method is performed in vitro or in vivo. In some embodiments, the target nucleic acid comprises any one of the target nucleic acid sequences listed in TABLE 10, a fragment thereof, a variant thereof, a promoter thereof, an enhancer thereof, or a combination thereof. In some embodiments, the modifying comprises cleaving the target nucleic acid, deleting a nucleic acid of the target nucleic acid, inserting a donor nucleic acid into the target nucleic acid, substituting a nucleic acid of the target nucleic acid with a donor nucleic acid, more than one of the foregoing, or combinations thereof. In some embodiments, the modifying results in upregulation of gene expression, downregulation of gene expression, expression of one or more proteins, or a combination thereof. In some embodiments, the first sufficient time comprises at least about 1 hour, at least about 2 hours, at least about 3 hours, at least about 4 hours, at least about 5 hours or at least about 6 hours. In some embodiments, the second sufficient time at least 1 day, at least 2 days, at least 3 days, at least 4 days, at least 5 days, or at least 6 days. In some embodiments, the method further comprises freezing the HSC. In some embodiments, the method comprises no other agent that alters ability of the engineered HSC to self-renew and/or differentiate into different types of cells.

### Genetically Modified Cells and Organisms

In some embodiments, methods of editing described herein is employed to generate a genetically modified cell. In some embodiments, the cell is a eukaryotic cell (e.g., a mammalian cell) or a prokaryotic cell (e.g., an archaeal cell). In some embodiments, the cell is derived from a multicellular organism and cultured as a unicellular entity. In some embodiments, the cell comprises a heritable genetic modification, such that progeny cells derived therefrom comprise the heritable genetic mutation. In some embodiments, the cell is progeny of a genetically modified cell comprising a genetic modification of the genetically modified parent cell. In some embodiments, the genetically modified cell comprises a deletion, insertion, mutation, or non-native sequence relative to a wild-type version of the cell or the organism from which the cell was derived.

In some embodiments, methods of editing described herein is performed in a cell. In some embodiments, the cell is in vivo. In some embodiments, the cell is ex vivo. In some embodiments, the cell is an isolated cell. In some embodiments, the cell is inside of an organism. In some embodiments, the cell is an organism. In some embodiments, the cell is in a cell culture. In some embodiments, the cell is one of a collection of cells. In some embodiments, the cell is a mammalian cell or derived there from. In some embodiments, the cell is a rodent cell or derived there from. In some embodiments, the cell is a human cell or derived there from. In some embodiments, the cell is a eukaryotic cell or derived there from. In some embodiments, the cell is a progenitor cell or derived there from. In some embodiments, the cell is a pluripotent stem cell or derived there from. In some embodiments, the cell is an animal cell or derived there from. In some embodiments, the cell is an invertebrate cell or derived there from. In some embodiments, the cell is a vertebrate cell or derived there from. In some embodiments, the cell is from a specific organ or tissue. In some embodiments, the cell is a hepatocyte. In some embodiments, the tissue is a subject's blood, bone marrow, or cord blood. In some embodiments, the tissue is a heterologous donor blood, cord blood, or bone marrow. In some embodiments, the tissue is an allogenic blood, cord blood, or bone marrow. In some embodiments, the tissue comprises a muscle. In some embodiments, the muscle comprises a skeletal muscle.

Described herein are methods of correcting a mutation that resulted in sickle cell disease (SCD). In some embodiments, the methods comprise: (a) contacting a target nucleic acid of the HSC with the systems or compositions described herein for a first sufficient period of time to allow for transfection of the HSC; and (b) culturing the HSC for a second sufficient period of time for indels to occur in the target nucleic acid for modifying target nucleic acid. In some embodiments, the method results in one or more of inducing homology directed repair (HDR) for the correction of a single mutation, conversing a base of a single mutation, and correcting common clustered mutations for β-thalassemia.

Described herein are methods of substituting a target sequence of β-globin encoding gene in a HSC. In some embodiments, the methods comprise: (a) contacting a target nucleic acid of the HSC with the systems or compositions described herein for a first sufficient period of time to allow for transfection of the HSC; and (b) culturing the HSC for a second sufficient period of time for indels to occur in the target nucleic acid for modifying target nucleic acid.

Described herein are methods of downregulating BCL11A in a HSC. In some embodiments, the methods comprise: (a) contacting a target nucleic acid of the HSC with the systems or compositions described herein for a first sufficient period of time to allow for transfection of the HSC; and (b) culturing the HSC for a second sufficient period of time for indels to occur in the target nucleic acid for modifying target nucleic acid. In some embodiments, the method results in one or more of inducing homology directed repair (HDR) for the correction of a single mutation, conversing a base of a single mutation, and correcting common clustered mutations for β-thalassemia. In some embodiments, the methods disrupt BCL11A gene by disrupting (e.g., cleaving) the binding motif of one or more transcriptional factors. In some embodiments, the methods disrupt the binding motif of the transcription inhibitor of γ-globin gene. In some embodiments, the methods convert the HBG1/2 promoters to create a novel transcriptional activator. In some embodiments, the methods disrupt BCL11A gene by disrupting (e.g., cleaving) the binding motif of one or more transcriptional factors, and wherein the method further upregulates γ-globin in the HSC. In some embodiments, the binding motif is GATA1.

In some embodiments, methods of editing described herein comprise contacting cells with compositions or systems described herein. In some embodiments, the contacting comprises electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof.

In some embodiments, methods of editing described herein are performed in a subject. In some embodiments, the methods comprise administering compositions described herein to the subject. In some embodiments, the subject is a human. In some embodiments, the subject is a mammal (e.g., rat, mouse, cow, dog, pig, sheep, horse). In some embodiments, the subject is a vertebrate or an invertebrate. In some embodiments, the subject is a laboratory animal. In some embodiments, the subject is a patient. In some embodiments, the subject is at risk of developing, suffering from, or displaying symptoms of a disease. In some embodiments, the subject has a mutation associated with a gene described herein. In some embodiments, the subject displays symptoms associated with a mutation of a gene described herein.

In some embodiments, compositions, systems and methods described herein are employed to generate a genetically modified cell. In some embodiments, the cell is a eukaryotic cell (e.g., a mammalian cell) or a prokaryotic cell (e.g., an archaeal cell). In some embodiments, the cell is a stem cell that self-renews and/or differentiates into different types of cells. In some embodiments, the cell is an allogeneic stem cell. In some embodiments, the cell is a hematopoietic stem cell (HSC). In some embodiments the HSC is derived from induced pluripotent stem cell (iPSC) (e.g., in vitro).

In some embodiments, a HSC is an allogeneic stem cell transplanted from stem cells harvested from any one of three general donor sources: (i) peripheral blood stem cells, as collected from a donor's bloodstream through a process called apheresis; (ii) bone marrow stem cells, as collected from a donor's bone marrow through a procedure called bone marrow harvesting; (ii) umbilical cord blood stem cells as collected from blood from the umbilical cord and placenta during birth.

In some embodiments, a HSC is a pluripotent stem cell. In some embodiments, a HSC is a population of hematopoietic stem and pluripotent cells. In some embodiments, a HSC self-renews and/or differentiates into different types of cells. In some embodiments, a HSC differentiates into any one of: B cell, T cell, NK cell, macrophage, microglia, neutrophils, lymphoid cell, blood cell, myeloid cell, lymphoid cell, hemopoietic stem or progenitor cell, myeloid common progenitor cell, megakaryocytes-erythrocyte progenitor cell, granulocytes-macrophages progenitor cell, monocytic-dendritic progenitor cell, a lymphoid common progenitor cell, an autoimmune cell, a red blood cell.

In some embodiments, a HSC comprising a genetic modification (e.g., engineered HSCs, modified HSCs) differentiates into any one of the cells described herein such that the resultant differentiated cells comprise the genetic modification. In some embodiments, the differentiated cells are used in the treatment of a disease or disorder. In some embodiments, engineered HSCs differentiate into myeloid common progenitor cells involved in the production of blood cells such as platelets, red blood cells (RBCs), and white blood cells. In some embodiments, the RBCs described herein comprise the same genetic modification comprised in the engineered HSCs. In some embodiments, the RBCs described herein for use in the treatment of a genetic blood disease or disorder.

Similarly, engineered HSCs differentiate into lymphocytes (e.g., B cells, T cells). In some embodiments, the B cells and/or T cells described herein for use in the treatment of leukemia.

In some embodiments, the engineered HSCs described herein for use in the treatment of any one of diseases or disorders listed in TABLE 12. In some embodiments, the engineered HSCs described herein for use in the treatment of a genetic blood disease or disorder. In some embodiments, the engineered HSCs described herein for use in the treatment of sickle cell anemia, sickle cell disease (SCD), and/or β-thalassemia.

In some embodiments, HSCs described herein or a population of HSCs described herein can naturally cross the blood-brain barrier and evenly distribute upon crossing. In some embodiments, engineered HSCs cross the blood-brain barrier and may differentiate into a cell involved in the treatment of a genetic blood disease or an autoimmune disease. In some embodiments, HSCs described herein for the use of reducing autoimmune activity in brain.

In some embodiments, HSCs are engineered with any one of the systems described herein. In some embodiments, HSCs are engineered with a system comprising a Cas protein fused to methyl transferase, wherein the methyl transferase permanently silences the transcription of a target nucleic acid. In some embodiments, the permanent silencing of the transcription of a target nucleic acid for the treatment of a disease or disorder described herein, such as sickle cell anemia. In some embodiments, pharmaceutical compositions comprise HSCs as described herein, and a pharmaceutically acceptable carrier. In some embodiments, HSCs undergo extended freezing prior to thawing. In some embodiments, the HSCs undergo in vivo engineering after thawing. In some embodiments any one of the methods described herein involve extended freezing and/or thawing.

### XIV. Methods of Treating a Disease or Disorder

Described herein are methods for treating a disease in a subject by contacting a target nucleic acid with a composition or system described herein, wherein the target nucleic acid is associated with a gene or expression of a gene related to the disease. In some embodiments, methods comprise treating, preventing, or inhibiting a disease or disorder associated with a mutation or aberrant expression of a gene. In some embodiments, methods for treating a disease or disorder comprise methods of editing a nucleic acid described herein.

In some embodiments, methods comprise administration of a composition(s) or component(s) of a system described herein. In some embodiments, the composition(s) or component(s) of the system comprises use of a recombinant nucleic acid (DNA or RNA), administered for the purpose to edit a nucleic acid. In some embodiments, the composition or component of the system comprises use of a vector to introduce a functional gene or transgene. In some embodiments, vectors comprise nonviral vectors, including cationic polymers, cationic lipids, or bio-responsive polymers. In some embodiments, the bio-responsive polymer exploits chemical-physical properties of the endosomal environment (e.g., pH) to preferentially release the genetic material in the intracellular space. In some embodiments, vectors comprise viral vectors, including retroviruses, adenoviruses, adeno-associated viruses, and herpes simplex viruses. In some embodiments, the vector comprises a replication-defective viral vector, comprising an insertion of a therapeutic gene inserted in genes essential to the lytic cycle, preventing the virus from replicating and exerting cytotoxic effects. By way of non-limiting example, in some embodiments, the composition(s) comprises pharmaceutical compositions described herein. Methods of gene therapy that are applicable to the compositions and systems described herein are described in more detail in Ingusci et al., “Gene Therapy Tools for Brain Diseases”, Front. Pharmacol. 10:724 (2019), which is hereby incorporated by reference in its entirety.

In some embodiments, treating, preventing, or inhibiting disease or disorder in a subject comprises contacting a target nucleic acid associated with a particular ailment with a composition described herein. In some embodiments, the methods of treating, preventing, or inhibiting a disease or disorder involves removing, editing, modifying, replacing, transposing, or affecting the regulation of a genomic sequence of a patient in need thereof. In some embodiments, the methods of treating, preventing, or inhibiting a disease or disorder involves modulating gene expression.

In some embodiments, the compositions and systems described herein are for use in therapy. In some embodiments, the compositions and systems described herein are for use in treating a disease or condition described herein. Also provided is the use of the compositions described herein in the manufacture of a medicament. Also provided is the use of the compositions described herein in the manufacture of a medicament for therapeutic and/or prophylactic treatment of a disease or condition described herein.

In some embodiments, the polypeptides (e.g., effector proteins, effector partners, fusion proteins, or combination thereof) described herein are for use in therapy. In some embodiments, the polypeptides described herein are for use in treating a disease or condition described herein. Also provided is the use of the polypeptides described herein in the manufacture of a medicament. Also provided is the use of the polypeptides described herein in the manufacture of a medicament for therapeutic and/or prophylactic treatment of a disease or condition described herein.

In some embodiments, the guide nucleic acids described herein are for use in therapy. In some embodiments, the guide nucleic acids described herein are for use in treating a disease or condition described herein. Also provided is the use of the guide nucleic acids described herein in the manufacture of a medicament. Also provided is the use of the guide nucleic acids described herein in the manufacture of a medicament for therapeutic and/or prophylactic treatment of a disease or condition described herein.

Described herein are compositions, systems and methods for treating a disease in a subject by editing a target nucleic acid associated with a gene or expression of a gene related to the disease. For example, in some embodiments, the editing comprises knock-out of a gene comprising the target nucleic acid. In some embodiments, the compositions, systems and methods comprise LNPs, wherein the LNPs comprise the effector proteins described herein or nucleic acids encoding the effector proteins, the effector partners described herein or nucleic acids encoding the effector partners, the fusion proteins described herein or nucleic acids encoding the fusion proteins, or combinations thereof. In some embodiments, the LNPs comprise chemically modified guide nucleic acids. In some embodiments, the LNPs described herein are used for delivering the compositions, or one or more components of the systems described herein to a specific organ (e.g., liver). Alternatively, in some embodiments, the compositions, systems and methods comprise AAV particles, wherein the AAV particles comprise nucleic acids encoding the effector proteins described herein, the effector partners described herein, the fusion proteins described herein, or combinations thereof. In some embodiments, the AAV particles comprise nucleic acids encoding guide nucleic acids described herein. In some embodiments, the AAV particles described herein are used for delivering the compositions, or one or more components of the systems described herein to a specific cells (e.g., nerve cells or muscle cells). In some embodiments, methods comprise administering a composition or cell described herein to a subject. By way of non-limiting example, in some embodiments, the disease comprises a cancer, an ophthalmological disorder, a neurological disorder, a neurodegenerative disease, a blood disorder, or a metabolic disorder, or a combination thereof. In some embodiments, the disease comprises an inherited disorder, also referred to as a genetic disorder. In some embodiments, the disease is the result of an infection or associated with an infection. Also, by way of non-limiting example, the compositions are pharmaceutical compositions described herein.

In some embodiments, the compositions and methods described herein are used for treating, preventing, or inhibiting a disease or syndrome in a subject. In some embodiments, treating, preventing, or inhibiting a disease or syndrome comprises modifying or editing a target nucleic acid, inserting a donor nucleic acid, or a combination thereof. In some embodiments the compositions and methods described herein are used for treating, preventing, or inhibiting a disease or syndrome listed in, but not limited to, TABLE 12.

In some embodiments, treating, preventing, or inhibiting a disease or syndrome comprises modifying or editing a target nucleic acid, wherein the target nucleic acid comprises any one of the genes recited in TABLE 11, a fragment thereof, a promoter thereof, an enhancer thereof, or a combination thereof. In some embodiments, the target nuclei acid comprises any one of the following genes: BCL11A, B2M, PNPLA2, CHD4, CIITA, GATA1, HBA, HBA1, HBA2, β-globin gene (HBB gene), HBD, HBE, HBE1, HBG, γ-globin 1 gene (HBG1 gene), γ-globin 2 gene (HBG2 gene), HBM, HBQ1, HBZ, HOXA9, KLF1, MBD3, MYB, TRAC1, ZBTB7A, a fragment thereof, a promoter thereof, an enhancer thereof, or a combination thereof. In some embodiments, the target nucleic acids described herein comprise a ε-globin gene, γ-globin gene (HBG gene), G γ-globin gene (HBG2 gene), A γ-globin gene (HBG1 gene), δ-globin gene, β-globin gene (HBB gene), a fragment thereof, a promoter thereof, an enhancer thereof, or a combination thereof. In some embodiments, the disease comprises sickle cell anemia or β-thalassemia and the target nucleic acid is the Beta globin gene (HBB). In some embodiments, the disease comprises acute myeloid leukemia and the target nucleic acid is the CHD4 gene). In some embodiments, the disease is related to immature blood cells and the target nucleic acid is the GATA1 gene. In some embodiments, the disease is related to fetal hemoglobin (HbF) and the target nucleic acid is the HBG1 and/or HBG2 gene. In some embodiments, the disease is alpha-thalassemia and/or T-Cell adult acute lymphocytic leukemia and the target nucleic acid is the HBZ gene. In some embodiments, the disease is related to early embryo development and the target nucleic acid is the HBE1 gene and/or HBZ gene. In some embodiments, the disease is alpha-thalassemia and/or thalassemia and the target nucleic acid is the HBM gene. In some embodiments, the disease is hemoglobin E disease and/or thalassemia and the target nucleic acid is the HBQ1 gene. In some embodiments, the disease is acute myeloid leukaemia (AML) and the target nucleic acid is the HOXA9 gene. In some embodiments, the disease is related to expression of adult beta-globin and other erythroid (red blood) genes and the target nucleic acid is the KLF1 gene. In some embodiments, the disease is leukemia and the target nucleic acid is the MBD3 gene. In some embodiments, the disease is leukemia, lymphoma, angiocentric glioma and/or acute basophilic leukemia and the target nucleic acid is the MYB gene. In some embodiments, the disease is immunodeficiency and/or T-Cell Receptor-Alpha/Beta deficiency and the target nucleic acid is the TRAC gene. In some embodiments, the disease is related to cell differentiation and proliferation, leukemia and/or persistent fetal hemoglobin and the target nucleic acid is the ZBTB7A gene.

In some embodiments, the target nucleic acid comprises a portion of a gene comprising a mutation associated with disease, a mutated gene whose expression is associated with disease, or both wherein the gene is selected from BCL11A, MYB, HOXA9, HBB, HBG1, HBG2, CHD4, KLF1, MBD3, ZBTB7A, TRAC and GATA1. In some embodiments, the target nucleic acid comprises a portion of the gene wherein the compositions, systems, and methods described herein may be used to selectively downregulate or upregulate expression of the gene. In some embodiments, the gene is HBB. In some embodiments, the target nucleic acid is a mutant allele of the gene. In some embodiments, the target nucleic acid is a wildtype allele of the gene. In some embodiments, the target nucleic acid comprises a portion of a HBB gene wherein the compositions, systems, and methods described herein are useful for treating a blood disease and/or genetic disease. In some embodiments, the disease is sickle cell disease, sickle cell anemia, and/or β-thalassemia.

In some embodiments, the methods for treating, preventing, or inhibiting a disease or syndrome described herein further comprise an antibody conjugated to one or more components of any one of the systems described herein. In some embodiments, the antibody recognizes and binds to HSCs. In some embodiments, the antibody recognizes and binds to CD34+ and/or CD117. In some embodiments, the methods described herein comprise harvesting cells from a patient, wherein the cells are harvested from the blood and/or from the bone marrow. In some embodiments, the harvesting comprises harvesting cells comprising CD34+. In some embodiments, the methods described herein further comprise CD117 antibody conjugated to the RNP or LNP/RNP to deliver cargo into HSC. In some embodiments, the methods described herein are performed in vivo.

In some embodiments, the method further comprises modifying the cells (e.g., HSCs), freezing the cells, thawing the cells, or combinations thereof. In some embodiments, the method of treating a disease or disorder further comprises transplanting the cells into a patient (e.g., bone marrow transplant). In some embodiments, modifying the cells comprises the use of any one of the AAV vectors described herein as described herein. In some embodiments, modifying the cells comprises the use of multiple AAV vectors described herein. In some embodiments, modifying the cells comprises the use of a single vector comprising a protein and more than one guide wherein each of the guide targets a different target nucleic acid (e.g., HBB gene, BCL11A gene, HBG1 gene, HBG2 gene). In some embodiments, transplanting the cells into a patient comprises any one of the relevant methods (e.g., injection) described herein.

In some embodiments, the methods for treating, preventing, or inhibiting a disease or syndrome described herein comprises administering a cell modified by a system in a subject in need thereof, wherein the cell comprises a hematopoietic stem cell (HSC), a population of hematopoietic stem and progenitor cells (HSPCs), a blood cell, a myeloid cell, a lymphoid cell, a myeloid common progenitor cell, a megakaryocytes-erythrocyte progenitor cell, a granulocytes-macrophages progenitor cell, a monocytic-dendritic progenitor cell, or a lymphoid common progenitor cell. In some embodiments, the system comprises: (a) an engineered guide nucleic acid, or a nucleic acid encoding the engineered guide nucleic acid, wherein: (i) the engineered guide nucleic acid comprises a first region and a second region, (ii) the second region comprises a nucleotide sequence that is at least 80% identical to any one of repeat sequences recited in TABLE 5 or intermediary sequences recited in TABLE 8, (iii) the second region comprises a nucleotide sequence that is at least 80% identical to any one of spacer sequences recited in TABLE 7, and (iv) the first region and the second region are heterologous to each other, and (b) a polypeptide, or a nucleic acid encoding the polypeptide, wherein (i) the polypeptide at least partially binds to the first region to form an RNP complex, and (ii) the RNP complex, upon hybridization of the second region to the target nucleic, modifies the target nucleic acid of the cell.

In some embodiments, the methods for treating, preventing, or inhibiting a disease or syndrome described herein comprises modifying a cell in vivo by contacting the cell with a system in a subject in need thereof, wherein the cell comprises a hematopoietic stem cell (HSC), a population of hematopoietic stem and progenitor cells (HSPCs), a blood cell, a myeloid cell, a lymphoid cell, a myeloid common progenitor cell, a megakaryocytes-erythrocyte progenitor cell, a granulocytes-macrophages progenitor cell, a monocytic-dendritic progenitor cell, or a lymphoid common progenitor cell. In some embodiments, the system comprises: (a) an engineered guide nucleic acid, or a nucleic acid encoding the engineered guide nucleic acid, wherein: (i) the engineered guide nucleic acid comprises a first region and a second region, (ii) the second region comprises a nucleotide sequence that is at least 80% identical to any one of repeat sequences recited in TABLE 5 or intermediary sequences recited in TABLE 8, (iii) the second region comprises a nucleotide sequence that is at least 80% identical to any one of spacer sequences recited in TABLE 7, and (iv) the first region and the second region are heterologous to each other, and (b) a polypeptide, or a nucleic acid encoding the polypeptide, wherein (i) the polypeptide at least partially binds to the first region to form an RNP complex, and (ii) the RNP complex, upon hybridization of the second region to the target nucleic, modifies the target nucleic acid of the cell.

In some embodiments, the method of treating one of the diseases or disorders listed in TABLE 12 with the engineered HSC described herein.

### XV. Illustrative Embodiments

Embodiment 1. A system for modifying a target nucleic acid of a hematopoietic stem cell (HSC), the system comprising:
(i) a polypeptide, or a nucleic acid encoding the polypeptide, wherein the polypeptide comprises an amino acid sequence that is at least 85% identical to any one of the amino acid sequences set forth in TABLE 1; and
(ii) an engineered guide nucleic acid, or a nucleic acid encoding the engineered guide nucleic acid, wherein; and
   a) the engineered guide nucleic acid comprises a first region and a second region, b) the polypeptide at least partially binds to the first region to form an RNP complex,
   c) the second region comprises a nucleotide sequence that is complementary or the reverse complement of a target sequence of the target nucleic acid,
   d) the RNP complex, upon hybridization of the second region to the target nucleic, modifies the target nucleic acid of the HSC,
   e) the first region and the second region are heterologous to each other, and
   f) the HSC following modification by the RNP complex retains at least one of cell viability, cell proliferation, and multi-lineage development potential relative to an unmodified HSC.

Embodiment 2. The system of Embodiment 1, wherein the target nucleic acid is within a gene of the HSC.

Embodiment 3. The system of claim 1 or 2, wherein the target nucleic acid comprises a nucleotide sequence comprising any one of genes set forth in TABLE 10, a variant thereof or a portion thereof.

Embodiment 4. The system of any one of Embodiments 1-3, wherein the target nucleic acid comprises a nucleotide sequence that is at least 85% identical to any one of the nucleotide sequences set forth in TABLE 11.

Embodiment 5. The system of any one of Embodiments 1-4, wherein the target nucleic acid comprises any one of the following genes: BCL11A, PNPLA2, CHD4, CIITA, GATA1, HBA, HBA1, HBA2, β-globin gene (HBB gene), 8-globin gene, ¿-globin gene, HBD, HBE, HBE1, γ-globin (HBG gene), HBM, HBQ1, HBZ, HOXA9, KLF1, MBD3, MYB, TRAC1, ZBTB7A, a fragment thereof, a promoter thereof, an enhancer thereof, or a combination thereof.

Embodiment 6. The system of any one of Embodiments 5, wherein the HBG gene comprises γ-globin 1 gene (HBG1 gene), γ-globin 2 gene (HBG2 gene), G γ-globin gene, A γ-globin gene, a fragment thereof, a promoter thereof, an enhancer thereof, or a combination thereof.

Embodiment 7. The system of any one of Embodiments 1-6, wherein the target nucleic acid comprises a HBB gene, a fragment thereof, an enhancer thereof or a promoter thereof.

Embodiment 8. The system of Embodiment 7, wherein the HBB gene comprises a EV6 mutation.

Embodiment 9. The system of any one of Embodiments 1-8, wherein the target nucleic acid is associated with any one of diseases or disorders set forth in TABLE 12.

Embodiment 10. The system of any one of Embodiments 1-9, wherein the target nucleic acid is associated with a genetic blood disease or disorder.

Embodiment 11. The system of any one of Embodiments 1-10, wherein the target nucleic acid is associated with sickle cell anemia, sickle cell disease (SCD), and/or β-thalassemia.

Embodiment 12. The system of Embodiment 1-11, wherein the target nucleic acid comprises a single nuclear polymorphism (SNP).

Embodiment 13. The system of any one of Embodiments 1-12, wherein the polypeptide comprises an amino acid sequence that is at least 90% identical, at least 95% identical, or at least 100% identical to any one of the amino acid sequences set forth in TABLE 1.

Embodiment 14. The system of any one of Embodiments 1-12, wherein the polypeptide comprises a Type V Cas effector protein.

Embodiment 15. The system of any one of Embodiments 1-12, wherein the polypeptide comprises a Cas.265466, a CasPhi. 12, or a variant thereof.

Embodiment 16. The system of any one of Embodiments 1-15, wherein the polypeptide comprises binding activity, nuclease activity, nickase activity, base editing activity, cleavage activity, or a combination thereof.

Embodiment 17. The system of any one of Embodiments 1-16, wherein the polypeptide cleaves within or near the target sequence.

Embodiment 18. The system of any one of Embodiments 1-17, wherein the polypeptide cleaves at least one strand of the target nucleic acid.

Embodiment 19. The system of any one of Embodiments 1-18, wherein the polypeptide comprises at least one mutation or amino acid alteration that results in retaining, enhancing or reducing activity relative to corresponding reference amino acid sequence set forth in TABLE 1.

Embodiment 20. The system of Embodiment 19, wherein the at least one mutation comprises an amino acid substitution at a position as set forth in TABLE 2 relative to corresponding reference amino acid sequence of TABLE 1.

Embodiment 21. The system of Embodiment any one of Embodiments 1-20, wherein the polypeptide has nickase activity or nuclease activity.

Embodiment 22. The system of any one of Embodiments 1-21, wherein the polypeptide comprises an effector protein, an effector partner, a fusion protein or a combination thereof.

Embodiment 23. The system of Embodiment 22, wherein the effector partner is selected from a polymerase, a deaminase, a reverse transcriptase, a transcriptional repressor, an integrase, a recombinase and a transcriptional activator.

Embodiment 24. The system of Embodiment 23, wherein the effector protein is fused to an effector partner, wherein the effector protein and the effector partner are heterologous to each other.

Embodiment 25. The system of Embodiment 23 or 24, wherein the effector protein is directly fused to N terminus or C terminus of the effector partner by an amide bond.

Embodiment 26. The system of any one of Embodiments 1-25, wherein the polypeptide comprises at least one nuclear localization signal sequence.

Embodiment 27. The system of Embodiment 26, wherein the at least one nuclear localization signal sequence independently comprises an amino acid sequence that is identical to any one of nucleotide sequences set forth in TABLE 3.

Embodiment 28. The system of any one of Embodiments 1-27, wherein the polypeptide recognizes a protospacer adjacent motif (PAM).

Embodiment 29. The system of Embodiment 28, wherein the polypeptide recognizes a PAM as set forth in TABLE 4.

Embodiment 30. The system of any one of Embodiments 1-29, wherein the polypeptide comprises an amino acid sequence that is at least 90% identical to any one of SEQ ID NO: 1, 569-631.

Embodiment 31. The system of any one of Embodiments 1-30, wherein the polypeptide comprises an amino acid sequence that is at least 90% identical to SEQ ID NO: 1.

Embodiment 32. The system of Embodiment 31, wherein the polypeptide comprises an amino acid substitution at position D220R, wherein the polypeptide comprises enhanced nuclease activity.

Embodiment 33. The system of any one of Embodiments 1-32, wherein the engineered guide nucleic acid comprises a sgRNA or a crRNA.

Embodiment 34. The system of any one of Embodiments 1-29, wherein the polypeptide comprises an amino acid sequence that is at least 90% identical to any one of SEQ ID NO: 2, 632-700.

Embodiment 35. The system of any one of Embodiments 1-29 and 33, wherein the polypeptide comprises an amino acid sequence that is at least 90% identical to SEQ ID NO: 2.

Embodiment 36. The system of Embodiment 34, wherein the polypeptide comprises an amino acid substitution at position L26R, wherein the polypeptide comprises enhanced nuclease activity.

Embodiment 37. The system of any one of Embodiments 1-29 and 34-36, wherein the engineered guide nucleic acid comprises a crRNA.

Embodiment 38. The system of any one of Embodiments 1-37, wherein the first region comprises a repeat sequence that at least partially binds to the polypeptide.

Embodiment 39. The system of Embodiment 38, wherein the repeat sequence comprises a nucleotide sequence that is at least 85% identical, at least 90% identical, at least 95% identical, or at least 100% identical to any one of the nucleotide sequences set forth in TABLE 5.

Embodiment 40. The system of any one of Embodiments 1-33, wherein the first region comprises a handle sequence that is at least partially bound by the polypeptide.

Embodiment 41. The system of Embodiment 40, wherein the handle sequence comprises a repeat sequence, an intermediary sequence, a linker, or combinations thereof.

Embodiment 42. The system of any one of Embodiments 1-41, wherein the second region comprises at least 10 contiguous nucleotides that are complementary to a eukaryotic sequence.

Embodiment 43. The system of any one of Embodiments 1-41, wherein the second region comprises at least 10 contiguous nucleotides that are the reverse complement of the target sequence.

Embodiment 44. The system of any one of Embodiments 1-43, wherein the second region comprises a spacer sequence that hybridizes to a target sequence of a target nucleic acid, and wherein optionally the spacer sequence comprises any one of the spacer sequences set forth in TABLE 7.

Embodiment 45. The system of any one of Embodiments 1-44, wherein the engineered guide nucleic acid comprises one or more phosphorothioate (PS) backbone modifications, 2′-fluoro (2′-F) sugar modifications, or 2′-O-Methyl (2′OMe) sugar modifications.

Embodiment 46. The system of any one of Embodiments 1-29, wherein the engineered guide nucleic acid comprises a nucleotide sequence that is at least 85% identical to any one of the nucleotide sequences set forth in TABLE 9.

Embodiment 47. The system of any one of Embodiments 1-46, wherein the system comprises at least two engineered guide nucleic acids selected from:
(i) an engineered guide nucleic acid comprising a spacer sequence that is at least 90% identical or complementary to an equal length portion of a target sequence of a gene encoding the HBB gene, a promoter thereof, an enhancer thereof, or a fragment thereof;
(ii) an engineered guide nucleic acid comprising a spacer sequence that is at least 90% identical or complementary to an equal length portion of a target sequence of a gene encoding the BCL11A gene, a promoter thereof, an enhancer thereof, or a fragment thereof;
(iii) an engineered guide nucleic acid comprising a spacer sequence that is at least 90% identical or complementary to an equal length portion of a target sequence of a gene encoding the HBG1 gene, a promoter thereof, an enhancer thereof, or a fragment thereof; and
(iv) an engineered guide nucleic acid comprising a spacer sequence that is at least 90% identical or complementary to an equal length portion of a target sequence of a gene encoding the HBG2 gene, a promoter thereof, an enhancer thereof, or a fragment thereof.

Embodiment 48. The system of any one of Embodiments 1-47, wherein the nucleic acid encoding the polypeptide, the nucleic acid encoding the engineered guide nucleic acid, or both are mRNA.

Embodiment 49. The system of any one of Embodiments 1-48, further comprising a donor nucleic acid, or a nucleic acid encoding a donor nucleic acid.

Embodiment 50. The system of Embodiment 49, wherein the donor nucleic acid comprises a nucleotide, a nucleotide sequence, a coding sequence, a gene, an exon, an intron, a gene regulatory region, a fragment thereof, or combinations thereof.

Embodiment 51. The system of Embodiment 49 or 50, wherein the donor nucleic acid comprises a nucleotide sequence of any one of the following genes: BCL11A, PNPLA2, CHD4, CIITA, GATA1, HBA, HBA1, HBA2, HBB, HBD, HBE, HBE1, HBG, HBG1, HBG2, HBM, HBQ1, HBZ, HOXA9, KLF1, MBD3, MYB, TRAC1, ZBTB7A, a fragment thereof, a promoter thereof, an enhancer thereof, or a combination thereof.

Embodiment 52. The system of any one of Embodiments 49-51, wherein the donor nucleic acid encodes a HBB gene, a BCL11A gene, a HBG1 gene and/or a HBG2 gene, and comprises one or more nucleotide sequences for directing integration into the γ-globin gene.

Embodiment 53. The system of any one of Embodiments 1-52, wherein the system further comprises an antibody, wherein the antibody is conjugated to one or more components of the system of any one of Embodiments 1-50, and wherein the antibody recognizes and binds HSC.

Embodiment 54. The system of Embodiment 53, wherein the antibody recognizes CD34+ and CD117.

Embodiment 55. A system for modifying a target nucleic acid of a cell, the system comprising: (a) an engineered guide nucleic acid, or a nucleic acid encoding the engineered guide nucleic acid, wherein: (i) the engineered guide nucleic acid comprises a first region and a second region, (ii) the second region comprises a nucleotide sequence that is at least 80% identical to any one of repeat sequences recited in TABLE 5 or intermediary sequences recited in TABLE 8, (iii) the second region comprises a nucleotide sequence that is at least 80% identical to any one of spacer sequences recited in TABLE 7, and (iv) the first region and the second region are heterologous to each other; and (b) a polypeptide, or a nucleic acid encoding the polypeptide, wherein: (i) the polypeptide at least partially binds to the first region to form an RNP complex, (ii) the RNP complex, upon hybridization of the second region to the target nucleic, modifies the target nucleic acid of the cell.

Embodiment 56. The system of Embodiment 55, wherein the polypeptide comprises an amino acid sequence that is at least 85% identical to any one of the amino acid sequences set forth in TABLE 1.

Embodiment 57. A vector that encodes one or more components of any one of the systems of Embodiments 1-56.

Embodiment 58. The vector of Embodiment 57 further comprises at least one HSC specific promoter.

Embodiment 59. The vectors of Embodiment 57 or 58, wherein the vector is a viral vector.

Embodiment 60. The vector of Embodiment 59, wherein the viral vector is an adeno associated viral (AAV) vector.

Embodiment 61. The vector of Embodiment 57, wherein the vector is formulated with a lipid or a lipid nanoparticle.

Embodiment 62. A library of nucleic acid vectors comprising at least one vector of any one of Embodiments 57-60.

Embodiment 63. A pharmaceutical composition comprising the system of any one of Embodiments 1-56, the vector of any one of Embodiments 57-61, and a pharmaceutically acceptable carrier.

Embodiment 64. A method of producing an engineered HSC, the method comprising:
(i) contacting a target nucleic acid of a HSC with the system of any one of Embodiments 1-56, the vector of any one of Embodiments 57-61, or the pharmaceutical composition of Embodiment 63 for a first sufficient period of time to allow for transfection of the HSC; and
(ii) culturing the HSC for a second sufficient period of time for indels to occur in the target nucleic acid for modifying target nucleic acid and, thereby, producing the engineered HSC.

Embodiment 65. A method of correcting a mutation that resulted in sickle cell disease (SCD), the method comprising:
(i) contacting a target nucleic acid of the HSC with the system of any one of Embodiments 1-54, the vector of any one of Embodiments 57-61, or the pharmaceutical composition of Embodiment 63 for a first sufficient period of time to allow for transfection of the HSC; and
(ii) culturing the HSC for a second sufficient period of time for indels to occur in the target nucleic acid for modifying target nucleic acid, wherein the method results in one or more of inducing homology directed repair (HDR) for the correction of a single mutation, conversing a base of a single mutation, and correcting common clustered mutations for β-thalassemia.

Embodiment 66. A method of substituting a target sequence of β-globin encoding gene in a HSC, the method comprising:
(i) contacting a target nucleic acid of the HSC with the system of any one of Embodiments 49-54, the vector of any one of Embodiments 57-61, or the pharmaceutical composition of Embodiment 63 for a first sufficient period of time to allow for transfection of the HSC; and
(ii) culturing the HSC for a second sufficient period of time for indels to occur in the target nucleic acid for modifying target nucleic acid.

Embodiment 67. A method of downregulating BCL11A in a HSC, the method comprising:
(i) contacting a target nucleic acid of the HSC with the system of any one of Embodiments 1-54, the vector of any one of Embodiments 57-61, or the pharmaceutical composition of Embodiment 63 for a first sufficient period of time to allow for transfection of the HSC; and
(ii) culturing the HSC for a second sufficient period of time for indels to occur in the target nucleic acid for modifying target nucleic acid,
   wherein the method:
disrupts BCL11A gene by disrupting (e.g., cleaving) the binding motif of one or more transcriptional factors,
disrupts the binding motif of the transcription inhibitor of γ-globin gene, and/or converts the HBG1 2 promoters to create a novel transcriptional activator.

Embodiment 68. The method of Embodiment 67, wherein the method disrupts BCL11A gene by disrupting (e.g., cleaving) the binding motif of one or more transcriptional factors, and wherein the method further upregulates γ-globin in the HSC.

Embodiment 69. The method of Embodiment 68, wherein the binding motif is GATA1.

Embodiment 70. The method of any one of Embodiments 64-69, wherein the transfection comprises electroporation, viral infection, transfection, lipofection, electroporation, calcium phosphate precipitation, direct microinjection, or combinations thereof.

Embodiment 71. The method of any one of Embodiments 64-70, wherein the method is performed in vitro or in vivo.

Embodiment 72. The method of Embodiment 64, wherein the target nucleic acid comprises any one of the target nucleic acid sequences set forth in TABLE 9, a fragment thereof, a variant thereof, a promoter thereof, an enhancer thereof, or a combination thereof.

Embodiment 73. The method of any one of Embodiments 64-72, wherein the modifying comprises cleaving the target nucleic acid, deleting a nucleic acid of the target nucleic acid, inserting a donor nucleic acid into the target nucleic acid, substituting a nucleic acid of the target nucleic acid with a donor nucleic acid, more than one of the foregoing, or combinations thereof.

Embodiment 74. The method of Embodiment 73, wherein the modifying results in upregulation of gene expression, downregulation of gene expression, expression of one or more proteins, or a combination thereof.

Embodiment 75. The method of any one of Embodiments 64-74, wherein the first sufficient time comprises at least about 1 hour, at least about 2 hours, at least about 3 hours, at least about 4 hours, at least about 5 hours or at least about 6 hours.

Embodiment 76. The method of any one of Embodiments 64-75, wherein the second sufficient time at least 1 day, at least 2 days, at least 3 days, at least 4 days, at least 5 days, or at least 6 days.

Embodiment 77. The method of any one of Embodiments 64-76, wherein the method further comprises freezing the HSC.

Embodiment 78. The method of any one of Embodiments 64-77, wherein the method comprises no other agent that alters ability of the engineered HSC to self-renew and/or differentiate into different types of cells.

Embodiment 79. An engineered HSC, wherein a HSC is modified by the method of any one of Embodiments 64-78.

Embodiment 80. The engineered HSC of Embodiment 79, wherein the HSC self-renews and/or differentiates into different types of cells.

Embodiment 81. The engineered HSC of Embodiment 80, wherein the different types of cells comprise a population of hematopoietic stem and progenitor cells (HSPCs), a blood cell, a myeloid cell, a lymphoid cell, a myeloid common progenitor cell, a megakaryocytes-erythrocyte progenitor cell, a granulocytes-macrophages progenitor cell, a monocytic-dendritic progenitor cell, or a lymphoid common progenitor cell.

Embodiment 82. A pharmaceutical composition comprising the engineered HSC of any one of Embodiments 79-81, and a pharmaceutically acceptable carrier.

Embodiment 83. The method of treating a disease or disorder comprising administering the engineered HSC of any one of Embodiments 79-81 or the pharmaceutical composition of Embodiment 80.

Embodiment 84. The method of Embodiment 83, wherein the disease or disorder is any one of the diseases or disorders set forth in TABLE 12.

### Sequences and Tables

TABLE 1 provides illustrative amino acid sequences of effector proteins that are useful in the compositions, systems and methods described herein.

TABLE 1
Exemplary Effector Protein Amino Acid Sequence For

| Name | Amino Acid Sequence |
|---|---|
| CasM. | MSVLTRKVQLIPVGDKEERDRVYKYLRDGIEAQNRAMNLYMSGL |
| 265466 WT | YFAAINEASKEDRKELNQLYSRIATSSKGSAYTTDIEFPTGLASTST |
| | LSMAVRQDFTKSLKDGLMYGRVSLPTYRKDNPLFVDVRFVALRG |
| | TKQKYNGLYHEYKSHTEFLDNLYSSDLKVYIKFANDITFQVIFGNP |
| | RKSSALRSEFQNIFEEYYKVCQSSIQFSGTKIILNMAMDIPDKEIEL |
| | DEDVCVGVDLGIAIPAVCALNKNRYSRVSIGSKEDFLRVRTKIRNQ |
| | RKRLQTNLKSSNGGHGRKKKMKPMDRFRDYEANWVQNYNHYV |
| | SRQVVDFAVKNKAKYINLENLEGIRDDVKNEWLLSNWSYYQLQQ |
| | YITYKAKTYGIEVRKINPYHTSQRCSCCGYEDAGNRPKKEKGQAY |
| | FKCLKCGEEMNADFNAARNIAMSTEFQSGKKTKKQKKEQHENK |
| | (SEQ ID NO: 1) |
| CasPhi.12 WT | MIKPTVSQFLTPGFKLIRNHSRTAGLKLKNEGEEACKKFVRENEIP |
| | KDECPNFQGGPAIANIIAKSREFTEWEIYQSSLAIQEVIFTLPKDKLP |
| | EPILKEEWRAQWLSEHGLDTVPYKEAAGLNLIIKNAVNTYKGVQ |
| | VKVDNKNKNNLAKINRKNEIAKLNGEQEISFEEIKAFDDKGYLLQ |
| | KPSPNKSIYCYQSVSPKPFITSKYHNVNLPEEYIGYYRKSNEPIVSP |
| | YQFDRLRIPIGEPGYVPKWQYTFLSKKENKRRKLSKRIKNVSPILGI |
| | ICIKKDWCVFDMRGLLRTNHWKKYHKPTDSINDLFDYFTGDPVID |
| | TKANVVRFRYKMENGIVNYKPVREKKGKELLENICDQNGSCKLA |
| | TVDVGQNNPVAIGLFELKKVNGELTKTLISRHPTPIDFCNKITAYR |
| | ERYDKLESSIKLDAIKQLTSEQKIEVDNYNNNFTPQNTKQIVCSKL |
| | NINPNDLPWDKMISGTHFISEKAQVSNKSEIYFTSTDKGKTKDVM |
| | KSDYKWFQDYKPKLSKEVRDALSDIEWRLRRESLEFNKLSKSREQ |
| | DARQLANWISSMCDVIGIENLVKKNNFFGGSGKREPGWDNFYKP |
| | KKENRWWINAIHKALTELSQNKGKRVILLPAMRTSITCPKCKYCD |
| | SKNRNGEKFNCLKCGIELNADIDVATENLATVAITAQSMPKPTCE |
| | RSGDAKKPVRARKAKAPEFHDKLAPSYTVVLREAV (SEQ ID NO: 2) |

| Name | Amino Acid Sequence | Name | Amino Acid Sequence |
|---|---|---|---|
| CasM.19498 | SEQ ID NO: 569 | CasM.284833 | SEQ ID NO: 636 |
| CasM.19548 | SEQ ID NO: 570 | CasM.286910 | SEQ ID NO: 637 |
| CasM.19948 | SEQ ID NO: 571 | CasM.287896 | SEQ ID NO: 638 |
| CasM. | SEQ ID NO: 572 | CasM.288518 | SEQ ID NO: 639 |
| 2390217 | | | |
| CasM.270012 | SEQ ID NO: 573 | CasM.288668 | SEQ ID NO: 640 |
| CasM.272451 | SEQ ID NO: 574 | CasM.288712 | SEQ ID NO: 641 |
| CasM.285333 | SEQ ID NO: 575 | CasM.289802 | SEQ ID NO: 642 |
| CasM.286285 | SEQ ID NO: 576 | CasM.292335 | SEQ ID NO: 643 |
| CasM.287128 | SEQ ID NO: 577 | CasM.293891 | SEQ ID NO: 644 |
| CasM.287826 | SEQ ID NO: 578 | CasM.294491 | SEQ ID NO: 645 |
| CasM.287936 | SEQ ID NO: 579 | CasM.294537 | SEQ ID NO: 646 |
| CasM.288450 | SEQ ID NO: 580 | CasM.294655 | SEQ ID NO: 647 |
| CasM.289248 | SEQ ID NO: 581 | CasM.295047 | SEQ ID NO: 648 |
| CasM.289726 | SEQ ID NO: 582 | CasM.297599 | SEQ ID NO: 649 |
| CasM.290380 | SEQ ID NO: 583 | CasM.297894 | SEQ ID NO: 650 |
| CasM.292901 | SEQ ID NO: 584 | CasM.298538 | SEQ ID NO: 651 |
| CasM.294406 | SEQ ID NO: 585 | CasM.299588 | SEQ ID NO: 652 |
| CasM.298142 | SEQ ID NO: 586 | CasM.19910 | SEQ ID NO: 653 |
| CasM.298248 | SEQ ID NO: 587 | CasPhi.1 | SEQ ID NO: 654 |
| CasM.298264 | SEQ ID NO: 588 | CasPhi.2 | SEQ ID NO: 655 |
| CasM.298446 | SEQ ID NO: 589 | CasPhi.3 | SEQ ID NO: 656 |
| CasM.298612 | SEQ ID NO: 590 | CasPhi.4 | SEQ ID NO: 657 |
| CasM.19924 | SEQ ID NO: 591 | CasPhi.5 | SEQ ID NO: 658 |
| CasM.19952 | SEQ ID NO: 592 | CasPhi.6 | SEQ ID NO: 659 |
| CasM.20054 | SEQ ID NO: 593 | CasPhi.7 | SEQ ID NO: 660 |
| CasM.265291 | SEQ ID NO: 594 | CasPhi.8 | SEQ ID NO: 661 |
| CasM.274429 | SEQ ID NO: 595 | CasPhi.9 | SEQ ID NO: 662 |
| CasM.274559 | SEQ ID NO: 596 | CasPhi.10 | SEQ ID NO: 663 |
| CasM.277378 | SEQ ID NO: 597 | CasPhi.11 | SEQ ID NO: 664 |
| CasM.279423 | SEQ ID NO: 598 | CasPhi.13 | SEQ ID NO: 665 |
| CasM.280852 | SEQ ID NO: 599 | CasPhi.14 | SEQ ID NO: 666 |
| CasM.282673 | SEQ ID NO: 600 | CasPhi.15 | SEQ ID NO: 667 |
| CasM.282952 | SEQ ID NO: 601 | CasPhi.16 | SEQ ID NO: 668 |
| CasM.283262 | SEQ ID NO: 602 | CasPhi.17 | SEQ ID NO: 669 |
| CasM.284933 | SEQ ID NO: 603 | CasPhi.18 | SEQ ID NO: 670 |
| CasM.286251 | SEQ ID NO: 604 | CasPhi.19 | SEQ ID NO: 671 |
| CasM.286588 | SEQ ID NO: 605 | CasPhi.20 | SEQ ID NO: 672 |
| CasM.286678 | SEQ ID NO: 606 | CasPhi.21 | SEQ ID NO: 673 |
| CasM.287700 | SEQ ID NO: 607 | CasPhi.22 | SEQ ID NO: 674 |
| CasM.287908 | SEQ ID NO: 608 | CasPhi.23 | SEQ ID NO: 675 |
| CasM.288480 | SEQ ID NO: 609 | CasPhi.24 | SEQ ID NO: 676 |
| CasM.289206 | SEQ ID NO: 610 | CasPhi.25 | SEQ ID NO: 677 |
| CasM.290598 | SEQ ID NO: 611 | CasPhi.26 | SEQ ID NO: 678 |
| CasM.290816 | SEQ ID NO: 612 | CasPhi.27 | SEQ ID NO: 679 |
| CasM.291449 | SEQ ID NO: 613 | CasPhi.28 | SEQ ID NO: 680 |
| CasM.291507 | SEQ ID NO: 614 | CasPhi.29 | SEQ ID NO: 681 |
| CasM.292139 | SEQ ID NO: 615 | CasPhi.30 | SEQ ID NO: 682 |
| CasM.293203 | SEQ ID NO: 616 | CasPhi.31 | SEQ ID NO: 683 |
| CasM.293410 | SEQ ID NO: 617 | CasPhi.32 | SEQ ID NO: 684 |
| CasM.293576 | SEQ ID NO: 618 | CasPhi.33 | SEQ ID NO: 685 |
| CasM.294190 | SEQ ID NO: 619 | CasPhi.34 | SEQ ID NO: 686 |
| CasM.294270 | SEQ ID NO: 620 | CasPhi.35 | SEQ ID NO: 687 |
| CasM.294601 | SEQ ID NO: 621 | CasPhi.36 | SEQ ID NO: 688 |
| CasM.295071 | SEQ ID NO: 622 | CasPhi.37 | SEQ ID NO: 689 |
| CasM.295105 | SEQ ID NO: 623 | CasPhi.38 | SEQ ID NO: 690 |
| CasM.295187 | SEQ ID NO: 624 | CasPhi.39 | SEQ ID NO: 691 |
| CasM.295201 | SEQ ID NO: 625 | CasPhi.41 | SEQ ID NO: 692 |
| CasM.295231 | SEQ ID NO: 626 | CasPhi.42 | SEQ ID NO: 693 |
| CasM.295929 | SEQ ID NO: 627 | CasPhi.43 | SEQ ID NO: 694 |
| CasM.296640 | SEQ ID NO: 628 | CasPhi.44 | SEQ ID NO: 695 |
| CasM.296642 | SEQ ID NO: 629 | CasPhi.45 | SEQ ID NO: 696 |
| CasM.298706 | SEQ ID NO: 630 | CasPhi.46 | SEQ ID NO: 697 |
| CasM.299584 | SEQ ID NO: 631 | CasPhi.47 | SEQ ID NO: 698 |
| CasM.277328 | SEQ ID NO: 632 | CasPhi.48 | SEQ ID NO: 699 |
| CasM.280604 | SEQ ID NO: 633 | CasPhi.49 | SEQ ID NO: 700 |
| CasM.281050 | SEQ ID NO: 634 | CasPhi12j. | SEQ ID NO: 701 |
| | | 12_L17_18_del1 | |
| CasM.281060 | SEQ ID NO: 635 | CasPhi12j. | SEQ ID NO: 702 |
| | | 12_L17_18_del2 | |

TABLE 2 provides illustrative sequences of exemplary variants of polypeptides described herein that are useful in the compositions, systems and methods described herein.

TABLE 2
Exemplary Amino Acid Substitutions

| | | Exemplary |
|---|---|---|
| | | Effector |
| | | Protein |
| Relative | | Activity |
| to SEQ | | Altered by |
| ID No. | Positions of Amino Acid Substitutions | Substitution |
| 1 | K58R, K58K, K58H, I80R, 180K, 180H, T84R, T84K, T84H, | Modified |
| | K105R, K105K, K105H, N193R, N193K, N193H, C202R, | nuclease |
| | C202K, C202H, S209R, S209K, S209H, G210R, G210K, | activity |
| | G210H, A218R, A218K, A218H, D220R, D220K, D220H, | |
| | E225R, E225K, E225H, C246R, C246K, C246H, N286R, | |
| | N286K, N286H, M295R, M295K, M295H, M298R, M298K, | |
| | M298H, A306R, A306K, A306H, Y315R, Y315K, Y315H, | |
| | Q360R, Q360K, Q360H, N58K | |
| 1 | I80R, T84R, K105R, C202R, G210R, A218R, D220R, E225R, | Enhanced |
| | C246R, Q360R, I80K, T84K, G210K, N193K, C202K, A218K, | nuclease |
| | D220K, E225K, C246K, N286K, A306K, Q360K, I80H, T84H, | activity |
| | K105H, G210H, C202H, A218H, D220H, E225H, C246H, | |
| | Q360H, K58W, S209F, M295W, M298L, Y315M | |
| 1 | D220R/A306K, D220R/K250N, D418A/K58W, | Enhanced |
| | D418N/K58W* | nuclease |
| | | activity |
| 1 | D237A, D418A, D418N, E335A, E335Q | Reduced |
| | | nuclease |
| | | activity; |
| | | Dead |
| | | nuclease |
| | | (dCas) |
| 2 | I2R, I2K, I2H, 5K, T5H, T11R, G13R, G13H, G13K, F14R, | Modified |
| | F14H, F14K, K15R, K15H, L16R, L16H, L16K, L16F, I17R, | nuclease |
| | I17H, I17K, R18H, R18K, R18R, N19R, N19H, N19K, N19V, | activity |
| | H20R, H20K, S21R, S21H, S21K, R22H, R22K, R22R, T23R, | |
| | T23H, T23K, A24R, A24H, A24K, G25R, G25H, G25K, L26H, | |
| | L28R, L28H, L28K, K29R, K29H, N30R, N30H, N30K, E31R, | |
| | E31H, E31K, G32R, G32H, G32K, E33R, E33H, E33K, E34R, | |
| | E34H, E34K, A35R, A35H, A35K, C36R, C36H, C36K, K37R, | |
| | K37H, K38R, K38H, F39R, F39H, F39K, V40R, V40H, V40K, | |
| | R41H, R41K, R41R, E42R, E42H, E42K, N43R, N43H, N43K, | |
| | N43A, E44R, E44H, E44K, P46S, D48Y, C50K, C50P, P51R, | |
| | P51H, P51K, N52R, N52H, N52K, F53R, F53H, F53K, F509A, | |
| | Q54H, Q54K, G55R, G55H, G55K, G56R, G56H, G56K, | |
| | P57R, P57H, P57K, A58W, A58F, I59K, I59R, I62K, K65W, | |
| | K65F, E68P, T70E, E71K, E73W, E73F, E73P, I74W, 174F, | |
| | Y75W, Y75F, S77V, S78K, S78D, L79R, A80K, E83K, V84Y, | |
| | V84K, T87G, T87D, P89T, P89K, K90E, P94E, P94Q, P94K, | |
| | E95K, P96K, I97R, K99R, K99H, K99S, E100K, E101K, | |
| | W102T, W106N, W106K, L107F, S108R, S108H, S108K, | |
| | E109R, E109H, E109K, E109A, H110R, H110K, H110T, | |
| | G111R, G111H, G111K, L112R, L112H, L112K, L112E, | |
| | D113R, D113H, D113K, T114R, T114H, T114K, V115R, | |
| | V115H, V115K, P116R, P116H, P116K, P116G, Y117R, | |
| | Y117H, Y117K, K118R, K118H, E119R, E119H, E119K, | |
| | A120R, A120H, A120K, A121R, A121H, A121K, G122R, | |
| | G122H, G122K, L123R, L123H, L123K, N124R, N124H, | |
| | N124K, L125R, L125H, L125K, I126R, I126H, I126K, I127R, | |
| | I127H, I127K, K128R, K128H, N129R, N129H, N129K, | |
| | A130R, A130H, A130K, V131R, V131H, V131K, N132R, | |
| | N132H, N132K, T133R, T133H, T133K, Y134R, Y134H, | |
| | Y134K, K135R, K135H, G136R, G136H, G136K, V137R, | |
| | V137H, V137K, Q138R, Q138H, Q138K, V139H, V139K, | |
| | K146N, N147K, N148E, L149R, L149H, L149K, A150K, | |
| | K151E, N153R, E157R, I158K, A159K, A159N, K160D, | |
| | G163E, S168W, F169F, E171K, F175K, G179R, G179H, | |
| | G179K, Y180R, Y180H, Y180K, L181R, L181H, L181K, | |
| | L182R, L182H, L182K, Q183R, Q183H, Q183K, K184R, | |
| | K184H, K184P, P185R, P185H, P185K, S186H, S186K, | |
| | S186G, S186E, S186R, P187R, P187H, P187K, P187I, N188R, | |
| | N188H, N188K, K189R, K189H, S190R, S190H, S190K, | |
| | S190V, I191R, I191H, I191K, Y192R, Y192H, Y192K, C193R, | |
| | C193H, C193K, Y194R, Y194H, Y194K, Q195R, Q195H, | |
| | Q195K, S196R, S196H, S196K, V197R, V197H, V197K, | |
| | S198H, S198K, S198E, P199R, P199H, P199K, K200R, | |
| | K200H, P201R, P201H, P201K, F202R, F202H, F202K, I203R, | |
| | I203H, I203K, I203G, T204R, T204H, T204K, S205R, S205H, | |
| | S205K, K206R, K206H, Y207R, Y207H, Y207K, H208R, | |
| | H208K, N209R, N209H, N209K, N209D, N209W, N209F, | |
| | V210R, V210H, V210K, G218S, G218K, Y220K, S223K, | |
| | S223V, E225K, I227R, V228R, S229R, P230R, Y231K, | |
| | Y231G, Y231R, Q232R, F233R, D234R, L236R, I238R, | |
| | P239R, I240K, G241R, E242R, P243R, G244R, Y245R, | |
| | V246K, V246R, P247R, K248E, K248R, Q250N, Q250R, | |
| | T252D, T252R, F253R, L254R, S255R, K256E, K256R, | |
| | K257R, E258R, N259R, K260R, R262D, L264R, S265R, | |
| | K266R, I268R, S272Q, P273A, G276V, C279R, I280K, | |
| | K281R, K281H, C285V, V286K, T295N, T295F, T295W, | |
| | H297A, H297R, W298L, Y301L, H302F, H302W, P304E, | |
| | P304K, D306K, D306F, L311T, F312L, F315M, F315K, | |
| | T316R, T316H, T316K, V328N, R329T, M334E, I338S, | |
| | N340S, N340F, K348R, K348H, N355R, N355H, N355K, | |
| | I356W, I356F, C357L, C357E, C357K, N360K, G361R, | |
| | C363V, A366V, V368K, V370L, K384T, L390H, T391W, | |
| | K392A, K392E, T393K, T393E, 1395Q, R397K, P399F, | |
| | P399Q, T400L, C405L, C405R, K407E, F445S, S472R, | |
| | K480L, V483G, G497K, G497R, D501K, M503K, W509A, | |
| | Q511R, Q511H, Q511K, D512R, D512H, D512K, Y513R, | |
| | Y513H, Y513K, K514R, K514H, P515R, P515H, P515K, | |
| | K516R, K516H, L517R, L517H, L517K, D523K, S526N, | |
| | E529K, E529L, E536A, R531E, S536A, N540R, N540H, | |
| | N540K, K541R, K541H, L542R, L542H, L542K, S543R, | |
| | S543H, S543K, K544R, K544H, S545R, S545H, S545K, | |
| | R546H, R546K, R546R, D549L, G577H, G585R, Y590R, | |
| | Y590H, Y590K, K591R, K591H, P592R, P592H, P592K, | |
| | K593R, K593H, K594R, K594H, E595R, E595H, E595K, | |
| | N596R, N596H, N596K, W599F, A602R, A602H, A602K, | |
| | I603R, I603H, I603K, H604R, H604K, K605R, K605H, | |
| | A606R, A606H, A606K, L607R, L607H, L607K, L607F, | |
| | T608R, T608H, T608K, Q612R, R617Y, L620E, M624A, | |
| | K634G, K639E, I653A, A673G, A673R, Q674R, Q674K, | |
| | K678R, P679R, E682R, S684R, G685R, A696R, P699R, | |
| | D703R, P707H, P707K, Y709R, E715R, A716R, K15K, H20H, | |
| | K37K, K38K, Q79K, Q79H, K92R, K92K, K92H, K99K, | |
| | H110H, K118K, K135K, N148R, N148K, N148H, E157K, | |
| | E157H, E164R, E164K, E164H, E166R, E166K, E166H, | |
| | E170R, E170K, E170H, K184K, K189K, K200K, K206K, | |
| | H208H, Y220R, Y220H, S223R, S223H, E258H, K281K, | |
| | K348K, S362R, S362K, S362H, N406R, N406H, K435R, | |
| | K435K, K435H, I471R, I471K, I471H, I489R, I489K, I489H, | |
| | Y490R, Y490K, Y490H, F491R, F491K, F491H, D495R, | |
| | D495K, D495H, K496R, K496K, K496H, K498R, K498K, | |
| | K498H, K500R, K500K, K500H, D501R, D501H, V502R, | |
| | V502K, V502H, K504R, K504K, K504H, S505R, S505K, | |
| | S505H, D506R, D506K, D506H, V521R, V521K, V521H, | |
| | N568R, N568K, N568H, S579K, S579H, Q612K, Q612H, | |
| | S638R, S638H, F701K, F701H | |
| 2 | L26R/E109R, L26R/H208R, L26R/K184R, L26R/K38R, | Modified |
| | L26R/L182R, L26R/Q183R, L26R/S108R, L26R/S198R, | nuclease |
| | L26R/T114R, E109R/H208R, E109R/K184R, E109R/K38R, | activity |
| | E109R/L182R, E109R/Q183R, E109R/S108R, E109R/S198R, | |
| | E109R/T114R, H208R/K184R, H208R/K38R, H208R/L182R, | |
| | H208R/Q183R, H208R/S108R, H208R/S198R, H208R/T114R, | |
| | K184R/K38R, K184R/E109R, K184R/H208R, K184R/L182R, | |
| | K184R/Q183R, K184R/S108R, K184R/S198R, K184R/T114R, | |
| | K38R/L182R, K38R/Q183R, K38R/S108R, K38R/S198R, | |
| | K38R/T114R, L182R/Q183R, L182R/S108R, L182R/S198R, | |
| | L182R/T114R, Q183R/S108R, Q183R/S198R, Q183R/T114R, | |
| | S108R/S198R, S108R/T114R, S198R/T114R, S198R/K92E, | |
| | L26R/1268R, L26R/G244R, F701R/K189P, Q612R/E119S, | |
| | F701R/N406K, L26R/E109K, F701R/K435Q, L26R/Y220S, | |
| | L26R/G57R, L26R/A716R, S579R/E119S, K348R/V521T, | |
| | Q612R/S638K, K118R/K189P, F701R/V521T, K118R/K92E, | |
| | Q612R/A121Q, F701R/E119S, L26R/P239R, L26R/Y231R, | |
| | K348R/S223P, L26R/C279R, K348R/E119S, L26R/E100K, | |
| | K348R/N406K, Q612R/E258K, L26R/S255R, F701R/K92E, | |
| | Q612R/N406K, K348R/K189P, S186R/E258K, Q612R/Y220S, | |
| | L26R/N568D, L26R/G241R, F701R/N568D, Q612R/K435Q, | |
| | T5R/N568D, Q612R/V521T, S186R/V521T, K348R/A121Q, | |
| | L26R/D234R, K348R/K92E, S579R/N568D, L26R/A59R, | |
| | Q612R/K92E, L26R/H297R, L26R/E258R, K348R/K435Q, | |
| | L26R/P94K, K348R/I471T, L26R/E119S, S579R/K435Q, | |
| | L26R/Y709R, S198R/N568D, S186R/N568D, L26R/G497R, | |
| | Q612R/N568D, K118R/N568D, L26R/L254R, K348R/E258K, | |
| | L26R/P96K, L26R/Y245R, L26R/1238R, L26R/P243R, | |
| | L26R/L16F, L26R/P230R, L26R/P199R, L26R/G585R, | |
| | K348R/N568D, L26R/A673R, L26R/V246R, L26R/S229R, | |
| | L26R/S265R, L26R/C50P, L26R/F253R, L26R/L264R, | |
| | L26R/1227R, L26R/P247R, L26R/Q250R, L26R/F253R, | |
| | L26R/L264R, L26R/1227R, L26R/P247R* | |
| 2 | L26R/E109R/H208R, L26R/E109R/K184R, | Modified |
| | L26R/E109R/K38R, L26R/E109R/L182R, | nuclease |
| | L26R/E109R/Q183R, L26R/E109R/S108R, | activity |
| | L26R/E109R/S198R, L26R/E109R/T114R, | |
| | L26R/H208R/K184R, L26R/H208R/K38R, | |
| | L26R/H208R/L182R, L26R/H208R/Q183R, | |
| | L26R/H208R/S108R, L26R/H208R/S198R, | |
| | L26R/H208R/T114R, L26R/K184R/H208R, | |
| | L26R/K184R/K38R, L26R/K184R/L182R, | |
| | L26R/K184R/Q183R, L26R/K184R/S108R, | |
| | L26R/K184R/S198R, L26R/K184R/T114R, | |
| | L26R/K38R/L182R, L26R/K38R/Q183R, L26R/K38R/S108R, | |
| | L26R/K38R/S198R, L26R/K38R/T114R, L26R/L182R/Q183R, | |
| | L26R/L182R/S108R, L26R/L182R/S198R, | |
| | L26R/L182R/T114R, L26R/Q183R/K184R, | |
| | L26R/Q183R/H208R, L26R/Q183R/S108R, | |
| | L26R/Q183R/S198R, L26R/Q183R/T114R, | |
| | L26R/S108R/S198R, L26R/S108R/T114R, | |
| | L26R/S198R/T114R, E109R/H208R/K184R, | |
| | E109R/H208R/K38R, E109R/H208R/L182R, | |
| | E109R/H208R/Q183R, E109R/H208R/S108R, | |
| | E109R/H208R/S198R, E109R/H208R/T114R, | |
| | E109R/K184R/K38R, E109R/K184R/L182R, | |
| | E109R/K184R/Q183R, E109R/K184R/S108R, | |
| | E109R/K184R/S198R, E109R/K184R/T114R, | |
| | E109R/K38R/L182R, E109R/K38R/Q183R, | |
| | E109R/K38R/S108R, E109R/K38R/S198R, | |
| | E109R/K38R/T114R, E109R/L182R/Q183R, | |
| | E109R/L182R/S108R, E109R/L182R/S198R, | |
| | E109R/L182R/T114R, E109R/Q183R/S108R, | |
| | E109R/Q183R/S198R, E109R/Q183R/T114R, | |
| | E109R/S108R/S198R, E109R/S108R/T114R, | |
| | E109R/S198R/T114R, H208R/K184R/K38R, | |
| | H208R/K184R/L182R, H208R/K184R/Q183R, | |
| | H208R/K184R/S108R, H208R/K184R/S198R, | |
| | H208R/K184R/T114R, H208R/K38R/L182R, | |
| | H208R/K38R/Q183R, H208R/K38R/S108R, | |
| | H208R/K38R/S198R, H208R/K38R/T114R, | |
| | H208R/L182R/Q183R, H208R/L182R/S108R, | |
| | H208R/L182R/S198R, H208R/L182R/T114R, | |
| | H208R/Q183R/S108R, H208R/Q183R/S198R, | |
| | H208R/Q183R/T114R, H208R/S108R/S198R, | |
| | H208R/S108R/T114R, H208R/S198R/T114R, | |
| | K184R/K38R/L182R, K184R/K38R/Q183R, | |
| | K184R/K38R/S108R, K184R/K38R/S198R, | |
| | K184R/K38R/T114R, K184R/L182R/Q183R, | |
| | K184R/L182R/S108R, K184R/L182R/S198R, | |
| | K184R/L182R/T114R, K184R/Q183R/S108R, | |
| | K184R/Q183R/S198R, K184R/Q183R/T114R, | |
| | K184R/S108R/S198R, K184R/S108R/T114R, | |
| | K184R/S198R/T114R, K38R/L182R/Q183R, | |
| | K38R/L182R/S108R, K38R/L182R/S198R, | |
| | K38R/L182R/T114R, K38R/Q183R/S108R, | |
| | K38R/Q183R/S198R, K38R/Q183R/T114R, | |
| | K38R/S108R/S198R, K38R/S108R/T114R, | |
| | K38R/S198R/T114R, L182R/Q183R/S108R, | |
| | L182R/Q183R/S198R, L182R/Q183R/T114R, | |
| | L182R/S108R/S198R, L182R/S108R/T114R, | |
| | L182R/S198R/T114R, Q183R/S108R/S198R, | |
| | Q183R/S108R/T114R, Q183R/S198R/T114R, | |
| | S108R/S198R/T114R* | |
| 2 | L26R/E109R/H208R/K184R, L26R/E109R/H208R/K38R, | Modified |
| | L26R/E109R/H208R/L182R, L26R/E109R/H208R/Q183R, | nuclease |
| | L26R/E109R/H208R/S108R, L26R/E109R/H208R/S198R, | activity |
| | L26R/E109R/H208R/T114R, L26R/E109R/K184R/K38R, | |
| | L26R/E109R/K184R/L182R, L26R/E109R/K184R/Q183R, | |
| | L26R/E109R/K184R/S108R, L26R/E109R/K184R/S198R, | |
| | L26R/E109R/K184R/T114R, L26R/E109R/K38R/L182R, | |
| | L26R/E109R/K38R/Q183R, L26R/E109R/K38R/S108R, | |
| | L26R/E109R/K38R/S198R, L26R/E109R/K38R/T114R, | |
| | L26R/E109R/L182R/Q183R, L26R/E109R/L182R/S108R, | |
| | L26R/E109R/L182R/S198R, L26R/E109R/L182R/T114R, | |
| | L26R/E109R/Q183R/S108R, L26R/E109R/Q183R/S198R, | |
| | L26R/E109R/Q183R/T114R, L26R/E109R/S108R/S198R, | |
| | L26R/E109R/S108R/T114R, L26R/E109R/S198R/T114R, | |
| | L26R/H208R/K184R/K38R, L26R/H208R/K184R/L182R, | |
| | L26R/H208R/K184R/Q183R, L26R/H208R/K184R/S108R, | |
| | L26R/H208R/K184R/S198R, L26R/H208R/K184R/T114R, | |
| | L26R/H208R/K38R/L182R, L26R/H208R/K38R/Q183R, | |
| | L26R/H208R/K38R/S108R, L26R/H208R/K38R/S198R, | |
| | L26R/H208R/K38R/T114R, L26R/H208R/L182R/Q183R, | |
| | L26R/H208R/L182R/S108R, L26R/H208R/L182R/S198R, | |
| | L26R/H208R/L182R/T114R, L26R/H208R/Q183R/S108R, | |
| | L26R/H208R/Q183R/S198R, L26R/H208R/Q183R/T114R, | |
| | L26R/H208R/S108R/S198R, L26R/H208R/S108R/T114R, | |
| | L26R/H208R/S198R/T114R, L26R/K184R/K38R/L182R, | |
| | L26R/K184R/K38R/Q183R, L26R/K184R/K38R/S108R, | |
| | L26R/K184R/K38R/S198R, L26R/K184R/K38R/T114R, | |
| | L26R/K184R/L182R/Q183R, L26R/K184R/L182R/S108R, | |
| | L26R/K184R/L182R/S198R, L26R/K184R/L182R/T114R, | |
| | L26R/K184R/Q183R/S108R, L26R/K184R/Q183R/S198R, | |
| | L26R/K184R/Q183R/T114R, L26R/K184R/S108R/S198R, | |
| | L26R/K184R/S108R/T114R, L26R/K184R/S198R/T114R, | |
| | L26R/K38R/L182R/Q183R, L26R/K38R/L182R/S108R, | |
| | L26R/K38R/L182R/S198R, L26R/K38R/L182R/T114R, | |
| | L26R/K38R/Q183R/S108R, L26R/K38R/Q183R/S198R, | |
| | L26R/K38R/Q183R/T114R, L26R/K38R/S108R/S198R, | |
| | L26R/K38R/S108R/T114R, L26R/K38R/S198R/T114R, | |
| | L26R/L182R/Q183R/S108R, L26R/L182R/Q183R/S198R, | |
| | L26R/L182R/Q183R/T114R, L26R/L182R/S108R/S198R, | |
| | L26R/L182R/S108R/T114R, L26R/L182R/S198R/T114R, | |
| | L26R/Q183R/S108R/S198R, L26R/Q183R/S108R/T114R, | |
| | L26R/Q183R/S198R/T114R, L26R/S108R/S198R/T114R, | |
| | E109R/H208R/K184R/K38R, E109R/H208R/K184R/L182R, | |
| | E109R/H208R/K184R/Q183R, E109R/H208R/K184R/S108R, | |
| | E109R/H208R/K184R/S198R, E109R/H208R/K184R/T114R, | |
| | E109R/H208R/K38R/L182R, E109R/H208R/K38R/Q183R, | |
| | E109R/H208R/K38R/S108R, E109R/H208R/K38R/S198R, | |
| | E109R/H208R/K38R/T114R, E109R/H208R/L182R/Q183R, | |
| | E109R/H208R/L182R/S108R, E109R/H208R/L182R/S198R, | |
| | E109R/H208R/L182R/T114R, E109R/H208R/Q183R/S108R, | |
| | E109R/H208R/Q183R/S198R, E109R/H208R/Q183R/T114R, | |
| | E109R/H208R/S108R/S198R, E109R/H208R/S108R/T114R, | |
| | E109R/H208R/S198R/T114R, E109R/K184R/K38R/L182R, | |
| | E109R/K184R/K38R/Q183R, E109R/K184R/K38R/S108R, | |
| | E109R/K184R/K38R/S198R, E109R/K184R/K38R/T114R, | |
| | E109R/K184R/L182R/Q183R, E109R/K184R/L182R/S108R, | |
| | E109R/K184R/L182R/S198R, E109R/K184R/L182R/T114R, | |
| | E109R/K184R/Q183R/S108R, E109R/K184R/Q183R/S198R, | |
| | E109R/K184R/Q183R/T114R, E109R/K184R/S108R/S198R, | |
| | E109R/K184R/S108R/T114R, E109R/K184R/S198R/T114R, | |
| | E109R/K38R/L182R/Q183R, E109R/K38R/L182R/S108R, | |
| | E109R/K38R/L182R/S198R, E109R/K38R/L182R/T114R, | |
| | E109R/K38R/Q183R/S108R, E109R/K38R/Q183R/S198R, | |
| | E109R/K38R/Q183R/T114R, E109R/K38R/S108R/S198R, | |
| | E109R/K38R/S108R/T114R, E109R/K38R/S198R/T114R, | |
| | E109R/L182R/Q183R/S108R, E109R/L182R/Q183R/S198R, | |
| | E109R/L182R/Q183R/T114R, E109R/L182R/S108R/S198R, | |
| | E109R/L182R/S108R/T114R, E109R/L182R/S198R/T114R, | |
| | E109R/Q183R/S108R/S198R, E109R/Q183R/S108R/T114R, | |
| | E109R/Q183R/S198R/T114R, E109R/S108R/S198R/T114R, | |
| | H208R/K184R/K38R/L182R, H208R/K184R/K38R/Q183R, | |
| | H208R/K184R/K38R/S108R, H208R/K184R/K38R/S198R, | |
| | H208R/K184R/K38R/T114R, H208R/K184R/L182R/Q183R, | |
| | H208R/K184R/L182R/S108R, H208R/K184R/L182R/S198R, | |
| | H208R/K184R/L182R/T114R, H208R/K184R/Q183R/S108R, | |
| | H208R/K184R/Q183R/S198R, H208R/K184R/Q183R/T114R, | |
| | H208R/K184R/S108R/S198R, H208R/K184R/S108R/T114R, | |
| | H208R/K184R/S198R/T114R, H208R/K38R/L182R/Q183R, | |
| | H208R/K38R/L182R/S108R, H208R/K38R/L182R/S198R, | |
| | H208R/K38R/L182R/T114R, H208R/K38R/Q183R/S108R, | |
| | H208R/K38R/Q183R/S198R, H208R/K38R/Q183R/T114R, | |
| | H208R/K38R/S108R/S198R, H208R/K38R/S108R/T114R, | |
| | H208R/K38R/S198R/T114R, H208R/L182R/Q183R/S108R, | |
| | H208R/L182R/Q183R/S198R, H208R/L182R/Q183R/T114R, | |
| | H208R/L182R/S108R/S198R, H208R/L182R/S108R/T114R, | |
| | H208R/L182R/S198R/T114R, H208R/Q183R/S108R/S198R, | |
| | H208R/Q183R/S108R/T114R, H208R/Q183R/S198R/T114R, | |
| | H208R/S108R/S198R/T114R, K184R/K38R/L182R/Q183R, | |
| | K184R/K38R/L182R/S108R, K184R/K38R/L182R/S198R, | |
| | K184R/K38R/L182R/T114R, K184R/K38R/Q183R/S108R, | |
| | K184R/K38R/Q183R/S198R, K184R/K38R/Q183R/T114R, | |
| | K184R/K38R/S108R/S198R, K184R/K38R/S108R/T114R, | |
| | K184R/K38R/S198R/T114R, K184R/L182R/Q183R/S108R, | |
| | K184R/L182R/Q183R/S198R, K184R/L182R/Q183R/T114R, | |
| | K184R/L182R/S108R/S198R, K184R/L182R/S108R/T114R, | |
| | K184R/L182R/S198R/T114R, K184R/Q183R/S108R/S198R, | |
| | K184R/Q183R/S108R/T114R, K184R/Q183R/S198R/T114R, | |
| | K184R/S108R/S198R/T114R, K38R/L182R/Q183R/S108R, | |
| | K38R/L182R/Q183R/S198R, K38R/L182R/Q183R/T114R, | |
| | K38R/L182R/S108R/S198R, K38R/L182R/S108R/T114R, | |
| | K38R/L182R/S198R/T114R, K38R/Q183R/S108R/S198R, | |
| | K38R/Q183R/S108R/T114R, K38R/Q183R/S198R/T114R, | |
| | K38R/S108R/S198R/T114R, L182R/Q183R/S108R/S198R, | |
| | L182R/Q183R/S108R/T114R, L182R/Q183R/S198R/T114R, | |
| | L182R/S108R/S198R/T114R, Q183R/S108R/S198R/T114R, | |
| | L26R/Q183R/K184R/T114R | |
| 2 | T5R, L26R, L26K, A121Q, V139R, S198R, S223P, E258K, | Enhanced |
| | I471T, S579R, F701R, P707R, K189P, S638K, Q54R, Q79R, | nuclease |
| | Y220S, N406K, E119S, K92E, K435Q, N568D, V521T | activity |
| 2 | L26K/A121Q, L26R/A121Q, K99R/L149R, K99R/N148R, | Enhanced |
| | L149R/H208R, S362R/L26R L26R/N148R, L26R/H208R, | nuclease |
| | N30R/N148R, L26R/K99R, L26R/P707R, L26R/L149R, | activity |
| | L26R/N30R, L26R/N355R, L26R/K281R, L26R/S108R, | |
| | L26R/K348R, T5R/V139R, I2R/V139R, K99R/S186R, | |
| | L26R/A673G, L26R/Q674R, S579R/L26K, F701R/E258K, | |
| | T5R/L26K, L26R/K435Q, L26R/G685R, L26R/Q674K, | |
| | L26R/P699R, L26R/T70E, L26R/Q232R, L26R/T252R, | |
| | L26R/P679R, L26R/E83K, L26R/E73P, L26R/K248E, L26R, | |
| | T5R/S223P, S579R/S223P, L26R/S223P, T5R/A121Q, | |
| | L26R/A696R, S198R/I471T, L26R/N153R, L26R/E682R, | |
| | L26R/D703R, Q612R/L26K, L26R/I471T, K348R/L26K, | |
| | S579R/I471T, L26R/V228R, T5R/S638K, S579R/K189P, | |
| | S579R/E258K, L26R/K260R, L26R/S638K, S579R/Y220S, | |
| | T5R/I471T, L26R/F233R, L26R/V521T, F701R/A121Q, | |
| | L26R/G361R, S198R/E258K, L26R/S472R, T5R/Y220S, | |
| | L26R/A150K, L26R/S684R, L26R/E157R, L26R/K248R, | |
| | F701R/L26K, S198R/N406K, S198R/Y220S, S198R/S638K, | |
| | S198R/V521T, S579R/A121Q, K348R/Y220S, S198R/K189P, | |
| | L26R/E242R, L26R/K678R, T5R/N406K, L26R/1158K, | |
| | T5R/V521T, L26R/N259R, L26R/K257R, L26R/K256R, | |
| | T5R/K189P, L26R/C405R, S579R/V521T, S579R/N406K, | |
| | T5R/K92E, T5R/E258K, L26R/197R, S579R/S638K, | |
| | T5R/K435Q, F701R/S638K, L26R/L236R, F701R/I471T, | |
| | Q612R/S223P, F701R/S223P, S198R/E119S, S579R/K92E, | |
| | L26R/E715R, Q612R/I471T, F701R/Y220S, S198R/S223P, | |
| | L26R/K266R * | |
| 2 | E157A, E164A, E164L, E166A, E166I, E170A, I489A, I489S, | Nickase |
| | Y490S, Y490A, F491A, F491S, F491G, D495G, D495R, | activity |
| | D495K, K496A, K496S, K498A, K498S, K500A, K500S, | |
| | D501R, D501G, D501K, V502A, V502S, K504A, K504S, | |
| | S505R, D506A | |
| 2 | deletion of S478-S505 and insertion of: | Nickase |
| | SDLYIERGGDPRDVHQQVETKPKGKRKSEIRILKIR (SEQ | activity |
| | ID NO: 897) or | |
| | SDYIVDHGGDPEKVFFETKSKKDKTKRYKRR (SEQ ID | |
| | NO: 898) | |
| 2 | D369A, D369N, D658A, D658N, E567A, E567Q | Reduced |
| | | nuclease |
| | | activity, |
| | | catalytically |
| | | inactive, |
| | | dead protein |
| | | (dCas) |

*wherein “/” indicates simultaneous substitutions

TABLE 3 provides illustrative sequences of exemplary heterologous polypeptide modifications of effector protein(s) that are useful in the compositions, systems and methods described herein.

TABLE 3
Exemplary Nuclear Localization Sequences

| SEQ ID No. | SEQUENCES |
|---|---|
| 3 | PKKKRKVGIHGVPAA |
| 4 | KRPAATKKAGQAKKKK |
| 5 | KR(K/R)R |
| 6 | (P/R)XXKR(^{∧}D/E)(K/R) |
| 7 | KRX(W/F/Y)XXAF |
| 8 | (R/P)XXKR(K/R)(^{∧}D/E) |
| 9 | LGKR(K/R)(W/F/Y) |
| 10 | KRX₁₀K(K/R)(K/R) |
| 11 | GLFXALLXLLXSLWXLLLXA |
| 12 | K(K/R)RK |
| 13 | KRX₁₁K(K/R)(K/R) |
| 14 | KRX₁₂K(K/R)(K/R) |
| 15 | KRX₁₀K(K/R)X(K/R) |
| 16 | KRX₁₁K(K/R)X(K/R) |
| 17 | KRX₁₂K(K/R)X(K/R) |
| 18 | APKKKRKVGIHGVPAA |

*wherein X is any naturally occurring amino acid; and ^{∧}D/E is any naturally occurring amino acid except Asp or Glu

TABLE 4 provides illustrative PAM sequences that are useful in the compositions, systems and methods described herein.

TABLE 4
PAM Sequences

| Effector | | |
|---|---|---|
| Protein | SEQ ID | PAM Sequence |
| SEQ ID No. | No. | (5′→3′) |
| 1 | 19 | NNTN |
| 1 | 20 | ANTR |
| 1 | 21 | AATA |
| 1 | 22 | AATC |
| 1 | 23 | AATG |
| 1 | 24 | AATT |
| 1 | 25 | ACTA |
| 1 | 26 | ACTC |
| 1 | 27 | ACTG |
| 1 | 28 | ACTT |
| 1 | 29 | AGTA |
| 1 | 30 | AGTC |
| 1 | 31 | AGTG |
| 1 | 32 | AGTT |
| 1 | 33 | ATTA |
| 1 | 34 | ATTC |
| 1 | 35 | ATTG |
| 1 | 36 | ATTT |
| 1 | 37 | CNTR |
| 1 | 38 | CATA |
| 1 | 39 | CATT |
| 1 | 40 | CCTA |
| 1 | 41 | CCTG |
| 1 | 42 | CCTT |
| 1 | 43 | CTTA |
| 1 | 44 | CTTC |
| 1 | 45 | CTTG |
| 1 | 46 | CTTT |
| 1 | 47 | GNTR |
| 1 | 48 | GATC |
| 1 | 49 | GATG |
| 1 | 50 | GATT |
| 1 | 51 | GCTG |
| 1 | 52 | GTTA |
| 1 or 2 | 53 | GTTC |
| 1 | 54 | GTTG |
| 1 | 55 | GTTT |
| 1 or 611 | 56 | RTTG |
| 1 | 57 | TATA |
| 1 | 58 | TATC |
| 1 | 59 | TATG |
| 1 | 60 | TATT |
| 1 | 61 | TATY |
| 1 | 62 | TCTA |
| 1 | 63 | TCTG |
| 1 | 64 | TCTT |
| 1 | 65 | TGTA |
| 1 | 66 | TGTG |
| 1 | 67 | TGTT |
| 1 | 68 | TNAR |
| 1 | 69 | TNCR |
| 1 | 70 | TNGR |
| 1 | 71 | TNTC |
| 1, 594, | 72 | TNTG |
| 619, or | | |
| 628 | | |
| 1 | 73 | TNTR |
| 1 | 74 | TNTT |
| 1 | 75 | TNVY |
| 1 | 76 | TTTA |
| 1 or 603 | 77 | TTTC |
| 1 or 2 | 78 | TTTG |
| 1 | 79 | TTTT |
| 1 | 80 | VNTY |
| 1 | 81 | NNTR |
| 2, 664, | 82 | TTN |
| 668, 672, | | |
| 678, 680, | | |
| 681, 683, | | |
| 689, 694, | | |
| or 696 | | |
| 2 | 83 | TTA |
| 2, 593, | 84 | TTC |
| 600, 602, | | |
| 607, 617, | | |
| 623, 627, | | |
| or 629 | | |
| 2 or 673 | 85 | TTG |
| 2 | 86 | TTT |
| 2 | 513 | NTTN |
| 569, 571, | 514 | RTTN |
| 578, 685, | | |
| or 691 | | |
| 569, 580, | 515 | TCG |
| 585, 587, | | |
| 589, 592, | | |
| 596, 609, | | |
| 612, or | | |
| 626 | | |
| 569, 574, | 516 | RTTR |
| 579, 581, | | |
| 585, 587, | | |
| 588, 598, | | |
| 604, 610, | | |
| 611, 612, | | |
| or 622 | | |
| 569 | 517 | KRTTN |
| 570, 595, | 518 | CCN |
| or 647 | | |
| 570 or | 519 | CCR |
| 647 | | |
| 590 | 520 | CC |
| 636 | 521 | CTTN |
| 594 | 522 | G |
| 645 | 523 | GNNN |
| 575 | 524 | GTYG |
| 580 or | 525 | KCG |
| 626 | | |
| 592 | 526 | KYG |
| 615 | 527 | KNTK |
| 615 | 528 | KNTT |
| 630 or | 529 | NCTT |
| 650 | | |
| 605, 613, | 530 | NNCC |
| 618, 643, | | |
| 646, or | | |
| 649 | | |
| 651 | 531 | NNTC |
| 591 | 532 | NTCG |
| 603 or | 533 | NTTC |
| 620 | | |
| 608 | 534 | NTTY |
| 620 | 535 | NYTN |
| 573 | 536 | RTT |
| 679 | 537 | RTTS |
| 577 | 538 | RTNG |
| 583, 606, | 539 | RTRG |
| or 631 | | |
| 575 | 540 | RGTYG |
| 636 | 541 | T |
| 594, 619, | 542 | TG |
| 628, or | | |
| 641 | | |
| 601 or | 543 | TY |
| 627 | | |
| 631 | 544 | TTR |
| 686, 687, | 545 | TTS |
| or 695 | | |
| 601, 624, | 546 | TTY |
| or 627 | | |
| 616, 625, | 547 | TYN |
| 657, 667, | | |
| 676, 677, | | |
| or 684 | | |
| 690 | 548 | TWN |
| 600 or | 549 | TTCN |
| 602 | | |
| 629 | 550 | TTNY |
| 634 | 551 | TTTN |
| 597, 617, | 552 | TTYN |
| 632, or | | |
| 653 | | |
| 636 | 553 | TYYT |
| 617 | 554 | TYYW |
| 614 | 555 | WNCT |
| 619 | 556 | WYTG |
| 631 | 557 | WTTR |
| 619, 628, | 558 | WNTG |
| or 641 | | |
| 653 | 559 | WTTYN |
| 597 or | 560 | YN |
| 636 | | |
| 614 | 561 | YT |
| 593 | 562 | YTC |
| | 563 | YTTN |
| 597 or | 564 | YTTR |
| 632 | | |
| 573 | 565 | ANRTT |
| 625 | 566 | WWTTN |
| 625 | 567 | TTTYN |
| 682 | 568 | VTTN |
| 574 | 859 | RTWG |

*wherein each Nis any nucleotide, each R is A or G, and each V is A, C or G.

TABLE 5 provides illustrative repeat sequences for use in guide nucleic acids that are useful in the compositions, systems and methods described herein.

TABLE 5
Exemplary Repeat Sequences

| Effector | | |
|---|---|---|
| Protein SEQ | SEQ | |
| ID No. | ID No. | Repeat sequence (5′→3′), shown as RNA |
| 1 | 251 | AAGGAUGCCAAAC |
| 2 | 252 | AUAGAUUGCUCCUUACGAGGAGAC |
| 2 | 253 | AUUGCUCCUUACGAGGAGAC |
| 2 | 254 | CUUUCAAGACUAAUAGAUUGCUCCUUACGAGGAGAC |
| 1 | 703 | GTTTGAGAACCTTATGAAATTACAAGGATGCCAAAC |
| 572 | 704 | AGCAGAATACAAC |
| 591 | 705 | GUUGUGAAUGCAGGCAUUUUUGAUGGUAAAUCCAAC |
| 592 | 706 | ACUGUCAGACAAUGCAAAAUGUGUGGUACAUCCAAC |
| 593 | 707 | GUUGAGCUCUGCAUUACGCAGAUGAAUGACGAG |
| 593, 600, 602, | 708 | GAUAUAUCUUGUAUGCAUAUGUAGGUUGUGAG |
| 614, 617, 623, | | |
| 624, 634, 636 | | |
| 594 | 709 | GGUGGAUAUCAUCUUAAAAAGUGAGGUACAUCCAAC |
| 595 | 710 | GGUGUGAACGACCUUUUUUUGCGGUGUAAUUCGAGG |
| 584, 596, 598 | 711 | GCUGUCAGUAGUAGUAAAAAUGGGGGUACAUCCAAC |
| 593, 597, 600, | 712 | GUUGCAACUUACGCAUAGGUGUAAAAUACGAGG |
| 602, 607, 614, | | |
| 617, 623 | | |
| 598, 599, 621 | 713 | GUUGCAGAACCCGAAUAGACGAAUGAAGGAAUGCAAC |
| 600 | 714 | GAUGCAACUUAGAUGCAUAUGUAAGUUGUGAG |
| 600, 601, 602, | 715 | GUUGCAAUGAACGUAUGUGCAUGAGGUGUGAG |
| 614, 617, 623, | | |
| 627 | | |
| 601 | 716 | GUUGCAAUCUGCGUACAGGCGUAAGAUGUGAG |
| 602, 623 | 717 | GAUCAUAUCUGCUUGUAUGGGUAUGCUGCGAG |
| 603, 648 | 718 | GUUGCAGCGUGCGCGAGCGUGUGGCUUGAAGG |
| 604, 622 | 719 | ACUGUCAGUACAUGCAAAAAUGAGGGUACAUCCAAC |
| 605 | 720 | GGUGUAUGUAACCGCAAUUUGAAGGGUGCAUACAGG |
| 605, 613, 649 | 721 | GUUGUAGUCGACCUGAAUCUGUGGGGUGCUUACAGG |
| 606 | 722 | GCUGUAAGUCAUGGAAAAAUGGUGAGUACAUCCAAC |
| 607 | 723 | GAUUAUAUCUGCUUGUAUGGGUAUACUGCGAG |
| 608 | 724 | GUUGCAACUCGCACGUGAAUGCGACUUGAAGG |
| 609 | 725 | ACUGUCAGACAAUGCAAAAUGAGUGGUACAUCCAAC |
| 610 | 726 | GCUGCAUGUCAUGGCAAAAGGAAAGGUACAUCCAAC |
| 611 | 727 | GCUGUCAGACACCUAAAAAAUGAGGGUACAUCCAAC |
| 612, 626 | 728 | GCUGUGAGUCACAGUAAAAAUGAAGGUAUAUCCAAC |
| 613 | 729 | GCUGUAGCCCUGCUCAAAUUGUAGGGCGCAUGCAGG |
| 614, 636 | 730 | GUUGCAACUUACGCAUAGGUGUAAAAUACGAG |
| 615 | 731 | GAUGUAUAUGCUAUGAUUUUGUAUGGUACAUCCAAC |
| 616 | 732 | GAUGUGAACGACCUUUUUUUGCGGUGUACUUCGAGG |
| 617 | 733 | UCAGCUCACAACCUACAUAUGCAUACAAGAUAUAUCG |
| | | U |
| 618 | 734 | GUUGGAGUCGGCUUGAAUCUGCGGGGUGCUUACAGG |
| 619 | 735 | GAUGUAAAUCAUCUAUAAAAGAAAGGUACAUCCAAC |
| 620 | 736 | GAUGCAUCUGACACAGCUGGGUGAGUUGAAGG |
| 625 | 737 | AUUGUAGGCGACCUUUUUUUGCGAUGUAGUUCGAGG |
| 628 | 738 | AGUGUAUGAUUACCUGUAGUAUGAGGUACAUCCAAC |
| 600, 602, 617, | 739 | GUUGCAAUUCGUAUGCGCAGGUAAGUUUCGAG |
| 623, 629-630 | | |
| 630 | 740 | CGUUGCAGCUCGCACGUUGGCACUGGUUGAAGG |
| 630 | 741 | CGUUGCAGCUCGCACGUUGGCACUGGGUUGAAGG |
| 631 | 742 | GAUAGUUUUAACUUCCAUUUGAAAUGUAAAUGCAAC |
| 632 | 743 | GCUGCAACACGCGCGGGUACGCGGGUUGAAGG |
| 633 | 744 | GUUGCAACUCACGCGCGUAUGUGGCUUGAAGG |
| 634 | 745 | AAUGUGAACGACCUUCUUUUGCGGUGUACUUCGAGG |
| 635 | 746 | GUUGCAAUUCAUAUCUCCGGGUGGAUUGAAGG |
| 637, 646 | 747 | GUUGGAAUCGACCUUAAUUUGAGGUGUGCUUACAGG |
| 638 | 748 | GUUGCGUUUGCCCGUGAUUUCGGGUGUGUAUACAGG |
| 639 | 749 | GAUGCAACUCGUGUGUAUGUGCGAGUUGAAGG |
| 640 | 750 | GCUGUUAGAACAUACAAAAUGAAAGGUACAUCCAAC |
| 641 | 751 | GGCGUAUGUCUACCUGAAAAAGAAGGUAUAUCCAAC |
| 642 | 752 | GGUGUAUGUGCACCAUAUAUGUAGGUGACAUACAGC |
| 643 | 753 | GCUGAAAGAGCAGAGAAUUUGUUGUGUGCAUACAGG |
| 644 | 754 | GACGCAACUCGCGCGCGGGCAUGUAUUGAGGG |
| 645 | 755 | GUUGCAACACAUGUAUGUGGGUGAGUUGAAGG |
| 647 | 756 | GCUGCACUGCACCGCCCAUUGAUGGUGUGCUCUAGG |
| 650 | 757 | GUUGCAACUCGCACGUUGGCACUGAUUGAAGG |
| 651 | 758 | GUUGUAAGAGACCCGAAUUUUAGCUGUGUAUACAGG |
| 652 | 759 | GUUGCAAUUUGUAUACGAGUGUGACUUGAAGG |
| 653 | 760 | GAUGUGAACGACCUUUUUUUGCGGUGUGCUUCGAGG |
| 654 | 761 | GGAGAGAUCUCAAACGAUUGCUCGAUUAGUCGAGAC |
| 655 | 762 | GUCGGAACGCUCAACGAUUGCCCCUCACGAGGGGAC |
| 657, 670 | 763 | ACCAAAACGACUAUUGAUUGCCCAGUACGCUGGGAC |
| 660, 666 | 764 | GGAUCCAAUCCUUUUUGAUUGCCCAAUUCGUUGGGAC |
| 663 | 765 | GGAUCUGAGGAUCAUUAUUGCUCGUUACGACGAGAC |
| 664, 668 | 766 | GUCUCAGCGUACUGAGCAAUCAAAAGGUUUCGCAGG |
| 665 | 767 | GUAGAAGACCUCGCUGAUUGCUCGGUGCGCCGAGAC |
| 669 | 768 | AUGGCAACAGACUCUCAUUGCGCGGUACGCCGCGAC |
| 671 | 769 | GUCGCUCUCUAACGCUUGCCCAGUACGCUGGGAC |
| 673, 674 | 770 | GGUUGAACCCUCAACAGAUUGCUCGGUAAGCCGAGAC |
| 675 | 771 | CUUGAAAUCCUGUCAGAUUGCUCCCUUCGGGGAGAC |
| 676, 677 | 772 | GCUGGAAGACUCAAUGAUGGCUCCUUACGAGGAGAC |
| 678 | 773 | CUAGGAACGCACGCAGAUUGCUCGGUACGCCGAGAC |
| 679 | 774 | AUUGCAACGCCUAAAGAUUGCUCGAUACGUCGAGAC |
| 680 | 775 | GUUCGGCRAYCCUUUGAUUGCUCAGUACGCUGAGAC |
| 681 | 776 | GUUGAACCUAGAUCAGAUGGCUCAGUACGCUGAGAC |
| 682 | 777 | CCCUCAACACGUCAGAAAUGCCCGGCACGCCGGGAC |
| 683 | 778 | GUCGCAAGACUCGAAUAAUUGCCCCUCUAUGGGGAC |
| 684 | 779 | GCUGGGGACCGAUCCUGAUUGCUCGCUGCGGCGAGAC |
| 685 | 780 | CUCUCAAUGGAUAACGAUUGCUCUCUACGGAGAGAC |
| 686 | 781 | GUUGAACCCUCAACAGAUUGCUCGGUAAGCCGAGAC |
| 696 | 782 | ACUGAAACCACCAACGAUUGCGCUCCUCGGAGCGAC |
| 569, 571, 575, | 783 | AGGUACAUCCAAC |
| 628 | | |
| 569, 573-574, | 784 | UGGUACAUCCAAC |
| 578-579, 581, | | |
| 585, 587-589, | | |
| 591-592, 596, | | |
| 598, 604, | | |
| 609-612, 615, | | |
| 622, 626 | | |
| 570, 595 | 785 | UGCGGUGUAAUUCGAGG |
| 576 | 786 | AAGGUUGAUACAGC |
| 577 | 787 | GAGCACAUCCAAC |
| 578, 583, 585 | 788 | GGGUACAUCCAAC |
| 580 | 789 | AGGUAUAUCCAAC |
| 582 | 790 | GGCUACAUACAGC |
| 584, 586 | 792 | AAAACAAGGAUUGAAAC |
| 590 | 793 | GCUGCAAGAGCUCCUAAUUUGAGGGGUGCAUACAGG |
| 592 | 794 | UGGUACAUCC |
| 592 | 795 | UGGGGCAGUUGGUUGCCCUUAGCCUGAGGCAUUUAUU |
| | | GCACUCGGGAAGUACCAUUACAUCCAAC |
| 592 | 796 | UGGUACAUCCAACUCUAGGCGCC |
| 592 | 797 | AAUGGUACAUCCAAC |
| 592 | 798 | UGGUACAUCCAACUCUAGGC |
| 592 | 799 | UGGUACAUCCAACUCUAGGCGC |
| 592 | 800 | UGGUACAUCCAACUCUAGGCG |
| 592 | 801 | UGGUACAUCCAACUCUAGG |
| 592 | 802 | AAAUGGUACAUCCAAC |
| 592 | 803 | UGGUACAUCCAACUCU |
| 592 | 804 | UGGUACAUCCAACUC |
| 592 | 805 | UGGUACAUCCAACU |
| 592 | 806 | UGGUACAUCCAACUCUAG |
| 592 | 807 | UGGUAUAUCCAAC |
| 592 | 808 | UGGUACAUCCAACUCUA |
| 592 | 809 | AUGGUACAUCCAAC |
| 592 | 810 | UGGUACAUCCAA |
| 592 | 811 | UGGUACAUCCA |
| 601 | 815 | CAGGCGUAAGAUGUGAG |
| 621 | 816 | CGUACGUGGAUUGAAAC |
| 624 | 817 | CAUAUGUAGGUUGUGAG |
| 627 | 818 | CAUGAGGUGUGAG |
| 630 | 819 | CACUGGUUGAAGG |
| 603 | 820 | AGCGUGUGGCUUGAAGG |
| 606 | 821 | AUGGUGAGUACAUCCAAC |
| 625 | 822 | AUGUAGUUCGAGG |
| 631 | 823 | AUGUAAAUGCAAC |
| 599 | 824 | AGAAGAAGGAUUGGGAC |
| 624 | 825 | UGUAGGUUGUGAG |
| 603 | 826 | UGUGGCUUGAAGG |
| 605, 613, 618 | 827 | UGUGGGGUGCUUACAGG |
| 600, 601, 602, | 828 | UGUGCAUGAGGUGUGAG |
| 617, 623, 627 | | |
| 630 | 829 | UUGGCACUGGUUGAAGG |
| 608 | 830 | UGAAUGCGACUUGAAGG |
| 602 | 831 | UAUGGGUAUGCUGCGAG |
| 595 | 832 | UUGCGGUGUACUUCGAGG |
| 616 | 833 | GUGUACUUCGAGG |
| 619 | 834 | GGUACAUCCAAC |
| 620 | 835 | GCUGGGUGAGUUGAAGG |

TABLE 6 provides an illustrative linker sequence for use in guide nucleic acids that are useful in the compositions, systems and methods described herein.

TABLE 6
Exemplary Linker Sequence

| | Linker sequence (5′→3′), |
|---|---|
| Seq ID No. | shown as RNA |
| 255 | GAAA |

TABLE 7 provides illustrative spacer sequences for use in guide nucleic acids that are useful in the compositions, systems and methods described herein.

TABLE 7
Exemplary Spacer Sequences for use in Guide Nucleic Acids

| Effector protein or | Spacer | | |
|---|---|---|---|
| a variant thereof | Sequence | Spacer Sequence (5′→3′), | |
| (SEQ ID No.) | SEQ ID No. | shown as RNA | Target Loci |
| 2 | 860 | CCCCACAGGGCAGUAAC | HBB E6 |
| 2 | 861 | GAGUAUCCAGUGAGGCC | BCL11A |
| | | | binding site in |
| | | | the promoter of |
| | | | HBG1 & HBG2 |
| 2 | 862 | GUCAAGGCAAGGCUGGC | BCL11A |
| | | | binding site in |
| | | | the promoter of |
| | | | HBG1 & HBG2 |
| 2 | 863 | CAUUGAGAUAGUGUGGG | LRF1/ZBTB7A |
| | | | binding site in |
| | | | the promoter of |
| | | | HBG1 & HBG2 |
| 2 | 864 | AGAUAGUGUGGGGAAGG | LRF1/ZBTB7A |
| | | | binding site in |
| | | | the promoter of |
| | | | HBG1 & HBG2 |
| 2 | 865 | CUGGAGCCUGUGAUAAA | BCL11A |
| | | | erythroid |
| | | | enhancer |
| 2 | 866 | GCCUUGUCAAGGCUAUU | BCL11A |
| | | | binding site in |
| | | | the promoter |
| | | | region of HBG1 |
| | | | & HBG2 |
| 2 | 867 | CCUUGUCAAGGCUAUUG | BCL11A |
| | | | binding site in |
| | | | the promoter of |
| | | | HBG1 & HBG2 |
| 2 | 868 | GCCAGCCUUGCCUUGAC | BCL11A |
| | | | binding site in |
| | | | the promoter of |
| | | | HBG1 & HBG2 |
| 2 | 869 | GCAUUGAGAUAGUGUGG | LRF1/ZBTB7A |
| | | | binding site in |
| | | | the promoter of |
| | | | HBG1 & HBG2 |
| 1 | 870 | CCGUUACUGCCCUGUGGGGC | HBB |
| 1 | 871 | CUUCUGACACAACUGUGUUC | HBB |
| 1 | 872 | AGGUUGCUAGUGAACACAGU | HBB |
| 1 | 873 | GGGCAAGGUGAACGUGGAUG | HBB |
| 1 | 874 | ACACAACUGUGUUCACUAGC | HBB |
| 1 | 875 | UUCACUAGCAACCUCAAACA | HBB |
| 1 | 876 | UUUGAGGUUGCUAGUGAACA | HBB |
| 1 | 877 | ACUCCUGAGGAGAAGUCUGC | HBB |
| 1 | 878 | AAGCUAGUCUAGUGCAAGCU | BCL11A Enh |
| 1 | 879 | GUGCAAGCUAACAGUUGCUU | BCL11A Enh |
| 1 | 880 | GCCUCUGAUUAGGGUGGGGG | BCL11A Enh |
| 1 | 881 | AUUAGGGUGGGGGCGUGGGU | BCL11A Enh |
| 1 | 882 | UCACAGGCUCCAGGAAGGGU | BCL11A Enh |
| 1 | 883 | AUAAAAGCAACUGUUAGCUU | BCL11A Enh |
| 1 | 884 | CCCCACCCACGCCCCCACCC | BCL11A Enh |
| 1 | 885 | GCCAGGGACCGUUUCAGACA | pHBG |
| 1 | 886 | UCUGAAACGGUCCCUGGCUA | pHBG |
| 1 | 887 | CCUUGUCAAGGCUAUUGGUC | pHBG |
| 1 | 888 | AAACGGUCCCUGGCUAAACU | pHBG |
| 1 | 889 | GGGAAGGGGCCCCCAAGAGG | pHBG |
| 1 | 890 | AGACUAUUGGUCAAGUUUGC | pHBG |
| 1 | 891 | CAUUGAGAUAGUGUGGGGAA | pHBG |
| 1 | 892 | AAAAAAAUUAAGCAGCAGUA | pHBG |
| 1 | 893 | UCACAGCCCAAGAUAGUUAA | B2M |

TABLE 8 provides an illustrative intermediary sequence for use in guide nucleic acids that are useful in the compositions, systems and methods described herein.

TABLE 8
Exemplary Intermediary Sequence

| Effector | | |
|---|---|---|
| Protein | SEQ | Intermediary sequence (5′→3′), |
| SEQ ID No. | ID No. | shown as RNA |
| 1 | 257 | ACAGCUUAUUUGGAAGCUGAAAUGUGAGG |
| | | UUUAUAACACUCACAAGAAUCCU |

TABLE 9 provides exemplary compositions comprising an effector protein described herein and gRNAs.

TABLE 9
Exemplary Compositions of Effector Protein and Guide Nucleic Acids

| | Effector | | | | |
|---|---|---|---|---|---|
| | Protein | PAM | Target | Intermediary | Repeat |
| Comp | SEQ ID | SEQ ID | SEQ ID | SEQ ID | SEQ ID |
| No. | No. | No. | No. | No. | No. |
| 1 | 1 | 19 | 836-858 | | 703 |
| 2 | 1 | 21 | 836-858 | 257 | |
| 3 | 1 | 22 | 836-858 | 257 | |
| 4 | 1 | 23 | 836-858 | 257 | |
| 5 | 1 | 24 | 836-858 | 257 | |
| 6 | 1 | 25 | 836-858 | 257 | |
| 7 | 1 | 26 | 836-858 | 257 | |
| 8 | 1 | 27 | 836-858 | 257 | |
| 9 | 1 | 28 | 836-858 | 257 | |
| 10 | 1 | 29 | 836-858 | 257 | |
| 11 | 1 | 30 | 836-858 | 257 | |
| 12 | 1 | 31 | 836-858 | 257 | |
| 13 | 1 | 32 | 836-858 | 257 | |
| 14 | 1 | 33 | 836-858 | 257 | |
| 15 | 1 | 34 | 836-858 | 257 | |
| 16 | 1 | 35 | 836-858 | 257 | |
| 17 | 1 | 36 | 836-858 | 257 | |
| 18 | 1 | 38 | 836-858 | 257 | |
| 19 | 1 | 39 | 836-858 | 257 | |
| 20 | 1 | 40 | 836-858 | 257 | |
| 21 | 1 | 41 | 836-858 | 257 | |
| 22 | 1 | 42 | 836-858 | 257 | |
| 23 | 1 | 43 | 836-858 | 257 | |
| 24 | 1 | 44 | 836-858 | 257 | |
| 25 | 1 | 45 | 836-858 | 257 | |
| 26 | 1 | 46 | 836-858 | 257 | |
| 27 | 1 | 48 | 836-858 | 257 | |
| 28 | 1 | 49 | 836-858 | 257 | |
| 29 | 1 | 50 | 836-858 | 257 | |
| 30 | 1 | 51 | 836-858 | 257 | |
| 31 | 1 | 52 | 836-858 | 257 | |
| 32 | 1 | 53 | 836-858 | 257 | |
| 33 | 1 | 54 | 836-858 | 257 | |
| 34 | 1 | 55 | 836-858 | 257 | |
| 35 | 1 | 56 | 836-858 | 257 | |
| 36 | 1 | 57 | 836-858 | 257 | |
| 37 | 1 | 58 | 836-858 | 257 | |
| 38 | 1 | 60 | 836-858 | 257 | |
| 39 | 1 | 61 | 836-858 | 257 | |
| 40 | 1 | 62 | 836-858 | 257 | |
| 41 | 1 | 64 | 836-858 | 257 | |
| 42 | 1 | 65 | 836-858 | 257 | |
| 43 | 1 | 67 | 836-858 | 257 | |
| 44 | 1 | 73 | 836-858 | | 703 |
| 45 | 1 | 76 | 836-858 | | 703 |
| 46 | 1 | 76 | 836-858 | 257 | |
| 47 | 1 | 77 | 836-858 | 257 | |
| 48 | 1 | 79 | 836-858 | 257 | |
| 49 | 1 | 72 | 836-858 | 257 | |
| 50 | 1 | 81 | 512 | 257 | |
| 51 | 2 | 82 | 836-858 | | 252 |
| 52 | 2 | 82 | 836-858 | | 252 |
| 53 | 2 | 513 | 836-858 | | 253 |
| 54 | 2 | 513 | 836-858 | | 254 |
| 55 | 569 | 514 | 836-858 | | 783 |
| 56 | 569 | 514 | 836-858 | | 784 |
| 57 | 569 | 515 | 836-858 | | 783 |
| 58 | 569 | 515 | 836-858 | | 784 |
| 59 | 569 | 516 | 836-858 | | 783 |
| 60 | 569 | 516 | 836-858 | | 784 |
| 61 | 569 | 517 | 836-858 | | 783 |
| 62 | 569 | 517 | 836-858 | | 784 |
| 63 | 570 | 518 | 836-858 | | 785 |
| 64 | 570 | 519 | 836-858 | | 785 |
| 65 | 571 | 514 | 836-858 | | 783 |
| 66 | 572 | | 836-858 | | 704 |
| 67 | 573 | 536 or 565 | 836-858 | | 784 |
| 68 | 574 | 516 or 859 | 836-858 | | 784 |
| 69 | 575 | 524 or 540 | 836-858 | | 783 |
| 70 | 576 | | 836-858 | | 786 |
| 71 | 577 | 538 | 836-858 | | 787 |
| 72 | 578 | 514 | 836-858 | | 788 |
| 73 | 578 | 514 | 836-858 | | 784 |
| 74 | 579 | 516 | 836-858 | | 784 |
| 75 | 580 | 515 or 525 | 836-858 | | 789 |
| 76 | 581 | 516 | 836-858 | | 784 |
| 77 | 582 | | 836-858 | | 790 |
| 78 | 583 | 539 | 836-858 | | 788 |
| 79 | 584 | | 836-858 | | 711 |
| 80 | 584 | | 836-858 | | 792 |
| 81 | 585 | 515 or 516 | 836-858 | | 788 |
| 82 | 585 | 515 or 516 | 836-858 | | 784 |
| 83 | 586 | | 836-858 | | 792 |
| 84 | 587 | 515 or 516 | 836-858 | | 784 |
| 85 | 588 | 516 | 836-858 | | 784 |
| 86 | 589 | 515 | 836-858 | | 784 |
| 87 | 590 | 520 | 836-858 | | 793 |
| 88 | 591 | 532 | 836-858 | | 705 |
| 89 | 591 | 532 | 836-858 | | 784 |
| 90 | 592 | 515 or 526 | 836-858 | | 706 |
| 91 | 592 | 515 or 526 | 836-858 | | 784 |
| 92 | 592 | 515 or 526 | 836-858 | | 794 |
| 93 | 592 | 515 or 526 | 836-858 | | 795 |
| 94 | 592 | 515 or 526 | 836-858 | | 796 |
| 95 | 592 | 515 or 526 | 836-858 | | 797 |
| 96 | 592 | 515 or 526 | 836-858 | | 798 |
| 97 | 592 | 515 or 526 | 836-858 | | 799 |
| 98 | 592 | 515 or 526 | 836-858 | | 800 |
| 99 | 592 | 515 or 526 | 836-858 | | 801 |
| 100 | 592 | 515 or 526 | 836-858 | | 802 |
| 101 | 592 | 515 or 526 | 836-858 | | 803 |
| 102 | 592 | 515 or 526 | 836-858 | | 804 |
| 103 | 592 | 515 or 526 | 836-858 | | 805 |
| 104 | 592 | 515 or 526 | 836-858 | | 806 |
| 105 | 592 | 515 or 526 | 836-858 | | 807 |
| 106 | 592 | 515 or 526 | 836-858 | | 808 |
| 107 | 592 | 515 or 526 | 836-858 | | 809 |
| 108 | 592 | 515 or 526 | 836-858 | | 810 |
| 109 | 592 | 515 or 526 | 836-858 | | 811 |
| 110 | 593 | 84 or 562 | 836-858 | | 707 |
| 111 | 593 | 84 or 562 | 836-858 | | 708 |
| 112 | 593 | 84 or 562 | 836-858 | | 712 |
| 113 | 594 | 542, 72, or | 836-858 | | 709 |
| | | 522 | | | |
| 114 | 594 | 542, 72, or | 836-858 | | 709 |
| | | 522 | | | |
| 115 | 595 | 518 | 836-858 | | 710 |
| 116 | 595 | 518 | 836-858 | | 785 |
| 117 | 595 | 518 | 836-858 | | 832 |
| 118 | 596 | 515 | 836-858 | | 711 |
| 119 | 596 | 515 | 836-858 | | 784 |
| 120 | 597 | 552, 560, | 836-858 | | 712 |
| | | or 564 | | | |
| 121 | 597 | 552, 560, | 836-858 | | 712 |
| | | or 564 | | | |
| 122 | 598 | 516 | 836-858 | | 711 |
| 123 | 598 | 516 | 836-858 | | 713 |
| 124 | 598 | 516 | 836-858 | | 784 |
| 125 | 599 | | 836-858 | | 713 |
| 126 | 599 | | 836-858 | | 824 |
| 127 | 600 | 84 or 549 | 836-858 | | 714 |
| 128 | 600 | 84 or 549 | 836-858 | | 715 |
| 129 | 600 | 84 or 549 | 836-858 | | 708 |
| 130 | 600 | 84 or 549 | 836-858 | | 712 |
| 131 | 600 | 84 or 549 | 836-858 | | 739 |
| 132 | 600 | 84 or 549 | 836-858 | | 828 |
| 133 | 601 | 546 or 543 | 836-858 | | 716 |
| 134 | 601 | 546 or 543 | 836-858 | | 715 |
| 135 | 601 | 546 or 543 | 836-858 | | 828 |
| 136 | 601 | 546 or 543 | 836-858 | | 815 |
| 137 | 602 | 84 or 549 | 836-858 | | 717 |
| 138 | 602 | 84 or 549 | 836-858 | | 708 |
| 139 | 602 | 84 or 549 | 836-858 | | 715 |
| 140 | 602 | 84 or 549 | 836-858 | | 712 |
| 141 | 602 | 84 or 549 | 836-858 | | 739 |
| 142 | 602 | 84 or 549 | 836-858 | | 828 |
| 143 | 602 | 84 or 549 | 836-858 | | 831 |
| 144 | 603 | 533 or 77 | 836-858 | | 718 |
| 145 | 603 | 533 or 77 | 836-858 | | 820 |
| 146 | 603 | 533 or 77 | 836-858 | | 826 |
| 147 | 604 | 516 | 836-858 | | 719 |
| 148 | 604 | 516 | 836-858 | | 784 |
| 149 | 605 | 530 | 836-858 | | 720 |
| 150 | 605 | 530 | 836-858 | | 721 |
| 151 | 605 | 530 | 836-858 | | 827 |
| 152 | 606 | 539 | 836-858 | | 722 |
| 153 | 606 | 539 | 836-858 | | 821 |
| 154 | 607 | 84 | 836-858 | | 723 |
| 155 | 607 | 84 | 836-858 | | 712 |
| 156 | 608 | 534 | 836-858 | | 724 |
| 157 | 608 | 534 | 836-858 | | 830 |
| 158 | 609 | 515 | 836-858 | | 725 |
| 159 | 609 | 515 | 836-858 | | 784 |
| 160 | 610 | 516 | 836-858 | | 726 |
| 161 | 610 | 516 | 836-858 | | 784 |
| 162 | 611 | 56 or 516 | 836-858 | | 727 |
| 163 | 611 | 56 or 516 | 836-858 | | 784 |
| 164 | 612 | 515 or 516 | 836-858 | | 728 |
| 165 | 612 | 515 or 516 | 836-858 | | 784 |
| 166 | 613 | 530 | 836-858 | | 729 |
| 167 | 613 | 530 | 836-858 | | 721 |
| 168 | 613 | 530 | 836-858 | | 827 |
| 169 | 614 | 561 or 555 | 836-858 | | 730 |
| 170 | 614 | 561 or 555 | 836-858 | | 715 |
| 171 | 614 | 561 or 555 | 836-858 | | 708 |
| 172 | 614 | 561 or 555 | 836-858 | | 712 |
| 173 | 615 | 527 or 528 | 836-858 | | 731 |
| 174 | 615 | 527 or 528 | 836-858 | | 784 |
| 175 | 616 | 547 | 836-858 | | 732 |
| 176 | 616 | 547 | 836-858 | | 833 |
| 177 | 617 | 84, 552, or | 836-858 | | 733 |
| | | 554 | | | |
| 178 | 617 | 84, 552, or | 836-858 | | 715 |
| | | 554 | | | |
| 179 | 617 | 84, 552, or | 836-858 | | 708 |
| | | 554 | | | |
| 180 | 617 | 84, 552, or | 836-858 | | 712 |
| | | 554 | | | |
| 181 | 617 | 84, 552, or | 836-858 | | 739 |
| | | 554 | | | |
| 182 | 617 | 84, 552, or | 836-858 | | 828 |
| | | 554 | | | |
| 183 | 618 | 530 | 836-858 | | 734 |
| 184 | 618 | 530 | 836-858 | | 827 |
| 185 | 619 | 542, 72, | 836-858 | | 735 |
| | | 556, or | | | |
| | | 558 | | | |
| 186 | 619 | 542, 72, | 836-858 | | 834 |
| | | 556, or | | | |
| | | 558 | | | |
| 187 | 620 | 533, or | 836-858 | | 736 |
| | | 535 | | | |
| 188 | 620 | 533, or | 836-858 | | 835 |
| | | 535 | | | |
| 189 | 621 | | 836-858 | | 713 |
| 190 | 621 | | 836-858 | | 816 |
| 191 | 622 | 516 | 836-858 | | 719 |
| 192 | 622 | 516 | 836-858 | | 784 |
| 193 | 623 | 84 | 836-858 | | 717 |
| 194 | 623 | 84 | 836-858 | | 708 |
| 195 | 623 | 84 | 836-858 | | 715 |
| 196 | 623 | 84 | 836-858 | | 712 |
| 197 | 623 | 84 | 836-858 | | 739 |
| 198 | 623 | 84 | 836-858 | | 828 |
| 199 | 624 | 546 | 836-858 | | 708 |
| 200 | 624 | 546 | 836-858 | | 817 |
| 201 | 624 | 546 | 836-858 | | 825 |
| 202 | 625 | 547, 566, | 836-858 | | 737 |
| | | or 567 | | | |
| 203 | 625 | 547, 566, | 836-858 | | 822 |
| | | or 567 | | | |
| 204 | 626 | 515, or | 836-858 | | 728 |
| | | 525 | | | |
| 205 | 626 | 515, or | 836-858 | | 784 |
| | | 525 | | | |
| 206 | 627 | 546, 543, | 836-858 | | 715 |
| | | or 84 | | | |
| 207 | 627 | 546, 543, | 836-858 | | 828 |
| | | or 84 | | | |
| 208 | 627 | 546, 543, | 836-858 | | 818 |
| | | or 84 | | | |
| 209 | 628 | 542, 72, or | 836-858 | | 738 |
| | | 558 | | | |
| 210 | 628 | 542, 72, or | 836-858 | | 783 |
| | | 558 | | | |
| 211 | 629 | 84, or 550 | 836-858 | | 739 |
| 212 | 629 | 84, or 550 | 836-858 | | 739 |
| 213 | 630 | 529 | 836-858 | | 739 |
| 214 | 630 | 529 | 836-858 | | 741 |
| 215 | 630 | 529 | 836-858 | | 829 |
| 216 | 630 | 529 | 836-858 | | 819 |
| 217 | 631 | 544, 557, | 836-858 | | 742 |
| | | or 539 | | | |
| 218 | 631 | 544, 557, | 836-858 | | 823 |
| | | or 539 | | | |
| 219 | 632 | 564 or 552 | 836-858 | | 743 |
| 220 | 633 | | 836-858 | | 744 |
| 221 | 634 | 551 | 836-858 | | 708 |
| 222 | 634 | 551 | 836-858 | | 745 |
| 223 | 635 | | 836-858 | | 746 |
| 224 | 636 | 553, 560, | 836-858 | | 730 |
| | | 521, or | | | |
| | | 541 | | | |
| 225 | 636 | 553, 560, | 836-858 | | 708 |
| | | 521, or | | | |
| | | 541 | | | |
| 226 | 637 | | 836-858 | | 747 |
| 227 | 638 | | 836-858 | | 748 |
| 228 | 639 | | 836-858 | | 749 |
| 229 | 640 | | 836-858 | | 750 |
| 230 | 641 | 542 or 558 | 836-858 | | 751 |
| 231 | 642 | | 836-858 | | 752 |
| 232 | 643 | 530 | 836-858 | | 753 |
| 233 | 644 | | 836-858 | | 754 |
| 234 | 645 | 523 | 836-858 | | 755 |
| 235 | 646 | 530 | 836-858 | | 747 |
| 236 | 647 | 518 or 519 | 836-858 | | 756 |
| 237 | 648 | | 836-858 | | 718 |
| 238 | 649 | 530 | 836-858 | | 721 |
| 239 | 650 | 529 | 836-858 | | 757 |
| 240 | 651 | 531 | 836-858 | | 758 |
| 241 | 652 | | 836-858 | | 759 |
| 242 | 653 | 552 or 559 | 836-858 | | 760 |
| 243 | 654 | | 836-858 | | 761 |
| 244 | 655 | | 836-858 | | 762 |
| 245 | 657 | 547 | 836-858 | | 763 |
| 246 | 660 | | 836-858 | | 764 |
| 247 | 663 | | 836-858 | | 765 |
| 248 | 664 | 82 | 836-858 | | 766 |
| 249 | 665 | | 836-858 | | 767 |
| 250 | 666 | | 836-858 | | 764 |
| 251 | 667 | 547 | 836-858 | | 254 |
| 252 | 667 | 547 | 836-858 | | 253 |
| 253 | 668 | 82 | 836-858 | | 766 |
| 254 | 669 | | 836-858 | | 768 |
| 255 | 670 | | 836-858 | | 763 |
| 256 | 671 | | 836-858 | | 769 |
| 257 | 672 | 82 | 836-858 | | |
| 258 | 673 | 85 | 836-858 | | 770 |
| 259 | 674 | | 836-858 | | 770 |
| 260 | 675 | | 836-858 | | 771 |
| 261 | 676 | 547 | 836-858 | | 772 |
| 262 | 677 | 547 | 836-858 | | 772 |
| 263 | 678 | 82 | 836-858 | | 773 |
| 264 | 679 | 537 | 836-858 | | 774 |
| 265 | 680 | 82 | 836-858 | | 775 |
| 266 | 681 | 82 | 836-858 | | 776 |
| 267 | 682 | 568 | 836-858 | | 777 |
| 268 | 683 | 82 | 836-858 | | 778 |
| 269 | 684 | 547 | 836-858 | | 779 |
| 270 | 685 | 514 | 836-858 | | 780 |
| 271 | 686 | 545 | 836-858 | | 781 |
| 272 | 687 | 545 | 836-858 | | |
| 273 | 689 | 82 | 836-858 | | |
| 274 | 690 | 548 | 836-858 | | |
| 275 | 691 | 514 | 836-858 | | |
| 276 | 694 | 82 | 836-858 | | |
| 277 | 695 | 545 | 836-858 | | |
| 278 | 696 | 82 | 836-858 | | 782 |

TABLE 10 provides illustrative target nucleic acid sequences for use with the compositions, systems, and methods of the disclosure.

TABLE 10
Target Nucleic Acid Sequences
Target Nucleic Acid Sequences

ADA, B2M, BCL11A, BLM, BRCA1, BRCA2, BRIP1, CCND2, CCR5, CHD4, CIITA,
CYBB, ERCC4, FANCA, FANCB, FANCC, FANCD1, FANCD2, FANCE, FANCF,
FANCG, FANCI, FANCJ, FANCL, FANCM, FANCN, FANCO, FANCP, FANCQ,
FANCR, FANCS, FANCT, FANCU, FANCV, FANCW, FIX, FVIII, G6PD GAA, GATA1,
GATA2, GATA-4, globin gene, HBA, HBA1, HBA2, HBB, HBD, HBE, HBE1, HBG,
HBG1, HBG2, HBM, HBQ1, HBZ, HOXA9, HOXB13, IDUA, IL2RG, KLF1, LMO2,
MBD3, MYB, PALB2, RAD51, RAD51C, REV7, RFWD3, SLX4, TRAC, UBE2T, XPF,
XRCC2, ZBTB7A

TABLE 11 provides illustrative target nucleic acid sequences for use with the compositions, systems, and methods of the disclosure.

TABLE 11
EXEMPLARY TARGET NUCLEIC ACID SEQUENCES

| Name | Ensembl Reference Sequence | Seq ID No. |
|---|---|---|
| BCL11A | ENSG00000119866 | 836 |
| PNPLA2 | ENSG00000177666 | 837 |
| CHD4 | ENSG00000111642 | 838 |
| CIITA | ENSG00000179583 | 839 |
| GATA1 | ENSG00000102145 | 840 |
| B2M | ENSG00000166710 | 841 |
| HBA1 | ENSG00000206172 | 842 |
| HBA2 | ENSG00000188536 | 843 |
| HBB | ENSG00000244734 | 844 |
| HBD | ENSG00000223609 | 845 |
| HBE1 | ENSG00000213931 | 846 |
| HBG1 | ENSG00000213934 | 847 |
| HBG2 | ENSG00000196565 | 848 |
| HBM | ENSG00000206177 | 849 |
| HBQ1 | ENSG00000086506 | 850 |
| HBZ | ENSG00000130656 | 851 |
| HOXA9 | ENSG00000078399 | 852 |
| KLF1 | ENSG00000105610 | 853 |
| CCR5 | ENSG00000160791 | 854 |
| MBD3 | ENSG00000071655 | 855 |
| MYB | ENSG00000118513 | 856 |
| TRAC | ENSG00000277734 | 857 |
| ZBTB7A | ENSG00000178951 | 858 |

TABLE 12 provides illustrative diseases and syndromes for compositions, systems and methods described herein.

TABLE 12
DISEASES AND SYNDROMES
Diseases
acanthocytosis; acute basophilic leukemia; acute erythroid leukemia; acute lymphoblastic
leukemia; acute myeloid leukemia (AML); acute panmyelosis with myelofibrosis
(APMF); acute posthemorrhagic anemia; anaplastic large cell lymphoma; anemia;
angiocentric glioma; aplastic anemia; autoimmune disorder; autoimmune hemolytic
anemia (AIHA); autoimmune lymphoproliferative syndrome; autoimmune polyglandular
syndrome type 1; alpha-thalassemia; β-thalassemia; Blackfan-Diamond anemia; bleeding
disorder (coagulation); blood cancer; T-cell immunodeficiency; chemotherapy; chorea-
acanthocytosis (ChAc); classic hemochromatosis; coagulation conditions; cold
hemagglutinin disease; cutaneous T-cell lymphoma; diffuse large B-cell lymphoma;
disseminated intravascular coagulation; dysphagia; familial hemophagocytic
lymphohistiocytosis; Fanconi disease (Fanconi anemia); folate-deficiency anemia;
follicular lymphoma; genetic blood disease; genetic disease; glaucoma; glucose-6-
phosphate dehydrogenase (G6PD) deficiency; hairy cell leukemia; hematopoietic genetic
disease; hemochromatosis type 3; hemochromatosis type 4; hemoglobinopathy;
hemoglobin disorder; hemoglobin C disease; hemoglobin E disease; hemolytic anemia;
hemolytic uremic syndrome; hemophilia; hemophilia A; hemophilia B; hereditary
elliptocytosis (HE); hemorrhagic conditions; hereditary hemorrhagic telangiectasia;
hereditary hypofibrinogenemia; hereditary intraosseous vascular malformation; hereditary
leiomyomatosis and renal cell cancer; hereditary persistence of fetal hemoglobin;
hereditary pyropoikilocytosis (HPP); hereditary spherocytosis; hereditary stomatocytosis;
hexokinase deficiency; histiocytic sarcoma; hormone refractory prostate cancer; human
immunodeficiency with microcephaly; hyperanaemia; hypochromic anemia;
immunodeficiency; immunodeficiency 13; immunodeficiency 17; immunodeficiency 25;
immunodeficiency with hyper-IgM type 1; immunodeficiency with hyper-IgM type 3;
immunodeficiency with hyper-IgM type 4; immunodeficiency with hyper-IgM type 5;
immunoglobulin alpha deficiency; inborn errors of thyroid metabolism; ineffective
erythropoiesis; iron-deficiency anemia; juvenile hemochromatosis; leukemia; lymphoma;
lymphomatoid granulomatosis; lymphoplasmacytic lymphoma; a lysosomal storage
disease (e.g., Hunter syndrome, Hurler syndrome); lipid storage disease; lipid storage
disease with myopathy; malignant blood disease; malignant immunoproliferative
diseases; mantle cell lymphoma; Marchiafava-Micheli syndrome; megaloblastic anemia
type 1; megaloblastic hereditary anemia; metabolic disorder; microglioma; Minkowski-
Chauffard syndrome; multiple cutaneous and mucosal venous malformations; multiple
myeloma; myelodysplastic syndrome; neuroacanthocytosis (Levine-Critchley syndrome);
non-malignant blood disease; nutritional megaloblastic anemia; paroxysmal cold
hemoglobinuria (PCH); pernicious anemia; persistent fetal hemoglobin; platelet
glycoprotein IV deficiency; pleuropulmonary blastoma and cystic nephroma; plummer-
vinson syndrome (PVS); PRKAG2 cardiac syndrome; primary hemochromatoses;
prostate cancer; protein-deficiency anemia; pyruvate kinase deficiency; reticulosis;
reticuloendotheliosis; severe combined immunodeficiency disorder (SCID); scurvy; sickle
cell anemia; sickle cell disease (SCD); storage diseases; stroke; subependymal glioma; T
Cell deficiency; T-Cell receptor-Alpha/Beta deficiency; thalassemia; thrombocytopenia;
thromboembolism; thrombosis; thrombophilia due to antithrombin III deficiency; thyroid
metabolism diseases; transcobalamin II deficiency (TCII); triosephosphate isomerase
(TPI); vitamin B12 deficiency anemia; Waldenstrom's macroglobulinemia; X-linked
lymphoproliferative disease

### Examples

The following examples are included for illustrative purposes only and are not intended to limit the scope of the invention.

### Example 1: Indel Activity of Effector Proteins in Eukaryotic Cells

Effector proteins (e.g., any one of the effector proteins recited in TABLE 1, or a variant thereof as recited in TABLE 2) are tested for their ability to produce indels at the targeted loci (e.g., HBB gene, HBG1 gene, HBG2 gene, BCL11A gene, or B2M gene, or regulatory sequence thereof (e.g., promoter, enhancer)) in eukaryotic cells (e.g., hematopoietic stem cell (HSC), or any other eukaryotic cell) in vitro. Plasmid pairs co-expressing the effector protein and a guide nucleic acid (e.g., guide RNA, sgRNA or crRNA) are delivered to eukaryotic cells via transfection, electroporation, or lipofection using a lipofection reagent. Transfected cells are first incubated with the effector protein and guide nucleic acid and then lysed to obtain the genomic DNA. The genomic DNA is subjected to PCR amplification to amplify the targeted loci. Indels are detected by next generation sequencing (NGS) of PCR amplicons at the targeted loci, and indel percentage is calculated as the fraction of sequencing reads containing insertions or deletions relative to an unedited reference sequence.

### Example 2: Indel Activity of Effector Proteins in Hematopoietic Stem Cells (HSCs)

Effector proteins (e.g., any one of the effector proteins recited in TABLE 1, or a variant thereof as recited in TABLE 2) are tested for their ability to produce indels at the targeted loci (e.g., HBB gene, HBG1 gene, HBG2 gene, BCL11A gene, or B2M gene, or regulatory sequence thereof (e.g., promoter, enhancer)) in hematopoietic stem cells (HSCs). An mRNA encoding the effector protein and a guide nucleic acid (e.g., guide RNA, sgRNA, crRNA) are delivered to HSCs using a neon electroporation system using a 1500 volts/30 ms/2 pulse protocol. Electroporated HSCs are first incubated at 37° C. in a culture medium and then lysed to obtain the genomic DNA. The genomic DNA is subjected to PCR amplification to amplify the targeted loci. Indels are detected by next generation sequencing (NGS) of PCR amplicons at the targeted loci, and indel percentage is calculated as the fraction of sequencing reads containing insertions or deletions relative to an unedited reference sequence.

### Example 3: Gene Editing of Eukaryotic Cells with AAV Vector Comprising Effector Protein

An AAV vector is constructed to contain a transgene between its ITRs. Such a transgene, for example, can provide or encode, in a 5′ to 3′ direction, a first promoter (e.g., U6), a guide nucleic acid (e.g., guide RNA, sgRNA, crRNA), a second promoter (e.g., EFS), an effector protein (e.g., any one of the effector proteins recited in TABLE 1, or a variant thereof as recited in TABLE 2), and a poly A signal (e.g., SV40 poly A tail). The AAV vector is expressed with supporting plasmids to produce an adeno-associated virus (AAV). Eukaryotic cells (e.g., hematopoietic stem cell (HSC), or any other eukaryotic cell) are contacted with the AAV for 24 hours. After about 96 hours, post AAV contact, DNA or RNA is isolated from the infected eukaryotic cells. Edits are detected at the targeted loci (e.g., HBB gene, HBG1 gene, HBG2 gene, BCL11A gene, or B2M gene, or regulatory sequence thereof (e.g., promoter, enhancer)) by next generation sequencing (NGS) and/or Q-PCR.

### Example 4: Gene Editing of HSCs with AAV Vector Comprising Effector Protein

An AAV vector is constructed to contain a transgene between its ITRs. Such a transgene, for example, can provide or encode, in a 5′ to 3′ direction, a first promoter (e.g., U6), a guide nucleic acid (e.g., guide RNA, sgRNA, crRNA), a second promoter (e.g., EFS), an effector protein (e.g., any one of the effector proteins recited in TABLE 1, or a variant thereof as recited in TABLE 2), and a poly A signal (e.g., SV40 poly A tail). Optionally, the AAV vector comprises additional promoters, guide nucleic acids (e.g., guide RNA, sgRNA, crRNA), transcriptional enhancers (e.g., WPRE), or combinations thereof. Additional guide nucleic acids comprise different repeat and/or handle sequences targeting different sequences in a target nucleic acid. The effector protein comprises an amino acid sequence with at least 85% identity to a sequence recited in TABLE 1 and TABLE 2. Additionally, contacting the effector protein-guide nucleic acid complex to the target nucleic acid results in modifying a target sequence (e.g., nuclease activity, nickase activity, base editing activity, cleavage activity, integrase activity, recombinase activity). Optionally, the effector protein can be expressed either ubiquitously or tissue-specifically based on the second promoter the AAV vector is engineered to have. The AAV vector is expressed with supporting plasmids to produce an adeno-associated virus (AAV). HSCs are contacted with the AAV for 24 hours. After about 96 hours, post AAV contact, DNA or RNA is isolated from the infected HSCs. Edits are detected at the targeted loci (e.g., HBB gene, HBG1 gene, HBG2 gene, BCL11A gene, or B2M gene, or regulatory sequence thereof (e.g., promoter, enhancer)) by next generation sequencing (NGS) and/or Q-PCR.

### Example 5: Base Editing with Fusion Protein in Lipofected Eukaryotic Cells

A nucleic acid vector encoding a polypeptide (e.g., effector protein, effector partner, fusion protein or a combination thereof) is construed for nucleic acid editing. The fusion protein comprises an effector protein or a variant thereof (e.g., TABLE 1 and TABLE 2) fused to an effector partner capable of modifying a target nucleic acid as described herein. Plasmid pairs co-expressing the fusion protein and guide nucleic acid (1 plasmid/target) are delivered to eukaryotic cells (e.g., hematopoietic stem cell (HSC), or any other eukaryotic cell) via lipofection using a lipofection reagent. Lipofected cells are first incubated with the fusion protein and guide nucleic acids (e.g., guide RNA, sgRNA, crRNA), prior to DNA being extracted from the lipofected cells. Editing is detected by next generation sequencing (NGS) of PCR amplicons of the targeted loci (e.g., HBB gene, HBG1 gene, HBG2 gene, BCL11A gene, or B2M gene, or regulatory sequence thereof (e.g., promoter, enhancer)) to assess whether the fusion protein edited the target sequence such that base editing is detected and recorded as a change in 1% base call relative to the negative control.

### Example 6: Gene Editing of Eukaryotic Cells with AAV Vector Comprising Fusion Proteins

An AAV vector is constructed to contain a transgene between its ITRs. In some embodiments, the transgene providing or encoding, in a 5′ to 3′ direction, a first promoter (e.g., U6), a guide nucleic acid (e.g., guide RNA, sgRNA, crRNA), a second promoter (e.g., EFS), an fusion protein (e.g., effector protein-fusion protein comprises an effector protein or a variant thereof (e.g., TABLE 1 and TABLE 2) fused to an effector partner), and a poly A signal (e.g., SV40 poly A tail). The AAV vector is expressed with supporting plasmids to produce an adeno-associated virus (AAV). Eukaryotic cells (e.g., HSC) are contacted with the AAV for 24 hours. After about 96 hours, post AAV contact, DNA or RNA is isolated from the infected eukaryotic cells. Edits are detected at the targeted loci (e.g., HBB gene, HBG1 gene, HBG2 gene, BCL11A gene, or B2M gene, or regulatory sequence thereof (e.g., promoter, enhancer)) by next generation sequencing (NGS) and/or Q-PCR.

### Example 7: Ex Vivo Editing Activity of L26R Variant of CasPhi.12 in HSCs

This experiment was performed to determine if variants of CasPhi. 12 (the L26R variant) can modify HSCs. First, the L26R variant of CasPhi.12 effector protein (SEQ ID NO: 2) was engineered and delivered as mRNA into HSCs using a neon electroporation system using various protocols (FIG. 1). The 1500 volts/30 ms/2 pulse protocol was selected.

mRNA sequences encoding the L26R variant or corresponding WT effector protein (SEQ ID NO: 2) targeting a TTN PAM (SEQ ID NO: 82) were prepared. Guide nucleic acids comprising a repeat sequence (SEQ ID NO: 253) and a spacer sequence (one of SEQ ID NOs: 860-864) as described in TABLE 13 were prepared.

HSCs were prepared for electroporation with an mRNA encoding an effector protein and a guide nucleic acid. 2×10⁵ HSCs were spun down at 300 g for 10 mins. The cells were washed with PBS and spin down at 300 g for 5 to 10 mins. The cell pellet was resuspended with 10 μl Buffer T. Next, 5 μg or 10 μg of the L26R variant mRNA and 500 pmol synthetic gRNA were mixed. The RNA mixture was added to the HSC cell suspension and delivered using a neon electroporation at 1500 volts, 10 ms and 3 pulses. HSCs were immediately transferred to culture medium and incubated at 37° C.

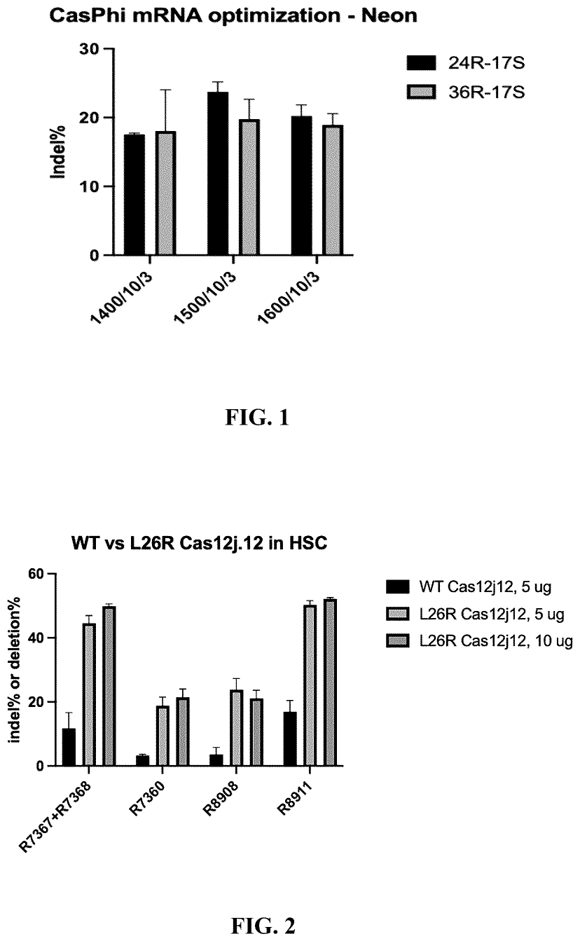

Indels were detected by NGS at the targeted loci. Indel percentage was calculated as the fraction of sequencing reads containing insertions or deletions relative to an unedited reference sequence. Results can be seen in FIG. 2. The L26R variant demonstrated increased editing activity compared to the WT effector protein (SEQ ID NO: 2).

TABLE 13
Guide nucleic acid sequences for the L26R
variant targeting HSCs

| | Repeat | Spacer |
|---|---|---|
| | Sequence | Sequences |
| | SEQ ID | SEQ ID |
| Name | No. | No. |
| R7360 | 253 | 860 |
| R7367 | 253 | 861 |
| R7368 | 253 | 862 |
| R8908 | 253 | 863 |
| R8911 | 253 | 864 |

### Example 8: HSCs Editing for SCD & β-Thalassemia

Hematopoietic stem cells (HSCs), the source of all blood cells, are self-renewing (i.e., able to divide and persist without losing stem cell characteristics) and multipotent (i.e., able to produce all types of blood cells). Only about 1% of hematopoietic stem and progenitor cells (HSPCs), a mixture of stem and progenitor cells, are HSCs. Human HSPC are defined by the surface marker CD34+.

HSC gene therapy evolved from the bone marrow transplant, a widely-used therapy for malignant and non-malignant blood disease. In HSC gene therapy, donor-derived HSPC are genetically manipulated ex vivo (e.g., using gene editing) prior to infusion of the cells into the intended recipient. HSC gene therapy is clinically-effective for genetic blood disorders including disorders of the β-globin gene (HBB), such as sickle-cell disease (SCD), β-thalassemia, and disorder of other genes. SCD is caused by E6V mutation in the β-globin gene, generating sickle-like, short-lived red blood cells (RBC), leading to the severe blocking of small blood vessels. β-thalassemia is caused by various mutations in β-globin gene, which results in absence of functional RBC. Annually, 300,000 babies are born with SCD, and 60,000 babies are born with β-thalassemia. Several clinical trials are ongoing for HSC gene therapy using different treatment strategies: a treatment of adult and pediatric patients with β-thalassemia was approved by the FDA in 2022; and two FDA approvals in SCD are expected in 2023.

Strategies targeting the BCL11A erythroid enhancer and HBG1 2 promoters were developed to induce gene correction or fetal hemoglobin (HbF) induction. One such strategy is the substitution of a target sequence of the β-globin encoding gene (e.g., the BS-globin gene or the HBB gene) with a donor nucleic acid. The present strategy may induce homology directed repair (HDR) for the correction of a single mutation, the base conversion of a single mutation, and/or the correction of common clustered mutations for β-thalassemia, all of which can result in the direct correction of the mutation for SCD. Another strategy is to disrupt the BCL11A erythroid enhancer by disrupting (e.g., cleaving) the binding motif of one or more transcriptional factors, such as GATA1, in the BC11A gene to downregulate BCL11A and upregulate γ-globin. Such a strategy may result in large indels and are preferred for disruption of binding motifs of multiple transcriptional activators. An additional strategy is to disrupt the binding motif of the transcription inhibitor of γ-globin gene and/or to base convert the HBG1 2 promoters to create a novel transcriptional activator to downregulate BCL11A.

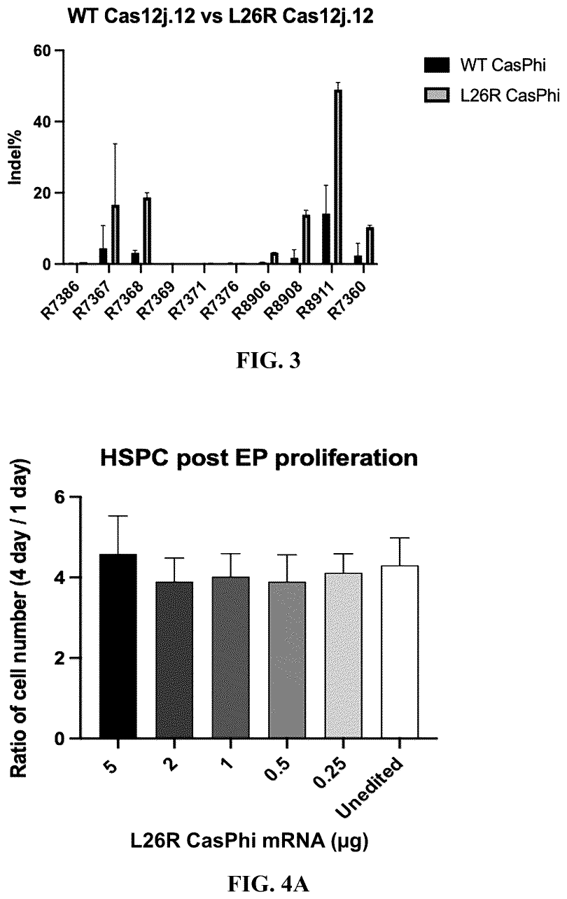

This experiment was performed to demonstrate the efficacy and safety of effector protein variants of CasPhi.12 (the L26R variant) to modify and edit HSCs in vitro and in vivo. First, conjugate guide nucleic acids were screened for accurate targeting and cleavage of the γ-globin gene (HBG1 or HBG2) promoter. Guide nucleic acids comprising a repeat sequence (SEQ ID NO: 253) and a spacer sequence (one of SEQ ID NO: 860-869) as set forth in TABLE 14 were prepared. HSPCs were cultured with X-VIVO 15+CC110 for 3 days post-thaw pre-electroporation. Then, 2×10⁵ cells were electroporated with 5 μg mRNA of L26R variant of CasPhi. 12 effector protein (the L26R variant) or CasPhi. 12 WT and 500 pmol guide nucleic acids by Neon system (1500v/10 ms/3pulse). Indels were detected by NGS at the targeted loci(s) 48 hours post-transfection. Indel percentage was calculated as the fraction of sequencing reads containing insertions or deletions relative to an unedited reference sequence. Results can be seen in FIG. 3. The R8911 guide nucleic acid comprising repeat sequence (SEQ ID NO: 253) and spacer sequence (SEQ ID NO: 864) demonstrated the highest indel activity.

TABLE 14
Guide nucleic acid sequences for L26R effector protein
(SEQ ID NO: 2) targeting HSCs

| | | Repeat | Spacer |
|---|---|---|---|
| | | Sequence | Sequences |
| | | SEQ ID | SEQ ID |
| Name | Target Locus | No. | No. |
| R7360 | HBB E6 | 253 | 860 |
| R7367 | BCL11A binding site in the promoter | 253 | 861 |
| | of HBG1 & HBG2 | | |
| R7368 | BCL11A binding site in the promoter | 253 | 862 |
| | of HBG1 & HBG2 | | |
| R8908 | LRF1/ZBTB7A binding site in the | 253 | 863 |
| | promoter of HBG1 & HBG2 | | |
| R8911 | LRF1/ZBTB7A binding site in the | 253 | 864 |
| | promoter of HBG1 & HBG2 | | |
| R7386 | BCL11A erythroid enhancer | 253 | 865 |
| R7369 | BCL11A binding site in the promoter | 253 | 866 |
| | region of HBG1 & HBG2 | | |
| R7371 | BCL11A binding site in the promoter | 253 | 867 |
| | of HBG1 & HBG2 | | |
| R7376 | BCL11A binding site in the promoter | 253 | 868 |
| | of HBG1 & HBG2 | | |
| R8906 | LRF1/ZBTB7A binding site in the | 253 | 869 |
| | promoter of HBG1 & HBG2 | | |

### Example 9: HSPC Editing Optimization

Next, HSPC editing was optimized using L26R variant of CasPhi.12 effector protein (SEQ ID NO: 2). HSPC were cultured with SFEM II+CC110 for 3 days pre-electroporation. On the day of electroporation, 2×10⁵ cells were electroporated with varied amounts of L26R variant of CasPhi.12 effector protein (SEQ ID NO: 2) mRNA and 500 pmol of R8911 guide nucleic acid comprising repeat sequence (SEQ ID NO: 253) and spacer sequence (SEQ ID NO: 864) by Neon system (1500v/10 ms/3pulse).

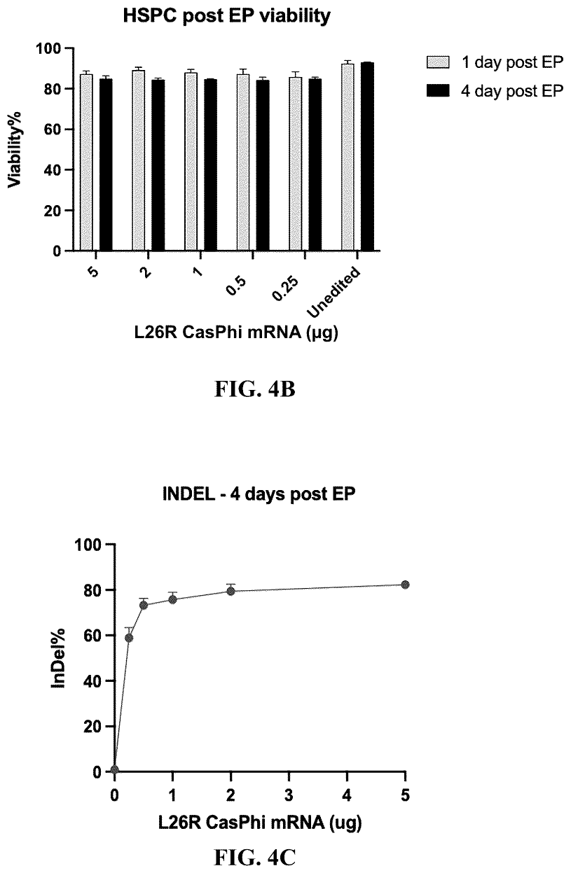

A day post-electroporation, the cells were mixed with CountBright beads for assessment of viability and cell count by manual or flow cytometry. About 2×10⁴ cells were then seeded for cell expansion in SFEMII and erythroid expansion supplement. Four days post-electroporation, the cells were counted again by flow cytometry and lysed for indel measurement by NGS. Proliferation (FIG. 4A) and viability (FIG. 4B) were not affected by L26R variant of CasPhi.12 effector protein (SEQ ID NO: 2) gene editing. DNA was extracted from each sample by QuickExtract for NGS library prep followed by Miseq and demonstrated high indel activity by L26R 4 days post electroporation (FIG. 4C). The totality of the data demonstrates that L26R CasPhi also showed high efficacy and no impact in HSPC viability and proliferation.

### Example 10: In Vivo Editing Activity of L26R CasPhi.12 Variants in HSCs and Engraftment Potential of Edited HSCs

The following experiments (i.e., 11, 12, 18, 19, 23) were performed, at least in part, to functionally validate HSC editing and persistence of edited HSC in vivo. Successful in vivo HSC engraftment (i.e., successful HSC homing to marrow (BM) niches, HSC survival, and proliferation), would facilitate the development of in vivo HSC targeting protocols (such as tissue processing and CD34+ cells editing efficacy).

### Example 11: Scaled Up HSPC Editing & Full Quality Control Analysis of Frozen HSPCs

This experiment is performed to demonstrate that frozen commercially available HSPCs can still be successfully edited and retain engraftment potential using of L26R variant of CasPhi.12 effector protein (SEQ ID NO: 2). Briefly, HSPCs are cultured for 3 days post-thaw and 2×10⁶ cells are electroporated with mRNA sequences encoding effector proteins and gRNA by the Neon system. One day after electroporation, cells are frozen in liquid nitrogen. Additionally, fresh cells are counted by flow cytometry, expanded in erythroid expansion medium, and are seeded for a colony formation unit (CFU) assay to establish multi-lineage development potential (e.g., differentiation into megakaryocytes, erythrocytes, macrophages, or neutrophils). Five days after electroporation, cells are counted again by flow cytometry and lysed for indel measurement by NGS. The frozen cells are then thawed to repeat the 1-day post electroporation protocol for quality control analysis.

### Example 12: In Vivo Engraftment of Unedited HSC into NBSGW Mice

The present assay was performed to establish a baseline of unedited HSC engraftment into NBSGW (NOD.Cg-Kit^{W-41J} Tyr Prkdc^{scid} I12rg^{im1Wjl}/ThomJ) mice which support engraftment of human hematopoietic stem cells without irradiation.

Briefly, one vial of HSPCs were thawed, spun down and resuspended in DPBS in low (220,000 cells) and high (700,000 cells) dose concentrations. The HSPCs were then injected into NBSGW mice under sterile conditions. Six weeks post injection, retro-orbital bleeding of peripheral blood was collected for RBC lysis and detection of human HSC engraftment by flow cytometry. Ten weeks post injection, BM flush, spleen, and peripheral blood was collected through terminal engraftment analysis of peripheral blood, bone marrow and the spleen, and analyzed for human HSC engraftment by flow cytometry.

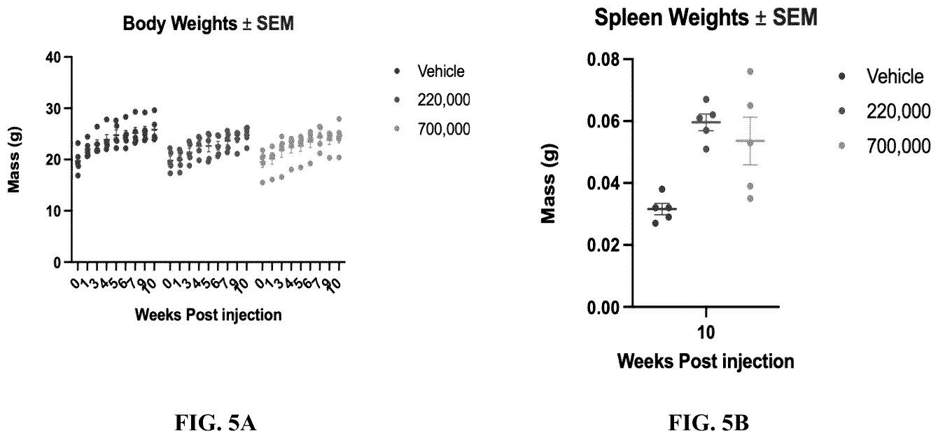

Results demonstrated that all mice were growing or in a healthy weight range (FIG. 5A). For 220,000 cells group dosed (low dose) there is a 88.6% increase and for 700,000 cells group (high dose) there is a 69.6% increase in spleen weight compared to the vehicle group (FIG. 5B).

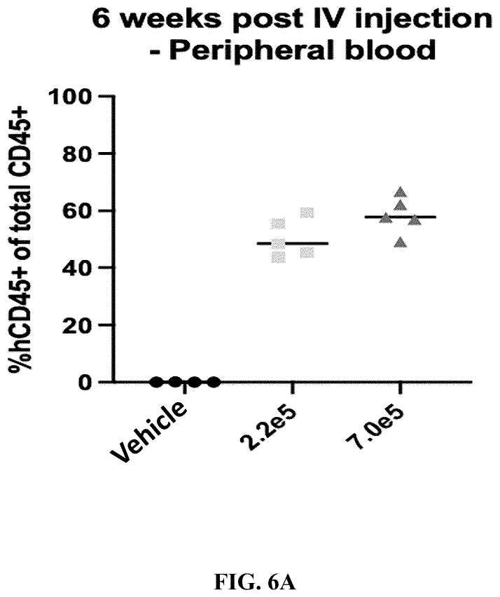

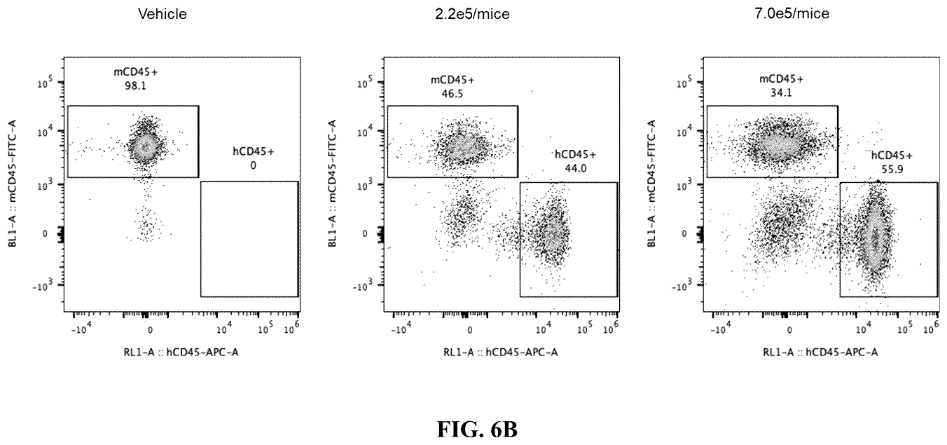

At the six-week, halfway analysis of hCD45% in peripheral blood was performed, wherein 100 to 200 μl of blood was lysed by RBC lysis buffer twice. White blood cells were then stained with live/dead zombie Violet dye, mCD45-FITC, and hCD45-APC conjugated antibody for 30 mins before analyzed by flow cytometry. Successful HSC engraftment was demonstrated by human CD45+% in peripheral blood 6 weeks post injection (FIGS. 6A-6B).

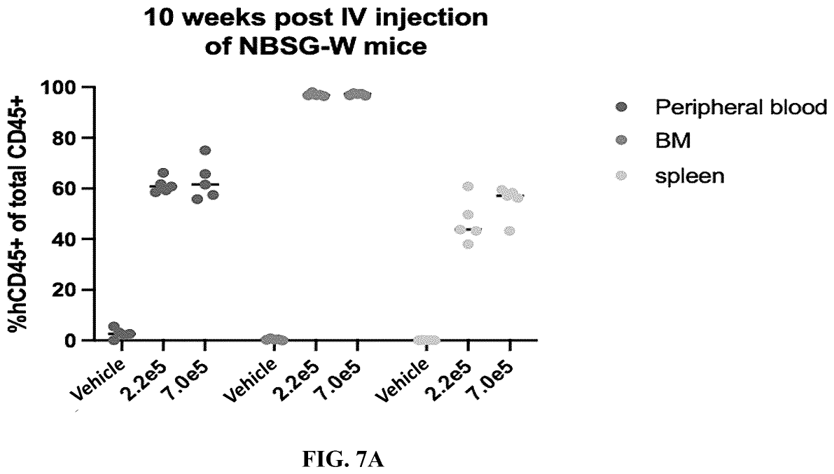

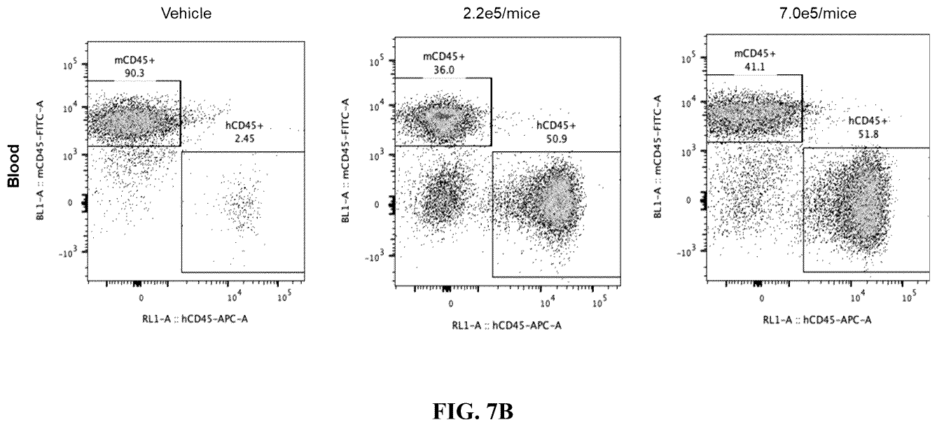

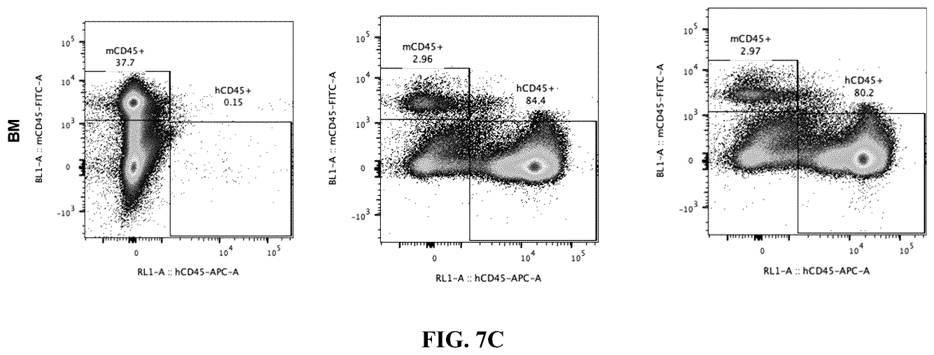

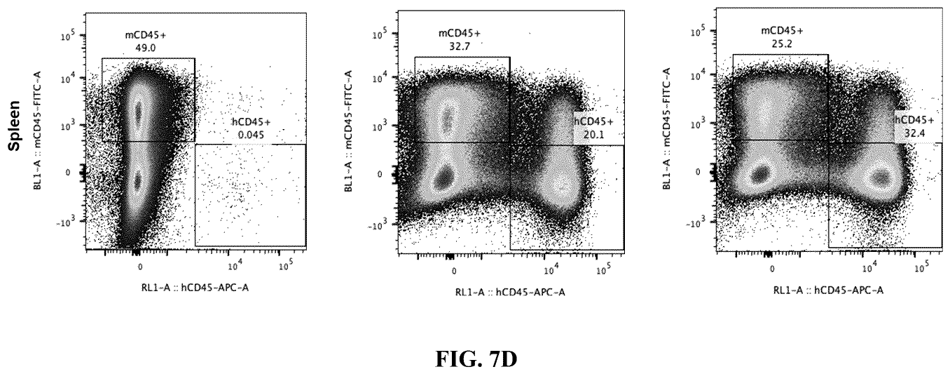

At the 10-week analysis, terminal hCD45% was measured in in peripheral blood, bone marrow, and the spleen. Briefly, 500 to 700 μl of blood was lysed by RBC lysis buffer twice. White blood cells were then stained with live/dead zombie Violet dye, mCD45-FITC, and hCD45-APC conjugated antibody for 30 mins before analyzed by flow cytometry (FIGS. 7A-7B).

Bone marrow flush were filtered through 70-μm cell strainer and spleen tissue was grinded on the 70-μm cell strainer. Cells filtered through the strainer were lysed with RBC lysis buffer before staining with live/dead zombie Violet dye, mCD45-FITC, and hCD45-APC conjugated antibody for 30 mins. Stained cells were then analyzed by flow cytometry (FIGS. 7C-7D).

Successful unedited human HSC engraftment in NBSGW mice demonstrated by blood human CD45+% six weeks post injection, as well as blood, bone marrow, and spleen human CD45+% 10 weeks after injection.

A similar experiment is carried out with HSCs edited by the L26R variant to demonstrate successful edited human HSC engraftment in NBSGW mice.

### Example 13: Homology Directed Repair (HDR) in HSPCs

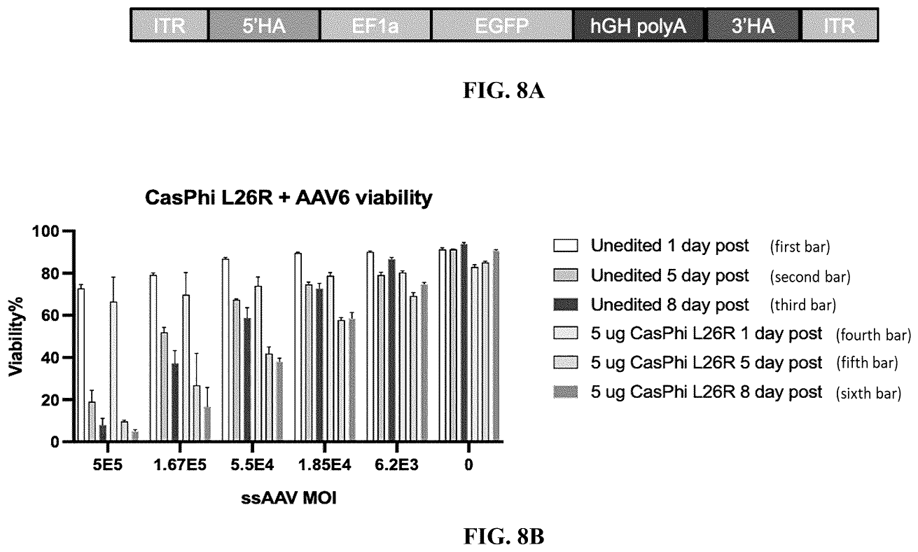

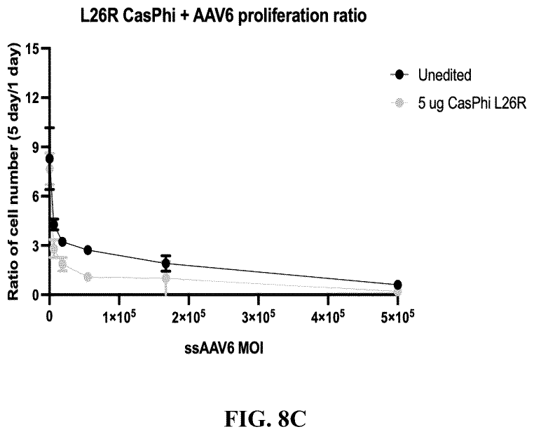

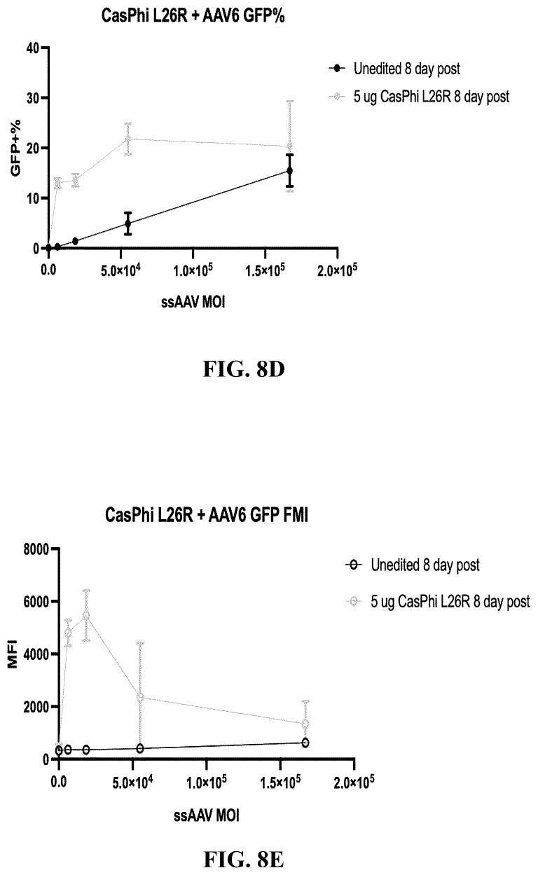

HSPCs were cultured with SFEM II+CC110 for 3 days post-thaw pre-EP. 2×10⁵ cells were electroporated with 5 μg of the L26R variant and 500 pmol guide nucleic acid targeting the TRAC locus by Neon system (1500 v/10 ms/3 pulse) followed by transduction of different doses of ssAAV6 (FIG. 8A). One day post electroporation, cells were mixed with CountBright beads for assessment of viability and cell count by flow cytometry or manually. Approximately 2×10⁴ cells were seeded for cell expansion in SFEM II+erythroid expansion supplement. Five days post electroporation, cells were counted again and lysed for indel percentage measurement by NGS. GFP integration is also measured by flow cytometry.

The results demonstrate that exposure to increasing doses of AAV6 leads to progressive loss of viability (FIG. 8B) and proliferative potential of HSPCs (FIG. 8C). Results also demonstrate that HDR was achieved in HSPCs where about 12% GFP integration was observed with L26R variant using a low down of AAV (FIGS. 8D and 8E). In terms of utilizing an AAV as a vector, overall progressive loss of viability and proliferative potential of HSPC was observed using AAV6 as donor template. The loss of viability and proliferative potential was amplified when combining with electroporation of effector protein mRNA and guide nucleic acids.

### Example 14: HSCs Editing for SCD & β-Thalassemia

Hematopoietic stem cells (HSCs), the source of all blood cells, are self-renewing (i.e., able to divide and persist without losing stem cell characteristics) and multipotent (i.e., able to produce all types of blood cells). Only about 1% of hematopoietic stem and progenitor cells (HSPCs), a mixture of stem and progenitor cells, are HSCs. Human HSPC are defined by the surface marker CD34+.

HSC gene therapy evolved from the bone marrow transplant, a widely-used therapy for malignant and non-malignant blood disease. In HSC gene therapy, donor-derived HSPC are genetically manipulated ex vivo (e.g., using gene editing) prior to infusion of the cells into the intended recipient. HSC gene therapy is clinically-effective for genetic blood disorders including disorders of the β-globin gene, such as sickle-cell disease (SCD), β-thalassemia, and disorder of other genes. SCD is caused by E6V mutation in the β-globin gene, generating sickle-like, short-lived red blood cells (RBC), leading to the severe blocking of small blood vessels. β-thalassemia is caused by various mutations in β-globin gene, which results in absence of functional RBC. Annually, 300,000 babies are born carrying SCD, and 60,000 babies are born with β-thalassemia. Several clinical trials are ongoing for HSC gene therapy using different treatment strategies: a treatment of adult and pediatric patients with β-thalassemia was approved by the FDA in 2022; and two FDA approvals in SCD are expected in 2023.

Three strategies were developed to induce gene correction or fetal hemoglobin (HbF) induction. The first strategy included substitution of a target sequence of the β-globin gene (e.g., the βS-globin gene or the HBB gene) with a donor nucleic acid, inducing homology directed repair (HDR) for the correction of a single mutation, the base conversion of a single mutation, the correction of common clustered mutations for β-thalassemia, or a combination thereof, all of which can result in the direct correction of the mutation for SCD. The second strategy was to disrupt the erythroid enhancer gene (e.g., BCL11A gene) by disrupting (e.g., cleaving) the binding motif of one or more transcriptional factors (e.g., GATA1 gene) for downregulating expression of BCL11A gene and upregulate expression of γ-globin gene. The third strategy was to downregulate BCL11A gene expression by disrupting the binding motif of the transcription inhibitor of γ-globin gene and converting a base within HBG1/2 promoters to create a novel transcriptional activator, or a combination thereof.

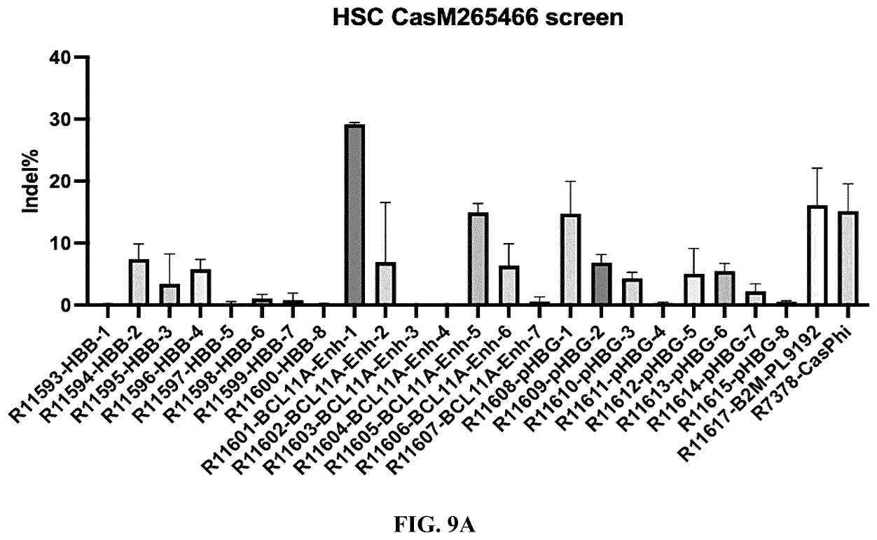

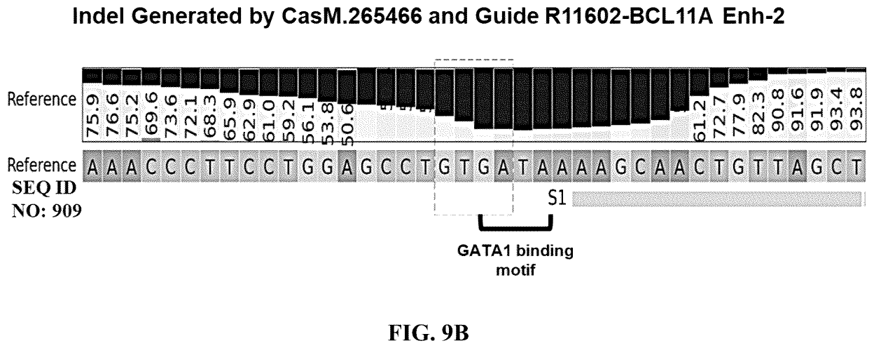

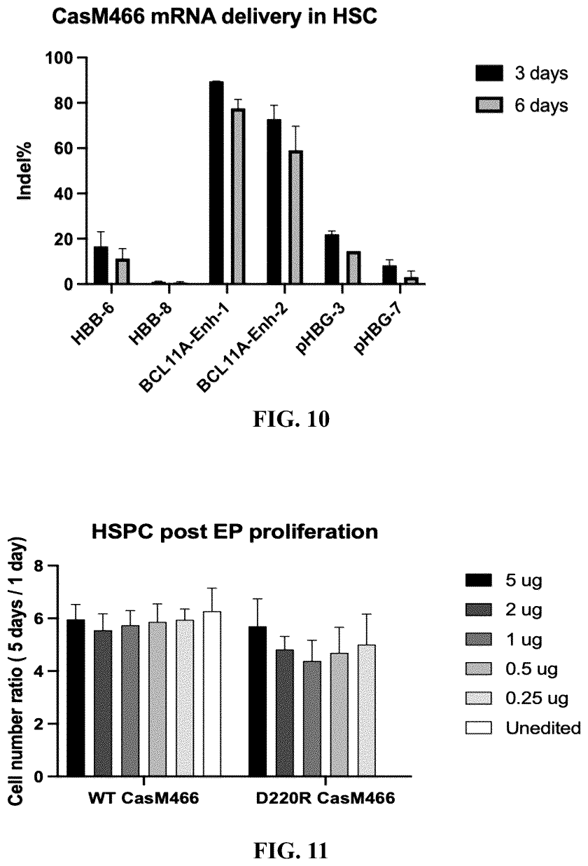

This experiment was performed to demonstrate the efficacy and safety of CasM.265466 effector protein to modify HSPCs in vitro. First, guide nucleic acids comprising a handle sequence having an intermediary sequence, a linker sequence, a repeat sequence as described herein, and a spacer sequence as described in TABLE 15 were screened for accurately targeting the HBB gene, the BCL11A gene (e.g., BCL11A Enh), and the HBG gene (e.g., HBG gene promoter, (pHBG)). A guide nucleic acid targeting the B2M gene (R11617-B2M-PL9192) was used as a positive control, a CasPhi guide nucleic acid (R7378-CasPhi), comprising the nucleotide sequence AUUGCUCCUUACGAGGAGACGAAGCUAGUCUAGUGCA (SEQ ID NO: 894)) was used as a positive control, and no guide nucleic acid was used as a negative control. Briefly, guide nucleic acids comprising an intermediary sequence (e.g., SEQ ID NO: 257), a linker sequence, a repeat sequence (e.g., SEQ ID NO: 251), and a spacer sequence (any one of SEQ ID NO: 870-893) as set forth in TABLE 15 were prepared. HSPCs were cultured with X-VIVO 15+CC110 for 3 days post-thaw pre-electroporation. Then, 2×10⁵ cells were electroporated with 5 μg of mRNA encoding CasM.265466 (SEQ ID NO: 1) or control mRNA and 500 pmol guide nucleic acids by Neon system (1500v/10 ms/3pulse). Indels were detected by NGS at the three targeted loci(s) 48 hours post-transfection. Indel percentage was calculated as the fraction of sequencing reads containing insertions or deletions relative to an unedited reference sequence. Results can be seen in FIG. 9A. FIG. 9B shows representative data for % indel generated by CasM.265466 and R11602-BCL11A Enh-2 guide nucleic acid. The experiment was repeated with a smaller guide nucleic acid group, and the results of 3 and 6 days post-transfection can be seen in FIG. 10.

TABLE 15
Exemplary Guide nucleic acid sequences for CasM.265466
effector protein (SEQ ID NO: 1) for editing HSCs

| | Spacer Sequence | |
|---|---|---|
| | SEQ ID No. | |
| | of Guide Nucleic | |
| Name | Acid Sequence | Target Loci |
| R11593-HBB-1 | 870 | HBB |
| R11594-HBB-2 | 871 | HBB |
| R11595-HBB-3 | 872 | HBB |
| R11596-HBB-4 | 873 | HBB |
| R11597-HBB-5 | 874 | HBB |
| R11598-HBB-6 | 875 | HBB |
| R11599-HBB-7 | 876 | HBB |
| R11600-HBB-8 | 877 | HBB |
| R11601-BCL11A Enh-1 | 878 | BCL11A Enh |
| R11602-BCL11A Enh-2 | 879 | BCL11A Enh |
| R11603-BCL11A Enh-3 | 880 | BCL11A Enh |
| R11604-BCL11A Enh-4 | 881 | BCL11A Enh |
| R11605-BCL11A Enh-5 | 882 | BCL11A Enh |
| R11606-BCL11A Enh-6 | 883 | BCL11A Enh |
| R11607-BCL11A Enh-7 | 884 | BCL11A Enh |
| R11608-pHBG-1 | 885 | pHBG |
| R11609-pHBG-2 | 886 | pHBG |
| R11610-pHBG-3 | 887 | pHBG |
| R11611-pHBG-4 | 888 | pHBG |
| R11612-pHBG-5 | 889 | pHBG |
| R11613-pHBG-6 | 890 | pHBG |
| R11614-pHBG-7 | 891 | pHBG |
| R11615-pHBG-8 | 892 | pHBG |
| R11617-B2M-PL9192 | 893 | B2M |

### Example 15. HSPC Editing Optimization Varying mRNA Encoding Effector Protein Concentration

HSPC editing was optimized using D220R variant of CasM.265466. HSPCs were cultured with SFEM II+CC110 for 3 days pre-electroporation. On the day of electroporation, 2×10⁵ cells were electroporated with varied amounts of D220R effector protein mRNA and 500 pmol of R11602-BCL11A Enh-2 guide nucleic acid by Neon system (1500v/10 ms/3pulse).

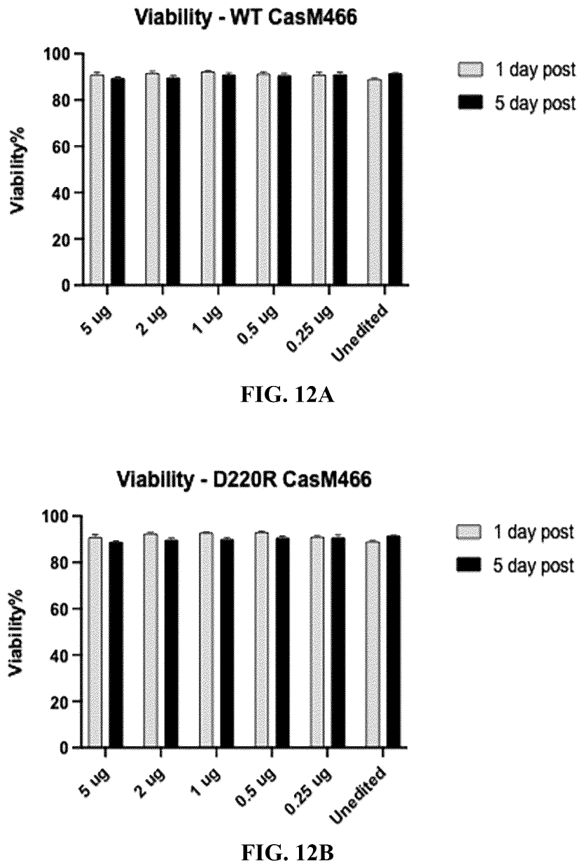

A day post-electroporation, the cells were mixed with CountBright beads for assessment of viability and cell count by manual or flow cytometry. About 2×10⁴ cells were then seeded for cell expansion in SFEMII and erythroid expansion supplement. Five days post-electroporation, the cells were counted again by flow cytometry and lysed for indel measurement by NGS. Both effector proteins, CasM.265466 and D220R variant, showed similar proliferation (FIG. 11) and viability (FIGS. 12A-12B) profiles.

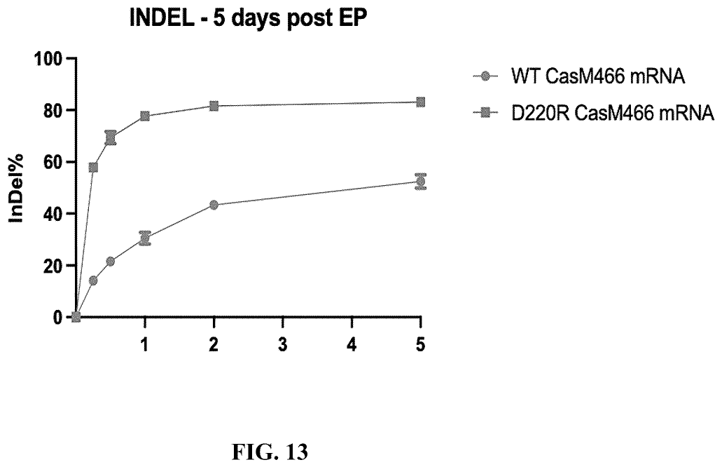

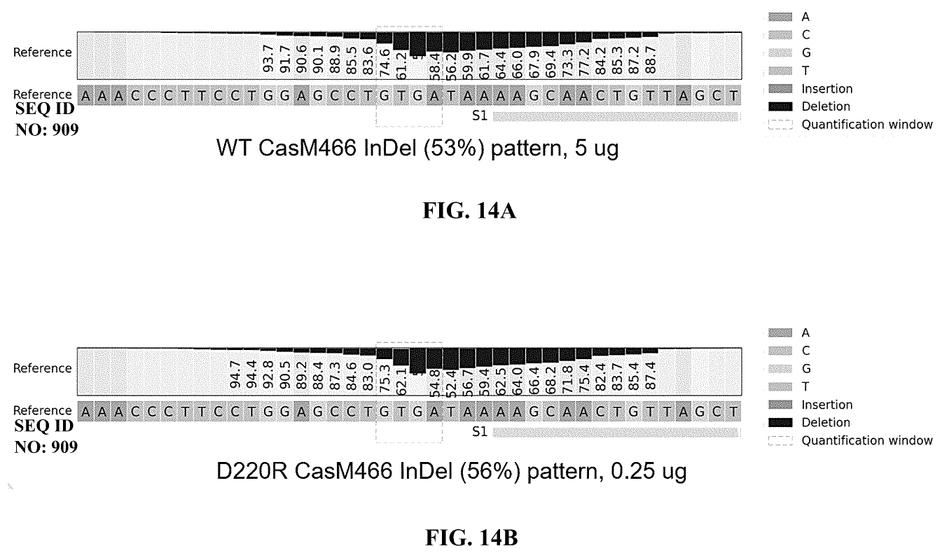

DNA was extracted from each sample by QuickExtract for NGS library prep followed by Miseq. Indel activity was assessed 5 days post electroporation. The D220R variant demonstrated higher indel percentage than corresponding wildtype CasM.265466 (FIG. 13). Both effector proteins, CasM.265466 and D220R variant thereof, showed similar indel patterns (FIGS. 14A-14B).

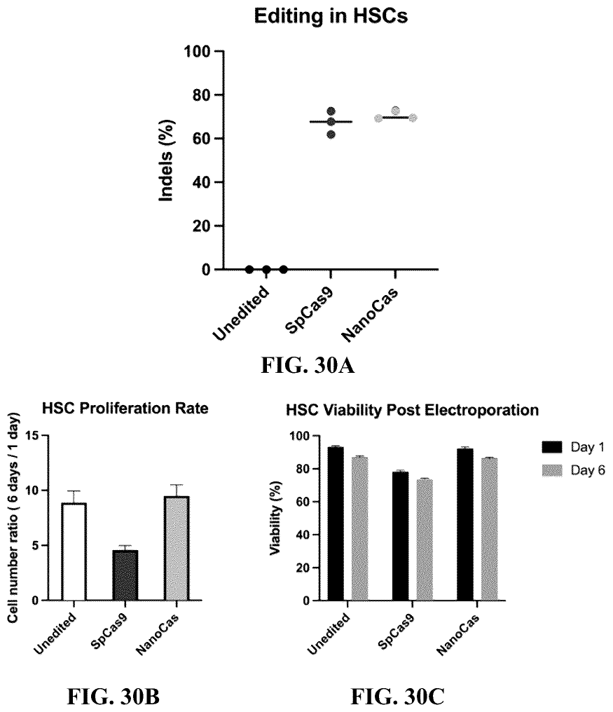

Using similar methods described herein, HSC editing, proliferation and viability for CasM.265466 was compared to Cas9 from Streptococcus pyogenes (SpCas9). Results are depicted in FIGS. 30A-30C. 6 days post electroporation, the % indels for SpCas9 and CasM.265466 were similar, whereas cells edited with CasM.265466 showed a proliferation rate and viability more similar to unedited control cells.

### Example 16. HSPC Editing Optimization Varying Guide Nucleic Acid Concentration

A dose titration assay is performed of the guide nucleic acid sequences paired with the CasM.265466 effector protein (SEQ ID NO: 1) to establish dose dependency of the guide nucleic acids.

HSPCs are cultured with SFEM II+CC110 for 3 days pre-electroporation. On the day of electroporation, 2×10⁵ cells are electroporated with 2 μg of mRNA encoding the effector protein and R11602-BCL11A Enh-2 guide nucleic acid at varying dose in a range from 0 pmol to 1000 pmol by Neon system (1500v/10 ms/3pulse).

A day post-electroporation, the cells are mixed with CountBright beads for assessment of viability and cell count by manual or flow cytometry. About 2×10⁴ cells are then seeded for cell expansion in SFEMII and erythroid expansion supplement. Five days post-electroporation, the cells are counted again by flow cytometry and lysed for indel measurement by NGS. Proliferation and viability profile of the HSPCs are determined. DNA is extracted from each sample by QuickExtract for NGS library prep followed by Miseq.

### Example 17. In Vivo Editing Activity of Effector Proteins in HSCs and Engraftment Potential of Edited HSCs

The experiments described herein were performed to functionally validate HSC editing and persistence of edited HSC in vivo. Successful in vivo HSC engraftment (i.e., successful HSC homing to bone marrow (BM) niches, HSC survival, and proliferation) would facilitate the development of in vivo HSC targeting protocols (such as tissue processing and CD34+ cells editing efficacy).

### Example 18. Scaled Up HSPC Editing & Full Quality Control Analysis of Frozen HSPCs

This experiment is performed to demonstrate that commercially available frozen HSPCs can be successfully edited and retain engraftment potential. Briefly, HSPCs were cultured for 3 days post-thaw and 2×10⁶ cells and were electroporated with mRNA sequences encoding CasM.265466 effector protein (SEQ ID NO: 1) and guide nucleic acids by the Neon system. One day after electroporation, cells were divided in two portions. A first portion of the electroporated cells was frozen in liquid nitrogen. A second portion of the electroporated cells was subjected to flow cytometry, expanded in erythroid expansion medium, and were seeded for a colony formation unit (CFU) assay to establish multi-lineage development potential (e.g., differentiation into megakaryocytes, erythrocytes, macrophages, or neutrophils). Five days after electroporation, the cells from the second portion were counted again by flow cytometry and lysed for indel measurement by NGS. The cells from the first portion were then thawed and treated similar to the cells from the second portion were treated 1-day post electroporation.

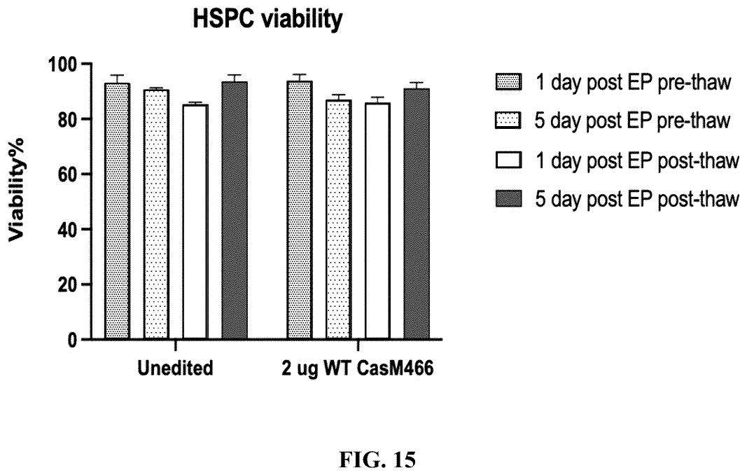

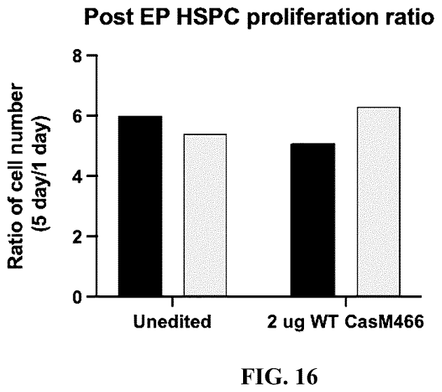

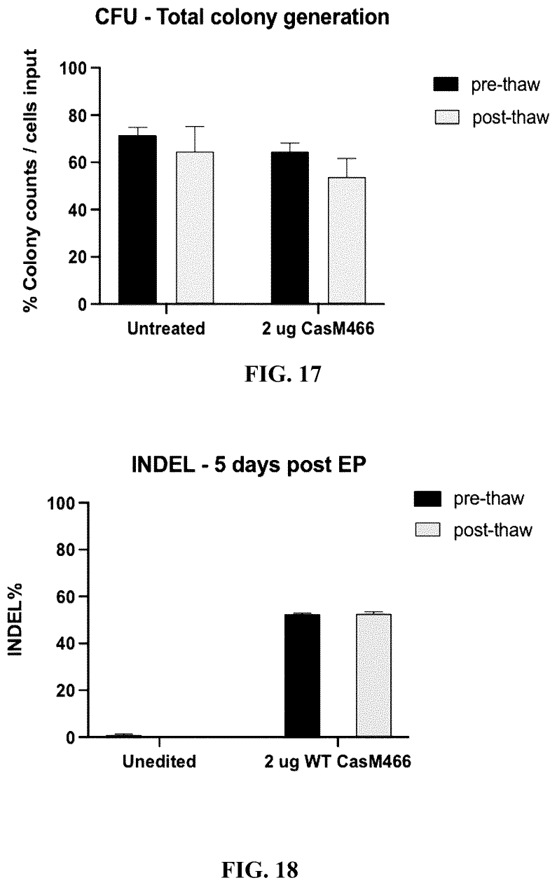

Results show that unedited HSPCs and the CasM.265466 effector protein (SEQ ID NO: 1) edited HSPCs showed similar HSPC viability (FIG. 15), proliferation (FIG. 16), and multi-lineage colony generation (FIG. 17). Furthermore, results show no significant difference in indel percentage between pre-thaw and post-thaw samples (FIG. 18). The totality of the data shows that scaled up editing of HSPCs using the mRNA encoding CasM.265466 effector protein (SEQ ID NO: 1) delivery achieved high proliferation and viability without compromising the editing outcome. Also, no difference was observed between pre-thaw and post-thaw samples in proliferation, viability, InDel %, and multi-lineage development of CasM.265466 effector protein edited HSPCs.

### Example 19. In Vivo Engraftment of Unedited HSC into NBSGW Mice

The present assay was performed to establish a baseline of unedited HSC engraftment into NBSGW (NOD.Cg-Kit^{W-41J} Tyr Prkdc^{scid} I12rg^{im1Wjl}/ThomJ) mice which support engraftment of human hematopoietic stem cells without irradiation.

Briefly, one vial of HSPCs were thawed, spun down and resuspended in DPBS in low (220,000 cells) and high (700,000 cells) dose concentrations. The HSPCs were then injected by IV into NBSGW mice under sterile conditions. Six weeks post injection, retro-orbital bleeding of peripheral blood was collected for RBC lysis and detection of human HSC engraftment by flow cytometry. Ten weeks post injection, bone marrow flush, spleen, and peripheral blood were collected and analyzed for human HSC engraftment by flow cytometry.

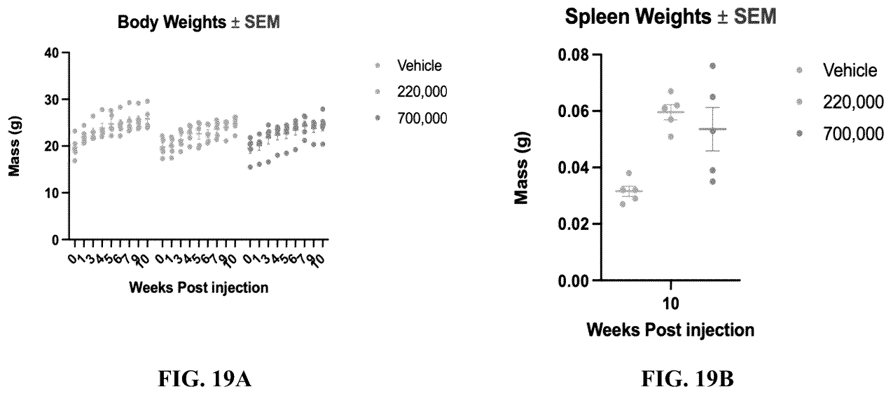

Results demonstrated that all mice were growing or in a healthy weight range (FIG. 19A). Mice dosed with 220,000 cells group (low dose) showed 88.6% increase and 700,000 cells group (high dose) showed 69.6% increase in spleen weight compared to the vehicle group (FIG. 19B).

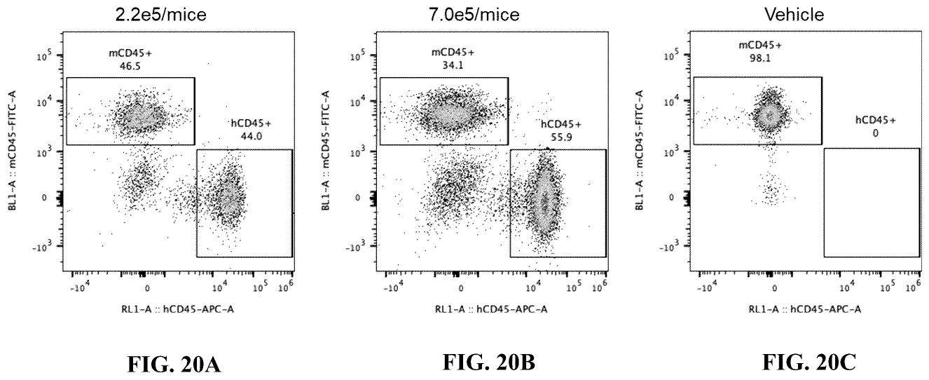

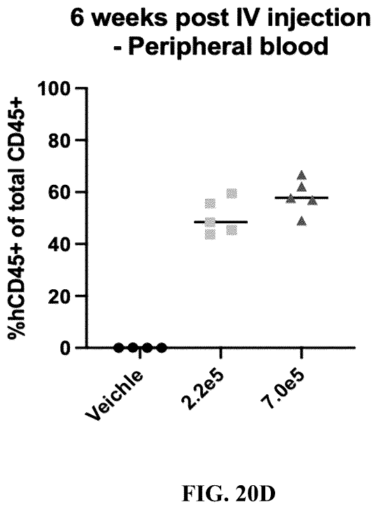

At the six-week point, an analysis of % hCD45 in peripheral blood, which was collected by Retro-orbital Bleeding, was performed. About 100 to 200 μl of blood was lysed by RBC lysis buffer twice. White blood cells were collected and then stained with live/dead zombie Violet dye, mCD45-FITC, and hCD45-APC conjugated antibody for 30 mins. Stained cells were then analyzed by flow cytometry (FIGS. 20A-20C). The results of FIGS. 20A-20C were used for calculating the presence of % hCD45+, which are summarized in FIG. 20D. An analysis of FIG. 20D indicated successful HSC engraftment in peripheral blood six-weeks post injection.

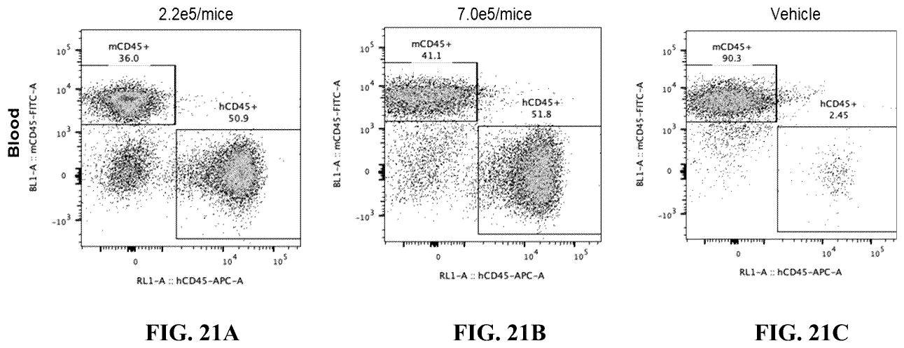

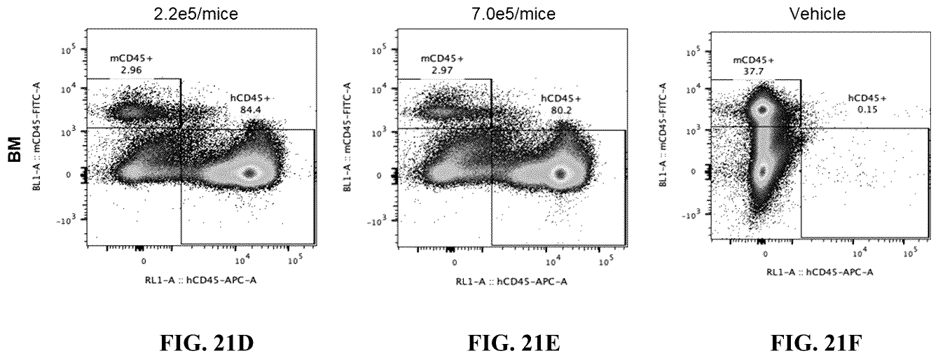

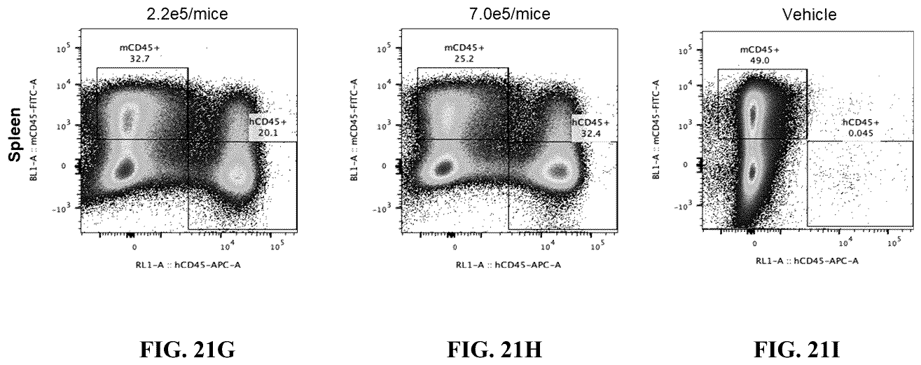

Similar analysis was performed 10-weeks post injection, and terminal % hCD45+ was measured in in peripheral blood, bone marrow, and the spleen. Briefly, 500 to 700 μl of blood was lysed by RBC lysis buffer twice. White blood cells were then stained with live/dead zombie Violet dye, mCD45-FITC, and hCD45-APC conjugated antibody for 30 mins. Stained cells were then analyzed by flow cytometry (FIG. 21A-21C).

Bone marrow flush were filtered through 70-μm cell strainer. Cells filtered through the strainer were lysed with RBC lysis buffer before staining with live/dead zombie Violet dye, mCD45-FITC, and hCD45-APC conjugated antibody for 30 mins. Stained cells were then analyzed by flow cytometry (FIG. 21D-21F).

Spleen tissue was grinded on the 70-μm cell strainer. Cells filtered through the strainer were lysed with RBC lysis buffer before staining with live/dead zombie Violet dye, mCD45-FITC, and hCD45-APC conjugated antibody for 30 mins. Stained cells were then analyzed by flow cytometry (FIG. 21G-21I).

The results of FIGS. 21A-21I were used for calculating the presence of % hCD45+, which are summarized in FIG. 21J. An analysis of FIG. 21J indicated successful HSC engraftment 10 weeks post injection in peripheral blood, bone marrow and spleen.

### Example 20. Homology Directed Repair (HDR) in HSPCs

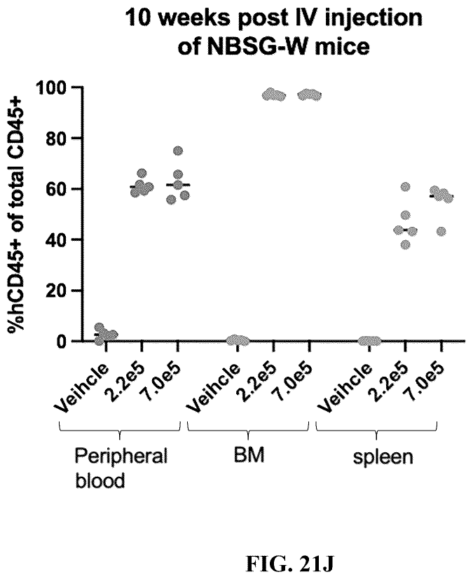

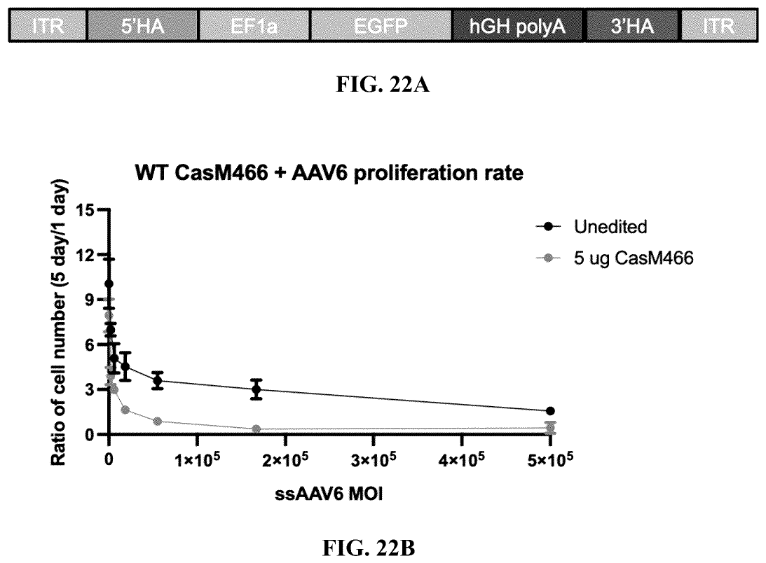

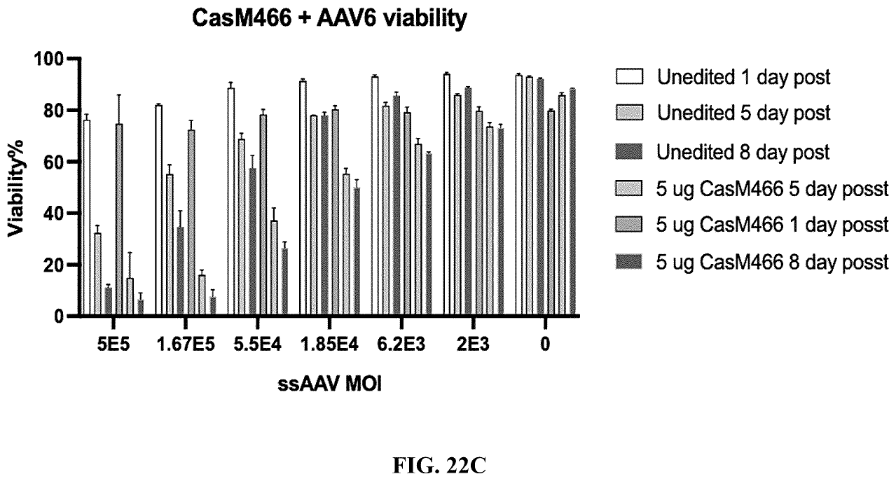

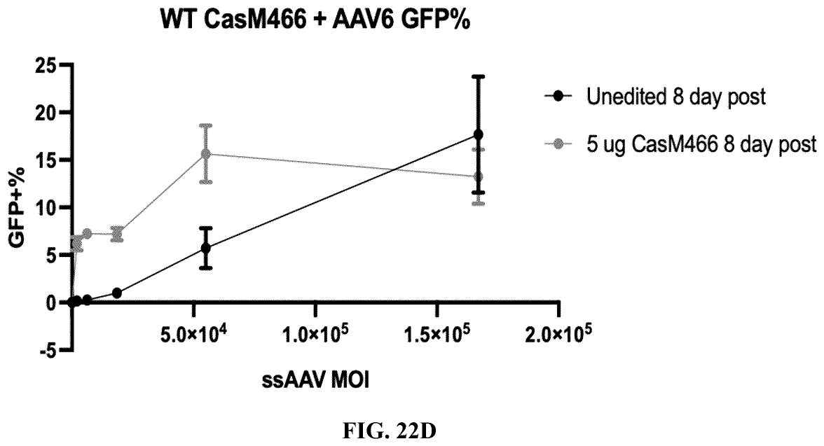

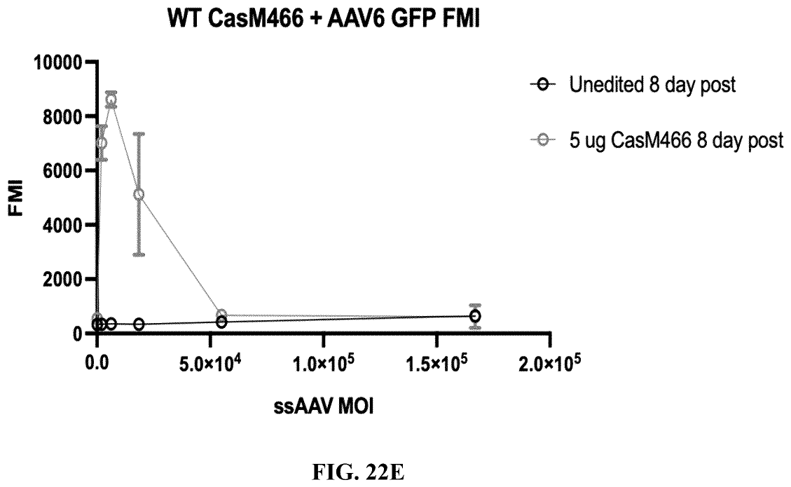

Frozen HSPCs were thawed and cultured with SFEM II+CC110 for 3 days. 2×10⁵ cells were electroporated with 5 μg of mRNA encoding CasM.265466 effector protein (SEQ ID NO: 1) and 500 pmol guide nucleic acid targeting the TRAC locus by Neon system (1500 v/10 ms/3 pulse). Following the electroporation, the cells were transduced with different doses of ssAAV6, wherein the ssAAV6 comprised a nucleotide sequence encoding GFP (FIG. 22A). One day post electroporation, cells were mixed with CountBright beads for assessment of viability and cell count by flow cytometry or manually. Approximately, 2×10⁴ cells were seeded for cell expansion in SFEM II+erythroid expansion supplement. Five days post electroporation, cells were counted again and lysed for indel percentage measurement by NGS. The results of proliferative potential of HSPCs and viability of HSPCs are summarized in FIG. 22B and FIG. 22C, respectively. An analysis of FIGS. 22B-22C indicated that exposure to lower doses of AAV6 leads to progressive improvement of viability and proliferative potential of HSPCs. GFP integration was also measured by flow cytometry and results were summarized in FIGS. 22D-22E. An analysis of FIG. 22D indicated about 6% GFP integration in TRAC locus was observed in CasM.265466 effector protein edited cells at low doses of AAV6.

### Example 21: HSPCs Cell Proliferation and Variability Assay

This experiment was performed to demonstrate that frozen commercially available HSPCs can be successfully edited using a D220R variant of CasM.265466 effector protein (SEQ ID NO: 1) or a L26R variant of CasPhi.12 effector protein (SEQ ID NO: 2) and retain engraftment potential. Briefly, frozen HSPCs were thawed and cultured with SFEM II+CC110 for 3 days pre-electroporation. On the day of electroporation, two batches of 2.5×10⁶ HSPCs were electroporated with mRNA sequences encoding effector proteins and gRNA using a neon electroporation system at 1500 volts, 10 ms, and 3 pulses. The first batch of HSPCs were electroporated with 25 μg of mRNA encoding CasM.265466 effector protein (SEQ ID NO: 1) and 6250 pmol R11602 guide nucleic acid targeting the BCL11A locus. The second batch of HSPCs were electroporated with 25 μg of mRNA encoding CasPhi. 12 effector protein (SEQ ID NO: 2) and 6250 pmol R8911 guide nucleic acid targeting the promoter of HBG1 and HBG2). After electroporation, the HSCs were immediately transferred to a culture medium and incubated at 37° C.

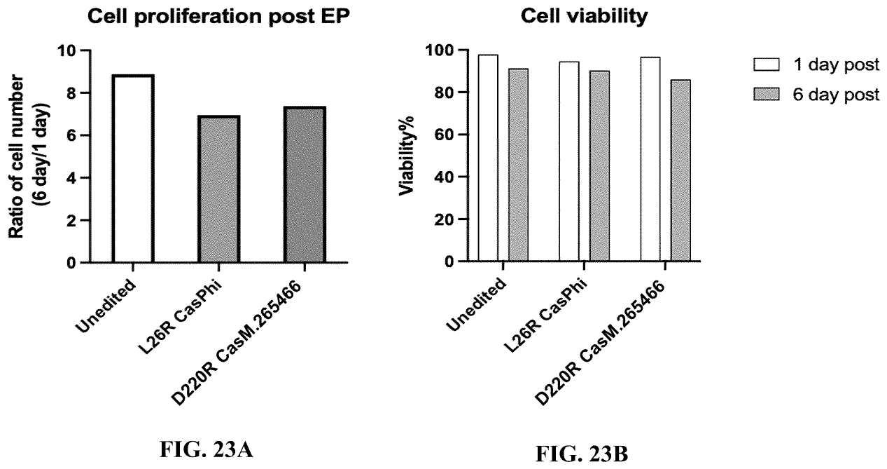

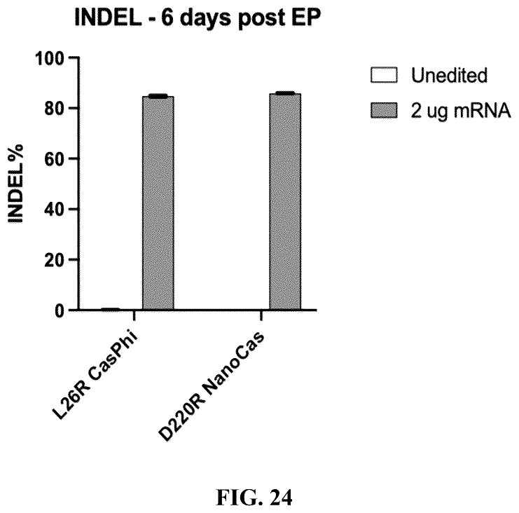

One day after electroporation, each batch of cells were divided in two portions. A first portion of the electroporated cells was frozen in liquid nitrogen. A second portion of the electroporated cells was subjected to flow cytometry. 2×10⁴ HSPCs from the second portion were expanded in SFEM II and erythroid expansion medium. Six days after electroporation, the cells from the second portion were counted again. The results of proliferative potential and viability of HSPCs are summarized in FIG. 23A and FIG. 23B, respectively. The cells from the first portion (e.g., unedited) were thawed and six days after electroporation, genomic DNA was extracted using QuickExtract followed by NGS analysis. The results of NGS are summarized in FIG. 24.

### Example 22: HSPCs Differentiation Assay

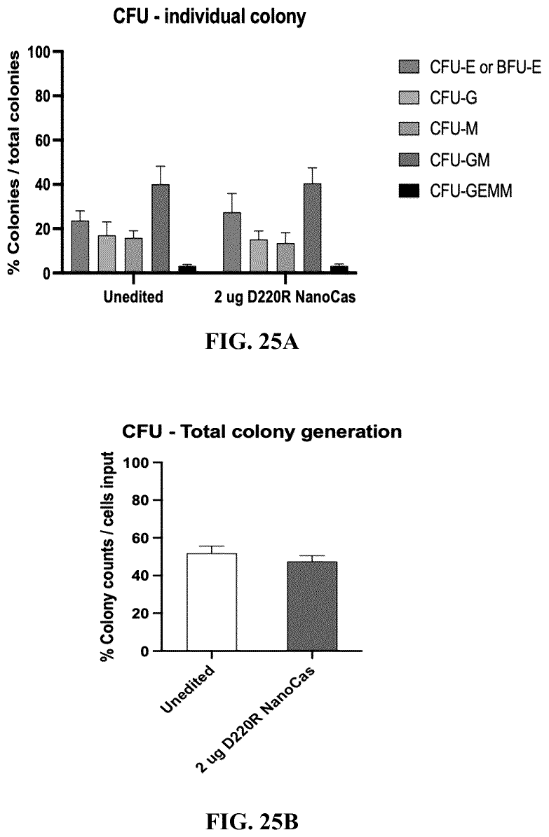

This experiment was performed to demonstrate that frozen commercially available HSPCs can be successfully edited using a D220R variant of CasM.265466 effector protein (SEQ ID NO: 1) and successfully differentiate. Briefly, frozen HSPCs were thawed and cultured with SFEM II+CC110 for 3 days pre-electroporation. On the day of electroporation, about 2.5×10⁶ HSPCs were electroporated with mRNA sequences encoding effector proteins and gRNA using a neon electroporation system at 1500 volts, 10 ms, and 3 pulses. The HSPCs were electroporated with 25 μg of mRNA encoding CasM.265466 effector protein (SEQ ID NO: 1) and 6250 pmol R11602 guide nucleic acid targeting the BCL11A locus. After electroporation, the HSCs were immediately transferred to a culture medium and incubated at 37° C. A day post-electroporation, the HSPCs were counted manually and cryopreserved. Additionally, three different densities (e.g., 75 cells, 75 cells, 150 cells) of HSPCs (e.g., erythroid colony-forming unit (CFU-E) or erythroid burst-forming unit (BFU-E), granulocyte colony-forming unit (CFU-G), megakaryocytic colony-forming unit (CFU-M), granulocyte macrophage colony-forming unit (CFU-GM), granulocyte-erythroid macrophage megakaryocytic colony-forming unit (CFU-GEMM)) were seeded for a colony formation unit (CFU) assay to establish multi-lineage development potential (e.g., differentiation into megakaryocytes, erythrocytes, macrophages, or neutrophils). The three different densities of HSPCs were plated onto methylcellulose gels for Myeloid-lineage differentiation for 14 days before individual colonies were manually counted. The results of the CFU assay of HSPCs are summarized in FIG. 25A and FIG. 25B, respectively.

### Example 23: In Vivo Engraftment of Edited HSC into NBSGW Mice

The present assay was performed to validate long term (i.e., over 3 months) HSC editing following in vivo engraftment of edited HSC into NBSGW (NOD.Cg-Kit^{W-41J} Tyr⁺ Prkdc^{scid} I12rg^{im1Wjl}/ThomJ).

Briefly, unedited HSPCs, HSPCs edited using a D220R variant of CasM.265466 effector protein (SEQ ID NO: 1) as described in Example 21, and HSPCs edited using a L26R variant of CasPhi.12 effector protein (SEQ ID NO: 2) as described in Example 21 were thawed, spun down and resuspended in DPBS. Approximately 450,000 unedited HSPCs, 450,000 HSPCs edited using the D220R variant, and 450,000 HSPCs edited using the L26R variant were independently injected into NBSGW mice under sterile conditions. Six weeks post injection, retro-orbital bleeding of peripheral blood was collected for RBC lysis and detection of human HSC engraftment by flow cytometry. Twelve weeks post injection, BM flush, spleen, and peripheral blood was collected through terminal engraftment analysis of peripheral blood, bone marrow and the spleen, and analyzed for human HSC engraftment by flow cytometry.

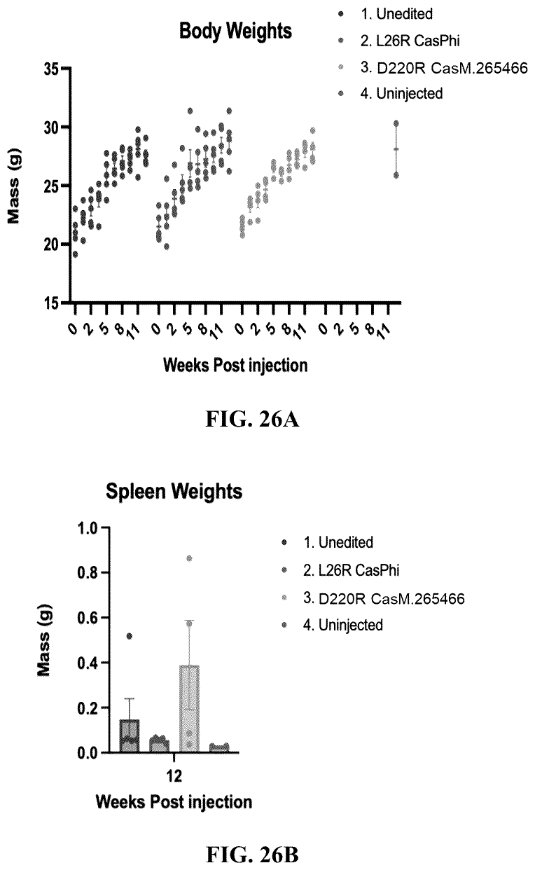

Results demonstrated that all mice were growing or in a healthy weight range, as summarized in FIG. 26A. Spleen weight for mice injected with D220R HSPCs increased considerably as compared to spleen weight for uninjected mice, mice injected with unedited HSPCs, and mice injected with L26R HSPCs, as summarized in FIG. 26B.

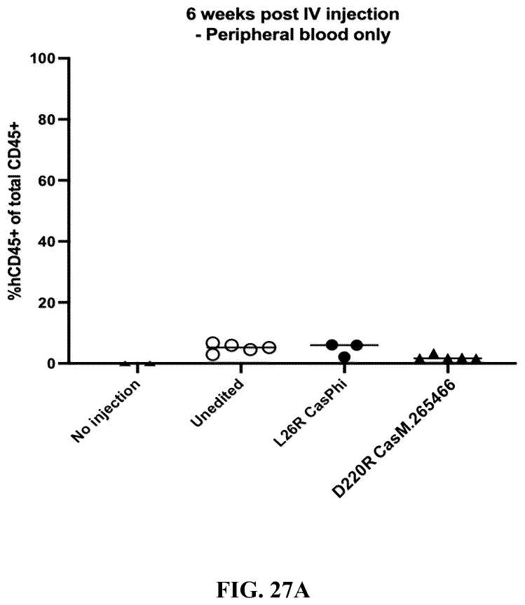

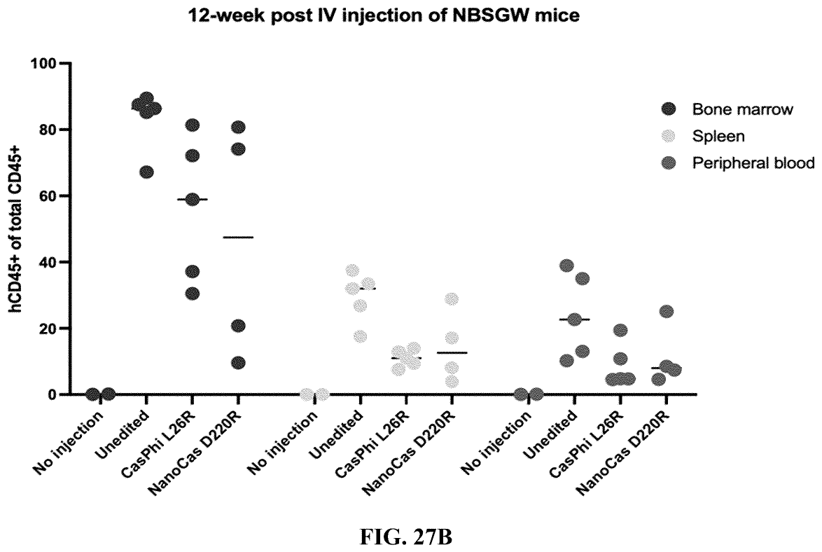

At the six-week, halfway analysis of hCD45% in peripheral blood was performed, wherein 100 to 200 μl of blood was lysed by RBC lysis buffer twice. White blood cells were then stained with live/dead zombie Violet dye, mCD45-FITC, and hCD45-APC conjugated antibody for 30 mins before analyzed by flow cytometry. Successful HSC engraftment was demonstrated by % of cells that are human CD45+ in peripheral blood 6 weeks post injection, as summarized in FIG. 27A.

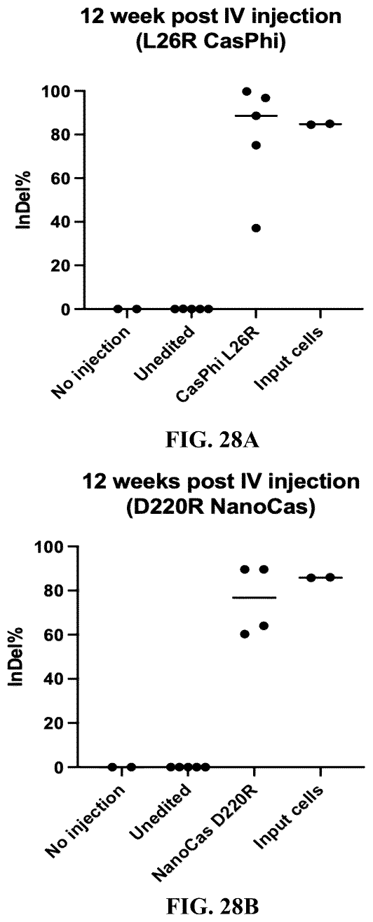

At the 12-week analysis, terminal hCD45% of total CD45+ was measured in bone marrow, spleen and peripheral blood. Briefly, 500 to 700 μl of blood was lysed by RBC lysis buffer twice. White blood cells were then stained with live/dead zombie Violet dye, mCD45-FITC, and hCD45-APC conjugated antibody for 30 mins before analyzed by flow cytometry. Bone marrow flush were filtered through 70-μm cell strainer and spleen tissue was grinded on the 70-μm cell strainer. Cells filtered through the strainer were lysed with RBC lysis buffer before staining with live/dead zombie Violet dye, mCD45-FITC, and hCD45-APC conjugated antibody for 30 mins. Stained cells were then analyzed by flow cytometry, as summarized in FIG. 27B. Genomic DNA was extracted from bone marrow and white blood cells by QuickExtract for NGS library prep followed by Miseq. The genomic DNA was subjected to PCR amplification to amplify the targeted loci. Indels were detected by NGS of PCR amplicons at the targeted loci, and indel percentage was calculated as the fraction of sequencing reads containing insertions or deletions relative to an unedited reference sequence. Results can be seen in FIG. 28A and FIG. 28B.

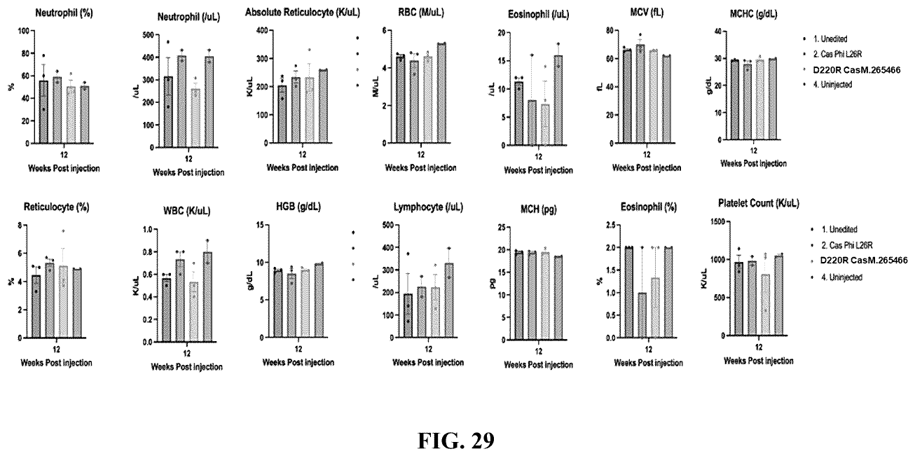

Additionally, ˜150 μl of peripheral blood was collected from all mice 12 weeks post injection and sent out for complete blood count (CBC) test. Results can be seen in FIG. 29. In brief, uninjected mice, mice injected with unedited HSPCs, mice injected with D220R HSPCs, and mice injected with L26R HSPCs all had comparable levels of blood cells.

Overall, successful unedited human HSC engraftment in NBSGW mice was demonstrated by blood having human CD45+ cells six weeks post injection, as well as blood, bone marrow, and spleen having human CD45+ cells 12 weeks after injection.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

1-59. (canceled)

60. A system for modifying a target nucleic acid of a hematopoietic stem cell (HSC), the system comprising:
(i) a polypeptide, or a nucleic acid encoding the polypeptide, wherein the polypeptide comprises an amino acid sequence that is at least 95% identical to any one of the amino acid sequences set forth in TABLE 1; and
(ii) an engineered guide nucleic acid, or a nucleic acid encoding the engineered guide nucleic acid, wherein:
a) the engineered guide nucleic acid comprises a first region and a second region,
b) the polypeptide at least partially binds to the first region to form an RNP complex,
c) the second region comprises a nucleotide sequence that is at least 80% complementary or at least 80% reverse complementary to a target sequence of the target nucleic acid,
d) the RNP complex, upon hybridization of the second region to the target nucleic, modifies the target nucleic acid of the HSC,
e) the first region and the second region are heterologous to each other, and
f) the HSC following modification by the RNP complex retains at least one of cell viability, cell proliferation, and multi-lineage development potential relative to an unmodified HSC.

61. The system of claim 60, wherein the target nucleic acid is within a gene of the HSC, and wherein the target nucleic acid comprises a nucleotide sequence of any one of genes set forth in TABLE 10, a variant thereof, a promoter thereof, an enhancer thereof, or a portion thereof.

62. The system of claim 60, wherein the polypeptide has nickase activity or nuclease activity.

63. The system of claim 60, wherein the polypeptide comprises an effector protein, an effector partner, a fusion protein or a combination thereof.

64. The system of claim 63, wherein the effector partner is selected from a polymerase, a deaminase, a reverse transcriptase, a transcriptional repressor, an integrase, a recombinase and a transcriptional activator.

65. The system of claim 64, wherein the effector protein is fused to an effector partner, wherein the effector protein and the effector partner are heterologous to each other.

66. The system of claim 60, wherein the polypeptide recognizes a protospacer adjacent motif (PAM) as set forth in TABLE 4.

67. The system of claim 60, wherein the engineered guide nucleic acid comprises a nucleotide sequence that is at least 85% identical to any one of the nucleotide sequences set forth in TABLE 9.

68. A viral vector that encodes one or more components of the system of claim 60, wherein the viral vector is an adeno associated viral (AAV) vector.

69. A pharmaceutical composition comprising the system of claim 60, and a pharmaceutically acceptable carrier.

70. A method of producing an engineered HSC, the method comprising:
(i) contacting a target nucleic acid of an HSC with the system of claim 60 for a first sufficient period of time to allow for transfection of the HSC; and
(ii) culturing the HSC for a second sufficient period of time for indels to occur in the target nucleic acid for modifying target nucleic acid and, thereby, producing the engineered HSC.

71. The method of claim 70, wherein modifying further comprises inserting a donor nucleic acid into the target nucleic acid.

72. An engineered HSC modified by the method of claim 71.

73. An engineered HSC modified by the system of claim 60.

74. A method of treating a disease or disorder comprising administering the engineered HSC of claim 73 to a subject in need thereof.

75. The method of claim 74, wherein the engineered HSC was previously frozen.

76. The method of claim 74, wherein the engineered HSC engrafts into the bone marrow of the subject.
